SEQUENCE LISTING (737 sequences)

SEQ ID NO: 1 - DNA, 173 bp, Artificial Sequence
  Synthetic oligonucleotide
ctttggtctcaccaaaaccaaatgaattaaggtgcaataatgttcaaatcaaagataatataagaggtgccaagagtaagcctggcacagctaagccggcggatttaacgcctcatctcaaaccttatactcaaagaggtttcaagagtaagcgttttagagagagacctttc

SEQ ID NO: 2 - DNA, 166 bp, Artificial Sequence
  Synthetic oligonucleotide
ctttggtctcaccaaaacatccaaaaattattaaacaagccacgttactttacttgaaaaaaacacttagagaagctgaagaaatgggcttgactaccacatctactatcatgagtctgcaatgtccttgaaaaaaacacttcggggttttagagagagacctttc

SEQ ID NO: 3 - DNA, 161 bp, Artificial Sequence
  Synthetic oligonucleotide
ctttggtctcaccaaaacagatgcttacaatttattgattttgaagggtatttcattcttgtgtacgatgctggacattgcagactcatgatagtagatgtggtagtcaagcccatttcttttcattcttgtgtacgaaatgttttagagagagacctttc

SEQ ID NO: 4 - DNA, 167 bp, Artificial Sequence
  Synthetic oligonucleotide
ctttggtctcaccaaaacctatatcaatttgacatactgggcattgccacgtaggaatttgtagttggtcgtgtagaaaccataatgcatcaaaacattgcagatgcttacaatttattgattttgagaatttgtagttgggagtgtgttttagagagagacctttc

SEQ ID NO: 5 - DNA, 321 bp, Artificial Sequence
  Synthetic oligonucleotide
ctttggtctcaccaaaaccctcttatattatctttgatttgaacattattgcaccttaattcatttggagctggaaattggatgggttcattacctcgggatcctaatggattaatcatcccaccttaattcatttgggaagttttagagctatgctgttttgaatggtcccaaaacggagtgatcatttctacaatgtacccaaatagcttgtattccttcgtggtagctgcatatatcagctccacattgttttgttgagtataaggtttgagatgaggcttgtattccttcgtggtgagttttagagagagacctttc

SEQ ID NO: 6 - DNA, 164 bp, Artificial Sequence
  Synthetic oligonucleotide
ctttggtctcaccaaaaccaaatgaattaaggtgcaataatgttcaaatcaaagataatataagaggtaagcctggcacagctaagccggcggatttaacgcctcatctcaaaccttatactcaaagaggtttcaagagtaagcgttttagagagagacctttc

SEQ ID NO: 7 - DNA, 182 bp, Artificial Sequence
  Synthetic oligonucleotide
ctttggtctcaccaaaaccaaatgaattaaggtgcaataatgttcaaatcaaagataatataagaggtgctgctgctttcaagagtaagcctggcacagctaagccggcggatttaacgcctcatctcaaaccttatactcaaagaggtttcaagagtaagcgttttagagagagacctttc

SEQ ID NO: 8 - DNA, 164 bp, Artificial Sequence
  Synthetic oligonucleotide
ctttggtctcaccaaaacagtggaactttgtacgtccaaaattgaatgacttggccaactacactaagagctaaggcaaaagtgattgcccaagaaaaccaatacatgtaaccattggccgcacggccaactacactaagttccgttttagagagagacctttc

SEQ ID NO: 9 - DNA, 165 bp, Artificial Sequence
  Synthetic oligonucleotide
ctttggtctcaccaaaacggtgcggccaatggttacatgtattggttttcttgggcaatcacttttgcactggctcttagtgtagttggccaagtcattcaattttggacgtacaaagttccactgccaactacactaagttccagttttagagagagacctttc

SEQ ID NO: 10 - DNA, 166 bp, Artificial Sequence
  Synthetic oligonucleotide
ctttggtctcaccaaaactggtgcggccaatggttacatgtattggttttcttgggcaatcacttttgcccttgctcttagtgtagttggccaagtcattcaattttggacgtacaaagttccactttgggcaatcacttttgcccgttttagagagagacctttc

SEQ ID NO: 11 - DNA, 168 bp, Artificial Sequence
  Synthetic oligonucleotide
ctttggtctcaccaaaaccaagatatatcatccaaatatcaatgccaatggtaacatcgctcttgacatcctaaaggatcaatggtcaccagctctaactctatcgaaggtcctattatccatctgtttgccaatggtaacatctgtcgttttagagagagacctttc

SEQ ID NO: 12 - DNA, 181 bp, Artificial Sequence
  Synthetic oligonucleotide
ctttggtctcaccaaaactctccttcacaaccaagatatatcatccaaatatcaatgctaatggtaacatcgctctggacatcctaaaggatcaatggtcaccagctctaactctatcgaaggtcctattatccatctgttcatccaaatatcaatgccaagttttagagagagacctttc

SEQ ID NO: 13 - DNA, 168 bp, Artificial Sequence
  Synthetic oligonucleotide
ctttggtctcaccaaaaccaagatatatcatccaaatatcaatgccaatggtaacatcgctctggacatcttgaaagatcaatggtcaccagctctaactctatcgaaggtcctattatccatctgttcatctgtctggacatcctaagttttagagagagacctttc

SEQ ID NO: 14 - DNA, 181 bp, Artificial Sequence
  Synthetic oligonucleotide
ctttggtctcaccaaaactctccttcacaaccaagatatatcatccaaatatcaatgcaaatggtaacatcgctctggacatcctaaaggatcaatggtcaccagctctaactctatcgaaggtcctattatccatctgttgtccagacagatgttaccatgttttagagagagacctttc

SEQ ID NO: 15 - DNA, 168 bp, Artificial Sequence
  Synthetic oligonucleotide
ctttggtctcaccaaaaccaagatatatcatccaaatatcaatgccaatggtaacatcgctctggacatactaaaggatcaatggtcaccagctctaactctatcgaaggtcctattatccatctgttctggagaccattgatcctttgttttagagagagacctttc

SEQ ID NO: 16 - DNA, 171 bp, Artificial Sequence
  Synthetic oligonucleotide
ctttgaagacgtcaccgagtacaaacggcagaagctggaggaggaagggcctgagtccgagcagaagcttaagggcagtgtagtgatcaaccggtggcgcattgccacgaagcaggccaatggggaggacatcgagagtccgagcagaagaagaagtttgggtcttctttc

SEQ ID NO: 17 - DNA, 145 bp, Artificial Sequence
  Synthetic oligonucleotide
ctttggtctcaccaaaactgtacgtccaaaattgaatgacttggccaactacactaagagctaaggcaaaagtgattgcccaagaaaaccaatacatgtaaccatggccaactacactaagttccgttttagagagagacctttc

SEQ ID NO: 18 - DNA, 146 bp, Artificial Sequence
  Synthetic oligonucleotide
ctttggtctcaccaaaactgtacgtccaaaattgaatgacttggccaactacactaagagccagtgcaaaagtgattgcccaagaaaaccaatacatgtaaccattgccaactacactaagttccagttttagagagagacctttc

SEQ ID NO: 19 - DNA, 143 bp, Artificial Sequence
  Synthetic oligonucleotide
ctttggtctcaccaaaacggttacatgtattggttttcttgggcaatcacttttgcccttgctcttagtgtagttggccaagtcattcaattttggacgtacattgggcaatcacttttgcccgttttagagagagacctttc

SEQ ID NO: 20 - DNA, 155 bp, Artificial Sequence
  Synthetic oligonucleotide
ctttggtctcaccaaaacttacatgtattggttttcttgggcaatcacttttgccctggctctttcagttgttggccaagtcattcaattttggacgtacaaagttccactggcggccctggaacttagtgtagtgttttagagagagacctttc

SEQ ID NO: 21 - DNA, 158 bp, Artificial Sequence
  Synthetic oligonucleotide
ctttggtctcaccaaaactgccgccagtggaactttgtacgtccaaaattgaatgacttgaccaactacactaagagccagggcaaaagtgattgcccaagaaaaccaatacatgtaaacgtccaaaattgaatgactgttttagagagagacctttc

SEQ ID NO: 22 - DNA, 165 bp, Artificial Sequence
  Synthetic oligonucleotide
ctttggtctcaccaaaactccagcatttggtgcggccaatggttacatgtattggtttagctgggcaatcacttttgccctggctcttagtgtagttggccaagtcattcaattttggacgtacattacatgtattggttttcttgttttagagagagacctttc

SEQ ID NO: 23 - DNA, 165 bp, Artificial Sequence
  Synthetic oligonucleotide
ctttggtctcaccaaaactccagcatttggtgcggccaatggttacatgtattggtttagctgggcaatcacttttgccctggctcttagtgtagttggccaagtcattcaattttggacgtacagttacatgtattggttttctgttttagagagagacctttc

SEQ ID NO: 24 - DNA, 174 bp, Artificial Sequence
  Synthetic oligonucleotide
ctttggtctcaccaaaacaaaaaatactaatccatgccgccagtggaactttgtacgtccagaactgaatgacttggccaactacactaagagccagggcaaaagtgattgcccaagaaaaccaatacatgtaattggccaagtcattcaatttgttttagagagagacctttc

SEQ ID NO: 25 - DNA, 172 bp, Artificial Sequence
  Synthetic oligonucleotide
ctttggtctcaccaaaactcctttctccagcatttggtgcggccaatggttacatgtactggttttcttgggcaatcacttttgccctggctcttagtgtagttggccaagtcattcaattttggacgtacacggccaatggttacatgtatgttttagagagagacctttc

SEQ ID NO: 26 - DNA, 188 bp, Artificial Sequence
  Synthetic oligonucleotide
ctttggtctcaccaaaactgtacgtccaaaattgaatgacttggccaactacactaagagccagggcaaaagtgattgcccaagaaaaccaatacatgtaaccatttgccgcaccaaatgctggagaaaggaatctttgtgagaaaacaaaccaatacatgtaaccatgttttagagagagacctttc

SEQ ID NO: 27 - DNA, 142 bp, Artificial Sequence
  Synthetic oligonucleotide
ctttggtctcaccaaaaccattcgtcttgaagtcgaggactttggcatacgatggaagataaaacttcgttgtaaagaataaggaaatgattccggaagcttactttggcatacgatggaaggttttagagagagacctttc

SEQ ID NO: 28 - DNA, 151 bp, Artificial Sequence
  Synthetic oligonucleotide
ctttggtctcaccaaaactgggttttccattcgtcttgaagtcgaggactttggcatatgatggaagataaaacttcgttgtaaagaataaggaaatgattccggaagctttcgaggactttggcatacgagttttagagagagacctttc

SEQ ID NO: 29 - DNA, 159 bp, Artificial Sequence
  Synthetic oligonucleotide
ctttggtctcaccaaaacaagagatttgggttttccattcgtcttgaagtcgaggactcttgcatacgatggaagataaaacttcgttgtaaagaataaggaaatgattccggaagcttcgtcttgaagtcgaggacttgttttagagagagacctttc

SEQ ID NO: 30 - DNA, 163 bp, Artificial Sequence
  Synthetic oligonucleotide
ctttggtctcaccaaaacattcgtcttgaagtcgaggactttggcatacgatggaagataaaacttcgttgtaaagaacaaagaaatgattccggaagctttggaagtactgaaggatcgtcctaacttcgttgtaaagaatagttttagagagagacctttc

SEQ ID NO: 31 - DNA, 165 bp, Artificial Sequence
  Synthetic oligonucleotide
ctttggtctcaccaaaactgttggaagagatttgggttttccattcgtcttgaagtcgagaactttggcatacgatggaagataaaacttcgttgtaaagaataaggaaatgattccggaagcttttccattcgtcttgaagtcggttttagagagagacctttc

SEQ ID NO: 32 - DNA, 177 bp, Artificial Sequence
  Synthetic oligonucleotide
ctttggtctcaccaaaactttcggcgtacaaaggacgatccttcagtacttccaaagcctccggaatcatttccttattctttacaacgaagttttatcttccatcgtatgccaaagtcctcgacttcaagacgaatttcagtacttccaaagcttcgttttagagagagacctttc

SEQ ID NO: 33 - DNA, 175 bp, Artificial Sequence
  Synthetic oligonucleotide
ctttggtctcaccaaaacattcgtcttgaagtcgaggactttggcatacgatggaagataaaacttcgttgtaaagaataaggaaatgattcctgaagctttggaagtactgaaggatcgtcctttgtacgccgaaaagaataaggaaatgattcgttttagagagagacctttc

SEQ ID NO: 34 - DNA, 184 bp, Artificial Sequence
  Synthetic oligonucleotide
ctttggtctcaccaaaacattcgtcttgaagtcgaggactttggcatacgatggaagataaaacttcgttgtaaagaataaggaaatgattccggaagctcttgaagtactgaaggatcgtcctttgtacgccgaaaaatgggcggaaatgattccggaagcttgttttagagagagacctttc

SEQ ID NO: 35 - DNA, 183 bp, Artificial Sequence
  Synthetic oligonucleotide
ctttggtctcaccaaaacaagcttccggaatcatttccttattctttacaacgaagttttatcttccatcgtatgccaaagtcctcgacttcaagacaaatggaaaacccaaatctcttccaacattcaatagggacgtctcagtcctcgacttcaagacgaagttttagagagagacctttc

SEQ ID NO: 36 - DNA, 192 bp, Artificial Sequence
  Synthetic oligonucleotide
ctttggtctcaccaaaacacaagccagtgagacgtccctattgaatgttggaagagatctaggttttccattcgtcttgaagtcgaggactttggcatacgatggaagataaaacttcgttgtaaagaataaggaaatgattccggaagcttttgaatgttggaagagatttgttttagagagagacctttc

SEQ ID NO: 37 - DNA, 141 bp, Artificial Sequence
  Synthetic oligonucleotide
ctttggtctcaccaaaacgtttatccccgtgacatcatctatcactgtcttttcgaagtaattcttatcacctgcattcggtgtttctaacggctacatgtctatcactgtcttttcgaaggttttagagagagacctttc

SEQ ID NO: 38 - DNA, 156 bp, Artificial Sequence
  Synthetic oligonucleotide
ctttggtctcaccaaaaccccaattgaaccagtacatgtagccgttagaaacaccgaaagcaggtgataagaattacttcgaaaagacagtgatagatgatgtcacggggataaacccgttagaaacaccgaatgcgttttagagagagacctttc

SEQ ID NO: 39 - DNA, 161 bp, Artificial Sequence
  Synthetic oligonucleotide
ctttggtctcaccaaaacgtttatccccgtgacatcatctatcactgtcttttcgaagtaattcttatcacctgctttcggtgtttctaacggctacatgtactggttcaattgggctattaggttcttatcacctgcattgttttagagagagacctttc

SEQ ID NO: 40 - DNA, 173 bp, Artificial Sequence
  Synthetic oligonucleotide
ctttggtctcaccaaaacgtttatccccgtgacatcatctatcactgtcttttcgaagtaattcttatcacctgcattcggtgttagcaacggctacatgtactggttcaattgggctattacttatgctgtgcctgcattcggtgtttctaagttttagagagagacctttc

SEQ ID NO: 41 - DNA, 186 bp, Artificial Sequence
  Synthetic oligonucleotide
ctttggtctcaccaaaacgtttatccccgtgacatcatctatcactgtcttttcgaagtaattcttatcacctgcattcggtgtttctaacggctacatgtattggttcaattgggctattacttatgctgtggaggtttctgtcatttctaacggctacatgtacgttttagagagagacctttc

SEQ ID NO: 42 - DNA, 173 bp, Artificial Sequence
  Synthetic oligonucleotide
ctttggtctcaccaaaacacatgtagccgttagaaacaccgaatgcaggtgataagaattacttcgaaaagacagtgatagatgatgtcactgggataaacgtagccatctcaccaagtgactgggtaacgaagacagtgatagatgatgtcagttttagagagagacctttc

SEQ ID NO: 43 - DNA, 174 bp, Artificial Sequence
  Synthetic oligonucleotide
ctttggtctcaccaaaacacatgtagccgttagaaacaccgaatgcaggtgataagaattacttcgaaaagacagtgatagatgatgtaacggggataaacgtagccatctcaccaagtgactgggtaacgaagacagtgatagatgatgtcacgttttagagagagacctttc

SEQ ID NO: 44 - DNA, 175 bp, Artificial Sequence
  Synthetic oligonucleotide
ctttggtctcaccaaaacacatgtagccgttagaaacaccgaatgcaggtgataagaattacttcgaaaagacagtgatagatgatgtcacgggaataaacgtagccatctcaccaagtgactgggtaacgaagtcagtgatagatgatgtcacggttttagagagagacctttc

SEQ ID NO: 45 - DNA, 190 bp, Artificial Sequence
  Synthetic oligonucleotide
ctttggtctcaccaaaactgggcaccattgtctacttcgttacccagtcacttggtgaaatggctacgtttatccccgtgacatcatctatcactgtcttttcgaagtaattcttatcacctgcattcggtgtttctaacggctacatgttacccagtcacttggtgagagttttagagagagacctttc

SEQ ID NO: 46 - DNA, 195 bp, Artificial Sequence
  Synthetic oligonucleotide
ctttggtctcaccaaaacgtttatccccgtgacatcatctatcactgtcttttcgaagtaattcttatcacctgcattcggtgtttctaacggctacatgtactggttcaactgggctattacttatgctgtggaggtttctgtcattggccaaggctacatgtactggttcaatgttttagagagagacctttc

SEQ ID NO: 47 - DNA, 158 bp, Artificial Sequence
  Synthetic oligonucleotide
ctttggtctcaccaaaacgaaacccaggtgcctggggtccaggtataatatctaaggataaaaacgaacttaggttgggtttcctctttgattaacgctgccttcacatttcaaggtactaaggataaaaacgaaggggttttagagagagacctttc

SEQ ID NO: 48 - DNA, 158 bp, Artificial Sequence
  Synthetic oligonucleotide
ctttggtctcaccaaaacctggggtccaggtataatatctaaggataaaaacgaagggaggttcttagtcctctttgattaacgctgccttcacatttcaaggtactgaactagttggcgaagggaggttcttaggttgttttagagagagacctttc

SEQ ID NO: 49 - DNA, 158 bp, Artificial Sequence
  Synthetic oligonucleotide
ctttggtctcaccaaaacgggaggttcttaggttgggtttcctctttgattaacgctgccttcacattctgaactagttggtatcactgctggtgaagctgcaaaccccagaaaatccaacgctgccttcacatttcagttttagagagagacctttc

SEQ ID NO: 50 - DNA, 158 bp, Artificial Sequence
  Synthetic oligonucleotide
ctttggtctcaccaaaacaccttgaataatgataatgatcgtcataaatgtggccgcataataagccaattaatttagctttaaatggtaactcgtcacgagagatgccacggtatttggccgcataataagccaagcgttttagagagagacctttc

SEQ ID NO: 51 - DNA, 158 bp, Artificial Sequence
  Synthetic oligonucleotide
ctttggtctcaccaaaacatgacgatcattatcattattcaaggtttcacggcttttgcaccaaaattttagctttgctgccgcctatatctctattttcctgttcttagctgtttgggcttttgcaccaaaattcaagttttagagagagacctttc

SEQ ID NO: 52 - DNA, 158 bp, Artificial Sequence
  Synthetic oligonucleotide
ctttggtctcaccaaaacatggtgttagctttgctgccgcctatatctctattttcctgttcttagctcttatttcaatgcatattcagatgcagatttatttggaagattggagatgttttcctgttcttagctgttgttttagagagagacctttc

SEQ ID NO: 53 - DNA, 157 bp, Artificial Sequence
  Synthetic oligonucleotide
ctttggtctcaccaaaacgtaaatggcgaggatacgttctctatggaggatggcataggtgatgaagaaagtacagaacgctgaagtgaagagagagcttaagcaaagacatattggtggcataggtgatgaagatgagttttagagagagaccttt

SEQ ID NO: 54 - DNA, 158 bp, Artificial Sequence
  Synthetic oligonucleotide
ctttggtctcaccaaaacttttggtgcaaaagccgtgaaaccttgaataatgataatgatcgtcataagcataataagccaagccgggcattaatttagctttaaatggtaactcgtcgataatgatcgtcataaatggttttagagagagacctttc

SEQ ID NO: 55 - DNA, 158 bp, Artificial Sequence
  Synthetic oligonucleotide
ctttggtctcaccaaaactcgtgacgagttaccatttaaagctaaattaatgcccggcttggcttattacatttatgacgatcattatcattattcaaggtttcacggcttttgcaccgcccggcttggcttattatggttttagagagagacctttc

SEQ ID NO: 56 - DNA, 158 bp, Artificial Sequence
  Synthetic oligonucleotide
ctttggtctcaccaaaacacacctctgaccaacgccggcccagtgggcgctcttatatcatatttattctttggcatattctgtcacgcagtccttgggtgaaatggctacattcatccttatatcatatttatttatgttttagagagagacctttc

SEQ ID NO: 57 - DNA, 158 bp, Artificial Sequence
  Synthetic oligonucleotide
ctttggtctcaccaaaacgatttgggttttccattcgtcttgaagtcgaggactttggcatacgatggacttcgttgtaaagaataaggaaatgattccggaagctttggaagtactgactttggcatacgatggaaggttttagagagagacctttc

SEQ ID NO: 58 - DNA, 158 bp, Artificial Sequence
  Synthetic oligonucleotide
ctttggtctcaccaaaacttttgtatgtttgtctccaagaacatttagcataatggcgttcgttgtaaaaagatgtgaaattctttggcattggcaaatccaatattgatctcaaatgaatggcgttcgttgtaatgggttttagagagagacctttc

SEQ ID NO: 59 - DNA, 158 bp, Artificial Sequence
  Synthetic oligonucleotide
ctttggtctcaccaaaacaatatcagttctacctgtaatgtagttcagcctttgttcacattccgccagcaataatatttatgtgacctacttttctgttaggtctagactcttttccttgttcacattccgccatacgttttagagagagacctttc

SEQ ID NO: 60 - DNA, 158 bp, Artificial Sequence
  Synthetic oligonucleotide
ctttggtctcaccaaaacaatttcacatctttctccaccattacaacgaacgccattatgctaaatgtacaaacatacaaaagataaagagctagaaacttgcgaaagagcattggcggccattatgctaaatgttctgttttagagagagacctttc

SEQ ID NO: 61 - DNA, 158 bp, Artificial Sequence; identical to SEQ ID NO: 60
  Synthetic oligonucleotide

SEQ ID NO: 62 - DNA, 158 bp, Artificial Sequence
  Synthetic oligonucleotide
ctttggtctcaccaaaacccttttacgggcacaccgatgacaggaagtggtgtcattgcagccaccatagtgagcagccccaccagctccagcgataattgttttaattccacgcttggtcattgcagccaccataccgttttagagagagacctttc

SEQ ID NO: 63 - DNA, 158 bp, Artificial Sequence
  Synthetic oligonucleotide
ctttggtctcaccaaaacacatttagcataatggcgttcgttgtaatggtggagaaagatgtgaaattttggcaaatccaatattgatctcaaatgagcttcaaattgagaagtgacggagaaagatgtgaaattcttgttttagagagagacctttc

SEQ ID NO: 64 - DNA, 158 bp, Artificial Sequence
  Synthetic oligonucleotide
ctttggtctcaccaaaacgccaagcagtctgacagccaacagcgcagcgttcgtactattattaataggctactggaacacctctaggcatttgcacaattgaatgtaaagaatctaccgtactattattaatagcgagttttagagagagacctttc

SEQ ID NO: 65 - DNA, 158 bp, Artificial Sequence
  Synthetic oligonucleotide
ctttggtctcaccaaaacaaaatctctgtcgctcaaaagttggacttggaagcaatggtcaaaccatttcatcatgggatcagactctgacttgccggtaatgtctgccgcatgtgcggcaatggtcaaaccattggtgttttagagagagacctttc

SEQ ID NO: 66 - DNA, 158 bp, Artificial Sequence
  Synthetic oligonucleotide
ctttggtctcaccaaaacagcgcagcgttcgtactattattaatagcgacggtagctactggaacacctttgcacaattgaatgtaaagaatctactccatctagacaagaaccttttgtagctactggaacacctctgttttagagagagacctttc

SEQ ID NO: 67 - DNA, 158 bp, Artificial Sequence
  Synthetic oligonucleotide
ctttggtctcaccaaaacgtgagatggctacgtttatccccgtgacatcatctatcactgtcttttcgcttatcacctgcattcggtgtttctaacggctacatgtactggttcaattctatcactgtcttttcgaaggttttagagagagacctttc

SEQ ID NO: 68 - DNA, 158 bp, Artificial Sequence
  Synthetic oligonucleotide
ctttggtctcaccaaaacccatccgagaaaacggccttcacttttatcactggagatgatgcctggcccctggatttctccagtacctgaaaccgatagggccctggtgggatccaccggagatgatgcctggcccccgttttagagagagacctttc

SEQ ID NO: 69 - DNA, 158 bp, Artificial Sequence
  Synthetic oligonucleotide
ctttggtctcaccaaaacgtcgtcttattacttggatctattgcttccatctcatgttctatctggtcattcctgcatgctctgttcgccaatgttgttttgtttctcgtcccatttatcatgttctatctggtcttcgttttagagagagacctttc

SEQ ID NO: 70 - DNA, 158 bp, Artificial Sequence
  Synthetic oligonucleotide
ctttggtctcaccaaaacaatagtacgattctaaagacgactttattgatagctcttggaacggtctttagccgcttcaccagcggtgatcccaaccagttcagtaccttggtacgtagctcttggaacggtctttctgttttagagagagacctttc

SEQ ID NO: 71 - DNA, 158 bp, Artificial Sequence
  Synthetic oligonucleotide
ctttggtctcaccaaaacacggtgctttaaagcttgcatgaacctaatatgtgccaaagagatgaatacataacccagccaaagtggaaatgttgatcaaccagttaaatgcagtgtttgccaaagagatgaataaccgttttagagagagacctttc

SEQ ID NO: 72 - DNA, 158 bp, Artificial Sequence
  Synthetic oligonucleotide
ctttggtctcaccaaaacgttaaagttttagccattatgggttacttgatatatgctttgattattgtgatcccaccagggccctatcggtttcaggtactggagaaatccaggagcctatgctttgattattgtctggttttagagagagacctttc

SEQ ID NO: 73 - DNA, 158 bp, Artificial Sequence
  Synthetic oligonucleotide
ctttggtctcaccaaaaccatgaaaatgtaagcaatcagggaccccacagggccagcattactcaaggataccaacgaaaagaccagtaccgattgtaccacctagtgcaatcataccgccagcattactcaagggaggttttagagagagacctttc

SEQ ID NO: 74 - DNA, 158 bp, Artificial Sequence
  Synthetic oligonucleotide
ctttggtctcaccaaaacgtggatcccaccagggccctatcggtttcaggtactggagaaatccaggagccaggcatcatctccagtgataaaagtgaaggccgttttctcggatgggactggagaaatccaggagccgttttagagagagacctttc

SEQ ID NO: 75 - DNA, 158 bp, Artificial Sequence
  Synthetic oligonucleotide
ctttggtctcaccaaaaccataatatagaatagtacgattctaaagacgactttattgatagctcttgtttcttgggttagccgcttcaccagcggtgatcccaaccagttcagtacctttattgatagctcttggaagttttagagagagacctttc

SEQ ID NO: 76 - DNA, 158 bp, Artificial Sequence
  Synthetic oligonucleotide
ctttggtctcaccaaaacagctagaagatattgacatcgattccgacagaagagaaatcgaagcaattagacgacgagcctaagaatttatgggagaaattctgggctgctgttgcatgagaaatcgaagcaattattgttttagagagagacctttc

SEQ ID NO: 77 - DNA, 19 bp, Artificial Sequence
  Synthetic oligonucleotide
tatctacacgggtctcacc

SEQ ID NO: 78 - DNA, 19 bp, Artificial Sequence
  Synthetic oligonucleotide
gagttacgctggtctctct

SEQ ID NO: 79 - DNA, 35 bp, Artificial Sequence
  Synthetic oligonucleotide
gtctcgtgggctcggagtgaaagataaatgatcgg

SEQ ID NO: 80 - DNA, 33 bp, Artificial Sequence
  Synthetic oligonucleotide
tcgtcggcagcgtcattttgaagctatgcagac

SEQ ID NO: 81 - DNA, 33 bp, Artificial Sequence
  Synthetic oligonucleotide
aagaagcgattatgatctctcctctagaaactc

SEQ ID NO: 82 - DNA, 21 bp, Artificial Sequence
  Synthetic oligonucleotide
gccaccggttgatcactacac

SEQ ID NO: 83 - DNA, 30 bp, Artificial Sequence
  Synthetic oligonucleotide
tacttgaaaaaaacacttcgggaggatgaa

SEQ ID NO: 84 - protein, 10 aa, Artificial Sequence
  Synthetic peptide
YLKKTLREDE

SEQ ID NO: 85 - DNA, 30 bp, Artificial Sequence
  Synthetic oligonucleotide
tacttgaaaaaaacacttagagaagctgaa

SEQ ID NO: 86 - protein, 10 aa, Artificial Sequence
  Synthetic peptide
YLKKTLREAE

SEQ ID NO: 87 - DNA, 170 bp, Artificial Sequence
  Synthetic oligonucleotide
  misc_feature (25)..(144): n is a, c, g, or t
tatctacacgggtctcaccaaaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttttagagagagaccagcgtaactc

SEQ ID NO: 88 - DNA, 30 bp, Artificial Sequence
  Synthetic oligonucleotide
ataagaggtttcaagagtaagccgggcaca

SEQ ID NO: 89 - protein, 10 aa, Artificial Sequence
  Synthetic peptide
IRGFKSKPGT

SEQ ID NO: 90 - DNA, 30 bp, Artificial Sequence
  Synthetic oligonucleotide
ataagaggtgccaagagtaagcctggcaca

SEQ ID NO: 91 - protein, 10 aa, Artificial Sequence
  Synthetic peptide
IRGAKSKPGT

SEQ ID NO: 92 - DNA, 30 bp, Artificial Sequence
  Synthetic oligonucleotide
caatgtccaatttcgtacacaagaatgaaa

SEQ ID NO: 93 - protein, 10 aa, Artificial Sequence
  Synthetic peptide
QCPISYTRMK

SEQ ID NO: 94 - DNA, 30 bp, Artificial Sequence
  Synthetic oligonucleotide
caatgtccagcatcgtacacaagaatgaaa

SEQ ID NO: 95 - protein, 10 aa, Artificial Sequence
  Synthetic peptide
QCPASYTRMK

SEQ ID NO: 96 - DNA, 30 bp, Artificial Sequence
  Synthetic oligonucleotide
tggttcctacactcccaactacaaattcct

SEQ ID NO: 97 - protein, 10 aa, Artificial Sequence
  Synthetic peptide
WFLHSQLQIP

SEQ ID NO: 98 - DNA, 30 bp, Artificial Sequence
  Synthetic oligonucleotide
tggtttctacacgaccaactacaaattcct

SEQ ID NO: 99 - protein, 10 aa, Artificial Sequence
  Synthetic peptide
WFLHDQLQIP

SEQ ID NO: 100 - DNA, 30 bp, Artificial Sequence
  Synthetic oligonucleotide
caatttccattcccaaatgaattaaggtgc

SEQ ID NO: 101 - protein, 10 aa, Artificial Sequence
  Synthetic peptide
QFPFPNELRC

SEQ ID NO: 102 - DNA, 30 bp, Artificial Sequence
  Synthetic oligonucleotide
caatttccagctccaaatgaattaaggtgc

SEQ ID NO: 103 - protein, 10 aa, Artificial Sequence
  Synthetic peptide
QFPAPNELRC

SEQ ID NO: 104 - DNA, 30 bp, Artificial Sequence
  Synthetic oligonucleotide
tatgccttcaccacgaaggaatacaagcta

SEQ ID NO: 105 - protein, 10 aa, Artificial Sequence
  Synthetic peptide
YAFTTKEYKL

SEQ ID NO: 106 - DNA, 30 bp, Artificial Sequence
  Synthetic oligonucleotide
tatgcagctaccacgaaggaatacaagcta

SEQ ID NO: 107 - protein, 10 aa, Artificial Sequence
  Synthetic peptide
YAATTKEYKL

SEQ ID NO: 108 - DNA, 30 bp, Artificial Sequence; identical to SEQ ID NO: 88
  Synthetic oligonucleotide

SEQ ID NO: 109 - protein, 10 aa, Artificial Sequence
  Synthetic peptide
IRGFKSKPGT

SEQ ID NO: 110 - DNA, 21 bp, Artificial Sequence
  Synthetic oligonucleotide
ataagaggtaagcctggcaca

SEQ ID NO: 111 - protein, 7 aa, Artificial Sequence
  Synthetic peptide
IRGKPGT

SEQ ID NO: 112 - DNA, 30 bp, Artificial Sequence; identical to SEQ ID NO: 88
  Synthetic oligonucleotide

SEQ ID NO: 113 - protein, 10 aa, Artificial Sequence
  Synthetic peptide
IRGFKSKPGT

SEQ ID NO: 114 - DNA, 39 bp, Artificial Sequence
  Synthetic oligonucleotide
ataagaggtgctgctgctttcaagagtaagcctggcaca

SEQ ID NO: 115 - protein, 13 aa, Artificial Sequence
  Synthetic peptide
IRGAAAFKSKPGT

SEQ ID NO: 116 - DNA, 30 bp, Artificial Sequence
  Synthetic oligonucleotide
tttgccctggaacttagtgtagttggccaa

SEQ ID NO: 117 - protein, 10 aa, Artificial Sequence
  Synthetic peptide
FALELSVVGQ

SEQ ID NO: 118 - DNA, 30 bp, Artificial Sequence
  Synthetic oligonucleotide
tttgccttagctcttagtgtagttggccaa

SEQ ID NO: 119 - protein, 10 aa, Artificial Sequence
  Synthetic peptide
FALALSVVGQ

SEQ ID NO: 120 - DNA, 30 bp, Artificial Sequence; identical to SEQ ID NO: 116
  Synthetic oligonucleotide

SEQ ID NO: 121 - DNA, 30 bp, Artificial Sequence
  Synthetic oligonucleotide
tttgcactggctcttagtgtagttggccaa

SEQ ID NO: 122 - DNA, 30 bp, Artificial Sequence
  Synthetic oligonucleotide
tcttgggcaatcacttttgccctggaactt

SEQ ID NO: 123 - protein, 10 aa, Artificial Sequence
  Synthetic peptide
SWAITFALEL

SEQ ID NO: 124 - DNA, 30 bp, Artificial Sequence
  Synthetic oligonucleotide
tcttgggcaatcacttttgcccttgctctt

SEQ ID NO: 125 - protein, 10 aa, Artificial Sequence
  Synthetic peptide
SWAITFALAL

SEQ ID NO: 126 - DNA, 51 bp, Artificial Sequence
  Synthetic oligonucleotide
aatgccaatggtaacatctgtctggacatcctaaaggatcaatggtctcca

SEQ ID NO: 127 - protein, 17 aa, Artificial Sequence
  Synthetic peptide
NANGNICLDILKDQWSP

SEQ ID NO: 128 - DNA, 51 bp, Artificial Sequence
  Synthetic oligonucleotide
aatgccaatggtaacatcgctcttgacatcctaaaggatcaatggtcacca

SEQ ID NO: 129 - protein, 17 aa, Artificial Sequence
  Synthetic peptide
NANGNIALDILKDQWSP

SEQ ID NO: 130 - DNA, 51 bp, Artificial Sequence
  Synthetic oligonucleotide
aatgccaatggtaacatcgctcttgacatcctaaaggatcaatggtctcca

SEQ ID NO: 131 - DNA, 66 bp, Artificial Sequence
  Synthetic oligonucleotide
tatcatccaaatatcaatgccaatggtaacatctgtctggacatcctaaaggatcaatggtctcca

SEQ ID NO: 132 - protein, 22 aa, Artificial Sequence
  Synthetic peptide
YHPNINANGNICLDILKDQWSP

SEQ ID NO: 133 - DNA, 66 bp, Artificial Sequence
  Synthetic oligonucleotide
tatcatccaaatatcaatgctaatggtaacatcgctctggacatcctaaaggatcaatggtcacca

SEQ ID NO: 134 - protein, 22 aa, Artificial Sequence
  Synthetic peptide
YHPNINANGNIALDILKDQWSP

SEQ ID NO: 135 - DNA, 66 bp, Artificial Sequence
  Synthetic oligonucleotide
tatcatccaaatatcaatgctaatggtaacatcgctctggacatcctaaaggatcaatggtctcca

SEQ ID NO: 136 - DNA, 51 bp, Artificial Sequence; identical to SEQ ID NO: 126
  Synthetic oligonucleotide

SEQ ID NO: 137 - protein, 17 aa, Artificial Sequence
  Synthetic peptide
NANGNICLDILKDQWSP

SEQ ID NO: 138 - DNA, 51 bp, Artificial Sequence
  Synthetic oligonucleotide
aatgccaatggtaacatcgctctggacatcttgaaagatcaatggtcacca

SEQ ID NO: 139 - protein, 17 aa, Artificial Sequence
  Synthetic peptide
NANGNIALDILKDQWSP

SEQ ID NO: 140 - DNA, 51 bp, Artificial Sequence
  Synthetic oligonucleotide
aatgccaatggtaacatcgctctggacatcttgaaagatcaatggtctcca

SEQ ID NO: 141 - DNA, 51 bp, Artificial Sequence; identical to SEQ ID NO: 126
  Synthetic oligonucleotide

SEQ ID NO: 142 - protein, 16 aa, Artificial Sequence
  Synthetic peptide
NANGNICLDILKDQSP

SEQ ID NO: 143 - DNA, 51 bp, Artificial Sequence
  Synthetic oligonucleotide
aatgcaaatggtaacatcgctctggacatcctaaaggatcaatggtcacca

SEQ ID NO: 144 - protein, 17 aa, Artificial Sequence
  Synthetic peptide
NANGNIALDILKDQWSP

SEQ ID NO: 145 - DNA, 51 bp, Artificial Sequence
  Synthetic oligonucleotide
aatgcaaatggtaacatcgctctggacatcctaaaggatcaatggtctcca

SEQ ID NO: 146 - DNA, 51 bp, Artificial Sequence
  Synthetic oligonucleotide
gccaatggtaacatctgtctggacatcctaaaggatcaatggtctccagct

SEQ ID NO: 147 - protein, 17 aa, Artificial Sequence
  Synthetic peptide
ANGNICLDILKDQWSPA

SEQ ID NO: 148 - DNA, 51 bp, Artificial Sequence
  Synthetic oligonucleotide
gccaatggtaacatcgctctggacatactaaaggatcaatggtcaccagct

SEQ ID NO: 149 - protein, 17 aa, Artificial Sequence
  Synthetic peptide
ANGNIALDILKDQWSPA

SEQ ID NO: 150 - DNA, 40 bp, Artificial Sequence
  Synthetic oligonucleotide
ctgagtccgagcagaagaagaagggctcccatcacatcaa

SEQ ID NO: 151 - DNA, 40 bp, Artificial Sequence
  Synthetic oligonucleotide
ctgagtccgagcagaagcttaagggcagtgtagtgatcaa

SEQ ID NO: 152 - DNA, 170 bp, Artificial Sequence
  Synthetic oligonucleotide
tatctacacgggtctcaccaaaacggagcaactcctggaaaaagtattacagcatccaaaaattgctaaacaagcgaccttactttacttgaaaaaaacacttcgggaggatgaagaaattttcaagtaaagtaacggttttagagtgagaccagcgtaactcgacgtgt

SEQ ID NO: 153 - DNA, 170 bp, Artificial Sequence
  Synthetic oligonucleotide
tatctacacgggtctcaccaaaacggagcaactcctggaaaaagtattacagcatccaaaaattagaaaacaagcgaccttactttacttgaaaaaaacacttcgggaggatgaagaaattttcaagtaaagtaacggttttagagtgagaccagcgtaactcttggtta

SEQ ID NO: 154 - DNA, 170 bp, Artificial Sequence
  Synthetic oligonucleotide
tatctacacgggtctcaccaaaacggagcaactcctggaaaaagtattacagcatccaaaaattaataaacaagcgaccttactttacttgaaaaaaacacttcgggaggatgaagaaattttcaagtaaagtaacggttttagagtgagaccagcgtaactcgggtgta

SEQ ID NO: 155 - DNA, 170 bp, Artificial Sequence
  Synthetic oligonucleotide
tatctacacgggtctcaccaaaacggagcaactcctggaaaaagtattacagcatccaaaaattgataaacaagcgaccttactttacttgaaaaaaacacttcgggaggatgaagaaattttcaagtaaagtaacggttttagagtgagaccagcgtaactccacaatg

SEQ ID NO: 156 - DNA, 170 bp, Artificial Sequence
  Synthetic oligonucleotide
tatctacacgggtctcaccaaaacggagcaactcctggaaaaagtattacagcatccaaaaatttgtaaacaagcgaccttactttacttgaaaaaaacacttcgggaggatgaagaaattttcaagtaaagtaacggttttagagtgagaccagcgtaactccgtcgct

SEQ ID NO: 157 - DNA, 170 bp, Artificial Sequence
  Synthetic oligonucleotide
tatctacacgggtctcaccaaaacggagcaactcctggaaaaagtattacagcatccaaaaattcaaaaacaagcgaccttactttacttgaaaaaaacacttcgggaggatgaagaaattttcaagtaaagtaacggttttagagtgagaccagcgtaactcctcgggg

SEQ ID NO: 158 - DNA, 170 bp, Artificial Sequence
  Synthetic oligonucleotide
tatctacacgggtctcaccaaaacggagcaactcctggaaaaagtattacagcatccaaaaattgaaaaacaagcgaccttactttacttgaaaaaaacacttcgggaggatgaagaaattttcaagtaaagtaacggttttagagtgagaccagcgtaactctggctgc

SEQ ID NO: 159 - DNA, 170 bp, Artificial Sequence
  Synthetic oligonucleotide
tatctacacgggtctcaccaaaacggagcaactcctggaaaaagtattacagcatccaaaaattggtaaacaagcgaccttactttacttgaaaaaaacacttcgggaggatgaagaaattttcaagtaaagtaacggttttagagtgagaccagcgtaactcactcctg

SEQ ID NO: 160 - DNA, 170 bp, Artificial Sequence
  Synthetic oligonucleotide
tatctacacgggtctcaccaaaacggagcaactcctggaaaaagtattacagcatccaaaaattcataaacaagcgaccttactttacttgaaaaaaacacttcgggaggatgaagaaattttcaagtaaagtaacggttttagagtgagaccagcgtaactcagaggac

SEQ ID NO: 161 - DNA, 170 bp, Artificial Sequence
  Synthetic oligonucleotide
tatctacacgggtctcaccaaaacggagcaactcctggaaaaagtattacagcatccaaaaattattaaacaagcgaccttactttacttgaaaaaaacacttcgggaggatgaagaaattttcaagtaaagtaacggttttagagtgagaccagcgtaactcacattgg

SEQ ID NO: 162 - DNA, 170 bp, Artificial Sequence
  Synthetic oligonucleotide
tatctacacgggtctcaccaaaacggagcaactcctggaaaaagtattacagcatccaaaaattttgaaacaagcgaccttactttacttgaaaaaaacacttcgggaggatgaagaaattttcaagtaaagtaacggttttagagtgagaccagcgtaactccatccta

SEQ ID NO: 163 - DNA, 170 bp, Artificial Sequence
  Synthetic oligonucleotide
tatctacacgggtctcaccaaaacggagcaactcctggaaaaagtattacagcatccaaaaattaaaaaacaagcgaccttactttacttgaaaaaaacacttcgggaggatgaagaaattttcaagtaaagtaacggttttagagtgagaccagcgtaactcgttaaat

SEQ ID NO: 164 - DNA, 170 bp, Artificial Sequence
  Synthetic oligonucleotide
tatctacacgggtctcaccaaaacggagcaactcctggaaaaagtattacagcatccaaaaattatgaaacaagcgaccttactttacttgaaaaaaacacttcgggaggatgaagaaattttcaagtaaagtaacggttttagagtgagaccagcgtaactccttataa

SEQ ID NO: 165 - DNA, 170 bp, Artificial Sequence
  Synthetic oligonucleotide
tatctacacgggtctcaccaaaacggagcaactcctggaaaaagtattacagcatccaaaaattttcaaacaagcgaccttactttacttgaaaaaaacacttcgggaggatgaagaaattttcaagtaaagtaacggttttagagtgagaccagcgtaactcagtgaca

SEQ ID NO: 166 - DNA, 170 bp, Artificial Sequence
  Synthetic oligonucleotide
tatctacacgggtctcaccaaaacggagcaactcctggaaaaagtattacagcatccaaaaattccaaaacaagcgaccttactttacttgaaaaaaacacttcgggaggatgaagaaattttcaagtaaagtaacggttttagagtgagaccagcgtaactctagtccc

SEQ ID NO: 167 - DNA, 170 bp, Artificial Sequence
  Synthetic oligonucleotide
tatctacacgggtctcaccaaaacggagcaactcctggaaaaagtattacagcatccaaaaatttctaaacaagcgaccttactttacttgaaaaaaacacttcgggaggatgaagaaattttcaagtaaagtaacggttttagagtgagaccagcgtaactcgtttcta

SEQ ID NO: 168 - DNA, 170 bp, Artificial Sequence
  Synthetic oligonucleotide
tatctacacgggtctcaccaaaacggagcaactcctggaaaaagtattacagcatccaaaaattactaaacaagcgaccttactttacttgaaaaaaacacttcgggaggatgaagaaattttcaagtaaagtaacggttttagagtgagaccagcgtaactcggatccg

SEQ ID NO: 169 - DNA, 170 bp, Artificial Sequence
  Synthetic oligonucleotide
tatctacacgggtctcaccaaaacggagcaactcctggaaaaagtattacagcatccaaaaatttggaaacaagcgaccttactttacttgaaaaaaacacttcgggaggatgaagaaattttcaagtaaagtaacggttttagagtgagaccagcgtaactctccgcct

SEQ ID NO: 170 - DNA, 170 bp, Artificial Sequence
  Synthetic oligonucleotide
tatctacacgggtctcaccaaaacggagcaactcctggaaaaagtattacagcatccaaaaatttataaacaagcgaccttactttacttgaaaaaaacacttcgggaggatgaagaaattttcaagtaaagtaacggttttagagtgagaccagcgtaactcgattctg

SEQ ID NO: 171 - DNA, 170 bp, Artificial Sequence
  Synthetic oligonucleotide
tatctacacgggtctcaccaaaacggagcaactcctggaaaaagtattacagcatccaaaaattgttaaacaagcgaccttactttacttgaaaaaaacacttcgggaggatgaagaaattttcaagtaaagtaacggttttagagtgagaccagcgtaactccacgcca

SEQ ID NO: 172 - DNA, 170 bp, Artificial Sequence
  Synthetic oligonucleotide
tatctacacgggtctcaccaaaacgcaactcctggaaaaagtattacagcatccaaaaattattgctcaagcgaccttactttacttgaaaaaaacacttcgggaggatgaagaaattttcaagtaaagtaacggttttagagtgagaccagcgtaactcccattatcaa

SEQ ID NO: 173 - DNA, 170 bp, Artificial Sequence
  Synthetic oligonucleotide
tatctacacgggtctcaccaaaacgcaactcctggaaaaagtattacagcatccaaaaattattagacaagcgaccttactttacttgaaaaaaacacttcgggaggatgaagaaattttcaagtaaagtaacggttttagagtgagaccagcgtaactcattcgcaaag

SEQ ID NO: 174 - DNA, 170 bp, Artificial Sequence
  Synthetic oligonucleotide
tatctacacgggtctcaccaaaacgcaactcctggaaaaagtattacagcatccaaaaattattaatcaagcgaccttactttacttgaaaaaaacacttcgggaggatgaagaaattttcaagtaaagtaacggttttagagtgagaccagcgtaactcctaccgacag

SEQ ID NO: 175 - DNA, 170 bp, Artificial Sequence
  Synthetic oligonucleotide
tatctacacgggtctcaccaaaacgcaactcctggaaaaagtattacagcatccaaaaattattgatcaagcgaccttactttacttgaaaaaaacacttcgggaggatgaagaaattttcaagtaaagtaacggttttagagtgagaccagcgtaactctccatgcatg

SEQ ID NO: 176 - DNA, 170 bp, Artificial Sequence
  Synthetic oligonucleotide
tatctacacgggtctcaccaaaacgcaactcctggaaaaagtattacagcatccaaaaattatttgtcaagcgaccttactttacttgaaaaaaacacttcgggaggatgaagaaattttcaagtaaagtaacggttttagagtgagaccagcgtaactccccttcatga

SEQ ID NO: 177 - DNA, 170 bp, Artificial Sequence
  Synthetic oligonucleotide
tatctacacgggtctcaccaaaacgcaactcctggaaaaagtattacagcatccaaaaattattcaacaagcgaccttactttacttgaaaaaaacacttcgggaggatgaagaaattttcaagtaaagtaacggttttagagtgagaccagcgtaactcgattacgtcc

SEQ ID NO: 178 - DNA, 170 bp, Artificial Sequence
  Synthetic oligonucleotide
tatctacacgggtctcaccaaaacgcaactcctggaaaaagtattacagcatccaaaaattattgaacaagcgaccttactttacttgaaaaaaacacttcgggaggatgaagaaattttcaagtaaagtaacggttttagagtgagaccagcgtaactcctatgctttt

SEQ ID NO: 179 - DNA, 170 bp, Artificial Sequence
  Synthetic oligonucleotide
tatctacacgggtctcaccaaaacgcaactcctggaaaaagtattacagcatccaaaaattattggtcaagcgaccttactttacttgaaaaaaacacttcgggaggatgaagaaattttcaagtaaagtaacggttttagagtgagaccagcgtaactctttctaattt

SEQ ID NO: 180 - DNA, 170 bp, Artificial Sequence
  Synthetic oligonucleotide
tatctacacgggtctcaccaaaacgcaactcctggaaaaagtattacagcatccaaaaattattcatcaagcgaccttactttacttgaaaaaaacacttcgggaggatgaagaaattttcaagtaaagtaacggttttagagtgagaccagcgtaactcaagcgcgacg

SEQ ID NO: 181 - DNA, 170 bp, Artificial Sequence
  Synthetic oligonucleotide
tatctacacgggtctcaccaaaacgcaactcctggaaaaagtattacagcatccaaaaattattattcaagcgaccttactttacttgaaaaaaacacttcgggaggatgaagaaattttcaagtaaagtaacggttttagagtgagaccagcgtaactcgacaatttcg

SEQ ID NO: 182 - DNA, 170 bp, Artificial Sequence
  Synthetic oligonucleotide
tatctacacgggtctcaccaaaacgcaactcctggaaaaagtattacagcatccaaaaattattttgcaagcgaccttactttacttgaaaaaaacacttcgggaggatgaagaaattttcaagtaaagtaacggttttagagtgagaccagcgtaactctcggaattcc

SEQ ID NO: 183 - DNA, 170 bp, Artificial Sequence
  Synthetic oligonucleotide
tatctacacgggtctcaccaaaacgcaactcctggaaaaagtattacagcatccaaaaattattaaacaagcgaccttactttacttgaaaaaaacacttcgggaggatgaagaaattttcaagtaaagtaacggttttagagtgagaccagcgtaactcgccacataca

SEQ ID NO: 184 - DNA, 170 bp, Artificial Sequence
  Synthetic oligonucleotide
tatctacacgggtctcaccaaaacgcaactcctggaaaaagtattacagcatccaaaaattattatgcaagcgaccttactttacttgaaaaaaacacttcgggaggatgaagaaattttcaagtaaagtaacggttttagagtgagaccagcgtaactcattgcgtctc

SEQ ID NO: 185 - DNA, 170 bp, Artificial Sequence
  Synthetic oligonucleotide
tatctacacgggtctcaccaaaacgcaactcctggaaaaagtattacagcatccaaaaattattttccaagcgaccttactttacttgaaaaaaacacttcgggaggatgaagaaattttcaagtaaagtaacggttttagagtgagaccagcgtaactccgcttcttgt

SEQ ID NO: 186 - DNA, 170 bp, Artificial Sequence
  Synthetic oligonucleotide
tatctacacgggtctcaccaaaacgcaactcctggaaaaagtattacagcatccaaaaattattccacaagcgaccttactttacttgaaaaaaacacttcgggaggatgaagaaattttcaagtaaagtaacggttttagagtgagaccagcgtaactcaatcgatcga

SEQ ID NO: 187 - DNA, 170 bp, Artificial Sequence
  Synthetic oligonucleotide
tatctacacgggtctcaccaaaacgcaactcctggaaaaagtattacagcatccaaaaattatttctcaagcgaccttactttacttgaaaaaaacacttcgggaggatgaagaaattttcaagtaaagtaacggttttagagtgagaccagcgtaactctggtctaaat

SEQ ID NO: 188 - DNA, 170 bp, Artificial Sequence
  Synthetic oligonucleotide
tatctacacgggtctcaccaaaacgcaactcctggaaaaagtattacagcatccaaaaattattactcaagcgaccttactttacttgaaaaaaacacttcgggaggatgaagaaattttcaagtaaagtaacggttttagagtgagaccagcgtaactcatctcattag

SEQ ID NO: 189 - DNA, 170 bp, Artificial Sequence
  Synthetic oligonucleotide
tatctacacgggtctcaccaaaacgcaactcctggaaaaagtattacagcatccaaaaattatttggcaagcgaccttactttacttgaaaaaaacacttcgggaggatgaagaaattttcaagtaaagtaacggttttagagtgagaccagcgtaactcacagaaccaa

SEQ ID NO: 190 - DNA, 170 bp, Artificial Sequence
  Synthetic oligonucleotide
tatctacacgggtctcaccaaaacgcaactcctggaaaaagtattacagcatccaaaaattatttatcaagcgaccttactttacttgaaaaaaacacttcgggaggatgaagaaattttcaagtaaagtaacggttttagagtgagaccagcgtaactcgcaggagcaa

SEQ ID NO: 191 - DNA, 170 bp, Artificial Sequence
  Synthetic oligonucleotide
tatctacacgggtctcaccaaaacgcaactcctggaaaaagtattacagcatccaaaaattattgttcaagcgaccttactttacttgaaaaaaacacttcgggaggatgaagaaattttcaagtaaagtaacggttttagagtgagaccagcgtaactcccacttttgg

SEQ ID NO: 192 - DNA, 170 bp, Artificial Sequence
  Synthetic oligonucleotide
tatctacacgggtctcaccaaaacactcctggaaaaagtattacagcatccaaaaattattaaagctgcgaccttactttacttgaaaaaaacacttcgggaggatgaagaaattttcaagtaaagtaacggttttagagtgagaccagcgtaactcttagctctggctc

SEQ ID NO: 193 - DNA, 170 bp, Artificial Sequence
  Synthetic oligonucleotide
tatctacacgggtctcaccaaaacactcctggaaaaagtattacagcatccaaaaattattaaaagagcgaccttactttacttgaaaaaaacacttcgggaggatgaagaaattttcaagtaaagtaacggttttagagtgagaccagcgtaactcaagaagttcagct

SEQ ID NO: 194 - DNA, 170 bp, Artificial Sequence
  Synthetic oligonucleotide
tatctacacgggtctcaccaaaacactcctggaaaaagtattacagcatccaaaaattattaaaaatgcgaccttactttacttgaaaaaaacacttcgggaggatgaagaaattttcaagtaaagtaacggttttagagtgagaccagcgtaactccgaacggatcggt

SEQ ID NO: 195 - DNA, 170 bp, Artificial Sequence
  Synthetic oligonucleotide
tatctacacgggtctcaccaaaacactcctggaaaaagtattacagcatccaaaaattattaaagatgcgaccttactttacttgaaaaaaacacttcgggaggatgaagaaattttcaagtaaagtaacggttttagagtgagaccagcgtaactccgaccctatcaac

SEQ ID NO: 196 - DNA, 170 bp, Artificial Sequence
  Synthetic oligonucleotide
tatctacacgggtctcaccaaaacactcctggaaaaagtattacagcatccaaaaattattaaatgtgcgaccttactttacttgaaaaaaacacttcgggaggatgaagaaattttcaagtaaagtaacggttttagagtgagaccagcgtaactcggatcacatgcac

SEQ ID NO: 197 - DNA, 170 bp, Artificial Sequence
  Synthetic oligonucleotide
tatctacacgggtctcaccaaaacactcctggaaaaagtattacagcatccaaaaattattaaacaagcgaccttactttacttgaaaaaaacacttcgggaggatgaagaaattttcaagtaaagtaacggttttagagtgagaccagcgtaactccaacaggcctgga

SEQ ID NO: 198 - DNA, 170 bp, Artificial Sequence
  Synthetic oligonucleotide
tatctacacgggtctcaccaaaacactcctggaaaaagtattacagcatccaaaaattattaaagaagcgaccttactttacttgaaaaaaacacttcgggaggatgaagaaattttcaagtaaagtaacggttttagagtgagaccagcgtaactcttgacgtagcagg

SEQ ID NO: 199 - DNA, 170 bp, Artificial Sequence
  Synthetic oligonucleotide
tatctacacgggtctcaccaaaacactcctggaaaaagtattacagcatccaaaaattattaaaggtgcgaccttactttacttgaaaaaaacacttcgggaggatgaagaaattttcaagtaaagtaacggttttagagtgagaccagcgtaactccacgcggtcatga

SEQ ID NO: 200 - DNA, 170 bp, Artificial Sequence
  Synthetic oligonucleotide
tatctacacgggtctcaccaaaacactcctggaaaaagtattacagcatccaaaaattattaaacatgcgaccttactttacttgaaaaaaacacttcgggaggatgaagaaattttcaagtaaagtaacggttttagagtgagaccagcgtaactcacattttcgtgaa

SEQ ID NO: 201 - DNA, 170 bp, Artificial Sequence
  Synthetic oligonucleotide
tatctacacgggtctcaccaaaacactcctggaaaaagtattacagcatccaaaaattattaaaattgcgaccttactttacttgaaaaaaacacttcgggaggatgaagaaattttcaagtaaagtaacggttttagagtgagaccagcgtaactccctgtagattccc

SEQ ID NO: 202 - DNA, 170 bp, Artificial Sequence
  Synthetic oligonucleotide
tatctacacgggtctcaccaaaacactcctggaaaaagtattacagcatccaaaaattattaaattggcgaccttactttacttgaaaaaaacacttcgggaggatgaagaaattttcaagtaaagtaacggttttagagtgagaccagcgtaactcgtgaggaagggct

SEQ ID NO: 203 - DNA, 170 bp, Artificial Sequence
  Synthetic oligonucleotide
tatctacacgggtctcaccaaaacactcctggaaaaagtattacagcatccaaaaattattaaaaaagcgaccttactttacttgaaaaaaacacttcgggaggatgaagaaattttcaagtaaagtaacggttttagagtgagaccagcgtaactcacggacagccgca

SEQ ID NO: 204 - DNA, 170 bp, Artificial Sequence
  Synthetic oligonucleotide
tatctacacgggtctcaccaaaacactcctggaaaaagtattacagcatccaaaaattattaaaatggcgaccttactttacttgaaaaaaacacttcgggaggatgaagaaattttcaagtaaagtaacggttttagagtgagaccagcgtaactcgggcacatccact

SEQ ID NO: 205 - DNA, 170 bp, Artificial Sequence
  Synthetic oligonucleotide
tatctacacgggtctcaccaaaacactcctggaaaaagtattacagcatccaaaaattattaaatttgcgaccttactttacttgaaaaaaacacttcgggaggatgaagaaattttcaagtaaagtaacggttttagagtgagaccagcgtaactccctcctgcccttt

SEQ ID NO: 206 - DNA, 170 bp, Artificial Sequence
  Synthetic oligonucleotide
tatctacacgggtctcaccaaaacactcctggaaaaagtattacagcatccaaaaattattaaaccagcgaccttactttacttgaaaaaaacacttcgggaggatgaagaaattttcaagtaaagtaacggttttagagtgagaccagcgtaactcgtctcgggtttag

SEQ ID NO: 207 - DNA, 170 bp, Artificial Sequence
  Synthetic oligonucleotide
tatctacacgggtctcaccaaaacactcctggaaaaagtattacagcatccaaaaattattaaatctgcgaccttactttacttgaaaaaaacacttcgggaggatgaagaaattttcaagtaaagtaacggttttagagtgagaccagcgtaactcagagtgttctacg

SEQ ID NO: 208 - DNA, 170 bp, Artificial Sequence
  Synthetic oligonucleotide
tatctacacgggtctcaccaaaacactcctggaaaaagtattacagcatccaaaaattattaaaactgcgaccttactttacttgaaaaaaacacttcgggaggatgaagaaattttcaagtaaagtaacggttttagagtgagaccagcgtaactcgcgtccttaacat

SEQ ID NO: 209 - DNA, 170 bp, Artificial Sequence
  Synthetic oligonucleotide
tatctacacgggtctcaccaaaacactcctggaaaaagtattacagcatccaaaaattattaaatgggcgaccttactttacttgaaaaaaacacttcgggaggatgaagaaattttcaagtaaagtaacggttttagagtgagaccagcgtaactcagaacgaaggacg

SEQ ID NO: 210 - DNA, 170 bp, Artificial Sequence
  Synthetic oligonucleotide
tatctacacgggtctcaccaaaacactcctggaaaaagtattacagcatccaaaaattattaaatatgcgaccttactttacttgaaaaaaacacttcgggaggatgaagaaattttcaagtaaagtaacggttttagagtgagaccagcgtaactcaccgcggccgtgc

SEQ ID NO: 211 - DNA, 170 bp, Artificial Sequence
  Synthetic oligonucleotide
tatctacacgggtctcaccaaaacactcctggaaaaagtattacagcatccaaaaattattaaagttgcgaccttactttacttgaaaaaaacacttcgggaggatgaagaaattttcaagtaaagtaacggttttagagtgagaccagcgtaactcaggttacaaaagc

SEQ ID NO: 212 - DNA, 170 bp, Artificial Sequence
  Synthetic oligonucleotide
tatctacacgggtctcaccaaaaccctggaaaaagtattacagcatccaaaaattattaaacaagcgaccttactttacttgaaaaaaacacttcgggaggatgaagaaattttcaagtaaagtaacggttttagagtgagaccagcgtaactcgagtgactcaagatcc

SEQ ID NO: 213 - DNA, 170 bp, Artificial Sequence
  Synthetic oligonucleotide
tatctacacgggtctcaccaaaaccctggaaaaagtattacagcatccaaaaattattaaacaacggaccttactttacttgaaaaaaacacttcgggaggatgaagaaattttcaagtaaagtaacggttttagagtgagaccagcgtaactcctcttatcacactgac

SEQ ID NO: 214 - DNA, 170 bp, Artificial Sequence
  Synthetic oligonucleotide
tatctacacgggtctcaccaaaaccctggaaaaagtattacagcatccaaaaattattaaacaaaacaccttactttacttgaaaaaaacacttcgggaggatgaagaaattttcaagtaaagtaacggttttagagtgagaccagcgtaactcaatattgacgtaacat

SEQ ID NO: 215 - DNA, 170 bp, Artificial Sequence
  Synthetic oligonucleotide
tatctacacgggtctcaccaaaaccctggaaaaagtattacagcatccaaaaattattaaacaagacaccttactttacttgaaaaaaacacttcgggaggatgaagaaattttcaagtaaagtaacggttttagagtgagaccagcgtaactccatcgctgcttcccgc

SEQ ID NO: 216 - DNA, 170 bp, Artificial Sequence
  Synthetic oligonucleotide
tatctacacgggtctcaccaaaaccctggaaaaagtattacagcatccaaaaattattaaacaatgcaccttactttacttgaaaaaaacacttcgggaggatgaagaaattttcaagtaaagtaacggttttagagtgagaccagcgtaactcaatataaagcttagcg

SEQ ID NO: 217 - DNA, 170 bp, Artificial Sequence
  Synthetic oligonucleotide
tatctacacgggtctcaccaaaaccctggaaaaagtattacagcatccaaaaattattaaacaacagaccttactttacttgaaaaaaacacttcgggaggatgaagaaattttcaagtaaagtaacggttttagagtgagaccagcgtaactctttaggagtgggttag

SEQ ID NO: 218 - DNA, 170 bp, Artificial Sequence
  Synthetic oligonucleotide
tatctacacgggtctcaccaaaaccctggaaaaagtattacagcatccaaaaattattaaacaagagatcttactttacttgaaaaaaacacttcgggaggatgaagaaattttcaagtaaagtaacggttttagagtgagaccagcgtaactctaaaattttatataca

SEQ ID NO: 219 - DNA, 170 bp, Artificial Sequence
  Synthetic oligonucleotide
tatctacacgggtctcaccaaaaccctggaaaaagtattacagcatccaaaaattattaaacaagggaccttactttacttgaaaaaaacacttcgggaggatgaagaaattttcaagtaaagtaacggttttagagtgagaccagcgtaactccatcatggaattagaa

SEQ ID NO: 220 - DNA, 170 bp, Artificial Sequence
  Synthetic oligonucleotide
tatctacacgggtctcaccaaaaccctggaaaaagtattacagcatccaaaaattattaaacaacacaccttactttacttgaaaaaaacacttcgggaggatgaagaaattttcaagtaaagtaacggttttagagtgagaccagcgtaactcggttactcggaaagac

SEQ ID NO: 221 - DNA, 170 bp, Artificial Sequence
  Synthetic oligonucleotide
tatctacacgggtctcaccaaaaccctggaaaaagtattacagcatccaaaaattattaaacaaatcaccttactttacttgaaaaaaacacttcgggaggatgaagaaattttcaagtaaagtaacggttttagagtgagaccagcgtaactctcgacgacagcccatg

SEQ ID NO: 222 - DNA, 170 bp, Artificial Sequence
  Synthetic oligonucleotide
tatctacacgggtctcaccaaaaccctggaaaaagtattacagcatccaaaaattattaaacaactcaccttactttacttgaaaaaaacacttcgggaggatgaagaaattttcaagtaaagtaacggttttagagtgagaccagcgtaactcggatgctacactctcc

SEQ ID NO: 223 - DNA, 170 bp, Artificial Sequence
  Synthetic oligonucleotide
tatctacacgggtctcaccaaaaccctggaaaaagtattacagcatccaaaaattattaaacaaaagaccttactttacttgaaaaaaacacttcgggaggatgaagaaattttcaagtaaagtaacggttttagagtgagaccagcgtaactctctcaacggtgagttg

SEQ ID NO: 224 - DNA, 170 bp, Artificial Sequence
  Synthetic oligonucleotide
tatctacacgggtctcaccaaaaccctggaaaaagtattacagcatccaaaaattattaaacaaatgaccttactttacttgaaaaaaacacttcgggaggatgaagaaattttcaagtaaagtaacggttttagagtgagaccagcgtaactctgggattgtgacctcc

SEQ ID NO: 225 - DNA, 170 bp, Artificial Sequence
  Synthetic oligonucleotide
tatctacacgggtctcaccaaaaccctggaaaaagtattacagcatccaaaaattattaaacaattcaccttactttacttgaaaaaaacacttcgggaggatgaagaaattttcaagtaaagtaacggttttagagtgagaccagcgtaactcctaaccgttttgatgc

SEQ ID NO: 226 - DNA, 170 bp, Artificial Sequence
  Synthetic oligonucleotide
tatctacacgggtctcaccaaaaccctggaaaaagtattacagcatccaaaaattattaaacaaccgaccttactttacttgaaaaaaacacttcgggaggatgaagaaattttcaagtaaagtaacggttttagagtgagaccagcgtaactctgaattttgattcaac

SEQ ID NO: 227 - DNA, 170 bp, Artificial Sequence
  Synthetic oligonucleotide
tatctacacgggtctcaccaaaaccctggaaaaagtattacagcatccaaaaattattaaacaaagcaccttactttacttgaaaaaaacacttcgggaggatgaagaaattttcaagtaaagtaacggttttagagtgagaccagcgtaactcaagtaataggtgggtc

SEQ ID NO: 228 - DNA, 170 bp, Artificial Sequence
  Synthetic oligonucleotide
tatctacacgggtctcaccaaaaccctggaaaaagtattacagcatccaaaaattattaaacaaacgaccttactttacttgaaaaaaacacttcgggaggatgaagaaattttcaagtaaagtaacggttttagagtgagaccagcgtaactcggtctggcctgttcga

SEQ ID NO: 229 - DNA, 170 bp, Artificial Sequence
  Synthetic oligonucleotide
tatctacacgggtctcaccaaaaccctggaaaaagtattacagcatccaaaaattattaaacaatggaccttactttacttgaaaaaaacacttcgggaggatgaagaaattttcaagtaaagtaacggttttagagtgagaccagcgtaactcgcacaaattgagtttg

SEQ ID NO: 230 - DNA, 170 bp, Artificial Sequence
  Synthetic oligonucleotide
tatctacacgggtctcaccaaaaccctggaaaaagtattacagcatccaaaaattattaaacaatacaccttactttacttgaaaaaaacacttcgggaggatgaagaaattttcaagtaaagtaacggttttagagtgagaccagcgtaactcttcgatcctggtaaca

SEQ ID NO: 231 - DNA, 170 bp, Artificial Sequence
  Synthetic oligonucleotide
tatctacacgggtctcaccaaaaccctggaaaaagtattacagcatccaaaaattattaaacaagtcaccttactttacttgaaaaaaacacttcgggaggatgaagaaattttcaagtaaagtaacggttttagagtgagaccagcgtaactcaccgcccgtggcatac

SEQ ID NO: 232 - DNA, 170 bp, Artificial Sequence
  Synthetic oligonucleotide
tatctacacgggtctcaccaaaacctggaaaaagtattacagcatccaaaaattattaaacaagcggcgttactttacttgaaaaaaacacttcgggaggatgaagaaaaattttcaagtaaagtaacggttttagagtgagaccagcgtaactcactatggtggttttc

SEQ ID NO: 233 - DNA, 170 bp, Artificial Sequence
  Synthetic oligonucleotide
tatctacacgggtctcaccaaaacctggaaaaagtattacagcatccaaaaattattaaacaagcgcggttactttacttgaaaaaaacacttcgggaggatgaagaaaaattttcaagtaaagtaacggttttagagtgagaccagcgtaactctctccaactccatac

SEQ ID NO: 234 - DNA, 170 bp, Artificial Sequence
  Synthetic oligonucleotide
tatctacacgggtctcaccaaaacctggaaaaagtattacagcatccaaaaattattaaacaagcgaacttactttacttgaaaaaaacacttcgggaggatgaagaaaaattttcaagtaaagtaacggttttagagtgagaccagcgtaactcgaagatgccagtgac

SEQ ID NO: 235 - DNA, 170 bp, Artificial Sequence
  Synthetic oligonucleotide
tatctacacgggtctcaccaaaacctggaaaaagtattacagcatccaaaaattattaaacaagcggacttactttacttgaaaaaaacacttcgggaggatgaagaaaaattttcaagtaaagtaacggttttagagtgagaccagcgtaactcggagaccgagcgccc

SEQ ID NO: 236 - DNA, 170 bp, Artificial Sequence
  Synthetic oligonucleotide
tatctacacgggtctcaccaaaacctggaaaaagtattacagcatccaaaaattattaaacaagcgtgcttactttacttgaaaaaaacacttcgggaggatgaagaaaaattttcaagtaaagtaacggttttagagtgagaccagcgtaactcttgattccgcgagag

SEQ ID NO: 237 - DNA, 170 bp, Artificial Sequence
  Synthetic oligonucleotide
tatctacacgggtctcaccaaaacctggaaaaagtattacagcatccaaaaattattaaacaagcgcagttactttacttgaaaaaaacacttcgggaggatgaagaaaaattttcaagtaaagtaacggttttagagtgagaccagcgtaactccttggtcggaatgat

SEQ ID NO: 238 - DNA, 170 bp, Artificial Sequence
  Synthetic oligonucleotide
tatctacacgggtctcaccaaaacctggaaaaagtattacagcatccaaaaattattaaacaagcggagttactttacttgaaaaaaacacttcgggaggatgaagaaaaattttcaagtaaagtaacggttttagagtgagaccagcgtaactcaccagagtgagtacc

SEQ ID NO: 239 - DNA, 170 bp, Artificial Sequence
  Synthetic oligonucleotide
tatctacacgggtctcaccaaaacctggaaaaagtattacagcatccaaaaattattaaacaagcggggttactttacttgaaaaaaacacttcgggaggatgaagaaaaattttcaagtaaagtaacggttttagagtgagaccagcgtaactcaccattgtatcaagc

SEQ ID NO: 240 - DNA, 170 bp, Artificial Sequence
  Synthetic oligonucleotide
tatctacacgggtctcaccaaaacctggaaaaagtattacagcatccaaaaattattaaacaagcgcacttactttacttgaaaaaaacacttcgggaggatgaagaaaaattttcaagtaaagtaacggttttagagtgagaccagcgtaactctgtagttacctatgt

SEQ ID NO: 241 - DNA, 170 bp, Artificial Sequence
  Synthetic oligonucleotide
tatctacacgggtctcaccaaaacctggaaaaagtattacagcatccaaaaattattaaacaagcgatcttactttacttgaaaaaaacacttcgggaggatgaagaaaaattttcaagtaaagtaacggttttagagtgagaccagcgtaactcaaatcaattttcgcc

SEQ ID NO: 242 - DNA, 170 bp, Artificial Sequence
  Synthetic oligonucleotide
tatctacacgggtctcaccaaaacctggaaaaagtattacagcatccaaaaattattaaacaagcgctcttactttacttgaaaaaaacacttcgggaggatgaagaaaaattttcaagtaaagtaacggttttagagtgagaccagcgtaactccacataggtgaggtt

SEQ ID NO: 243 - DNA, 170 bp, Artificial Sequence
  Synthetic oligonucleotide
tatctacacgggtctcaccaaaacctggaaaaagtattacagcatccaaaaattattaaacaagcgaagttactttacttgaaaaaaacacttcgggaggatgaagaaaaattttcaagtaaagtaacggttttagagtgagaccagcgtaactcctcgttgtctggccc

SEQ ID NO: 244 - DNA, 170 bp, Artificial Sequence
  Synthetic oligonucleotide
tatctacacgggtctcaccaaaacctggaaaaagtattacagcatccaaaaattattaaacaagcgatgttactttacttgaaaaaaacacttcgggaggatgaagaaaaattttcaagtaaagtaacggttttagagtgagaccagcgtaactcttccgcctaataggc

SEQ ID NO: 245 - DNA, 170 bp, Artificial Sequence
  Synthetic oligonucleotide
tatctacacgggtctcaccaaaacctggaaaaagtattacagcatccaaaaattattaaacaagcgttcttactttacttgaaaaaaacacttcgggaggatgaagaaaaattttcaagtaaagtaacggttttagagtgagaccagcgtaactcttcggatgaatcgcg

SEQ ID NO: 246 - DNA, 170 bp, Artificial Sequence
  Synthetic oligonucleotide
tatctacacgggtctcaccaaaacctggaaaaagtattacagcatccaaaaattattaaacaagcgccgttactttacttgaaaaaaacacttcgggaggatgaagaaaaattttcaagtaaagtaacggttttagagtgagaccagcgtaactccattggaatgcgacc

SEQ ID NO: 247 - DNA, 170 bp, Artificial Sequence
  Synthetic oligonucleotide
tatctacacgggtctcaccaaaacctggaaaaagtattacagcatccaaaaattattaaacaagcgagcttactttacttgaaaaaaacacttcgggaggatgaagaaaaattttcaagtaaagtaacggttttagagtgagaccagcgtaactcgtaccctgctccccc

SEQ ID NO: 248 - DNA, 170 bp, Artificial Sequence
  Synthetic oligonucleotide
tatctacacgggtctcaccaaaacctggaaaaagtattacagcatccaaaaattattaaacaagcgacgttactttacttgaaaaaaacacttcgggaggatgaagaaaaattttcaagtaaagtaacggttttagagtgagaccagcgtaactcgacacctgcgaagac

SEQ ID NO: 249 - DNA, 170 bp, Artificial Sequence
  Synthetic oligonucleotide
tatctacacgggtctcaccaaaacctggaaaaagtattacagcatccaaaaattattaaacaagcgtggttactttacttgaaaaaaacacttcgggaggatgaagaaaaattttcaagtaaagtaacggttttagagtgagaccagcgtaactctgaaacattaagaag

SEQ ID NO: 250 - DNA, 170 bp, Artificial Sequence
  Synthetic oligonucleotide
tatctacacgggtctcaccaaaacctggaaaaagtattacagcatccaaaaattattaaacaagcgtacttactttacttgaaaaaaacacttcgggaggatgaagaaaaattttcaagtaaagtaacggttttagagtgagaccagcgtaactcatctgtcacgtcgtg

SEQ ID NO: 251 - DNA, 170 bp, Artificial Sequence
  Synthetic oligonucleotide
tatctacacgggtctcaccaaaacctggaaaaagtattacagcatccaaaaattattaaacaagcggtcttactttacttgaaaaaaacacttcgggaggatgaagaaaaattttcaagtaaagtaacggttttagagtgagaccagcgtaactcagaggaaactctcag

SEQ ID NO: 252 - DNA, 170 bp, Artificial Sequence
  Synthetic oligonucleotide
tatctacacgggtctcaccaaaacctggaaaaagtattacagcatccaaaaattattaaacaagcgaccgctctttacttgaaaaaaacacttcgggaggatgaagaaatggaattttcaagtaaagtaacggttttagagtgagaccagcgtaactctggacatatcat

SEQ ID NO: 253 - DNA, 170 bp, Artificial Sequence
  Synthetic oligonucleotide
tatctacacgggtctcaccaaaacctggaaaaagtattacagcatccaaaaattattaaacaagcgaccagactttacttgaaaaaaacacttcgggaggatgaagaaatggaattttcaagtaaagtaacggttttagagtgagaccagcgtaactcgtgtgcgggata

SEQ ID NO: 254 - DNA, 170 bp, Artificial Sequence
  Synthetic oligonucleotide
tatctacacgggtctcaccaaaacctggaaaaagtattacagcatccaaaaattattaaacaagcgaccaatctttacttgaaaaaaacacttcgggaggatgaagaaatggaattttcaagtaaagtaacggttttagagtgagaccagcgtaactcaacctcctaatg

SEQ ID NO: 255 - DNA, 170 bp, Artificial Sequence
  Synthetic oligonucleotide
tatctacacgggtctcaccaaaacctggaaaaagtattacagcatccaaaaattattaaacaagcgaccgatctttacttgaaaaaaacacttcgggaggatgaagaaatggaattttcaagtaaagtaacggttttagagtgagaccagcgtaactcttcctccttcat

SEQ ID NO: 256 - DNA, 170 bp, Artificial Sequence
  Synthetic oligonucleotide
tatctacacgggtctcaccaaaacctggaaaaagtattacagcatccaaaaattattaaacaagcgacctgtctttacttgaaaaaaacacttcgggaggatgaagaaatggaattttcaagtaaagtaacggttttagagtgagaccagcgtaactcggtatgcgcggt

SEQ ID NO: 257 - DNA, 170 bp, Artificial Sequence
  Synthetic oligonucleotide
tatctacacgggtctcaccaaaacctggaaaaagtattacagcatccaaaaattattaaacaagcgacccaactttacttgaaaaaaacacttcgggaggatgaagaaatggaattttcaagtaaagtaacggttttagagtgagaccagcgtaactcaaccatcacgcg

SEQ ID NO: 258 - DNA, 170 bp, Artificial Sequence
  Synthetic oligonucleotide
tatctacacgggtctcaccaaaacctggaaaaagtattacagcatccaaaaattattaaacaagcgaccgaactttacttgaaaaaaacacttcgggaggatgaagaaatggaattttcaagtaaagtaacggttttagagtgagaccagcgtaactcccaggcggtcgg

SEQ ID NO: 259 - DNA, 170 bp, Artificial Sequence
  Synthetic oligonucleotide
tatctacacgggtctcaccaaaacctggaaaaagtattacagcatccaaaaattattaaacaagcgaccggtctttacttgaaaaaaacacttcgggaggatgaagaaatggaattttcaagtaaagtaacggttttagagtgagaccagcgtaactctggttgtcaacg

SEQ ID NO: 260 - DNA, 170 bp, Artificial Sequence
  Synthetic oligonucleotide
tatctacacgggtctcaccaaaacctggaaaaagtattacagcatccaaaaattattaaacaagcgacccatctttacttgaaaaaaacacttcgggaggatgaagaaatggaattttcaagtaaagtaacggttttagagtgagaccagcgtaactctagattgccagg

SEQ ID NO: 261 - DNA, 170 bp, Artificial Sequence
  Synthetic oligonucleotide
tatctacacgggtctcaccaaaacctggaaaaagtattacagcatccaaaaattattaaacaagcgaccattctttacttgaaaaaaacacttcgggaggatgaagaaatggaattttcaagtaaagtaacggttttagagtgagaccagcgtaactcggcacaccagtg

SEQ ID NO: 262 - DNA, 170 bp, Artificial Sequence
  Synthetic oligonucleotide
tatctacacgggtctcaccaaaacctggaaaaagtattacagcatccaaaaattattaaacaagcgaccttgctttacttgaaaaaaacacttcgggaggatgaagaaatggaattttcaagtaaagtaacggttttagagtgagaccagcgtaactcgccaggttttag

SEQ ID NO: 263 - DNA, 170 bp, Artificial Sequence
  Synthetic oligonucleotide
tatctacacgggtctcaccaaaacctggaaaaagtattacagcatccaaaaattattaaacaagcgaccaaactttacttgaaaaaaacacttcgggaggatgaagaaatggaattttcaagtaaagtaacggttttagagtgagaccagcgtaactctacgtcttgcca

SEQ ID NO: 264 - DNA, 170 bp, Artificial Sequence
  Synthetic oligonucleotide
tatctacacgggtctcaccaaaacctggaaaaagtattacagcatccaaaaattattaaacaagcgaccatgctttacttgaaaaaaacacttcgggaggatgaagaaatggaattttcaagtaaagtaacggttttagagtgagaccagcgtaactcggacgaatgcgg

SEQ ID NO: 265 - DNA, 170 bp, Artificial Sequence
  Synthetic oligonucleotide
tatctacacgggtctcaccaaaacctggaaaaagtattacagcatccaaaaattattaaacaagcgacctttctttacttgaaaaaaacacttcgggaggatgaagaaatggaattttcaagtaaagtaacggttttagagtgagaccagcgtaactcctgacacatggg

SEQ ID NO: 266 - DNA, 170 bp, Artificial Sequence
  Synthetic oligonucleotide
tatctacacgggtctcaccaaaacctggaaaaagtattacagcatccaaaaattattaaacaagcgaccccactttacttgaaaaaaacacttcgggaggatgaagaaatggaattttcaagtaaagtaacggttttagagtgagaccagcgtaactcgcccccgtaaag

SEQ ID NO: 267 - DNA, 170 bp, Artificial Sequence
  Synthetic oligonucleotide
tatctacacgggtctcaccaaaacctggaaaaagtattacagcatccaaaaattattaaacaagcgacctctctttacttgaaaaaaacacttcgggaggatgaagaaatggaattttcaagtaaagtaacggttttagagtgagaccagcgtaactcgaagcagctaca

SEQ ID NO: 268 - DNA, 170 bp, Artificial Sequence
  Synthetic oligonucleotide
tatctacacgggtctcaccaaaacctggaaaaagtattacagcatccaaaaattattaaacaagcgaccactctttacttgaaaaaaacacttcgggaggatgaagaaatggaattttcaagtaaagtaacggttttagagtgagaccagcgtaactctatccacggtca

SEQ ID NO: 269 - DNA, 170 bp, Artificial Sequence
  Synthetic oligonucleotide
tatctacacgggtctcaccaaaacctggaaaaagtattacagcatccaaaaattattaaacaagcgacctggctttacttgaaaaaaacacttcgggaggatgaagaaatggaattttcaagtaaagtaacggttttagagtgagaccagcgtaactcgtacacgtatgg

SEQ ID NO: 270 - DNA, 170 bp, Artificial Sequence
  Synthetic oligonucleotide
tatctacacgggtctcaccaaaacctggaaaaagtattacagcatccaaaaattattaaacaagcgacctatctttacttgaaaaaaacacttcgggaggatgaagaaatggaattttcaagtaaagtaacggttttagagtgagaccagcgtaactccgccgagcctgc

SEQ ID NO: 271 - DNA, 170 bp, Artificial Sequence
  Synthetic oligonucleotide
tatctacacgggtctcaccaaaacctggaaaaagtattacagcatccaaaaattattaaacaagcgaccgttctttacttgaaaaaaacacttcgggaggatgaagaaatggaattttcaagtaaagtaacggttttagagtgagaccagcgtaactccatagcccttga

SEQ ID NO: 272 - DNA, 170 bp, Artificial Sequence
  Synthetic oligonucleotide
tatctacacgggtctcaccaaaacctggaaaaagtattacagcatccaaaaattattaaacaagcgaccttagcttacttgaaaaaaacacttcgggaggatgaagaaatgggctaattttcaagtaaagtaacggttttagagtgagaccagcgtaactccctatggga

SEQ ID NO: 273 - DNA, 170 bp, Artificial Sequence
  Synthetic oligonucleotide
tatctacacgggtctcaccaaaacctggaaaaagtattacagcatccaaaaattattaaacaagcgaccttaagatacttgaaaaaaacacttcgggaggatgaagaaatgggctaattttcaagtaaagtaacggttttagagtgagaccagcgtaactcaacctagac

SEQ ID NO: 274 - DNA, 170 bp, Artificial Sequence
  Synthetic oligonucleotide
tatctacacgggtctcaccaaaacctggaaaaagtattacagcatccaaaaattattaaacaagcgaccttaaattacttgaaaaaaacacttcgggaggatgaagaaatgggctaattttcaagtaaagtaacggttttagagtgagaccagcgtaactccacgctaaa

SEQ ID NO: 275 - DNA, 170 bp, Artificial Sequence
  Synthetic oligonucleotide
tatctacacgggtctcaccaaaacctggaaaaagtattacagcatccaaaaattattaaacaagcgaccttagattacttgaaaaaaacacttcgggaggatgaagaaatgggctaattttcaagtaaagtaacggttttagagtgagaccagcgtaactcgcccaatcc

SEQ ID NO: 276 - DNA, 170 bp, Artificial Sequence
  Synthetic oligonucleotide
tatctacacgggtctcaccaaaacctggaaaaagtattacagcatccaaaaattattaaacaagcgaccttatgttacttgaaaaaaacacttcgggaggatgaagaaatgggctaattttcaagtaaagtaacggttttagagtgagaccagcgtaactcgtgaagaac

SEQ ID NO: 277 - DNA, 170 bp, Artificial Sequence
  Synthetic oligonucleotide
tatctacacgggtctcaccaaaacctggaaaaagtattacagcatccaaaaattattaaacaagcgaccttacaatacttgaaaaaaacacttcgggaggatgaagaaatgggctaattttcaagtaaagtaacggttttagagtgagaccagcgtaactcccattggtc

SEQ ID NO: 278 - DNA, 170 bp, Artificial Sequence
  Synthetic oligonucleotide
tatctacacgggtctcaccaaaacctggaaaaagtattacagcatccaaaaattattaaacaagcgaccttagaatacttgaaaaaaacacttcgggaggatgaagaaatgggctaattttcaagtaaagtaacggttttagagtgagaccagcgtaactcaagtaggga

SEQ ID NO: 279 - DNA, 170 bp, Artificial Sequence
  Synthetic oligonucleotide
tatctacacgggtctcaccaaaacctggaaaaagtattacagcatccaaaaattattaaacaagcgaccttaggttacttgaaaaaaacacttcgggaggatgaagaaatgggctaattttcaagtaaagtaacggttttagagtgagaccagcgtaactcatgtccgca

SEQ ID NO: 280 - DNA, 170 bp, Artificial Sequence
  Synthetic oligonucleotide
tatctacacgggtctcaccaaaacctggaaaaagtattacagcatccaaaaattattaaacaagcgaccttacattacttgaaaaaaacacttcgggaggatgaagaaatgggctaattttcaagtaaagtaacggttttagagtgagaccagcgtaactcaactcgcag

SEQ ID NO: 281 - DNA, 170 bp, Artificial Sequence
  Synthetic oligonucleotide
tatctacacgggtctcaccaaaacctggaaaaagtattacagcatccaaaaattattaaacaagcgaccttaatttacttgaaaaaaacacttcgggaggatgaagaaatgggctaattttcaagtaaagtaacggttttagagtgagaccagcgtaactcatattcctc

SEQ ID NO: 282 - DNA, 170 bp, Artificial Sequence
  Synthetic oligonucleotide
tatctacacgggtctcaccaaaacctggaaaaagtattacagcatccaaaaattattaaacaagcgaccttattgtacttgaaaaaaacacttcgggaggatgaagaaatgggctaattttcaagtaaagtaacggttttagagtgagaccagcgtaactcatccgtgaa

SEQ ID NO: 283 - DNA, 170 bp, Artificial Sequence
  Synthetic oligonucleotide
tatctacacgggtctcaccaaaacctggaaaaagtattacagcatccaaaaattattaaacaagcgaccttaaaatacttgaaaaaaacacttcgggaggatgaagaaatgggctaattttcaagtaaagtaacggttttagagtgagaccagcgtaactcggtccacag

SEQ ID NO: 284 - DNA, 170 bp, Artificial Sequence
  Synthetic oligonucleotide
tatctacacgggtctcaccaaaacctggaaaaagtattacagcatccaaaaattattaaacaagcgaccttaatgtacttgaaaaaaacacttcgggaggatgaagaaatgggctaattttcaagtaaagtaacggttttagagtgagaccagcgtaactcaggttacgc

SEQ ID NO: 285 - DNA, 170 bp, Artificial Sequence
  Synthetic oligonucleotide
tatctacacgggtctcaccaaaacctggaaaaagtattacagcatccaaaaattattaaacaagcgaccttattttacttgaaaaaaacacttcgggaggatgaagaaatgggctaattttcaagtaaagtaacggttttagagtgagaccagcgtaactcaagtgttta

SEQ ID NO: 286 - DNA, 170 bp, Artificial Sequence
  Synthetic oligonucleotide
tatctacacgggtctcaccaaaacctggaaaaagtattacagcatccaaaaattattaaacaagcgaccttaccatacttgaaaaaaacacttcgggaggatgaagaaatgggctaattttcaagtaaagtaacggttttagagtgagaccagcgtaactcggcgtcgtc

SEQ ID NO: 287 - DNA, 170 bp, Artificial Sequence
  Synthetic oligonucleotide
tatctacacgggtctcaccaaaacctggaaaaagtattacagcatccaaaaattattaaacaagcgaccttatcttacttgaaaaaaacacttcgggaggatgaagaaatgggctaattttcaagtaaagtaacggttttagagtgagaccagcgtaactcgacgttcga

SEQ ID NO: 288 - DNA, 170 bp, Artificial Sequence
  Synthetic oligonucleotide
tatctacacgggtctcaccaaaacctggaaaaagtattacagcatccaaaaattattaaacaagcgaccttaacttacttgaaaaaaacacttcgggaggatgaagaaatgggctaattttcaagtaaagtaacggttttagagtgagaccagcgtaactctcaatgctt

SEQ ID NO: 289 - DNA, 170 bp, Artificial Sequence
  Synthetic oligonucleotide
tatctacacgggtctcaccaaaacctggaaaaagtattacagcatccaaaaattattaaacaagcgaccttatggtacttgaaaaaaacacttcgggaggatgaagaaatgggctaattttcaagtaaagtaacggttttagagtgagaccagcgtaactctggaactat

SEQ ID NO: 290 - DNA, 170 bp, Artificial Sequence
  Synthetic oligonucleotide
tatctacacgggtctcaccaaaacctggaaaaagtattacagcatccaaaaattattaaacaagcgaccttatattacttgaaaaaaacacttcgggaggatgaagaaatgggctaattttcaagtaaagtaacggttttagagtgagaccagcgtaactcaaggcggca

SEQ ID NO: 291 - DNA, 170 bp, Artificial Sequence
  Synthetic oligonucleotide
tatctacacgggtctcaccaaaacctggaaaaagtattacagcatccaaaaattattaaacaagcgaccttagtttacttgaaaaaaacacttcgggaggatgaagaaatgggctaattttcaagtaaagtaacggttttagagtgagaccagcgtaactcctagcacgc

SEQ ID NO: 292 - DNA, 170 bp, Artificial Sequence
  Synthetic oligonucleotide
tatctacacgggtctcaccaaaacctggaaaaagtattacagcatccaaaaattattaaacaagcgaccttacttgctttgaaaaaaacacttcgggaggatgaagaaatgggcttgaaattttcaagtaaagtaacggttttagagtgagaccagcgtaactccacggc

SEQ ID NO: 293 - DNA, 170 bp, Artificial Sequence
  Synthetic oligonucleotide
tatctacacgggtctcaccaaaacctggaaaaagtattacagcatccaaaaattattaaacaagcgaccttacttagattgaaaaaaacacttcgggaggatgaagaaatgggcttgaaattttcaagtaaagtaacggttttagagtgagaccagcgtaactcccgtat

SEQ ID NO: 294 - DNA, 170 bp, Artificial Sequence
  Synthetic oligonucleotide
tatctacacgggtctcaccaaaacctggaaaaagtattacagcatccaaaaattattaaacaagcgaccttacttaatttgaaaaaaacacttcgggaggatgaagaaatgggcttgaaattttcaagtaaagtaacggttttagagtgagaccagcgtaactcaactcg

SEQ ID NO: 295 - DNA, 170 bp, Artificial Sequence
  Synthetic oligonucleotide
tatctacacgggtctcaccaaaacctggaaaaagtattacagcatccaaaaattattaaacaagcgaccttacttgatttgaaaaaaacacttcgggaggatgaagaaatgggcttgaaattttcaagtaaagtaacggttttagagtgagaccagcgtaactccaggtc

SEQ ID NO: 296 - DNA, 170 bp, Artificial Sequence
  Synthetic oligonucleotide
tatctacacgggtctcaccaaaacctggaaaaagtattacagcatccaaaaattattaaacaagcgaccttactttgtttgaaaaaaacacttcgggaggatgaagaaatgggcttgaaattttcaagtaaagtaacggttttagagtgagaccagcgtaactcctcagt

SEQ ID NO: 297 - DNA, 170 bp, Artificial Sequence
  Synthetic oligonucleotide
tatctacacgggtctcaccaaaacctggaaaaagtattacagcatccaaaaattattaaacaagcgaccttacttcaattgaaaaaaacacttcgggaggatgaagaaatgggcttgaaattttcaagtaaagtaacggttttagagtgagaccagcgtaactcacggct

SEQ ID NO: 298 - DNA, 170 bp, Artificial Sequence
  Synthetic oligonucleotide
tatctacacgggtctcaccaaaacctggaaaaagtattacagcatccaaaaattattaaacaagcgaccttacttgaattgaaaaaaacacttcgggaggatgaagaaatgggcttgaaattttcaagtaaagtaacggttttagagtgagaccagcgtaactcctcatt

SEQ ID NO: 299 - DNA, 170 bp, Artificial Sequence
  Synthetic oligonucleotide
tatctacacgggtctcaccaaaacctggaaaaagtattacagcatccaaaaattattaaacaagcgaccttacttggtttgaaaaaaacacttcgggaggatgaagaaatgggcttgaaattttcaagtaaagtaacggttttagagtgagaccagcgtaactcgcgggg

SEQ ID NO: 300 - DNA, 170 bp, Artificial Sequence
  Synthetic oligonucleotide
tatctacacgggtctcaccaaaacctggaaaaagtattacagcatccaaaaattattaaacaagcgaccttacttcatttgaaaaaaacacttcgggaggatgaagaaatgggcttgaaattttcaagtaaagtaacggttttagagtgagaccagcgtaactcgcacca

SEQ ID NO: 301 - DNA, 170 bp, Artificial Sequence
  Synthetic oligonucleotide
tatctacacgggtctcaccaaaacctggaaaaagtattacagcatccaaaaattattaaacaagcgaccttacttattttgaaaaaaacacttcgggaggatgaagaaatgggcttgaaattttcaagtaaagtaacggttttagagtgagaccagcgtaactcctaatt

SEQ ID NO: 302 - DNA, 170 bp, Artificial Sequence
  Synthetic oligonucleotide
tatctacacgggtctcaccaaaacctggaaaaagtattacagcatccaaaaattattaaacaagcgaccttacttttgttgaaaaaaacacttcgggaggatgaagaaatgggcttgaaattttcaagtaaagtaacggttttagagtgagaccagcgtaactcgcgtag

SEQ ID NO: 303 - DNA, 170 bp, Artificial Sequence
  Synthetic oligonucleotide
tatctacacgggtctcaccaaaacctggaaaaagtattacagcatccaaaaattattaaacaagcgaccttacttaaattgaaaaaaacacttcgggaggatgaagaaatgggcttgaaattttcaagtaaagtaacggttttagagtgagaccagcgtaactctgtttg

SEQ ID NO: 304 - DNA, 170 bp, Artificial Sequence
  Synthetic oligonucleotide
tatctacacgggtctcaccaaaacctggaaaaagtattacagcatccaaaaattattaaacaagcgaccttacttatgttgaaaaaaacacttcgggaggatgaagaaatgggcttgaaattttcaagtaaagtaacggttttagagtgagaccagcgtaactcaagtat

SEQ ID NO: 305 - DNA, 170 bp, Artificial Sequence
  Synthetic oligonucleotide
tatctacacgggtctcaccaaaacctggaaaaagtattacagcatccaaaaattattaaacaagcgaccttacttttcttgaaaaaaacacttcgggaggatgaagaaatgggcttgaaattttcaagtaaagtaacggttttagagtgagaccagcgtaactcaataaa

SEQ ID NO: 306 - DNA, 170 bp, Artificial Sequence
  Synthetic oligonucleotide
tatctacacgggtctcaccaaaacctggaaaaagtattacagcatccaaaaattattaaacaagcgaccttacttccattgaaaaaaacacttcgggaggatgaagaaatgggcttgaaattttcaagtaaagtaacggttttagagtgagaccagcgtaactctggttg

SEQ ID NO: 307 - DNA, 170 bp, Artificial Sequence
  Synthetic oligonucleotide
tatctacacgggtctcaccaaaacctggaaaaagtattacagcatccaaaaattattaaacaagcgaccttactttctttgaaaaaaacacttcgggaggatgaagaaatgggcttgaaattttcaagtaaagtaacggttttagagtgagaccagcgtaactcatagct

SEQ ID NO: 308 - DNA, 170 bp, Artificial Sequence
  Synthetic oligonucleotide
tatctacacgggtctcaccaaaacctggaaaaagtattacagcatccaaaaattattaaacaagcgaccttacttactttgaaaaaaacacttcgggaggatgaagaaatgggcttgaaattttcaagtaaagtaacggttttagagtgagaccagcgtaactcagctaa

SEQ ID NO: 309 - DNA, 170 bp, Artificial Sequence
  Synthetic oligonucleotide
tatctacacgggtctcaccaaaacctggaaaaagtattacagcatccaaaaattattaaacaagcgaccttactttggttgaaaaaaacacttcgggaggatgaagaaatgggcttgaaattttcaagtaaagtaacggttttagagtgagaccagcgtaactccgtaac

SEQ ID NO: 310 - DNA, 170 bp, Artificial Sequence
  Synthetic oligonucleotide
tatctacacgggtctcaccaaaacctggaaaaagtattacagcatccaaaaattattaaacaagcgaccttactttatttgaaaaaaacacttcgggaggatgaagaaatgggcttgaaattttcaagtaaagtaacggttttagagtgagaccagcgtaactcaacaag

SEQ ID NO: 311 - DNA, 170 bp, Artificial Sequence
  Synthetic oligonucleotide
tatctacacgggtctcaccaaaacctggaaaaagtattacagcatccaaaaattattaaacaagcgaccttacttgttttgaaaaaaacacttcgggaggatgaagaaatgggcttgaaattttcaagtaaagtaacggttttagagtgagaccagcgtaactctctgat

SEQ ID NO: 312 - DNA, 170 bp, Artificial Sequence
  Synthetic oligonucleotide
tatctacacgggtctcaccaaaacctggaaaaagtattacagcatccaaaaattattaaacaagcgaccttactttacgctaaaaaaacacttcgggaggatgaagaaatgggcttgactaaattttcaagtaaagtaacggttttagagtgagaccagcgtaactctgg

SEQ ID NO: 313 - DNA, 170 bp, Artificial Sequence
  Synthetic oligonucleotide
tatctacacgggtctcaccaaaacctggaaaaagtattacagcatccaaaaattattaaacaagcgaccttactttacagaaaaaaaacacttcgggaggatgaagaaatgggcttgactaaattttcaagtaaagtaacggttttagagtgagaccagcgtaactcgta

SEQ ID NO: 314 - DNA, 170 bp, Artificial Sequence
  Synthetic oligonucleotide
tatctacacgggtctcaccaaaacctggaaaaagtattacagcatccaaaaattattaaacaagcgaccttactttacaataaaaaaacacttcgggaggatgaagaaatgggcttgactaaattttcaagtaaagtaacggttttagagtgagaccagcgtaactcgac

SEQ ID NO: 315 - DNA, 170 bp, Artificial Sequence
  Synthetic oligonucleotide
tatctacacgggtctcaccaaaacctggaaaaagtattacagcatccaaaaattattaaacaagcgaccttactttacgataaaaaaacacttcgggaggatgaagaaatgggcttgactaaattttcaagtaaagtaacggttttagagtgagaccagcgtaactctta

SEQ ID NO: 316 - DNA, 170 bp, Artificial Sequence
  Synthetic oligonucleotide
tatctacacgggtctcaccaaaacctggaaaaagtattacagcatccaaaaattattaaacaagcgaccttactttactgtaaaaaaacacttcgggaggatgaagaaatgggcttgactaaattttcaagtaaagtaacggttttagagtgagaccagcgtaactctga

SEQ ID NO: 317 - DNA, 170 bp, Artificial Sequence
  Synthetic oligonucleotide
tatctacacgggtctcaccaaaacctggaaaaagtattacagcatccaaaaattattaaacaagcgaccttactttaccaaaaaaaaacacttcgggaggatgaagaaatgggcttgactaaattttcaagtaaagtaacggttttagagtgagaccagcgtaactcaaa

SEQ ID NO: 318 - DNA, 170 bp, Artificial Sequence
  Synthetic oligonucleotide
tatctacacgggtctcaccaaaacctggaaaaagtattacagcatccaaaaattattaaacaagcgaccttactttacgaaaaaaaaacacttcgggaggatgaagaaatgggcttgactaaattttcaagtaaagtaacggttttagagtgagaccagcgtaactccgt

SEQ ID NO: 319 - DNA, 170 bp, Artificial Sequence
  Synthetic oligonucleotide
tatctacacgggtctcaccaaaacctggaaaaagtattacagcatccaaaaattattaaacaagcgaccttactttacggtaaaaaaacacttcgggaggatgaagaaatgggcttgactaaattttcaagtaaagtaacggttttagagtgagaccagcgtaactcttc

SEQ ID NO: 320 - DNA, 170 bp, Artificial Sequence
  Synthetic oligonucleotide
tatctacacgggtctcaccaaaacctggaaaaagtattacagcatccaaaaattattaaacaagcgaccttactttaccataaaaaaacacttcgggaggatgaagaaatgggcttgactaaattttcaagtaaagtaacggttttagagtgagaccagcgtaactcagc

SEQ ID NO: 321 - DNA, 170 bp, Artificial Sequence
  Synthetic oligonucleotide
tatctacacgggtctcaccaaaacctggaaaaagtattacagcatccaaaaattattaaacaagcgaccttactttacattaaaaaaacacttcgggaggatgaagaaatgggcttgactaaattttcaagtaaagtaacggttttagagtgagaccagcgtaactcgtg

SEQ ID NO: 322 - DNA, 170 bp, Artificial Sequence
  Synthetic oligonucleotide
tatctacacgggtctcaccaaaacctggaaaaagtattacagcatccaaaaattattaaacaagcgaccttactttacttgaaaaaaacacttcgggaggatgaagaaatgggcttgactaaattttcaagtaaagtaacggttttagagtgagaccagcgtaactccac

SEQ ID NO: 323 - DNA, 170 bp, Artificial Sequence
  Synthetic oligonucleotide
tatctacacgggtctcaccaaaacctggaaaaagtattacagcatccaaaaattattaaacaagcgaccttactttacaaaaaaaaaacacttcgggaggatgaagaaatgggcttgactaaattttcaagtaaagtaacggttttagagtgagaccagcgtaactcatc

SEQ ID NO: 324 - DNA, 170 bp, Artificial Sequence
  Synthetic oligonucleotide
tatctacacgggtctcaccaaaacctggaaaaagtattacagcatccaaaaattattaaacaagcgaccttactttacatgaaaaaaacacttcgggaggatgaagaaatgggcttgactaaattttcaagtaaagtaacggttttagagtgagaccagcgtaactccaa

SEQ ID NO: 325 - DNA, 170 bp, Artificial Sequence
  Synthetic oligonucleotide
tatctacacgggtctcaccaaaacctggaaaaagtattacagcatccaaaaattattaaacaagcgaccttactttactttaaaaaaacacttcgggaggatgaagaaatgggcttgactaaattttcaagtaaagtaacggttttagagtgagaccagcgtaactcacc

SEQ ID NO: 326 - DNA, 170 bp, Artificial Sequence
  Synthetic oligonucleotide
tatctacacgggtctcaccaaaacctggaaaaagtattacagcatccaaaaattattaaacaagcgaccttactttacccaaaaaaaacacttcgggaggatgaagaaatgggcttgactaaattttcaagtaaagtaacggttttagagtgagaccagcgtaactcatt

SEQ ID NO: 327 - DNA, 170 bp, Artificial Sequence
  Synthetic oligonucleotide
tatctacacgggtctcaccaaaacctggaaaaagtattacagcatccaaaaattattaaacaagcgaccttactttactctaaaaaaacacttcgggaggatgaagaaatgggcttgactaaattttcaagtaaagtaacggttttagagtgagaccagcgtaactcaga

SEQ ID NO: 328 - DNA, 170 bp, Artificial Sequence
  Synthetic oligonucleotide
tatctacacgggtctcaccaaaacctggaaaaagtattacagcatccaaaaattattaaacaagcgaccttactttacactaaaaaaacacttcgggaggatgaagaaatgggcttgactaaattttcaagtaaagtaacggttttagagtgagaccagcgtaactcgtc

SEQ ID NO: 329 - DNA, 170 bp, Artificial Sequence
  Synthetic oligonucleotide
tatctacacgggtctcaccaaaacctggaaaaagtattacagcatccaaaaattattaaacaagcgaccttactttactggaaaaaaacacttcgggaggatgaagaaatgggcttgactaaattttcaagtaaagtaacggttttagagtgagaccagcgtaactcgac

SEQ ID NO: 330 - DNA, 170 bp, Artificial Sequence
  Synthetic oligonucleotide
tatctacacgggtctcaccaaaacctggaaaaagtattacagcatccaaaaattattaaacaagcgaccttactttactataaaaaaacacttcgggaggatgaagaaatgggcttgactaaattttcaagtaaagtaacggttttagagtgagaccagcgtaactcaaa

SEQ ID NO: 331 - DNA, 170 bp, Artificial Sequence
  Synthetic oligonucleotide
tatctacacgggtctcaccaaaacctggaaaaagtattacagcatccaaaaattattaaacaagcgaccttactttacgttaaaaaaacacttcgggaggatgaagaaatgggcttgactaaattttcaagtaaagtaacggttttagagtgagaccagcgtaactccga

SEQ ID NO: 332 - DNA, 170 bp, Artificial Sequence
  Synthetic oligonucleotide
tatctacacgggtctcaccaaaacgcatccaaaaattattaaacaagccacgttactttacttggctaaaacacttagagaagacgaagaaatgggcttgactaccacatctactatcatgagcttgaaaaaaacacttcggggttttagagtgagaccagcgtaactct

SEQ ID NO: 333 - DNA, 170 bp, Artificial Sequence
  Synthetic oligonucleotide
tatctacacgggtctcaccaaaacgcatccaaaaattattaaacaagccacgttactttacttgagaaaaacacttagagaagacgaagaaatgggcttgactaccacatctactatcatgagcttgaaaaaaacacttcggggttttagagtgagaccagcgtaactct

SEQ ID NO: 334 - DNA, 170 bp, Artificial Sequence
  Synthetic oligonucleotide
tatctacacgggtctcaccaaaacgcatccaaaaattattaaacaagccacgttactttacttgaataaaacacttagagaagacgaagaaatgggcttgactaccacatctactatcatgagcttgaaaaaaacacttcggggttttagagtgagaccagcgtaactct

SEQ ID NO: 335 - DNA, 170 bp, Artificial Sequence
  Synthetic oligonucleotide
tatctacacgggtctcaccaaaacgcatccaaaaattattaaacaagccacgttactttacttggataaaacacttagagaagacgaagaaatgggcttgactaccacatctactatcatgagcttgaaaaaaacacttcggggttttagagtgagaccagcgtaactcc

SEQ ID NO: 336 - DNA, 170 bp, Artificial Sequence
  Synthetic oligonucleotide
tatctacacgggtctcaccaaaacgcatccaaaaattattaaacaagccacgttactttacttgtgtaaaacacttagagaagacgaagaaatgggcttgactaccacatctactatcatgagcttgaaaaaaacacttcggggttttagagtgagaccagcgtaactcg

SEQ ID NO: 337 - DNA, 170 bp, Artificial Sequence
  Synthetic oligonucleotide
tatctacacgggtctcaccaaaacgcatccaaaaattattaaacaagccacgttactttacttgcaaaaaacacttagagaagacgaagaaatgggcttgactaccacatctactatcatgagcttgaaaaaaacacttcggggttttagagtgagaccagcgtaactct

SEQ ID NO: 338 - DNA, 170 bp, Artificial Sequence
  Synthetic oligonucleotide
tatctacacgggtctcaccaaaacgcatccaaaaattattaaacaagccacgttactttacttggaaaaaacacttagagaagacgaagaaatgggcttgactaccacatctactatcatgagcttgaaaaaaacacttcggggttttagagtgagaccagcgtaactcg

SEQ ID NO: 339 - DNA, 170 bp, Artificial Sequence
  Synthetic oligonucleotide
tatctacacgggtctcaccaaaacgcatccaaaaattattaaacaagccacgttactttacttgggtaaaacacttagagaagacgaagaaatgggcttgactaccacatctactatcatgagcttgaaaaaaacacttcggggttttagagtgagaccagcgtaactca

SEQ ID NO: 340 - DNA, 170 bp, Artificial Sequence
  Synthetic oligonucleotide
tatctacacgggtctcaccaaaacgcatccaaaaattattaaacaagccacgttactttacttgcataaaacacttagagaagacgaagaaatgggcttgactaccacatctactatcatgagcttgaaaaaaacacttcggggttttagagtgagaccagcgtaactct

SEQ ID NO: 341 - DNA, 170 bp, Artificial Sequence
  Synthetic oligonucleotide
tatctacacgggtctcaccaaaacgcatccaaaaattattaaacaagccacgttactttacttgattaaaacacttagagaagacgaagaaatgggcttgactaccacatctactatcatgagcttgaaaaaaacacttcggggttttagagtgagaccagcgtaactcg

SEQ ID NO: 342 - DNA, 170 bp, Artificial Sequence
  Synthetic oligonucleotide
tatctacacgggtctcaccaaaacgcatccaaaaattattaaacaagccacgttactttacttgttgaaaacacttagagaagacgaagaaatgggcttgactaccacatctactatcatgagcttgaaaaaaacacttcggggttttagagtgagaccagcgtaactcc

SEQ ID NO: 343 - DNA, 170 bp, Artificial Sequence
  Synthetic oligonucleotide
tatctacacgggtctcaccaaaacgcatccaaaaattattaaacaagccacgttactttacttgaaaaaaacacttagagaagacgaagaaatgggcttgactaccacatctactatcatgagcttgaaaaaaacacttcggggttttagagtgagaccagcgtaactct

SEQ ID NO: 344 - DNA, 170 bp, Artificial Sequence
  Synthetic oligonucleotide
tatctacacgggtctcaccaaaacgcatccaaaaattattaaacaagccacgttactttacttgatgaaaacacttagagaagacgaagaaatgggcttgactaccacatctactatcatgagcttgaaaaaaacacttcggggttttagagtgagaccagcgtaactca

SEQ ID NO: 345 - DNA, 170 bp, Artificial Sequence
  Synthetic oligonucleotide
tatctacacgggtctcaccaaaacgcatccaaaaattattaaacaagccacgttactttacttgtttaaaacacttagagaagacgaagaaatgggcttgactaccacatctactatcatgagcttgaaaaaaacacttcggggttttagagtgagaccagcgtaactcc

SEQ ID NO: 346 - DNA, 170 bp, Artificial Sequence
  Synthetic oligonucleotide
tatctacacgggtctcaccaaaacgcatccaaaaattattaaacaagccacgttactttacttgccaaaaacacttagagaagacgaagaaatgggcttgactaccacatctactatcatgagcttgaaaaaaacacttcggggttttagagtgagaccagcgtaactcg

SEQ ID NO: 347 - DNA, 170 bp, Artificial Sequence
  Synthetic oligonucleotide
tatctacacgggtctcaccaaaacgcatccaaaaattattaaacaagccacgttactttacttgtctaaaacacttagagaagacgaagaaatgggcttgactaccacatctactatcatgagcttgaaaaaaacacttcggggttttagagtgagaccagcgtaactcc

SEQ ID NO: 348 - DNA, 170 bp, Artificial Sequence
  Synthetic oligonucleotide
tatctacacgggtctcaccaaaacgcatccaaaaattattaaacaagccacgttactttacttgactaaaacacttagagaagacgaagaaatgggcttgactaccacatctactatcatgagcttgaaaaaaacacttcggggttttagagtgagaccagcgtaactca

SEQ ID NO: 349 - DNA, 170 bp, Artificial Sequence
  Synthetic oligonucleotide
tatctacacgggtctcaccaaaacgcatccaaaaattattaaacaagccacgttactttacttgtggaaaacacttagagaagacgaagaaatgggcttgactaccacatctactatcatgagcttgaaaaaaacacttcggggttttagagtgagaccagcgtaactct

SEQ ID NO: 350 - DNA, 170 bp, Artificial Sequence
  Synthetic oligonucleotide
tatctacacgggtctcaccaaaacgcatccaaaaattattaaacaagccacgttactttacttgtataaaacacttagagaagacgaagaaatgggcttgactaccacatctactatcatgagcttgaaaaaaacacttcggggttttagagtgagaccagcgtaactcc

SEQ ID NO: 351 - DNA, 170 bp, Artificial Sequence
  Synthetic oligonucleotide
tatctacacgggtctcaccaaaacgcatccaaaaattattaaacaagccacgttactttacttggttaaaacacttagagaagacgaagaaatgggcttgactaccacatctactatcatgagcttgaaaaaaacacttcggggttttagagtgagaccagcgtaactct

SEQ ID NO: 352 - DNA, 170 bp, Artificial Sequence
  Synthetic oligonucleotide
tatctacacgggtctcaccaaaactccaaaaattattaaacaagccacgttactttacttgaaagctacacttagagaagacgaagaaatgggcttgactaccacatctactatcatgagcttgaaaaaaacacttcggggttttagagtgagaccagcgtaactcaaaa

SEQ ID NO: 353 - DNA, 170 bp, Artificial Sequence
  Synthetic oligonucleotide
tatctacacgggtctcaccaaaactccaaaaattattaaacaagccacgttactttacttgaaaagaacacttagagaagacgaagaaatgggcttgactaccacatctactatcatgagcttgaaaaaaacacttcggggttttagagtgagaccagcgtaactcacgt

SEQ ID NO: 354 - DNA, 170 bp, Artificial Sequence
  Synthetic oligonucleotide
tatctacacgggtctcaccaaaactccaaaaattattaaacaagccacgttactttacttgaaaaatacacttagagaagacgaagaaatgggcttgactaccacatctactatcatgagcttgaaaaaaacacttcggggttttagagtgagaccagcgtaactccaag

SEQ ID NO: 355 - DNA, 170 bp, Artificial Sequence
  Synthetic oligonucleotide
tatctacacgggtctcaccaaaactccaaaaattattaaacaagccacgttactttacttgaaagatacacttagagaagacgaagaaatgggcttgactaccacatctactatcatgagcttgaaaaaaacacttcggggttttagagtgagaccagcgtaactctgca

SEQ ID NO: 356 - DNA, 170 bp, Artificial Sequence
  Synthetic oligonucleotide
tatctacacgggtctcaccaaaactccaaaaattattaaacaagccacgttactttacttgaaatgtacacttagagaagacgaagaaatgggcttgactaccacatctactatcatgagcttgaaaaaaacacttcggggttttagagtgagaccagcgtaactcaaag

SEQ ID NO: 357 - DNA, 170 bp, Artificial Sequence
  Synthetic oligonucleotide
tatctacacgggtctcaccaaaactccaaaaattattaaacaagccacgttactttacttgaaacaaacacttagagaagacgaagaaatgggcttgactaccacatctactatcatgagcttgaaaaaaacacttcggggttttagagtgagaccagcgtaactcagta

SEQ ID NO: 358 - DNA, 170 bp, Artificial Sequence
  Synthetic oligonucleotide
tatctacacgggtctcaccaaaactccaaaaattattaaacaagccacgttactttacttgaaagaaacacttagagaagacgaagaaatgggcttgactaccacatctactatcatgagcttgaaaaaaacacttcggggttttagagtgagaccagcgtaactctctc

SEQ ID NO: 359 - DNA, 170 bp, Artificial Sequence
  Synthetic oligonucleotide
tatctacacgggtctcaccaaaactccaaaaattattaaacaagccacgttactttacttgaaaggtacacttagagaagacgaagaaatgggcttgactaccacatctactatcatgagcttgaaaaaaacacttcggggttttagagtgagaccagcgtaactcgcta

SEQ ID NO: 360 - DNA, 170 bp, Artificial Sequence
  Synthetic oligonucleotide
tatctacacgggtctcaccaaaactccaaaaattattaaacaagccacgttactttacttgaaacatacacttagagaagacgaagaaatgggcttgactaccacatctactatcatgagcttgaaaaaaacacttcggggttttagagtgagaccagcgtaactcaaat

SEQ ID NO: 361 - DNA, 170 bp, Artificial Sequence
  Synthetic oligonucleotide
tatctacacgggtctcaccaaaactccaaaaattattaaacaagccacgttactttacttgaaaattacacttagagaagacgaagaaatgggcttgactaccacatctactatcatgagcttgaaaaaaacacttcggggttttagagtgagaccagcgtaactcctgt

SEQ ID NO: 362 - DNA, 170 bp, Artificial Sequence
  Synthetic oligonucleotide
tatctacacgggtctcaccaaaactccaaaaattattaaacaagccacgttactttacttgaaattgacacttagagaagacgaagaaatgggcttgactaccacatctactatcatgagcttgaaaaaaacacttcggggttttagagtgagaccagcgtaactcggat

SEQ ID NO: 363 - DNA, 170 bp, Artificial Sequence
  Synthetic oligonucleotide
tatctacacgggtctcaccaaaactccaaaaattattaaacaagccacgttactttacttgaaaaaaacacttagagaagacgaagaaatgggcttgactaccacatctactatcatgagcttgaaaaaaacacttcggggttttagagtgagaccagcgtaactcttat

SEQ ID NO: 364 - DNA, 170 bp, Artificial Sequence
  Synthetic oligonucleotide
tatctacacgggtctcaccaaaactccaaaaattattaaacaagccacgttactttacttgaaaatgacacttagagaagacgaagaaatgggcttgactaccacatctactatcatgagcttgaaaaaaacacttcggggttttagagtgagaccagcgtaactctaca

SEQ ID NO: 365 - DNA, 170 bp, Artificial Sequence
  Synthetic oligonucleotide
tatctacacgggtctcaccaaaactccaaaaattattaaacaagccacgttactttacttgaaatttacacttagagaagacgaagaaatgggcttgactaccacatctactatcatgagcttgaaaaaaacacttcggggttttagagtgagaccagcgtaactcccat

SEQ ID NO: 366 - DNA, 170 bp, Artificial Sequence
  Synthetic oligonucleotide
tatctacacgggtctcaccaaaactccaaaaattattaaacaagccacgttactttacttgaaaccaacacttagagaagacgaagaaatgggcttgactaccacatctactatcatgagcttgaaaaaaacacttcggggttttagagtgagaccagcgtaactcttct

SEQ ID NO: 367 - DNA, 170 bp, Artificial Sequence
  Synthetic oligonucleotide
tatctacacgggtctcaccaaaactccaaaaattattaaacaagccacgttactttacttgaaatctacacttagagaagacgaagaaatgggcttgactaccacatctactatcatgagcttgaaaaaaacacttcggggttttagagtgagaccagcgtaactcttcg

SEQ ID NO: 368 - DNA, 170 bp, Artificial Sequence
  Synthetic oligonucleotide
tatctacacgggtctcaccaaaactccaaaaattattaaacaagccacgttactttacttgaaaactacacttagagaagacgaagaaatgggcttgactaccacatctactatcatgagcttgaaaaaaacacttcggggttttagagtgagaccagcgtaactctagc

SEQ ID NO: 369 - DNA, 170 bp, Artificial Sequence
  Synthetic oligonucleotide
tatctacacgggtctcaccaaaactccaaaaattattaaacaagccacgttactttacttgaaatggacacttagagaagacgaagaaatgggcttgactaccacatctactatcatgagcttgaaaaaaacacttcggggttttagagtgagaccagcgtaactcggat

SEQ ID NO: 370 - DNA, 170 bp, Artificial Sequence
  Synthetic oligonucleotide
tatctacacgggtctcaccaaaactccaaaaattattaaacaagccacgttactttacttgaaatatacacttagagaagacgaagaaatgggcttgactaccacatctactatcatgagcttgaaaaaaacacttcggggttttagagtgagaccagcgtaactcatat

SEQ ID NO: 371 - DNA, 170 bp, Artificial Sequence
  Synthetic oligonucleotide
tatctacacgggtctcaccaaaactccaaaaattattaaacaagccacgttactttacttgaaagttacacttagagaagacgaagaaatgggcttgactaccacatctactatcatgagcttgaaaaaaacacttcggggttttagagtgagaccagcgtaactctaaa

SEQ ID NO: 372 - DNA, 170 bp, Artificial Sequence
  Synthetic oligonucleotide
tatctacacgggtctcaccaaaacaaaaattattaaacaagccacgttactttacttgaaaaaagctcttagagaagacgaagaaatgggcttgactaccacatctactatcatgagcttgaaaaaaacacttcggggttttagagtgagaccagcgtaactccttattt

SEQ ID NO: 373 - DNA, 170 bp, Artificial Sequence
  Synthetic oligonucleotide
tatctacacgggtctcaccaaaacaaaaattattaaacaagccacgttactttacttgaaaaaaagacttagagaagacgaagaaatgggcttgactaccacatctactatcatgagcttgaaaaaaacacttcggggttttagagtgagaccagcgtaactctccacgc

SEQ ID NO: 374 - DNA, 170 bp, Artificial Sequence
  Synthetic oligonucleotide
tatctacacgggtctcaccaaaacaaaaattattaaacaagccacgttactttacttgaaaaaaaatcttagagaagacgaagaaatgggcttgactaccacatctactatcatgagcttgaaaaaaacacttcggggttttagagtgagaccagcgtaactcatttgcg

SEQ ID NO: 375 - DNA, 170 bp, Artificial Sequence
  Synthetic oligonucleotide
tatctacacgggtctcaccaaaacaaaaattattaaacaagccacgttactttacttgaaaaaagatcttagagaagacgaagaaatgggcttgactaccacatctactatcatgagcttgaaaaaaacacttcggggttttagagtgagaccagcgtaactctcagcct

SEQ ID NO: 376 - DNA, 170 bp, Artificial Sequence
  Synthetic oligonucleotide
tatctacacgggtctcaccaaaacaaaaattattaaacaagccacgttactttacttgaaaaaatgtcttagagaagacgaagaaatgggcttgactaccacatctactatcatgagcttgaaaaaaacacttcggggttttagagtgagaccagcgtaactctccagtg

SEQ ID NO: 377 - DNA, 170 bp, Artificial Sequence
  Synthetic oligonucleotide
tatctacacgggtctcaccaaaacaaaaattattaaacaagccacgttactttacttgaaaaaacaacttagagaagacgaagaaatgggcttgactaccacatctactatcatgagcttgaaaaaaacacttcggggttttagagtgagaccagcgtaactcaagcttt

SEQ ID NO: 378 - DNA, 170 bp, Artificial Sequence
  Synthetic oligonucleotide
tatctacacgggtctcaccaaaacaaaaattattaaacaagccacgttactttacttgaaaaaagaacttagagaagacgaagaaatgggcttgactaccacatctactatcatgagcttgaaaaaaacacttcggggttttagagtgagaccagcgtaactcatgtatc

SEQ ID NO: 379 - DNA, 170 bp, Artificial Sequence
  Synthetic oligonucleotide
tatctacacgggtctcaccaaaacaaaaattattaaacaagccacgttactttacttgaaaaaaggtcttagagaagacgaagaaatgggcttgactaccacatctactatcatgagcttgaaaaaaacacttcggggttttagagtgagaccagcgtaactcatgctgg

SEQ ID NO: 380 - DNA, 170 bp, Artificial Sequence
  Synthetic oligonucleotide
tatctacacgggtctcaccaaaacaaaaattattaaacaagccacgttactttacttgaaaaaacatcttagagaagacgaagaaatgggcttgactaccacatctactatcatgagcttgaaaaaaacacttcggggttttagagtgagaccagcgtaactcctcgcgg

SEQ ID NO: 381 - DNA, 170 bp, Artificial Sequence
  Synthetic oligonucleotide
tatctacacgggtctcaccaaaacaaaaattattaaacaagccacgttactttacttgaaaaaaattcttagagaagacgaagaaatgggcttgactaccacatctactatcatgagcttgaaaaaaacacttcggggttttagagtgagaccagcgtaactccttccgc

SEQ ID NO: 382 - DNA, 170 bp, Artificial Sequence
  Synthetic oligonucleotide
tatctacacgggtctcaccaaaacaaaaattattaaacaagccacgttactttacttgaaaaaattgcttagagaagacgaagaaatgggcttgactaccacatctactatcatgagcttgaaaaaaacacttcggggttttagagtgagaccagcgtaactcacgaact

SEQ ID NO: 383 - DNA, 170 bp, Artificial Sequence
  Synthetic oligonucleotide
tatctacacgggtctcaccaaaacaaaaattattaaacaagccacgttactttacttgaaaaaaaaacttagagaagacgaagaaatgggcttgactaccacatctactatcatgagcttgaaaaaaacacttcggggttttagagtgagaccagcgtaactccctcttt

SEQ ID NO: 384 - DNA, 170 bp, Artificial Sequence
  Synthetic oligonucleotide
tatctacacgggtctcaccaaaacaaaaattattaaacaagccacgttactttacttgaaaaaaatgcttagagaagacgaagaaatgggcttgactaccacatctactatcatgagcttgaaaaaaacacttcggggttttagagtgagaccagcgtaactccctaatc

SEQ ID NO: 385 - DNA, 170 bp, Artificial Sequence
  Synthetic oligonucleotide
tatctacacgggtctcaccaaaacaaaaattattaaacaagccacgttactttacttgaaaaaatttcttagagaagacgaagaaatgggcttgactaccacatctactatcatgagcttgaaaaaaacacttcggggttttagagtgagaccagcgtaactctcgcccc

SEQ ID NO: 386 - DNA, 170 bp, Artificial Sequence
  Synthetic oligonucleotide
tatctacacgggtctcaccaaaacaaaaattattaaacaagccacgttactttacttgaaaaaaccacttagagaagacgaagaaatgggcttgactaccacatctactatcatgagcttgaaaaaaacacttcggggttttagagtgagaccagcgtaactctatacga

SEQ ID NO: 387 - DNA, 170 bp, Artificial Sequence
  Synthetic oligonucleotide
tatctacacgggtctcaccaaaacaaaaattattaaacaagccacgttactttacttgaaaaaatctcttagagaagacgaagaaatgggcttgactaccacatctactatcatgagcttgaaaaaaacacttcggggttttagagtgagaccagcgtaactcgttcagg

SEQ ID NO: 388 - DNA, 170 bp, Artificial Sequence
  Synthetic oligonucleotide
tatctacacgggtctcaccaaaacaaaaattattaaacaagccacgttactttacttgaaaaaaactcttagagaagacgaagaaatgggcttgactaccacatctactatcatgagcttgaaaaaaacacttcggggttttagagtgagaccagcgtaactcgtttaca

SEQ ID NO: 389 - DNA, 170 bp, Artificial Sequence
  Synthetic oligonucleotide
tatctacacgggtctcaccaaaacaaaaattattaaacaagccacgttactttacttgaaaaaatggcttagagaagacgaagaaatgggcttgactaccacatctactatcatgagcttgaaaaaaacacttcggggttttagagtgagaccagcgtaactctcccggc

SEQ ID NO: 390 - DNA, 170 bp, Artificial Sequence
  Synthetic oligonucleotide
tatctacacgggtctcaccaaaacaaaaattattaaacaagccacgttactttacttgaaaaaatatcttagagaagacgaagaaatgggcttgactaccacatctactatcatgagcttgaaaaaaacacttcggggttttagagtgagaccagcgtaactcggattgt

SEQ ID NO: 391 - DNA, 170 bp, Artificial Sequence
  Synthetic oligonucleotide
tatctacacgggtctcaccaaaacaaaaattattaaacaagccacgttactttacttgaaaaaagttcttagagaagacgaagaaatgggcttgactaccacatctactatcatgagcttgaaaaaaacacttcggggttttagagtgagaccagcgtaactcgcgtccg

SEQ ID NO: 392 - DNA, 170 bp, Artificial Sequence
  Synthetic oligonucleotide
tatctacacgggtctcaccaaaacaattattaaacaagccacgttactttacttgaaaaaaacagctagagaagacgaagaaatgggcttgactaccacatctactatcatgagcttgaaaaaaacacttcggggttttagagtgagaccagcgtaactctccgcatgtc

SEQ ID NO: 393 - DNA, 170 bp, Artificial Sequence
  Synthetic oligonucleotide
tatctacacgggtctcaccaaaacaattattaaacaagccacgttactttacttgaaaaaaacaagaagagaagacgaagaaatgggcttgactaccacatctactatcatgagcttgaaaaaaacacttcggggttttagagtgagaccagcgtaactcctattctccg

SEQ ID NO: 394 - DNA, 170 bp, Artificial Sequence
  Synthetic oligonucleotide
tatctacacgggtctcaccaaaacaattattaaacaagccacgttactttacttgaaaaaaacaaatagagaagacgaagaaatgggcttgactaccacatctactatcatgagcttgaaaaaaacacttcggggttttagagtgagaccagcgtaactcggatgggccg

SEQ ID NO: 395 - DNA, 170 bp, Artificial Sequence
  Synthetic oligonucleotide
tatctacacgggtctcaccaaaacaattattaaacaagccacgttactttacttgaaaaaaacagatagagaagacgaagaaatgggcttgactaccacatctactatcatgagcttgaaaaaaacacttcggggttttagagtgagaccagcgtaactcgtttctctaa

SEQ ID NO: 396 - DNA, 170 bp, Artificial Sequence
  Synthetic oligonucleotide
tatctacacgggtctcaccaaaacaattattaaacaagccacgttactttacttgaaaaaaacatgtagagaagacgaagaaatgggcttgactaccacatctactatcatgagcttgaaaaaaacacttcggggttttagagtgagaccagcgtaactcacttttggcg

SEQ ID NO: 397 - DNA, 170 bp, Artificial Sequence
  Synthetic oligonucleotide
tatctacacgggtctcaccaaaacaattattaaacaagccacgttactttacttgaaaaaaacacaaagagaagacgaagaaatgggcttgactaccacatctactatcatgagcttgaaaaaaacacttcggggttttagagtgagaccagcgtaactcttgagctggt

SEQ ID NO: 398 - DNA, 170 bp, Artificial Sequence
  Synthetic oligonucleotide
tatctacacgggtctcaccaaaacaattattaaacaagccacgttactttacttgaaaaaaacagaaagagaagacgaagaaatgggcttgactaccacatctactatcatgagcttgaaaaaaacacttcggggttttagagtgagaccagcgtaactccgaggttatt

SEQ ID NO: 399 - DNA, 170 bp, Artificial Sequence
  Synthetic oligonucleotide
tatctacacgggtctcaccaaaacaattattaaacaagccacgttactttacttgaaaaaaacaggtagagaagacgaagaaatgggcttgactaccacatctactatcatgagcttgaaaaaaacacttcggggttttagagtgagaccagcgtaactctagggggtgt

SEQ ID NO: 400 - DNA, 170 bp, Artificial Sequence
  Synthetic oligonucleotide
tatctacacgggtctcaccaaaacaattattaaacaagccacgttactttacttgaaaaaaacacatagagaagacgaagaaatgggcttgactaccacatctactatcatgagcttgaaaaaaacacttcggggttttagagtgagaccagcgtaactctccaacgttc

SEQ ID NO: 401 - DNA, 170 bp, Artificial Sequence
  Synthetic oligonucleotide
tatctacacgggtctcaccaaaacaattattaaacaagccacgttactttacttgaaaaaaacaattagagaagacgaagaaatgggcttgactaccacatctactatcatgagcttgaaaaaaacacttcggggttttagagtgagaccagcgtaactcgtgaacacgg

SEQ ID NO: 402 - DNA, 170 bp, Artificial Sequence
  Synthetic oligonucleotide
tatctacacgggtctcaccaaaacaattattaaacaagccacgttactttacttgaaaaaaacattgagagaagacgaagaaatgggcttgactaccacatctactatcatgagcttgaaaaaaacacttcggggttttagagtgagaccagcgtaactctctaaaagat

SEQ ID NO: 403 - DNA, 170 bp, Artificial Sequence
  Synthetic oligonucleotide
tatctacacgggtctcaccaaaacaattattaaacaagccacgttactttacttgaaaaaaacaaaaagagaagacgaagaaatgggcttgactaccacatctactatcatgagcttgaaaaaaacacttcggggttttagagtgagaccagcgtaactcgcctccgagc

SEQ ID NO: 404 - DNA, 170 bp, Artificial Sequence
  Synthetic oligonucleotide
tatctacacgggtctcaccaaaacaattattaaacaagccacgttactttacttgaaaaaaacaatgagagaagacgaagaaatgggcttgactaccacatctactatcatgagcttgaaaaaaacacttcggggttttagagtgagaccagcgtaactccctaaggcgc

SEQ ID NO: 405 - DNA, 170 bp, Artificial Sequence
  Synthetic oligonucleotide
tatctacacgggtctcaccaaaacaattattaaacaagccacgttactttacttgaaaaaaacatttagagaagacgaagaaatgggcttgactaccacatctactatcatgagcttgaaaaaaacacttcggggttttagagtgagaccagcgtaactcgtcaactgac

SEQ ID NO: 406 - DNA, 170 bp, Artificial Sequence
  Synthetic oligonucleotide
tatctacacgggtctcaccaaaacaattattaaacaagccacgttactttacttgaaaaaaacaccaagagaagacgaagaaatgggcttgactaccacatctactatcatgagcttgaaaaaaacacttcggggttttagagtgagaccagcgtaactcggtatatccc

SEQ ID NO: 407 - DNA, 170 bp, Artificial Sequence
  Synthetic oligonucleotide
tatctacacgggtctcaccaaaacaattattaaacaagccacgttactttacttgaaaaaaacatctagagaagacgaagaaatgggcttgactaccacatctactatcatgagcttgaaaaaaacacttcggggttttagagtgagaccagcgtaactcccgttgtgtc

SEQ ID NO: 408 - DNA, 170 bp, Artificial Sequence
  Synthetic oligonucleotide
tatctacacgggtctcaccaaaacaattattaaacaagccacgttactttacttgaaaaaaacaactagagaagacgaagaaatgggcttgactaccacatctactatcatgagcttgaaaaaaacacttcggggttttagagtgagaccagcgtaactcggaccttaac

SEQ ID NO: 409 - DNA, 170 bp, Artificial Sequence
  Synthetic oligonucleotide
tatctacacgggtctcaccaaaacaattattaaacaagccacgttactttacttgaaaaaaacatggagagaagacgaagaaatgggcttgactaccacatctactatcatgagcttgaaaaaaacacttcggggttttagagtgagaccagcgtaactcttatgcctgc

SEQ ID NO: 410 - DNA, 170 bp, Artificial Sequence
  Synthetic oligonucleotide
tatctacacgggtctcaccaaaacaattattaaacaagccacgttactttacttgaaaaaaacatatagagaagacgaagaaatgggcttgactaccacatctactatcatgagcttgaaaaaaacacttcggggttttagagtgagaccagcgtaactcagcgagatag

SEQ ID NO: 411 - DNA, 170 bp, Artificial Sequence
  Synthetic oligonucleotide
tatctacacgggtctcaccaaaacaattattaaacaagccacgttactttacttgaaaaaaacagttagagaagacgaagaaatgggcttgactaccacatctactatcatgagcttgaaaaaaacacttcggggttttagagtgagaccagcgtaactccttcgatgga

SEQ ID NO: 412 - DNA, 170 bp, Artificial Sequence
  Synthetic oligonucleotide
tatctacacgggtctcaccaaaactattaaacaagccacgttactttacttgaaaaaaacacttgctgaggatgaagagatggggttgactaccacatctactatcatgagtctgcaatgtccacttcgggaggatgaagaaagttttagagtgagaccagcgtaactcc

SEQ ID NO: 413 - DNA, 170 bp, Artificial Sequence
  Synthetic oligonucleotide
tatctacacgggtctcaccaaaactattaaacaagccacgttactttacttgaaaaaaacacttagagaggatgaagagatggggttgactaccacatctactatcatgagtctgcaatgtccacttcgggaggatgaagaaagttttagagtgagaccagcgtaactct

SEQ ID NO: 414 - DNA, 170 bp, Artificial Sequence
  Synthetic oligonucleotide
tatctacacgggtctcaccaaaactattaaacaagccacgttactttacttgaaaaaaacacttaatgaggatgaagagatggggttgactaccacatctactatcatgagtctgcaatgtccacttcgggaggatgaagaaagttttagagtgagaccagcgtaactcc

SEQ ID NO: 415 - DNA, 170 bp, Artificial Sequence
  Synthetic oligonucleotide
tatctacacgggtctcaccaaaactattaaacaagccacgttactttacttgaaaaaaacacttgatgaggatgaagagatggggttgactaccacatctactatcatgagtctgcaatgtccacttcgggaggatgaagaaagttttagagtgagaccagcgtaactcc

SEQ ID NO: 416 - DNA, 170 bp, Artificial Sequence
  Synthetic oligonucleotide
tatctacacgggtctcaccaaaactattaaacaagccacgttactttacttgaaaaaaacactttgtgaggatgaagagatggggttgactaccacatctactatcatgagtctgcaatgtccacttcgggaggatgaagaaagttttagagtgagaccagcgtaactcc

SEQ ID NO: 417 - DNA, 170 bp, Artificial Sequence
  Synthetic oligonucleotide
tatctacacgggtctcaccaaaactattaaacaagccacgttactttacttgaaaaaaacacttcaagaggatgaagagatggggttgactaccacatctactatcatgagtctgcaatgtccacttcgggaggatgaagaaagttttagagtgagaccagcgtaactct

SEQ ID NO: 418 - DNA, 170 bp, Artificial Sequence
  Synthetic oligonucleotide
tatctacacgggtctcaccaaaactattaaacaagccacgttactttacttgaaaaaaacacttgaagaggatgaagagatggggttgactaccacatctactatcatgagtctgcaatgtccacttcgggaggatgaagaaagttttagagtgagaccagcgtaactct

SEQ ID NO: 419 - DNA, 170 bp, Artificial Sequence
  Synthetic oligonucleotide
tatctacacgggtctcaccaaaactattaaacaagccacgttactttacttgaaaaaaacacttggtgaggatgaagagatggggttgactaccacatctactatcatgagtctgcaatgtccacttcgggaggatgaagaaagttttagagtgagaccagcgtaactca

SEQ ID NO: 420 - DNA, 170 bp, Artificial Sequence
  Synthetic oligonucleotide
tatctacacgggtctcaccaaaactattaaacaagccacgttactttacttgaaaaaaacacttcatgaggatgaagagatggggttgactaccacatctactatcatgagtctgcaatgtccacttcgggaggatgaagaaagttttagagtgagaccagcgtaactcg

SEQ ID NO: 421 - DNA, 170 bp, Artificial Sequence
  Synthetic oligonucleotide
tatctacacgggtctcaccaaaactattaaacaagccacgttactttacttgaaaaaaacacttattgaggatgaagagatggggttgactaccacatctactatcatgagtctgcaatgtccacttcgggaggatgaagaaagttttagagtgagaccagcgtaactcg

SEQ ID NO: 422 - DNA, 170 bp, Artificial Sequence
  Synthetic oligonucleotide
tatctacacgggtctcaccaaaactattaaacaagccacgttactttacttgaaaaaaacacttttggaggatgaagagatggggttgactaccacatctactatcatgagtctgcaatgtccacttcgggaggatgaagaaagttttagagtgagaccagcgtaactct

SEQ ID NO: 423 - DNA, 170 bp, Artificial Sequence
  Synthetic oligonucleotide
tatctacacgggtctcaccaaaactattaaacaagccacgttactttacttgaaaaaaacacttaaagaggatgaagagatggggttgactaccacatctactatcatgagtctgcaatgtccacttcgggaggatgaagaaagttttagagtgagaccagcgtaactcc

SEQ ID NO: 424 - DNA, 170 bp, Artificial Sequence
  Synthetic oligonucleotide
tatctacacgggtctcaccaaaactattaaacaagccacgttactttacttgaaaaaaacacttatggaggatgaagagatggggttgactaccacatctactatcatgagtctgcaatgtccacttcgggaggatgaagaaagttttagagtgagaccagcgtaactcg

SEQ ID NO: 425 - DNA, 170 bp, Artificial Sequence
  Synthetic oligonucleotide
tatctacacgggtctcaccaaaactattaaacaagccacgttactttacttgaaaaaaacacttttcgaggatgaagagatggggttgactaccacatctactatcatgagtctgcaatgtccacttcgggaggatgaagaaagttttagagtgagaccagcgtaactcg

SEQ ID NO: 426 - DNA, 170 bp, Artificial Sequence
  Synthetic oligonucleotide
tatctacacgggtctcaccaaaactattaaacaagccacgttactttacttgaaaaaaacacttccagaggatgaagagatggggttgactaccacatctactatcatgagtctgcaatgtccacttcgggaggatgaagaaagttttagagtgagaccagcgtaactca

SEQ ID NO: 427 - DNA, 170 bp, Artificial Sequence
  Synthetic oligonucleotide
tatctacacgggtctcaccaaaactattaaacaagccacgttactttacttgaaaaaaacactttctgaggatgaagagatggggttgactaccacatctactatcatgagtctgcaatgtccacttcgggaggatgaagaaagttttagagtgagaccagcgtaactca

SEQ ID NO: 428 - DNA, 170 bp, Artificial Sequence
  Synthetic oligonucleotide
tatctacacgggtctcaccaaaactattaaacaagccacgttactttacttgaaaaaaacacttactgaggatgaagagatggggttgactaccacatctactatcatgagtctgcaatgtccacttcgggaggatgaagaaagttttagagtgagaccagcgtaactca

SEQ ID NO: 429 - DNA, 170 bp, Artificial Sequence
  Synthetic oligonucleotide
tatctacacgggtctcaccaaaactattaaacaagccacgttactttacttgaaaaaaacactttgggaggatgaagagatggggttgactaccacatctactatcatgagtctgcaatgtccacttcgggaggatgaagaaagttttagagtgagaccagcgtaactca

SEQ ID NO: 430 - DNA, 170 bp, Artificial Sequence
  Synthetic oligonucleotide
tatctacacgggtctcaccaaaactattaaacaagccacgttactttacttgaaaaaaacactttatgaggatgaagagatggggttgactaccacatctactatcatgagtctgcaatgtccacttcgggaggatgaagaaagttttagagtgagaccagcgtaactcc

SEQ ID NO: 431 - DNA, 170 bp, Artificial Sequence
  Synthetic oligonucleotide
tatctacacgggtctcaccaaaactattaaacaagccacgttactttacttgaaaaaaacacttgttgaggatgaagagatggggttgactaccacatctactatcatgagtctgcaatgtccacttcgggaggatgaagaaagttttagagtgagaccagcgtaactct

SEQ ID NO: 432 - DNA, 170 bp, Artificial Sequence
  Synthetic oligonucleotide
tatctacacgggtctcaccaaaactaaacaagccacgttactttacttgaaaaaaacacttcgggctgatgaagagatggggttgactaccacatctactatcatgagtctgcaatgtccacttcgggaggatgaagaaagttttagagtgagaccagcgtaactctcaa

SEQ ID NO: 433 - DNA, 170 bp, Artificial Sequence
  Synthetic oligonucleotide
tatctacacgggtctcaccaaaactaaacaagccacgttactttacttgaaaaaaacacttcggagagatgaagagatggggttgactaccacatctactatcatgagtctgcaatgtccacttcgggaggatgaagaaagttttagagtgagaccagcgtaactctgta

SEQ ID NO: 434 - DNA, 170 bp, Artificial Sequence
  Synthetic oligonucleotide
tatctacacgggtctcaccaaaactaaacaagccacgttactttacttgaaaaaaacacttcggaatgatgaagagatggggttgactaccacatctactatcatgagtctgcaatgtccacttcgggaggatgaagaaagttttagagtgagaccagcgtaactcgttc

SEQ ID NO: 435 - DNA, 170 bp, Artificial Sequence
  Synthetic oligonucleotide
tatctacacgggtctcaccaaaactaaacaagccacgttactttacttgaaaaaaacacttcgggatgatgaagagatggggttgactaccacatctactatcatgagtctgcaatgtccacttcgggaggatgaagaaagttttagagtgagaccagcgtaactctgtg

SEQ ID NO: 436 - DNA, 170 bp, Artificial Sequence
  Synthetic oligonucleotide
tatctacacgggtctcaccaaaactaaacaagccacgttactttacttgaaaaaaacacttcggtgtgatgaagagatggggttgactaccacatctactatcatgagtctgcaatgtccacttcgggaggatgaagaaagttttagagtgagaccagcgtaactcgtct

SEQ ID NO: 437 - DNA, 170 bp, Artificial Sequence
  Synthetic oligonucleotide
tatctacacgggtctcaccaaaactaaacaagccacgttactttacttgaaaaaaacacttcggcaagatgaagagatggggttgactaccacatctactatcatgagtctgcaatgtccacttcgggaggatgaagaaagttttagagtgagaccagcgtaactctctc

SEQ ID NO: 438 - DNA, 170 bp, Artificial Sequence
  Synthetic oligonucleotide
tatctacacgggtctcaccaaaactaaacaagccacgttactttacttgaaaaaaacacttcgggaagatgaagagatggggttgactaccacatctactatcatgagtctgcaatgtccacttcgggaggatgaagaaagttttagagtgagaccagcgtaactcaacg

SEQ ID NO: 439 - DNA, 170 bp, Artificial Sequence
  Synthetic oligonucleotide
tatctacacgggtctcaccaaaactaaacaagccacgttactttacttgaaaaaaacacttcggggtgatgaagagatggggttgactaccacatctactatcatgagtctgcaatgtccacttcgggaggatgaagaaagttttagagtgagaccagcgtaactcaacg

SEQ ID NO: 440 - DNA, 170 bp, Artificial Sequence
  Synthetic oligonucleotide
tatctacacgggtctcaccaaaactaaacaagccacgttactttacttgaaaaaaacacttcggcatgatgaagagatggggttgactaccacatctactatcatgagtctgcaatgtccacttcgggaggatgaagaaagttttagagtgagaccagcgtaactccaga

SEQ ID NO: 441 - DNA, 170 bp, Artificial Sequence
  Synthetic oligonucleotide
tatctacacgggtctcaccaaaactaaacaagccacgttactttacttgaaaaaaacacttcggattgatgaagagatggggttgactaccacatctactatcatgagtctgcaatgtccacttcgggaggatgaagaaagttttagagtgagaccagcgtaactcacct

SEQ ID NO: 442 - DNA, 170 bp, Artificial Sequence
  Synthetic oligonucleotide
tatctacacgggtctcaccaaaactaaacaagccacgttactttacttgaaaaaaacacttcggttggatgaagagatggggttgactaccacatctactatcatgagtctgcaatgtccacttcgggaggatgaagaaagttttagagtgagaccagcgtaactcattg

SEQ ID NO: 443 - DNA, 170 bp, Artificial Sequence
  Synthetic oligonucleotide
tatctacacgggtctcaccaaaactaaacaagccacgttactttacttgaaaaaaacacttcggaaagatgaagagatggggttgactaccacatctactatcatgagtctgcaatgtccacttcgggaggatgaagaaagttttagagtgagaccagcgtaactctcct

SEQ ID NO: 444 - DNA, 170 bp, Artificial Sequence
  Synthetic oligonucleotide
tatctacacgggtctcaccaaaactaaacaagccacgttactttacttgaaaaaaacacttcggatggatgaagagatggggttgactaccacatctactatcatgagtctgcaatgtccacttcgggaggatgaagaaagttttagagtgagaccagcgtaactctccc

SEQ ID NO: 445 - DNA, 170 bp, Artificial Sequence
  Synthetic oligonucleotide
tatctacacgggtctcaccaaaactaaacaagccacgttactttacttgaaaaaaacacttcggtttgatgaagagatggggttgactaccacatctactatcatgagtctgcaatgtccacttcgggaggatgaagaaagttttagagtgagaccagcgtaactctagg

SEQ ID NO: 446 - DNA, 170 bp, Artificial Sequence
  Synthetic oligonucleotide
tatctacacgggtctcaccaaaactaaacaagccacgttactttacttgaaaaaaacacttcggccagatgaagagatggggttgactaccacatctactatcatgagtctgcaatgtccacttcgggaggatgaagaaagttttagagtgagaccagcgtaactctttc

SEQ ID NO: 447 - DNA, 170 bp, Artificial Sequence
  Synthetic oligonucleotide
tatctacacgggtctcaccaaaactaaacaagccacgttactttacttgaaaaaaacacttcggtctgatgaagagatggggttgactaccacatctactatcatgagtctgcaatgtccacttcgggaggatgaagaaagttttagagtgagaccagcgtaactccctg

SEQ ID NO: 448 - DNA, 170 bp, Artificial Sequence
  Synthetic oligonucleotide
tatctacacgggtctcaccaaaactaaacaagccacgttactttacttgaaaaaaacacttcggactgatgaagagatggggttgactaccacatctactatcatgagtctgcaatgtccacttcgggaggatgaagaaagttttagagtgagaccagcgtaactcgttt

SEQ ID NO: 449 - DNA, 170 bp, Artificial Sequence
  Synthetic oligonucleotide
tatctacacgggtctcaccaaaactaaacaagccacgttactttacttgaaaaaaacacttcggtgggatgaagagatggggttgactaccacatctactatcatgagtctgcaatgtccacttcgggaggatgaagaaagttttagagtgagaccagcgtaactcaggc

SEQ ID NO: 450 - DNA, 170 bp, Artificial Sequence
  Synthetic oligonucleotide
tatctacacgggtctcaccaaaactaaacaagccacgttactttacttgaaaaaaacacttcggtatgatgaagagatggggttgactaccacatctactatcatgagtctgcaatgtccacttcgggaggatgaagaaagttttagagtgagaccagcgtaactccata

SEQ ID NO: 451 - DNA, 170 bp, Artificial Sequence
  Synthetic oligonucleotide
tatctacacgggtctcaccaaaactaaacaagccacgttactttacttgaaaaaaacacttcgggttgatgaagagatggggttgactaccacatctactatcatgagtctgcaatgtccacttcgggaggatgaagaaagttttagagtgagaccagcgtaactctttt

SEQ ID NO: 452 - DNA, 170 bp, Artificial Sequence
  Synthetic oligonucleotide
tatctacacgggtctcaccaaaacacaagccacgttactttacttgaaaaaaacacttcgggaggctgaagagatggggttgactaccacatctactatcatgagtctgcaatgtccacttcgggaggatgaagaaagttttagagtgagaccagcgtaactcggcgctc

SEQ ID NO: 453 - DNA, 170 bp, Artificial Sequence
  Synthetic oligonucleotide
tatctacacgggtctcaccaaaacacaagccacgttactttacttgaaaaaaacacttcgggagagagaagagatggggttgactaccacatctactatcatgagtctgcaatgtccacttcgggaggatgaagaaagttttagagtgagaccagcgtaactcccagctc

SEQ ID NO: 454 - DNA, 170 bp, Artificial Sequence
  Synthetic oligonucleotide
tatctacacgggtctcaccaaaacacaagccacgttactttacttgaaaaaaacacttcgggagaatgaagagatggggttgactaccacatctactatcatgagtctgcaatgtccacttcgggaggatgaagaaagttttagagtgagaccagcgtaactcggaaagc

SEQ ID NO: 455 - DNA, 170 bp, Artificial Sequence
  Synthetic oligonucleotide
tatctacacgggtctcaccaaaacacaagccacgttactttacttgaaaaaaacacttcgggaggatgaagagatggggttgactaccacatctactatcatgagtctgcaatgtccacttcgggaggatgaagaaagttttagagtgagaccagcgtaactccacattc

SEQ ID NO: 456 - DNA, 170 bp, Artificial Sequence
  Synthetic oligonucleotide
tatctacacgggtctcaccaaaacacaagccacgttactttacttgaaaaaaacacttcgggagtgtgaagagatggggttgactaccacatctactatcatgagtctgcaatgtccacttcgggaggatgaagaaagttttagagtgagaccagcgtaactcgtatgct

SEQ ID NO: 457 - DNA, 170 bp, Artificial Sequence
  Synthetic oligonucleotide
tatctacacgggtctcaccaaaacacaagccacgttactttacttgaaaaaaacacttcgggagcaagaagagatggggttgactaccacatctactatcatgagtctgcaatgtccacttcgggaggatgaagaaagttttagagtgagaccagcgtaactccactatc

SEQ ID NO: 458 - DNA, 170 bp, Artificial Sequence
  Synthetic oligonucleotide
tatctacacgggtctcaccaaaacacaagccacgttactttacttgaaaaaaacacttcgggaggaagaagagatggggttgactaccacatctactatcatgagtctgcaatgtccacttcgggaggatgaagaaagttttagagtgagaccagcgtaactctgcgtac

SEQ ID NO: 459 - DNA, 170 bp, Artificial Sequence
  Synthetic oligonucleotide
tatctacacgggtctcaccaaaacacaagccacgttactttacttgaaaaaaacacttcgggagggtgaagagatggggttgactaccacatctactatcatgagtctgcaatgtccacttcgggaggatgaagaaagttttagagtgagaccagcgtaactctgaggag

SEQ ID NO: 460 - DNA, 170 bp, Artificial Sequence
  Synthetic oligonucleotide
tatctacacgggtctcaccaaaacacaagccacgttactttacttgaaaaaaacacttcgggagcatgaagagatggggttgactaccacatctactatcatgagtctgcaatgtccacttcgggaggatgaagaaagttttagagtgagaccagcgtaactcgtggtgg

SEQ ID NO: 461 - DNA, 170 bp, Artificial Sequence
  Synthetic oligonucleotide
tatctacacgggtctcaccaaaacacaagccacgttactttacttgaaaaaaacacttcgggagattgaagagatggggttgactaccacatctactatcatgagtctgcaatgtccacttcgggaggatgaagaaagttttagagtgagaccagcgtaactccgtagta

SEQ ID NO: 462 - DNA, 170 bp, Artificial Sequence
  Synthetic oligonucleotide
tatctacacgggtctcaccaaaacacaagccacgttactttacttgaaaaaaacacttcgggagttggaagagatggggttgactaccacatctactatcatgagtctgcaatgtccacttcgggaggatgaagaaagttttagagtgagaccagcgtaactctcgacct

SEQ ID NO: 463 - DNA, 170 bp, Artificial Sequence
  Synthetic oligonucleotide
tatctacacgggtctcaccaaaacacaagccacgttactttacttgaaaaaaacacttcgggagaaagaagagatggggttgactaccacatctactatcatgagtctgcaatgtccacttcgggaggatgaagaaagttttagagtgagaccagcgtaactcgttatgg

SEQ ID NO: 464 - DNA, 170 bp, Artificial Sequence
  Synthetic oligonucleotide
tatctacacgggtctcaccaaaacacaagccacgttactttacttgaaaaaaacacttcgggagatggaagagatggggttgactaccacatctactatcatgagtctgcaatgtccacttcgggaggatgaagaaagttttagagtgagaccagcgtaactcatcatga

SEQ ID NO: 465 - DNA, 170 bp, Artificial Sequence
  Synthetic oligonucleotide
tatctacacgggtctcaccaaaacacaagccacgttactttacttgaaaaaaacacttcgggagtttgaagagatggggttgactaccacatctactatcatgagtctgcaatgtccacttcgggaggatgaagaaagttttagagtgagaccagcgtaactccattcat

SEQ ID NO: 466 - DNA, 170 bp, Artificial Sequence
  Synthetic oligonucleotide
tatctacacgggtctcaccaaaacacaagccacgttactttacttgaaaaaaacacttcgggagccagaagagatggggttgactaccacatctactatcatgagtctgcaatgtccacttcgggaggatgaagaaagttttagagtgagaccagcgtaactcacaacag

SEQ ID NO: 467 - DNA, 170 bp, Artificial Sequence
  Synthetic oligonucleotide
tatctacacgggtctcaccaaaacacaagccacgttactttacttgaaaaaaacacttcgggagtctgaagagatggggttgactaccacatctactatcatgagtctgcaatgtccacttcgggaggatgaagaaagttttagagtgagaccagcgtaactcatatcat

SEQ ID NO: 468 - DNA, 170 bp, Artificial Sequence
  Synthetic oligonucleotide
tatctacacgggtctcaccaaaacacaagccacgttactttacttgaaaaaaacacttcgggagactgaagagatggggttgactaccacatctactatcatgagtctgcaatgtccacttcgggaggatgaagaaagttttagagtgagaccagcgtaactcagactca

SEQ ID NO: 469 - DNA, 170 bp, Artificial Sequence
  Synthetic oligonucleotide
tatctacacgggtctcaccaaaacacaagccacgttactttacttgaaaaaaacacttcgggagtgggaagagatggggttgactaccacatctactatcatgagtctgcaatgtccacttcgggaggatgaagaaagttttagagtgagaccagcgtaactcgcggaga

SEQ ID NO: 470 - DNA, 170 bp, Artificial Sequence
  Synthetic oligonucleotide
tatctacacgggtctcaccaaaacacaagccacgttactttacttgaaaaaaacacttcgggagtatgaagagatggggttgactaccacatctactatcatgagtctgcaatgtccacttcgggaggatgaagaaagttttagagtgagaccagcgtaactcctggagg

SEQ ID NO: 471 - DNA, 170 bp, Artificial Sequence
  Synthetic oligonucleotide
tatctacacgggtctcaccaaaacacaagccacgttactttacttgaaaaaaacacttcgggaggttgaagagatggggttgactaccacatctactatcatgagtctgcaatgtccacttcgggaggatgaagaaagttttagagtgagaccagcgtaactccatgaca

SEQ ID NO: 472 - DNA, 170 bp, Artificial Sequence
  Synthetic oligonucleotide
tatctacacgggtctcaccaaaacagccacgttactttacttgaaaaaaacacttcgggaggatgctgagatggggttgactaccacatctactatcatgagtctgcaatgtccacttcgggaggatgaagaaagttttagagtgagaccagcgtaactcacccaccggg

SEQ ID NO: 473 - DNA, 170 bp, Artificial Sequence
  Synthetic oligonucleotide
tatctacacgggtctcaccaaaacagccacgttactttacttgaaaaaaacacttcgggaggatagagagatggggttgactaccacatctactatcatgagtctgcaatgtccacttcgggaggatgaagaaagttttagagtgagaccagcgtaactcgcccatgact

SEQ ID NO: 474 - DNA, 170 bp, Artificial Sequence
  Synthetic oligonucleotide
tatctacacgggtctcaccaaaacagccacgttactttacttgaaaaaaacacttcgggaggataatgagatggggttgactaccacatctactatcatgagtctgcaatgtccacttcgggaggatgaagaaagttttagagtgagaccagcgtaactccctcctgcgt

SEQ ID NO: 475 - DNA, 170 bp, Artificial Sequence
  Synthetic oligonucleotide
tatctacacgggtctcaccaaaacagccacgttactttacttgaaaaaaacacttcgggaggatgatgagatggggttgactaccacatctactatcatgagtctgcaatgtccacttcgggaggatgaagaaagttttagagtgagaccagcgtaactcgtccctatgc

SEQ ID NO: 476 - DNA, 170 bp, Artificial Sequence
  Synthetic oligonucleotide
tatctacacgggtctcaccaaaacagccacgttactttacttgaaaaaaacacttcgggaggattgtgagatggggttgactaccacatctactatcatgagtctgcaatgtccacttcgggaggatgaagaaagttttagagtgagaccagcgtaactctggtagtcta

SEQ ID NO: 477 - DNA, 170 bp, Artificial Sequence
  Synthetic oligonucleotide
tatctacacgggtctcaccaaaacagccacgttactttacttgaaaaaaacacttcgggaggatcaagagatggggttgactaccacatctactatcatgagtctgcaatgtccacttcgggaggatgaagaaagttttagagtgagaccagcgtaactctagaaaagtc

SEQ ID NO: 478 - DNA, 170 bp, Artificial Sequence
  Synthetic oligonucleotide
tatctacacgggtctcaccaaaacagccacgttactttacttgaaaaaaacacttcgggaggatgaagagatggggttgactaccacatctactatcatgagtctgcaatgtccacttcgggaggatgaagaaagttttagagtgagaccagcgtaactcgtacacagaa

SEQ ID NO: 479 - DNA, 170 bp, Artificial Sequence
  Synthetic oligonucleotide
tatctacacgggtctcaccaaaacagccacgttactttacttgaaaaaaacacttcgggaggatggtgagatggggttgactaccacatctactatcatgagtctgcaatgtccacttcgggaggatgaagaaagttttagagtgagaccagcgtaactcgacctccctg

SEQ ID NO: 480 - DNA, 170 bp, Artificial Sequence
  Synthetic oligonucleotide
tatctacacgggtctcaccaaaacagccacgttactttacttgaaaaaaacacttcgggaggatcatgagatggggttgactaccacatctactatcatgagtctgcaatgtccacttcgggaggatgaagaaagttttagagtgagaccagcgtaactcaccacgttat

SEQ ID NO: 481 - DNA, 170 bp, Artificial Sequence
  Synthetic oligonucleotide
tatctacacgggtctcaccaaaacagccacgttactttacttgaaaaaaacacttcgggaggatattgagatggggttgactaccacatctactatcatgagtctgcaatgtccacttcgggaggatgaagaaagttttagagtgagaccagcgtaactcattgcgggcc

SEQ ID NO: 482 - DNA, 170 bp, Artificial Sequence
  Synthetic oligonucleotide
tatctacacgggtctcaccaaaacagccacgttactttacttgaaaaaaacacttcgggaggatttggagatggggttgactaccacatctactatcatgagtctgcaatgtccacttcgggaggatgaagaaagttttagagtgagaccagcgtaactctataaccgaa

SEQ ID NO: 483 - DNA, 170 bp, Artificial Sequence
  Synthetic oligonucleotide
tatctacacgggtctcaccaaaacagccacgttactttacttgaaaaaaacacttcgggaggataaagagatggggttgactaccacatctactatcatgagtctgcaatgtccacttcgggaggatgaagaaagttttagagtgagaccagcgtaactcatcagggtcc

SEQ ID NO: 484 - DNA, 170 bp, Artificial Sequence
  Synthetic oligonucleotide
tatctacacgggtctcaccaaaacagccacgttactttacttgaaaaaaacacttcgggaggatatggagatggggttgactaccacatctactatcatgagtctgcaatgtccacttcgggaggatgaagaaagttttagagtgagaccagcgtaactccgagaacgta

SEQ ID NO: 485 - DNA, 170 bp, Artificial Sequence
  Synthetic oligonucleotide
tatctacacgggtctcaccaaaacagccacgttactttacttgaaaaaaacacttcgggaggattttgagatggggttgactaccacatctactatcatgagtctgcaatgtccacttcgggaggatgaagaaagttttagagtgagaccagcgtaactctccatcattg

SEQ ID NO: 486 - DNA, 170 bp, Artificial Sequence
  Synthetic oligonucleotide
tatctacacgggtctcaccaaaacagccacgttactttacttgaaaaaaacacttcgggaggatccagagatggggttgactaccacatctactatcatgagtctgcaatgtccacttcgggaggatgaagaaagttttagagtgagaccagcgtaactccgcacggggt

SEQ ID NO: 487 - DNA, 170 bp, Artificial Sequence
  Synthetic oligonucleotide
tatctacacgggtctcaccaaaacagccacgttactttacttgaaaaaaacacttcgggaggattctgagatggggttgactaccacatctactatcatgagtctgcaatgtccacttcgggaggatgaagaaagttttagagtgagaccagcgtaactcaagtatcaac

SEQ ID NO: 488 - DNA, 170 bp, Artificial Sequence
  Synthetic oligonucleotide
tatctacacgggtctcaccaaaacagccacgttactttacttgaaaaaaacacttcgggaggatactgagatggggttgactaccacatctactatcatgagtctgcaatgtccacttcgggaggatgaagaaagttttagagtgagaccagcgtaactcgggcttacaa

SEQ ID NO: 489 - DNA, 170 bp, Artificial Sequence
  Synthetic oligonucleotide
tatctacacgggtctcaccaaaacagccacgttactttacttgaaaaaaacacttcgggaggattgggagatggggttgactaccacatctactatcatgagtctgcaatgtccacttcgggaggatgaagaaagttttagagtgagaccagcgtaactcaatttgagta

SEQ ID NO: 490 - DNA, 170 bp, Artificial Sequence
  Synthetic oligonucleotide
tatctacacgggtctcaccaaaacagccacgttactttacttgaaaaaaacacttcgggaggattatgagatggggttgactaccacatctactatcatgagtctgcaatgtccacttcgggaggatgaagaaagttttagagtgagaccagcgtaactctcagtacata

SEQ ID NO: 491 - DNA, 170 bp, Artificial Sequence
  Synthetic oligonucleotide
tatctacacgggtctcaccaaaacagccacgttactttacttgaaaaaaacacttcgggaggatgttgagatggggttgactaccacatctactatcatgagtctgcaatgtccacttcgggaggatgaagaaagttttagagtgagaccagcgtaactccactcagtct

SEQ ID NO: 492 - DNA, 170 bp, Artificial Sequence
  Synthetic oligonucleotide
tatctacacgggtctcaccaaaaccacgttactttacttgaaaaaaacacttcgggaggatgaagcgatggggttgactaccacatctactatcatgagtctgcaatgtccacttcgggaggatgaagaaagttttagagtgagaccagcgtaactctagtgactatgct

SEQ ID NO: 493 - DNA, 170 bp, Artificial Sequence
  Synthetic oligonucleotide
tatctacacgggtctcaccaaaaccacgttactttacttgaaaaaaacacttcgggaggatgaacggatggggttgactaccacatctactatcatgagtctgcaatgtccacttcgggaggatgaagaaagttttagagtgagaccagcgtaactccgtttccaccgta

SEQ ID NO: 494 - DNA, 170 bp, Artificial Sequence
  Synthetic oligonucleotide
tatctacacgggtctcaccaaaaccacgttactttacttgaaaaaaacacttcgggaggatgaaaacatggggttgactaccacatctactatcatgagtctgcaatgtccacttcgggaggatgaagaaagttttagagtgagaccagcgtaactcttaccaacaacca

SEQ ID NO: 495 - DNA, 170 bp, Artificial Sequence
  Synthetic oligonucleotide
tatctacacgggtctcaccaaaaccacgttactttacttgaaaaaaacacttcgggaggatgaagacatggggttgactaccacatctactatcatgagtctgcaatgtccacttcgggaggatgaagaaagttttagagtgagaccagcgtaactcgttcagaattaaa

SEQ ID NO: 496 - DNA, 170 bp, Artificial Sequence
  Synthetic oligonucleotide
tatctacacgggtctcaccaaaaccacgttactttacttgaaaaaaacacttcgggaggatgaatgcatggggttgactaccacatctactatcatgagtctgcaatgtccacttcgggaggatgaagaaagttttagagtgagaccagcgtaactctagggacatttca

SEQ ID NO: 497 - DNA, 170 bp, Artificial Sequence
  Synthetic oligonucleotide
tatctacacgggtctcaccaaaaccacgttactttacttgaaaaaaacacttcgggaggatgaacagatggggttgactaccacatctactatcatgagtctgcaatgtccacttcgggaggatgaagaaagttttagagtgagaccagcgtaactctgatgggtgacca

SEQ ID NO: 498 - DNA, 170 bp, Artificial Sequence
  Synthetic oligonucleotide
tatctacacgggtctcaccaaaaccacgttactttacttgaaaaaaacacttcgggaggatgaagagatggggttgactaccacatctactatcatgagtctgcaatgtccacttcgggaggatgaagaaagttttagagtgagaccagcgtaactcttggtctaccttg

SEQ ID NO: 499 - DNA, 170 bp, Artificial Sequence
  Synthetic oligonucleotide
tatctacacgggtctcaccaaaaccacgttactttacttgaaaaaaacacttcgggaggatgaagggatggggttgactaccacatctactatcatgagtctgcaatgtccacttcgggaggatgaagaaagttttagagtgagaccagcgtaactcctgtatgctttgc

SEQ ID NO: 500 - DNA, 170 bp, Artificial Sequence
  Synthetic oligonucleotide
tatctacacgggtctcaccaaaaccacgttactttacttgaaaaaaacacttcgggaggatgaacacatggggttgactaccacatctactatcatgagtctgcaatgtccacttcgggaggatgaagaaagttttagagtgagaccagcgtaactctttttcctcgact

SEQ ID NO: 501 - DNA, 170 bp, Artificial Sequence
  Synthetic oligonucleotide
tatctacacgggtctcaccaaaaccacgttactttacttgaaaaaaacacttcgggaggatgaaatcatggggttgactaccacatctactatcatgagtctgcaatgtccacttcgggaggatgaagaaagttttagagtgagaccagcgtaactcacacaaatggcgg

SEQ ID NO: 502 - DNA, 170 bp, Artificial Sequence
  Synthetic oligonucleotide
tatctacacgggtctcaccaaaaccacgttactttacttgaaaaaaacacttcgggaggatgaactcatggggttgactaccacatctactatcatgagtctgcaatgtccacttcgggaggatgaagaaagttttagagtgagaccagcgtaactcttatacgccatgg

SEQ ID NO: 503 - DNA, 170 bp, Artificial Sequence
  Synthetic oligonucleotide
tatctacacgggtctcaccaaaaccacgttactttacttgaaaaaaacacttcgggaggatgaaaagatggggttgactaccacatctactatcatgagtctgcaatgtccacttcgggaggatgaagaaagttttagagtgagaccagcgtaactctcttccctaggcc

SEQ ID NO: 504 - DNA, 170 bp, Artificial Sequence
  Synthetic oligonucleotide
tatctacacgggtctcaccaaaaccacgttactttacttgaaaaaaacacttcgggaggatgaaatgatggggttgactaccacatctactatcatgagtctgcaatgtccacttcgggaggatgaagaaagttttagagtgagaccagcgtaactcgagtctcatccgc

SEQ ID NO: 505 - DNA, 170 bp, Artificial Sequence
  Synthetic oligonucleotide
tatctacacgggtctcaccaaaaccacgttactttacttgaaaaaaacacttcgggaggatgaattcatggggttgactaccacatctactatcatgagtctgcaatgtccacttcgggaggatgaagaaagttttagagtgagaccagcgtaactcggtggtaatataa

SEQ ID NO: 506 - DNA, 170 bp, Artificial Sequence
  Synthetic oligonucleotide
tatctacacgggtctcaccaaaaccacgttactttacttgaaaaaaacacttcgggaggatgaaccgatggggttgactaccacatctactatcatgagtctgcaatgtccacttcgggaggatgaagaaagttttagagtgagaccagcgtaactcgtgataataggca

SEQ ID NO: 507 - DNA, 170 bp, Artificial Sequence
  Synthetic oligonucleotide
tatctacacgggtctcaccaaaaccacgttactttacttgaaaaaaacacttcgggaggatgaaagcatggggttgactaccacatctactatcatgagtctgcaatgtccacttcgggaggatgaagaaagttttagagtgagaccagcgtaactcagatacatatgag

SEQ ID NO: 508 - DNA, 170 bp, Artificial Sequence
  Synthetic oligonucleotide
tatctacacgggtctcaccaaaaccacgttactttacttgaaaaaaacacttcgggaggatgaaacgatggggttgactaccacatctactatcatgagtctgcaatgtccacttcgggaggatgaagaaagttttagagtgagaccagcgtaactctgttatttatgcc

SEQ ID NO: 509 - DNA, 170 bp, Artificial Sequence
  Synthetic oligonucleotide
tatctacacgggtctcaccaaaaccacgttactttacttgaaaaaaacacttcgggaggatgaatggatggggttgactaccacatctactatcatgagtctgcaatgtccacttcgggaggatgaagaaagttttagagtgagaccagcgtaactccgtgtaatcgcac

SEQ ID NO: 510 - DNA, 170 bp, Artificial Sequence
  Synthetic oligonucleotide
tatctacacgggtctcaccaaaaccacgttactttacttgaaaaaaacacttcgggaggatgaatacatggggttgactaccacatctactatcatgagtctgcaatgtccacttcgggaggatgaagaaagttttagagtgagaccagcgtaactcccgtgctggaaga

SEQ ID NO: 511 - DNA, 170 bp, Artificial Sequence
  Synthetic oligonucleotide
tatctacacgggtctcaccaaaaccacgttactttacttgaaaaaaacacttcgggaggatgaagtcatggggttgactaccacatctactatcatgagtctgcaatgtccacttcgggaggatgaagaaagttttagagtgagaccagcgtaactcccagctagataga

SEQ ID NO: 512 - DNA, 170 bp, Artificial Sequence
  Synthetic oligonucleotide
tatctacacgggtctcaccaaaacgttactttacttgaaaaaaacacttcgggaggatgaagaggcggggttgactaccacatctactatcatgagtctgcaatgtccacttcgggaggatgaagaaagttttagagtgagaccagcgtaactccataccacaaaattat

SEQ ID NO: 513 - DNA, 170 bp, Artificial Sequence
  Synthetic oligonucleotide
tatctacacgggtctcaccaaaacgttactttacttgaaaaaaacacttcgggaggatgaagagcgggggttgactaccacatctactatcatgagtctgcaatgtccacttcgggaggatgaagaaagttttagagtgagaccagcgtaactcgcggctcagtgcacca

SEQ ID NO: 514 - DNA, 170 bp, Artificial Sequence
  Synthetic oligonucleotide
tatctacacgggtctcaccaaaacgttactttacttgaaaaaaacacttcgggaggatgaagaaaacgggttgactaccacatctactatcatgagtctgcaatgtccacttcgggaggatgaagaaagttttagagtgagaccagcgtaactcgaagctatggtagcca

SEQ ID NO: 515 - DNA, 170 bp, Artificial Sequence
  Synthetic oligonucleotide
tatctacacgggtctcaccaaaacgttactttacttgaaaaaaacacttcgggaggatgaagaagacgggttgactaccacatctactatcatgagtctgcaatgtccacttcgggaggatgaagaaagttttagagtgagaccagcgtaactccgtcagctagcagcac

SEQ ID NO: 516 - DNA, 170 bp, Artificial Sequence
  Synthetic oligonucleotide
tatctacacgggtctcaccaaaacgttactttacttgaaaaaaacacttcgggaggatgaagaatgcgggttgactaccacatctactatcatgagtctgcaatgtccacttcgggaggatgaagaaagttttagagtgagaccagcgtaactcggcgtgaaaaaccttc

SEQ ID NO: 517 - DNA, 170 bp, Artificial Sequence
  Synthetic oligonucleotide
tatctacacgggtctcaccaaaacgttactttacttgaaaaaaacacttcgggaggatgaagagcaggggttgactaccacatctactatcatgagtctgcaatgtccacttcgggaggatgaagaaagttttagagtgagaccagcgtaactctagccacctgccactg

SEQ ID NO: 518 - DNA, 170 bp, Artificial Sequence
  Synthetic oligonucleotide
tatctacacgggtctcaccaaaacgttactttacttgaaaaaaacacttcgggaggatgaagaggaggggttgactaccacatctactatcatgagtctgcaatgtccacttcgggaggatgaagaaagttttagagtgagaccagcgtaactcatacatttaatagcca

SEQ ID NO: 519 - DNA, 170 bp, Artificial Sequence
  Synthetic oligonucleotide
tatctacacgggtctcaccaaaacgttactttacttgaaaaaaacacttcgggaggatgaagagggggggttgactaccacatctactatcatgagtctgcaatgtccacttcgggaggatgaagaaagttttagagtgagaccagcgtaactccccgcggcctattagc

SEQ ID NO: 520 - DNA, 170 bp, Artificial Sequence
  Synthetic oligonucleotide
tatctacacgggtctcaccaaaacgttactttacttgaaaaaaacacttcgggaggatgaagaacacgggttgactaccacatctactatcatgagtctgcaatgtccacttcgggaggatgaagaaagttttagagtgagaccagcgtaactcgtaaagtgacgaggat

SEQ ID NO: 521 - DNA, 170 bp, Artificial Sequence
  Synthetic oligonucleotide
tatctacacgggtctcaccaaaacgttactttacttgaaaaaaacacttcgggaggatgaagaaatcgggttgactaccacatctactatcatgagtctgcaatgtccacttcgggaggatgaagaaagttttagagtgagaccagcgtaactcgtttgtatcgccactg

SEQ ID NO: 522 - DNA, 170 bp, Artificial Sequence
  Synthetic oligonucleotide
tatctacacgggtctcaccaaaacgttactttacttgaaaaaaacacttcgggaggatgaagaactcgggttgactaccacatctactatcatgagtctgcaatgtccacttcgggaggatgaagaaagttttagagtgagaccagcgtaactcccagcctcgcgaccag

SEQ ID NO: 523 - DNA, 170 bp, Artificial Sequence
  Synthetic oligonucleotide
tatctacacgggtctcaccaaaacgttactttacttgaaaaaaacacttcgggaggatgaagagaaggggttgactaccacatctactatcatgagtctgcaatgtccacttcgggaggatgaagaaagttttagagtgagaccagcgtaactctaacgccgagaagctt

SEQ ID NO: 524 - DNA, 170 bp, Artificial Sequence
  Synthetic oligonucleotide
tatctacacgggtctcaccaaaacgttactttacttgaaaaaaacacttcgggaggatgaagagatggggttgactaccacatctactatcatgagtctgcaatgtccacttcgggaggatgaagaaagttttagagtgagaccagcgtaactccgtatgtgccagttat

SEQ ID NO: 525 - DNA, 170 bp, Artificial Sequence
  Synthetic oligonucleotide
tatctacacgggtctcaccaaaacgttactttacttgaaaaaaacacttcgggaggatgaagaattcgggttgactaccacatctactatcatgagtctgcaatgtccacttcgggaggatgaagaaagttttagagtgagaccagcgtaactcaaacataagaacgtcg

SEQ ID NO: 526 - DNA, 170 bp, Artificial Sequence
  Synthetic oligonucleotide
tatctacacgggtctcaccaaaacgttactttacttgaaaaaaacacttcgggaggatgaagagccggggttgactaccacatctactatcatgagtctgcaatgtccacttcgggaggatgaagaaagttttagagtgagaccagcgtaactcagatacccgatgggag

SEQ ID NO: 527 - DNA, 170 bp, Artificial Sequence
  Synthetic oligonucleotide
tatctacacgggtctcaccaaaacgttactttacttgaaaaaaacacttcgggaggatgaagaaagcgggttgactaccacatctactatcatgagtctgcaatgtccacttcgggaggatgaagaaagttttagagtgagaccagcgtaactcgcgcacatagaccaat

SEQ ID NO: 528 - DNA, 170 bp, Artificial Sequence
  Synthetic oligonucleotide
tatctacacgggtctcaccaaaacgttactttacttgaaaaaaacacttcgggaggatgaagagacggggttgactaccacatctactatcatgagtctgcaatgtccacttcgggaggatgaagaaagttttagagtgagaccagcgtaactcgggtcaccgataagaa

SEQ ID NO: 529 - DNA, 170 bp, Artificial Sequence
  Synthetic oligonucleotide
tatctacacgggtctcaccaaaacgttactttacttgaaaaaaacacttcgggaggatgaagagtgggggttgactaccacatctactatcatgagtctgcaatgtccacttcgggaggatgaagaaagttttagagtgagaccagcgtaactctgatacgtgtgtacat

SEQ ID NO: 530 - DNA, 170 bp, Artificial Sequence
  Synthetic oligonucleotide
tatctacacgggtctcaccaaaacgttactttacttgaaaaaaacacttcgggaggatgaagaatacgggttgactaccacatctactatcatgagtctgcaatgtccacttcgggaggatgaagaaagttttagagtgagaccagcgtaactctgaaacgccaggtcgg

SEQ ID NO: 531 - DNA, 170 bp, Artificial Sequence
  Synthetic oligonucleotide
tatctacacgggtctcaccaaaacgttactttacttgaaaaaaacacttcgggaggatgaagaagtcgggttgactaccacatctactatcatgagtctgcaatgtccacttcgggaggatgaagaaagttttagagtgagaccagcgtaactcgttccgttaccacagt

SEQ ID NO: 532 - DNA, 170 bp, Artificial Sequence
  Synthetic oligonucleotide
tatctacacgggtctcaccaaaacttactttacttgaaaaaaacacttcgggaggatgaagagatggcgttgactaccacatctactatcatgagtctgcaatgtccaaacttcgggaggatgaagaaagttttagagtgagaccagcgtaactcggactggaataaaga

SEQ ID NO: 533 - DNA, 170 bp, Artificial Sequence
  Synthetic oligonucleotide
tatctacacgggtctcaccaaaacttactttacttgaaaaaaacacttcgggaggatgaagaaatgcggttgactaccacatctactatcatgagtctgcaatgtccaaacttcgggaggatgaagaaagttttagagtgagaccagcgtaactccaagaaagtagcaag

SEQ ID NO: 534 - DNA, 170 bp, Artificial Sequence
  Synthetic oligonucleotide
tatctacacgggtctcaccaaaacttactttacttgaaaaaaacacttcgggaggatgaagaaatgaacttgactaccacatctactatcatgagtctgcaatgtccaaacttcgggaggatgaagaaagttttagagtgagaccagcgtaactcaacctagttcagttc

SEQ ID NO: 535 - DNA, 170 bp, Artificial Sequence
  Synthetic oligonucleotide
tatctacacgggtctcaccaaaacttactttacttgaaaaaaacacttcgggaggatgaagagatggacttgactaccacatctactatcatgagtctgcaatgtccaaacttcgggaggatgaagaaagttttagagtgagaccagcgtaactcatgccgagctatgcc

SEQ ID NO: 536 - DNA, 170 bp, Artificial Sequence
  Synthetic oligonucleotide
tatctacacgggtctcaccaaaacttactttacttgaaaaaaacacttcgggaggatgaagaaatgtgcttgactaccacatctactatcatgagtctgcaatgtccaaacttcgggaggatgaagaaagttttagagtgagaccagcgtaactccggggaagatagcaa

SEQ ID NO: 537 - DNA, 170 bp, Artificial Sequence
  Synthetic oligonucleotide
tatctacacgggtctcaccaaaacttactttacttgaaaaaaacacttcgggaggatgaagaaatgcagttgactaccacatctactatcatgagtctgcaatgtccaaacttcgggaggatgaagaaagttttagagtgagaccagcgtaactcacatgggggggatgc

SEQ ID NO: 538 - DNA, 170 bp, Artificial Sequence
  Synthetic oligonucleotide
tatctacacgggtctcaccaaaacttactttacttgaaaaaaacacttcgggaggatgaagagatggagttgactaccacatctactatcatgagtctgcaatgtccaaacttcgggaggatgaagaaagttttagagtgagaccagcgtaactcccgggcctcagccgt

SEQ ID NO: 539 - DNA, 170 bp, Artificial Sequence
  Synthetic oligonucleotide
tatctacacgggtctcaccaaaacttactttacttgaaaaaaacacttcgggaggatgaagagatgggtttgactaccacatctactatcatgagtctgcaatgtccaaacttcgggaggatgaagaaagttttagagtgagaccagcgtaactctgggtcggagtgctt

SEQ ID NO: 540 - DNA, 170 bp, Artificial Sequence
  Synthetic oligonucleotide
tatctacacgggtctcaccaaaacttactttacttgaaaaaaacacttcgggaggatgaagaaatgcacttgactaccacatctactatcatgagtctgcaatgtccaaacttcgggaggatgaagaaagttttagagtgagaccagcgtaactccaagtgtttctcgct

SEQ ID NO: 541 - DNA, 170 bp, Artificial Sequence
  Synthetic oligonucleotide
tatctacacgggtctcaccaaaacttactttacttgaaaaaaacacttcgggaggatgaagaaatgatcttgactaccacatctactatcatgagtctgcaatgtccaaacttcgggaggatgaagaaagttttagagtgagaccagcgtaactctggctgaatgcgttc

SEQ ID NO: 542 - DNA, 170 bp, Artificial Sequence
  Synthetic oligonucleotide
tatctacacgggtctcaccaaaacttactttacttgaaaaaaacacttcgggaggatgaagaaatgctcttgactaccacatctactatcatgagtctgcaatgtccaaacttcgggaggatgaagaaagttttagagtgagaccagcgtaactcgcgactcttgcccca

SEQ ID NO: 543 - DNA, 170 bp, Artificial Sequence
  Synthetic oligonucleotide
tatctacacgggtctcaccaaaacttactttacttgaaaaaaacacttcgggaggatgaagaaatgaagttgactaccacatctactatcatgagtctgcaatgtccaaacttcgggaggatgaagaaagttttagagtgagaccagcgtaactctcgtgattaagttgt

SEQ ID NO: 544 - DNA, 170 bp, Artificial Sequence
  Synthetic oligonucleotide
tatctacacgggtctcaccaaaacttactttacttgaaaaaaacacttcgggaggatgaagaaatgatgttgactaccacatctactatcatgagtctgcaatgtccaaacttcgggaggatgaagaaagttttagagtgagaccagcgtaactcgtcgtagtaatgcag

SEQ ID NO: 545 - DNA, 170 bp, Artificial Sequence
  Synthetic oligonucleotide
tatctacacgggtctcaccaaaacttactttacttgaaaaaaacacttcgggaggatgaagaaatgttcttgactaccacatctactatcatgagtctgcaatgtccaaacttcgggaggatgaagaaagttttagagtgagaccagcgtaactcgcggcgtcaaaacgg

SEQ ID NO: 546 - DNA, 170 bp, Artificial Sequence
  Synthetic oligonucleotide
tatctacacgggtctcaccaaaacttactttacttgaaaaaaacacttcgggaggatgaagaaatgccgttgactaccacatctactatcatgagtctgcaatgtccaaacttcgggaggatgaagaaagttttagagtgagaccagcgtaactcctttaccttaattcg

SEQ ID NO: 547 - DNA, 170 bp, Artificial Sequence
  Synthetic oligonucleotide
tatctacacgggtctcaccaaaacttactttacttgaaaaaaacacttcgggaggatgaagaaatgagcttgactaccacatctactatcatgagtctgcaatgtccaaacttcgggaggatgaagaaagttttagagtgagaccagcgtaactctgctgaaggcagatg

SEQ ID NO: 548 - DNA, 170 bp, Artificial Sequence
  Synthetic oligonucleotide
tatctacacgggtctcaccaaaacttactttacttgaaaaaaacacttcgggaggatgaagaaatgacgttgactaccacatctactatcatgagtctgcaatgtccaaacttcgggaggatgaagaaagttttagagtgagaccagcgtaactcgatccacccctgttt

SEQ ID NO: 549 - DNA, 170 bp, Artificial Sequence
  Synthetic oligonucleotide
tatctacacgggtctcaccaaaacttactttacttgaaaaaaacacttcgggaggatgaagaaatgtggttgactaccacatctactatcatgagtctgcaatgtccaaacttcgggaggatgaagaaagttttagagtgagaccagcgtaactcgggaaacaaaaggtg

SEQ ID NO: 550 - DNA, 170 bp, Artificial Sequence
  Synthetic oligonucleotide
tatctacacgggtctcaccaaaacttactttacttgaaaaaaacacttcgggaggatgaagaaatgtacttgactaccacatctactatcatgagtctgcaatgtccaaacttcgggaggatgaagaaagttttagagtgagaccagcgtaactcgtcttatcgcaaatc

SEQ ID NO: 551 - DNA, 170 bp, Artificial Sequence
  Synthetic oligonucleotide
tatctacacgggtctcaccaaaacttactttacttgaaaaaaacacttcgggaggatgaagagatggtcttgactaccacatctactatcatgagtctgcaatgtccaaacttcgggaggatgaagaaagttttagagtgagaccagcgtaactcaaggtatgcccggat

SEQ ID NO: 552 - DNA, 170 bp, Artificial Sequence
  Synthetic oligonucleotide
tatctacacgggtctcaccaaaacttactttacttgaaaaaaacacttcgggaggatgaagagatgggggctactaccacatctactatcatgagtctgcaatgtccaatttacttcgggaggatgaagaaagttttagagtgagaccagcgtaactcgatccagtccga

SEQ ID NO: 553 - DNA, 170 bp, Artificial Sequence
  Synthetic oligonucleotide
tatctacacgggtctcaccaaaacttactttacttgaaaaaaacacttcgggaggatgaagagatggggagaactaccacatctactatcatgagtctgcaatgtccaatttacttcgggaggatgaagaaagttttagagtgagaccagcgtaactccaaaattcaaag

SEQ ID NO: 554 - DNA, 170 bp, Artificial Sequence
  Synthetic oligonucleotide
tatctacacgggtctcaccaaaacttactttacttgaaaaaaacacttcgggaggatgaagagatggggaatactaccacatctactatcatgagtctgcaatgtccaatttacttcgggaggatgaagaaagttttagagtgagaccagcgtaactcttcacggcagac

SEQ ID NO: 555 - DNA, 170 bp, Artificial Sequence
  Synthetic oligonucleotide
tatctacacgggtctcaccaaaacttactttacttgaaaaaaacacttcgggaggatgaagagatgggggatactaccacatctactatcatgagtctgcaatgtccaatttacttcgggaggatgaagaaagttttagagtgagaccagcgtaactctaaggccctgcc

SEQ ID NO: 556 - DNA, 170 bp, Artificial Sequence
  Synthetic oligonucleotide
tatctacacgggtctcaccaaaacttactttacttgaaaaaaacacttcgggaggatgaagagatggggtgtactaccacatctactatcatgagtctgcaatgtccaatttacttcgggaggatgaagaaagttttagagtgagaccagcgtaactcgaagccctccac

SEQ ID NO: 557 - DNA, 170 bp, Artificial Sequence
  Synthetic oligonucleotide
tatctacacgggtctcaccaaaacttactttacttgaaaaaaacacttcgggaggatgaagagatggggcaaactaccacatctactatcatgagtctgcaatgtccaatttacttcgggaggatgaagaaagttttagagtgagaccagcgtaactcccccaaaaatag

SEQ ID NO: 558 - DNA, 170 bp, Artificial Sequence
  Synthetic oligonucleotide
tatctacacgggtctcaccaaaacttactttacttgaaaaaaacacttcgggaggatgaagagatgggggaaactaccacatctactatcatgagtctgcaatgtccaatttacttcgggaggatgaagaaagttttagagtgagaccagcgtaactctgggatcgagtg

SEQ ID NO: 559 - DNA, 170 bp, Artificial Sequence
  Synthetic oligonucleotide
tatctacacgggtctcaccaaaacttactttacttgaaaaaaacacttcgggaggatgaagagatggggggtactaccacatctactatcatgagtctgcaatgtccaatttacttcgggaggatgaagaaagttttagagtgagaccagcgtaactccgtcgtaaggat

SEQ ID NO: 560 - DNA, 170 bp, Artificial Sequence
  Synthetic oligonucleotide
tatctacacgggtctcaccaaaacttactttacttgaaaaaaacacttcgggaggatgaagagatggggcatactaccacatctactatcatgagtctgcaatgtccaatttacttcgggaggatgaagaaagttttagagtgagaccagcgtaactcccggcagagggc

SEQ ID NO: 561 - DNA, 170 bp, Artificial Sequence
  Synthetic oligonucleotide
tatctacacgggtctcaccaaaacttactttacttgaaaaaaacacttcgggaggatgaagagatggggattactaccacatctactatcatgagtctgcaatgtccaatttacttcgggaggatgaagaaagttttagagtgagaccagcgtaactcgtgtcgaccagt

SEQ ID NO: 562 - DNA, 170 bp, Artificial Sequence
  Synthetic oligonucleotide
tatctacacgggtctcaccaaaacttactttacttgaaaaaaacacttcgggaggatgaagagatggggttaactaccacatctactatcatgagtctgcaatgtccaatttacttcgggaggatgaagaaagttttagagtgagaccagcgtaactcgcgaacaactcg

SEQ ID NO: 563 - DNA, 170 bp, Artificial Sequence
  Synthetic oligonucleotide
tatctacacgggtctcaccaaaacttactttacttgaaaaaaacacttcgggaggatgaagagatggggaaaactaccacatctactatcatgagtctgcaatgtccaatttacttcgggaggatgaagaaagttttagagtgagaccagcgtaactcgggggtacactt

SEQ ID NO: 564 - DNA, 170 bp, Artificial Sequence
  Synthetic oligonucleotide
tatctacacgggtctcaccaaaacttactttacttgaaaaaaacacttcgggaggatgaagagatggggatgactaccacatctactatcatgagtctgcaatgtccaatttacttcgggaggatgaagaaagttttagagtgagaccagcgtaactcgcataccaaata

SEQ ID NO: 565 - DNA, 170 bp, Artificial Sequence
  Synthetic oligonucleotide
tatctacacgggtctcaccaaaacttactttacttgaaaaaaacacttcgggaggatgaagagatggggtttactaccacatctactatcatgagtctgcaatgtccaatttacttcgggaggatgaagaaagttttagagtgagaccagcgtaactctaaaccactcag

SEQ ID NO: 566 - DNA, 170 bp, Artificial Sequence
  Synthetic oligonucleotide
tatctacacgggtctcaccaaaacttactttacttgaaaaaaacacttcgggaggatgaagagatggggccaactaccacatctactatcatgagtctgcaatgtccaatttacttcgggaggatgaagaaagttttagagtgagaccagcgtaactctcggacaatacg

SEQ ID NO: 567 - DNA, 170 bp, Artificial Sequence
  Synthetic oligonucleotide
tatctacacgggtctcaccaaaacttactttacttgaaaaaaacacttcgggaggatgaagagatggggtctactaccacatctactatcatgagtctgcaatgtccaatttacttcgggaggatgaagaaagttttagagtgagaccagcgtaactcgaggttgacctc

SEQ ID NO: 568 - DNA, 170 bp, Artificial Sequence
  Synthetic oligonucleotide
tatctacacgggtctcaccaaaacttactttacttgaaaaaaacacttcgggaggatgaagagatggggactactaccacatctactatcatgagtctgcaatgtccaatttacttcgggaggatgaagaaagttttagagtgagaccagcgtaactcttccaggttgga

SEQ ID NO: 569 - DNA, 170 bp, Artificial Sequence
  Synthetic oligonucleotide
tatctacacgggtctcaccaaaacttactttacttgaaaaaaacacttcgggaggatgaagagatggggtggactaccacatctactatcatgagtctgcaatgtccaatttacttcgggaggatgaagaaagttttagagtgagaccagcgtaactcactgtacacctg

SEQ ID NO: 570 - DNA, 170 bp, Artificial Sequence
  Synthetic oligonucleotide
tatctacacgggtctcaccaaaacttactttacttgaaaaaaacacttcgggaggatgaagagatggggtatactaccacatctactatcatgagtctgcaatgtccaatttacttcgggaggatgaagaaagttttagagtgagaccagcgtaactcgtgtgattgcgc

SEQ ID NO: 571 - DNA, 170 bp, Artificial Sequence
  Synthetic oligonucleotide
tatctacacgggtctcaccaaaacttactttacttgaaaaaaacacttcgggaggatgaagagatgggggttactaccacatctactatcatgagtctgcaatgtccaatttacttcgggaggatgaagaaagttttagagtgagaccagcgtaactcacgtggggtccc

SEQ ID NO: 572 - DNA, 170 bp, Artificial Sequence
  Synthetic oligonucleotide
tatctacacgggtctcaccaaaacttactttacttgaaaaaaacacttcgggaggatgaagagatggggttggctaccacatctactatcatgagtctgcaatgtccaatttcgtacttcgggaggatgaagaaagttttagagtgagaccagcgtaactcgcgtggatc

SEQ ID NO: 573 - DNA, 170 bp, Artificial Sequence
  Synthetic oligonucleotide
tatctacacgggtctcaccaaaacttactttacttgaaaaaaacacttcgggaggatgaagagatggggttgagaaccacatctactatcatgagtctgcaatgtccaatttcgtacttcgggaggatgaagaaagttttagagtgagaccagcgtaactctactgagta

SEQ ID NO: 574 - DNA, 170 bp, Artificial Sequence
  Synthetic oligonucleotide
tatctacacgggtctcaccaaaacttactttacttgaaaaaaacacttcgggaggatgaagagatggggttgaataccacatctactatcatgagtctgcaatgtccaatttcgtacttcgggaggatgaagaaagttttagagtgagaccagcgtaactcctaagaatg

SEQ ID NO: 575 - DNA, 170 bp, Artificial Sequence
  Synthetic oligonucleotide
tatctacacgggtctcaccaaaacttactttacttgaaaaaaacacttcgggaggatgaagagatggggttggataccacatctactatcatgagtctgcaatgtccaatttcgtacttcgggaggatgaagaaagttttagagtgagaccagcgtaactctgaagagta

SEQ ID NO: 576 - DNA, 170 bp, Artificial Sequence
  Synthetic oligonucleotide
tatctacacgggtctcaccaaaacttactttacttgaaaaaaacacttcgggaggatgaagagatggggttgtgtaccacatctactatcatgagtctgcaatgtccaatttcgtacttcgggaggatgaagaaagttttagagtgagaccagcgtaactctatttacgg

SEQ ID NO: 577 - DNA, 170 bp, Artificial Sequence
  Synthetic oligonucleotide
tatctacacgggtctcaccaaaacttactttacttgaaaaaaacacttcgggaggatgaagagatggggttgcaaaccacatctactatcatgagtctgcaatgtccaatttcgtacttcgggaggatgaagaaagttttagagtgagaccagcgtaactcattagctaa

SEQ ID NO: 578 - DNA, 170 bp, Artificial Sequence
  Synthetic oligonucleotide
tatctacacgggtctcaccaaaacttactttacttgaaaaaaacacttcgggaggatgaagagatggggttggaaaccacatctactatcatgagtctgcaatgtccaatttcgtacttcgggaggatgaagaaagttttagagtgagaccagcgtaactcttccacatg

SEQ ID NO: 579 - DNA, 170 bp, Artificial Sequence
  Synthetic oligonucleotide
tatctacacgggtctcaccaaaacttactttacttgaaaaaaacacttcgggaggatgaagagatggggttgggtaccacatctactatcatgagtctgcaatgtccaatttcgtacttcgggaggatgaagaaagttttagagtgagaccagcgtaactcccgacgtac

SEQ ID NO: 580 - DNA, 170 bp, Artificial Sequence
  Synthetic oligonucleotide
tatctacacgggtctcaccaaaacttactttacttgaaaaaaacacttcgggaggatgaagagatggggttgcataccacatctactatcatgagtctgcaatgtccaatttcgtacttcgggaggatgaagaaagttttagagtgagaccagcgtaactctcataatca

SEQ ID NO: 581 - DNA, 170 bp, Artificial Sequence
  Synthetic oligonucleotide
tatctacacgggtctcaccaaaacttactttacttgaaaaaaacacttcgggaggatgaagagatggggttgattaccacatctactatcatgagtctgcaatgtccaatttcgtacttcgggaggatgaagaaagttttagagtgagaccagcgtaactctataacacc

SEQ ID NO: 582 - DNA, 170 bp, Artificial Sequence
  Synthetic oligonucleotide
tatctacacgggtctcaccaaaacttactttacttgaaaaaaacacttcgggaggatgaagagatggggttgttgaccacatctactatcatgagtctgcaatgtccaatttcgtacttcgggaggatgaagaaagttttagagtgagaccagcgtaactcaatactgaa

SEQ ID NO: 583 - DNA, 170 bp, Artificial Sequence
  Synthetic oligonucleotide
tatctacacgggtctcaccaaaacttactttacttgaaaaaaacacttcgggaggatgaagagatggggttgaaaaccacatctactatcatgagtctgcaatgtccaatttcgtacttcgggaggatgaagaaagttttagagtgagaccagcgtaactccccggtgac

SEQ ID NO: 584 - DNA, 170 bp, Artificial Sequence
  Synthetic oligonucleotide
tatctacacgggtctcaccaaaacttactttacttgaaaaaaacacttcgggaggatgaagagatggggttgatgaccacatctactatcatgagtctgcaatgtccaatttcgtacttcgggaggatgaagaaagttttagagtgagaccagcgtaactccttctgacg

SEQ ID NO: 585 - DNA, 170 bp, Artificial Sequence
  Synthetic oligonucleotide
tatctacacgggtctcaccaaaacttactttacttgaaaaaaacacttcgggaggatgaagagatggggttgtttaccacatctactatcatgagtctgcaatgtccaatttcgtacttcgggaggatgaagaaagttttagagtgagaccagcgtaactccagcgtacg

SEQ ID NO: 586 - DNA, 170 bp, Artificial Sequence
  Synthetic oligonucleotide
tatctacacgggtctcaccaaaacttactttacttgaaaaaaacacttcgggaggatgaagagatggggttgccaaccacatctactatcatgagtctgcaatgtccaatttcgtacttcgggaggatgaagaaagttttagagtgagaccagcgtaactcgaggatacg

SEQ ID NO: 587 - DNA, 170 bp, Artificial Sequence
  Synthetic oligonucleotide
tatctacacgggtctcaccaaaacttactttacttgaaaaaaacacttcgggaggatgaagagatggggttgtctaccacatctactatcatgagtctgcaatgtccaatttcgtacttcgggaggatgaagaaagttttagagtgagaccagcgtaactcagagcttta

SEQ ID NO: 588 - DNA, 170 bp, Artificial Sequence
  Synthetic oligonucleotide
tatctacacgggtctcaccaaaacttactttacttgaaaaaaacacttcgggaggatgaagagatggggttgacaaccacatctactatcatgagtctgcaatgtccaatttcgtacttcgggaggatgaagaaagttttagagtgagaccagcgtaactcccgttttgc

SEQ ID NO: 589 - DNA, 170 bp, Artificial Sequence
  Synthetic oligonucleotide
tatctacacgggtctcaccaaaacttactttacttgaaaaaaacacttcgggaggatgaagagatggggttgtggaccacatctactatcatgagtctgcaatgtccaatttcgtacttcgggaggatgaagaaagttttagagtgagaccagcgtaactccggaaatac

SEQ ID NO: 590 - DNA, 170 bp, Artificial Sequence
  Synthetic oligonucleotide
tatctacacgggtctcaccaaaacttactttacttgaaaaaaacacttcgggaggatgaagagatggggttgtataccacatctactatcatgagtctgcaatgtccaatttcgtacttcgggaggatgaagaaagttttagagtgagaccagcgtaactccaagtctct

SEQ ID NO: 591 - DNA, 170 bp, Artificial Sequence
  Synthetic oligonucleotide
tatctacacgggtctcaccaaaacttactttacttgaaaaaaacacttcgggaggatgaagagatggggttggttaccacatctactatcatgagtctgcaatgtccaatttcgtacttcgggaggatgaagaaagttttagagtgagaccagcgtaactcggtatggtg

SEQ ID NO: 592 - DNA, 170 bp, Artificial Sequence
  Synthetic oligonucleotide
tatctacacgggtctcaccaaaacttactttacttgaaaaaaacacttcgggaggatgaagagatggggttgactgctacatctactatcatgagtctgcaatgtccaatttcgtacaacttcgggaggatgaagaaagttttagagtgagaccagcgtaactctaggca

SEQ ID NO: 593 - DNA, 170 bp, Artificial Sequence
  Synthetic oligonucleotide
tatctacacgggtctcaccaaaacttactttacttgaaaaaaacacttcgggaggatgaagagatggggttgactagaacatctactatcatgagtctgcaatgtccaatttcgtacaacttcgggaggatgaagaaagttttagagtgagaccagcgtaactcctctag

SEQ ID NO: 594 - DNA, 170 bp, Artificial Sequence
  Synthetic oligonucleotide
tatctacacgggtctcaccaaaacttactttacttgaaaaaaacacttcgggaggatgaagagatggggttgactaatacatctactatcatgagtctgcaatgtccaatttcgtacaacttcgggaggatgaagaaagttttagagtgagaccagcgtaactcttttca

SEQ ID NO: 595 - DNA, 170 bp, Artificial Sequence
  Synthetic oligonucleotide
tatctacacgggtctcaccaaaacttactttacttgaaaaaaacacttcgggaggatgaagagatggggttgactgatacatctactatcatgagtctgcaatgtccaatttcgtacaacttcgggaggatgaagaaagttttagagtgagaccagcgtaactcgccata

SEQ ID NO: 596 - DNA, 170 bp, Artificial Sequence
  Synthetic oligonucleotide
tatctacacgggtctcaccaaaacttactttacttgaaaaaaacacttcgggaggatgaagagatggggttgacttgtacatctactatcatgagtctgcaatgtccaatttcgtacaacttcgggaggatgaagaaagttttagagtgagaccagcgtaactcagtaga

SEQ ID NO: 597 - DNA, 170 bp, Artificial Sequence
  Synthetic oligonucleotide
tatctacacgggtctcaccaaaacttactttacttgaaaaaaacacttcgggaggatgaagagatggggttgactcaaacatctactatcatgagtctgcaatgtccaatttcgtacaacttcgggaggatgaagaaagttttagagtgagaccagcgtaactccgccat

SEQ ID NO: 598 - DNA, 170 bp, Artificial Sequence
  Synthetic oligonucleotide
tatctacacgggtctcaccaaaacttactttacttgaaaaaaacacttcgggaggatgaagagatggggttgactgaaacatctactatcatgagtctgcaatgtccaatttcgtacaacttcgggaggatgaagaaagttttagagtgagaccagcgtaactcaggctc

SEQ ID NO: 599 - DNA, 170 bp, Artificial Sequence
  Synthetic oligonucleotide
tatctacacgggtctcaccaaaacttactttacttgaaaaaaacacttcgggaggatgaagagatggggttgactggtacatctactatcatgagtctgcaatgtccaatttcgtacaacttcgggaggatgaagaaagttttagagtgagaccagcgtaactcatttct

SEQ ID NO: 600 - DNA, 170 bp, Artificial Sequence
  Synthetic oligonucleotide
tatctacacgggtctcaccaaaacttactttacttgaaaaaaacacttcgggaggatgaagagatggggttgactcatacatctactatcatgagtctgcaatgtccaatttcgtacaacttcgggaggatgaagaaagttttagagtgagaccagcgtaactccagtag

SEQ ID NO: 601 - DNA, 170 bp, Artificial Sequence
  Synthetic oligonucleotide
tatctacacgggtctcaccaaaacttactttacttgaaaaaaacacttcgggaggatgaagagatggggttgactattacatctactatcatgagtctgcaatgtccaatttcgtacaacttcgggaggatgaagaaagttttagagtgagaccagcgtaactctcttgt

SEQ ID NO: 602 - DNA, 170 bp, Artificial Sequence
  Synthetic oligonucleotide
tatctacacgggtctcaccaaaacttactttacttgaaaaaaacacttcgggaggatgaagagatggggttgactttgacatctactatcatgagtctgcaatgtccaatttcgtacaacttcgggaggatgaagaaagttttagagtgagaccagcgtaactcgagtat

SEQ ID NO: 603 - DNA, 170 bp, Artificial Sequence
  Synthetic oligonucleotide
tatctacacgggtctcaccaaaacttactttacttgaaaaaaacacttcgggaggatgaagagatggggttgactaaaacatctactatcatgagtctgcaatgtccaatttcgtacaacttcgggaggatgaagaaagttttagagtgagaccagcgtaactctcagtg

SEQ ID NO: 604 - DNA, 170 bp, Artificial Sequence
  Synthetic oligonucleotide
tatctacacgggtctcaccaaaacttactttacttgaaaaaaacacttcgggaggatgaagagatggggttgactatgacatctactatcatgagtctgcaatgtccaatttcgtacaacttcgggaggatgaagaaagttttagagtgagaccagcgtaactccaagta

SEQ ID NO: 605 - DNA, 170 bp, Artificial Sequence
  Synthetic oligonucleotide
tatctacacgggtctcaccaaaacttactttacttgaaaaaaacacttcgggaggatgaagagatggggttgacttttacatctactatcatgagtctgcaatgtccaatttcgtacaacttcgggaggatgaagaaagttttagagtgagaccagcgtaactctgttgg

SEQ ID NO: 606 - DNA, 170 bp, Artificial Sequence
  Synthetic oligonucleotide
tatctacacgggtctcaccaaaacttactttacttgaaaaaaacacttcgggaggatgaagagatggggttgactccaacatctactatcatgagtctgcaatgtccaatttcgtacaacttcgggaggatgaagaaagttttagagtgagaccagcgtaactcactatc

SEQ ID NO: 607 - DNA, 170 bp, Artificial Sequence
  Synthetic oligonucleotide
tatctacacgggtctcaccaaaacttactttacttgaaaaaaacacttcgggaggatgaagagatggggttgacttctacatctactatcatgagtctgcaatgtccaatttcgtacaacttcgggaggatgaagaaagttttagagtgagaccagcgtaactcacttag

SEQ ID NO: 608 - DNA, 170 bp, Artificial Sequence
  Synthetic oligonucleotide
tatctacacgggtctcaccaaaacttactttacttgaaaaaaacacttcgggaggatgaagagatggggttgactactacatctactatcatgagtctgcaatgtccaatttcgtacaacttcgggaggatgaagaaagttttagagtgagaccagcgtaactcgctatc

SEQ ID NO: 609 - DNA, 170 bp, Artificial Sequence
  Synthetic oligonucleotide
tatctacacgggtctcaccaaaacttactttacttgaaaaaaacacttcgggaggatgaagagatggggttgacttggacatctactatcatgagtctgcaatgtccaatttcgtacaacttcgggaggatgaagaaagttttagagtgagaccagcgtaactctggcgc

SEQ ID NO: 610 - DNA, 170 bp, Artificial Sequence
  Synthetic oligonucleotide
tatctacacgggtctcaccaaaacttactttacttgaaaaaaacacttcgggaggatgaagagatggggttgacttatacatctactatcatgagtctgcaatgtccaatttcgtacaacttcgggaggatgaagaaagttttagagtgagaccagcgtaactccgtagt

SEQ ID NO: 611 - DNA, 170 bp, Artificial Sequence
  Synthetic oligonucleotide
tatctacacgggtctcaccaaaacttactttacttgaaaaaaacacttcgggaggatgaagagatggggttgactgttacatctactatcatgagtctgcaatgtccaatttcgtacaacttcgggaggatgaagaaagttttagagtgagaccagcgtaactcctgatt

SEQ ID NO: 612 - DNA, 170 bp, Artificial Sequence
  Synthetic oligonucleotide
tatctacacgggtctcaccaaaacttactttacttgaaaaaaacacttcgggaggatgaagagatggggttgactaccgcttctactatcatgagtctgcaatgtccaatttcgtacacaaacttcgggaggatgaagaaagttttagagtgagaccagcgtaactcgat

SEQ ID NO: 613 - DNA, 170 bp, Artificial Sequence
  Synthetic oligonucleotide
tatctacacgggtctcaccaaaacttactttacttgaaaaaaacacttcgggaggatgaagagatggggttgactaccagatctactatcatgagtctgcaatgtccaatttcgtacacaaacttcgggaggatgaagaaagttttagagtgagaccagcgtaactctgt

SEQ ID NO: 614 - DNA, 170 bp, Artificial Sequence
  Synthetic oligonucleotide
tatctacacgggtctcaccaaaacttactttacttgaaaaaaacacttcgggaggatgaagagatggggttgactaccaattctactatcatgagtctgcaatgtccaatttcgtacacaaacttcgggaggatgaagaaagttttagagtgagaccagcgtaactcaaa

SEQ ID NO: 615 - DNA, 170 bp, Artificial Sequence
  Synthetic oligonucleotide
tatctacacgggtctcaccaaaacttactttacttgaaaaaaacacttcgggaggatgaagagatggggttgactaccgattctactatcatgagtctgcaatgtccaatttcgtacacaaacttcgggaggatgaagaaagttttagagtgagaccagcgtaactcacc

SEQ ID NO: 616 - DNA, 170 bp, Artificial Sequence
  Synthetic oligonucleotide
tatctacacgggtctcaccaaaacttactttacttgaaaaaaacacttcgggaggatgaagagatggggttgactacctgttctactatcatgagtctgcaatgtccaatttcgtacacaaacttcgggaggatgaagaaagttttagagtgagaccagcgtaactctct

SEQ ID NO: 617 - DNA, 170 bp, Artificial Sequence
  Synthetic oligonucleotide
tatctacacgggtctcaccaaaacttactttacttgaaaaaaacacttcgggaggatgaagagatggggttgactacccaatctactatcatgagtctgcaatgtccaatttcgtacacaaacttcgggaggatgaagaaagttttagagtgagaccagcgtaactcctg

SEQ ID NO: 618 - DNA, 170 bp, Artificial Sequence
  Synthetic oligonucleotide
tatctacacgggtctcaccaaaacttactttacttgaaaaaaacacttcgggaggatgaagagatggggttgactaccgaatctactatcatgagtctgcaatgtccaatttcgtacacaaacttcgggaggatgaagaaagttttagagtgagaccagcgtaactcgct

SEQ ID NO: 619 - DNA, 170 bp, Artificial Sequence
  Synthetic oligonucleotide
tatctacacgggtctcaccaaaacttactttacttgaaaaaaacacttcgggaggatgaagagatggggttgactaccggttctactatcatgagtctgcaatgtccaatttcgtacacaaacttcgggaggatgaagaaagttttagagtgagaccagcgtaactcagt

SEQ ID NO: 620 - DNA, 170 bp, Artificial Sequence
  Synthetic oligonucleotide
tatctacacgggtctcaccaaaacttactttacttgaaaaaaacacttcgggaggatgaagagatggggttgactacccattctactatcatgagtctgcaatgtccaatttcgtacacaaacttcgggaggatgaagaaagttttagagtgagaccagcgtaactcaag

SEQ ID NO: 621 - DNA, 170 bp, Artificial Sequence
  Synthetic oligonucleotide
tatctacacgggtctcaccaaaacttactttacttgaaaaaaacacttcgggaggatgaagagatggggttgactaccatttctactatcatgagtctgcaatgtccaatttcgtacacaaacttcgggaggatgaagaaagttttagagtgagaccagcgtaactccgc

SEQ ID NO: 622 - DNA, 170 bp, Artificial Sequence
  Synthetic oligonucleotide
tatctacacgggtctcaccaaaacttactttacttgaaaaaaacacttcgggaggatgaagagatggggttgactaccttgtctactatcatgagtctgcaatgtccaatttcgtacacaaacttcgggaggatgaagaaagttttagagtgagaccagcgtaactcact

SEQ ID NO: 623 - DNA, 170 bp, Artificial Sequence
  Synthetic oligonucleotide
tatctacacgggtctcaccaaaacttactttacttgaaaaaaacacttcgggaggatgaagagatggggttgactaccaaatctactatcatgagtctgcaatgtccaatttcgtacacaaacttcgggaggatgaagaaagttttagagtgagaccagcgtaactccag

SEQ ID NO: 624 - DNA, 170 bp, Artificial Sequence
  Synthetic oligonucleotide
tatctacacgggtctcaccaaaacttactttacttgaaaaaaacacttcgggaggatgaagagatggggttgactaccatgtctactatcatgagtctgcaatgtccaatttcgtacacaaacttcgggaggatgaagaaagttttagagtgagaccagcgtaactcagc

SEQ ID NO: 625 - DNA, 170 bp, Artificial Sequence
  Synthetic oligonucleotide
tatctacacgggtctcaccaaaacttactttacttgaaaaaaacacttcgggaggatgaagagatggggttgactaccttttctactatcatgagtctgcaatgtccaatttcgtacacaaacttcgggaggatgaagaaagttttagagtgagaccagcgtaactcggc

SEQ ID NO: 626 - DNA, 170 bp, Artificial Sequence
  Synthetic oligonucleotide
tatctacacgggtctcaccaaaacttactttacttgaaaaaaacacttcgggaggatgaagagatggggttgactaccccatctactatcatgagtctgcaatgtccaatttcgtacacaaacttcgggaggatgaagaaagttttagagtgagaccagcgtaactcatt

SEQ ID NO: 627 - DNA, 170 bp, Artificial Sequence
  Synthetic oligonucleotide
tatctacacgggtctcaccaaaacttactttacttgaaaaaaacacttcgggaggatgaagagatggggttgactacctcttctactatcatgagtctgcaatgtccaatttcgtacacaaacttcgggaggatgaagaaagttttagagtgagaccagcgtaactccgc

SEQ ID NO: 628 - DNA, 170 bp, Artificial Sequence
  Synthetic oligonucleotide
tatctacacgggtctcaccaaaacttactttacttgaaaaaaacacttcgggaggatgaagagatggggttgactaccacttctactatcatgagtctgcaatgtccaatttcgtacacaaacttcgggaggatgaagaaagttttagagtgagaccagcgtaactcagt

SEQ ID NO: 629 - DNA, 170 bp, Artificial Sequence
  Synthetic oligonucleotide
tatctacacgggtctcaccaaaacttactttacttgaaaaaaacacttcgggaggatgaagagatggggttgactacctggtctactatcatgagtctgcaatgtccaatttcgtacacaaacttcgggaggatgaagaaagttttagagtgagaccagcgtaactcgac

SEQ ID NO: 630 - DNA, 170 bp, Artificial Sequence
  Synthetic oligonucleotide
tatctacacgggtctcaccaaaacttactttacttgaaaaaaacacttcgggaggatgaagagatggggttgactacctattctactatcatgagtctgcaatgtccaatttcgtacacaaacttcgggaggatgaagaaagttttagagtgagaccagcgtaactcagc

SEQ ID NO: 631 - DNA, 170 bp, Artificial Sequence
  Synthetic oligonucleotide
tatctacacgggtctcaccaaaacttactttacttgaaaaaaacacttcgggaggatgaagagatggggttgactaccgtttctactatcatgagtctgcaatgtccaatttcgtacacaaacttcgggaggatgaagaaagttttagagtgagaccagcgtaactctgt

SEQ ID NO: 632 - DNA, 170 bp, Artificial Sequence
  Synthetic oligonucleotide
tatctacacgggtctcaccaaaacaaaaaaacacttcgggaggatgaagaaatgggcttgactacgacggctactatcatgagtctgcaatgtccaatttcgtacacaagaacagactcatgatagtagatggttttagagtgagaccagcgtaactcttaaaggtgtta

SEQ ID NO: 633 - DNA, 170 bp, Artificial Sequence
  Synthetic oligonucleotide
tatctacacgggtctcaccaaaacaaaaaaacacttcgggaggatgaagaaatgggcttgactacgacgagaactatcatgagtctgcaatgtccaatttcgtacacaagaacagactcatgatagtagatggttttagagtgagaccagcgtaactcaaacacggggat

SEQ ID NO: 634 - DNA, 170 bp, Artificial Sequence
  Synthetic oligonucleotide
tatctacacgggtctcaccaaaacaaaaaaacacttcgggaggatgaagaaatgggcttgactacgacgaatactatcatgagtctgcaatgtccaatttcgtacacaagaacagactcatgatagtagatggttttagagtgagaccagcgtaactcgtctctgggagc

SEQ ID NO: 635 - DNA, 170 bp, Artificial Sequence
  Synthetic oligonucleotide
tatctacacgggtctcaccaaaacaaaaaaacacttcgggaggatgaagaaatgggcttgactacgacggatactatcatgagtctgcaatgtccaatttcgtacacaagaacagactcatgatagtagatggttttagagtgagaccagcgtaactcaaagtatttcat

SEQ ID NO: 636 - DNA, 170 bp, Artificial Sequence
  Synthetic oligonucleotide
tatctacacgggtctcaccaaaacaaaaaaacacttcgggaggatgaagaaatgggcttgactacgacgtgtactatcatgagtctgcaatgtccaatttcgtacacaagaacagactcatgatagtagatggttttagagtgagaccagcgtaactcctcgactatcga

SEQ ID NO: 637 - DNA, 170 bp, Artificial Sequence
  Synthetic oligonucleotide
tatctacacgggtctcaccaaaacaaaaaaacacttcgggaggatgaagaaatgggcttgactacgacgcaaactatcatgagtctgcaatgtccaatttcgtacacaagaacagactcatgatagtagatggttttagagtgagaccagcgtaactcccctcgtggtcg

SEQ ID NO: 638 - DNA, 170 bp, Artificial Sequence
  Synthetic oligonucleotide
tatctacacgggtctcaccaaaacaaaaaaacacttcgggaggatgaagaaatgggcttgactacgacggaaactatcatgagtctgcaatgtccaatttcgtacacaagaacagactcatgatagtagatggttttagagtgagaccagcgtaactcaggcggcgtcac

SEQ ID NO: 639 - DNA, 170 bp, Artificial Sequence
  Synthetic oligonucleotide
tatctacacgggtctcaccaaaacaaaaaaacacttcgggaggatgaagaaatgggcttgactacgacgggtactatcatgagtctgcaatgtccaatttcgtacacaagaacagactcatgatagtagatggttttagagtgagaccagcgtaactctcatcctgttag

SEQ ID NO: 640 - DNA, 170 bp, Artificial Sequence
  Synthetic oligonucleotide
tatctacacgggtctcaccaaaacaaaaaaacacttcgggaggatgaagaaatgggcttgactacgacgcatactatcatgagtctgcaatgtccaatttcgtacacaagaacagactcatgatagtagatggttttagagtgagaccagcgtaactcgagtgtaattta

SEQ ID NO: 641 - DNA, 170 bp, Artificial Sequence
  Synthetic oligonucleotide
tatctacacgggtctcaccaaaacaaaaaaacacttcgggaggatgaagaaatgggcttgactacgacgattactatcatgagtctgcaatgtccaatttcgtacacaagaacagactcatgatagtagatggttttagagtgagaccagcgtaactcgacaaagaaacc

SEQ ID NO: 642 - DNA, 170 bp, Artificial Sequence
  Synthetic oligonucleotide
tatctacacgggtctcaccaaaacaaaaaaacacttcgggaggatgaagaaatgggcttgactacgacgttgactatcatgagtctgcaatgtccaatttcgtacacaagaacagactcatgatagtagatggttttagagtgagaccagcgtaactcggccaggtgcga

SEQ ID NO: 643 - DNA, 170 bp, Artificial Sequence
  Synthetic oligonucleotide
tatctacacgggtctcaccaaaacaaaaaaacacttcgggaggatgaagaaatgggcttgactacgacgaaaactatcatgagtctgcaatgtccaatttcgtacacaagaacagactcatgatagtagatggttttagagtgagaccagcgtaactcagatgggcgggc

SEQ ID NO: 644 - DNA, 170 bp, Artificial Sequence
  Synthetic oligonucleotide
tatctacacgggtctcaccaaaacaaaaaaacacttcgggaggatgaagaaatgggcttgactacgacgatgactatcatgagtctgcaatgtccaatttcgtacacaagaacagactcatgatagtagatggttttagagtgagaccagcgtaactcttcttaaaccct

SEQ ID NO: 645 - DNA, 170 bp, Artificial Sequence
  Synthetic oligonucleotide
tatctacacgggtctcaccaaaacaaaaaaacacttcgggaggatgaagaaatgggcttgactacgacgtttactatcatgagtctgcaatgtccaatttcgtacacaagaacagactcatgatagtagatggttttagagtgagaccagcgtaactcgactggtaagca

SEQ ID NO: 646 - DNA, 170 bp, Artificial Sequence
  Synthetic oligonucleotide
tatctacacgggtctcaccaaaacaaaaaaacacttcgggaggatgaagaaatgggcttgactacgacgccaactatcatgagtctgcaatgtccaatttcgtacacaagaacagactcatgatagtagatggttttagagtgagaccagcgtaactcatcttcgtctct

SEQ ID NO: 647 - DNA, 170 bp, Artificial Sequence
  Synthetic oligonucleotide
tatctacacgggtctcaccaaaacaaaaaaacacttcgggaggatgaagaaatgggcttgactacgacgagtactatcatgagtctgcaatgtccaatttcgtacacaagaacagactcatgatagtagatggttttagagtgagaccagcgtaactcgcgaccccttga

SEQ ID NO: 648 - DNA, 170 bp, Artificial Sequence
  Synthetic oligonucleotide
tatctacacgggtctcaccaaaacaaaaaaacacttcgggaggatgaagaaatgggcttgactacgacgactactatcatgagtctgcaatgtccaatttcgtacacaagaacagactcatgatagtagatggttttagagtgagaccagcgtaactcctcattgtctca

SEQ ID NO: 649 - DNA, 170 bp, Artificial Sequence
  Synthetic oligonucleotide
tatctacacgggtctcaccaaaacaaaaaaacacttcgggaggatgaagaaatgggcttgactacgacgtggactatcatgagtctgcaatgtccaatttcgtacacaagaacagactcatgatagtagatggttttagagtgagaccagcgtaactctcagcgatctta

SEQ ID NO: 650 - DNA, 170 bp, Artificial Sequence
  Synthetic oligonucleotide
tatctacacgggtctcaccaaaacaaaaaaacacttcgggaggatgaagaaatgggcttgactacgacgtatactatcatgagtctgcaatgtccaatttcgtacacaagaacagactcatgatagtagatggttttagagtgagaccagcgtaactctgggtccggttg

SEQ ID NO: 651 - DNA, 170 bp, Artificial Sequence
  Synthetic oligonucleotide
tatctacacgggtctcaccaaaacaaaaaaacacttcgggaggatgaagaaatgggcttgactacgacggttactatcatgagtctgcaatgtccaatttcgtacacaagaacagactcatgatagtagatggttttagagtgagaccagcgtaactcgtccgggagttg

SEQ ID NO: 652 - DNA, 170 bp, Artificial Sequence
  Synthetic oligonucleotide
tatctacacgggtctcaccaaaacaaaaaaacacttcgggaggatgaagaaatgggcttgactacgacgtctgctatcatgagtctgcaatgtccaatttcgtacacaagaatgacagactcatgatagtagatggttttagagtgagaccagcgtaactctgtcggatt

SEQ ID NO: 653 - DNA, 170 bp, Artificial Sequence
  Synthetic oligonucleotide
tatctacacgggtctcaccaaaacaaaaaaacacttcgggaggatgaagaaatgggcttgactacgacgtctagaatcatgagtctgcaatgtccaatttcgtacacaagaatgacagactcatgatagtagatggttttagagtgagaccagcgtaactcactgagccc

SEQ ID NO: 654 - DNA, 170 bp, Artificial Sequence
  Synthetic oligonucleotide
tatctacacgggtctcaccaaaacaaaaaaacacttcgggaggatgaagaaatgggcttgactacgacgtctaatatcatgagtctgcaatgtccaatttcgtacacaagaatgacagactcatgatagtagatggttttagagtgagaccagcgtaactctcggagagc

SEQ ID NO: 655 - DNA, 170 bp, Artificial Sequence
  Synthetic oligonucleotide
tatctacacgggtctcaccaaaacaaaaaaacacttcgggaggatgaagaaatgggcttgactacgacgtctgatatcatgagtctgcaatgtccaatttcgtacacaagaatgacagactcatgatagtagatggttttagagtgagaccagcgtaactcacagacacg

SEQ ID NO: 656 - DNA, 170 bp, Artificial Sequence
  Synthetic oligonucleotide
tatctacacgggtctcaccaaaacaaaaaaacacttcgggaggatgaagaaatgggcttgactacgacgtcttgtatcatgagtctgcaatgtccaatttcgtacacaagaatgacagactcatgatagtagatggttttagagtgagaccagcgtaactcgtgtgatcg

SEQ ID NO: 657 - DNA, 170 bp, Artificial Sequence
  Synthetic oligonucleotide
tatctacacgggtctcaccaaaacaaaaaaacacttcgggaggatgaagaaatgggcttgactacgacgtctcaaatcatgagtctgcaatgtccaatttcgtacacaagaatgacagactcatgatagtagatggttttagagtgagaccagcgtaactcaaaagtccc

SEQ ID NO: 658 - DNA, 170 bp, Artificial Sequence
  Synthetic oligonucleotide
tatctacacgggtctcaccaaaacaaaaaaacacttcgggaggatgaagaaatgggcttgactacgacgtctgaaatcatgagtctgcaatgtccaatttcgtacacaagaatgacagactcatgatagtagatggttttagagtgagaccagcgtaactcccaaaacgc

SEQ ID NO: 659 - DNA, 170 bp, Artificial Sequence
  Synthetic oligonucleotide
tatctacacgggtctcaccaaaacaaaaaaacacttcgggaggatgaagaaatgggcttgactacgacgtctggtatcatgagtctgcaatgtccaatttcgtacacaagaatgacagactcatgatagtagatggttttagagtgagaccagcgtaactcaggctcatt

SEQ ID NO: 660 - DNA, 170 bp, Artificial Sequence
  Synthetic oligonucleotide
tatctacacgggtctcaccaaaacaaaaaaacacttcgggaggatgaagaaatgggcttgactacgacgtctcatatcatgagtctgcaatgtccaatttcgtacacaagaatgacagactcatgatagtagatggttttagagtgagaccagcgtaactctcaacgctt

SEQ ID NO: 661 - DNA, 170 bp, Artificial Sequence
  Synthetic oligonucleotide
tatctacacgggtctcaccaaaacaaaaaaacacttcgggaggatgaagaaatgggcttgactacgacgtctattatcatgagtctgcaatgtccaatttcgtacacaagaatgacagactcatgatagtagatggttttagagtgagaccagcgtaactctggtatact

SEQ ID NO: 662 - DNA, 170 bp, Artificial Sequence
  Synthetic oligonucleotide
tatctacacgggtctcaccaaaacaaaaaaacacttcgggaggatgaagaaatgggcttgactacgacgtctttgatcatgagtctgcaatgtccaatttcgtacacaagaatgacagactcatgatagtagatggttttagagtgagaccagcgtaactcgtatagcgt

SEQ ID NO: 663 - DNA, 170 bp, Artificial Sequence
  Synthetic oligonucleotide
tatctacacgggtctcaccaaaacaaaaaaacacttcgggaggatgaagaaatgggcttgactacgacgtctaaaatcatgagtctgcaatgtccaatttcgtacacaagaatgacagactcatgatagtagatggttttagagtgagaccagcgtaactccggctaaag

SEQ ID NO: 664 - DNA, 170 bp, Artificial Sequence
  Synthetic oligonucleotide
tatctacacgggtctcaccaaaacaaaaaaacacttcgggaggatgaagaaatgggcttgactacgacgtctatgatcatgagtctgcaatgtccaatttcgtacacaagaatgacagactcatgatagtagatggttttagagtgagaccagcgtaactccgccgtatg

SEQ ID NO: 665 - DNA, 170 bp, Artificial Sequence
  Synthetic oligonucleotide
tatctacacgggtctcaccaaaacaaaaaaacacttcgggaggatgaagaaatgggcttgactacgacgtcttttatcatgagtctgcaatgtccaatttcgtacacaagaatgacagactcatgatagtagatggttttagagtgagaccagcgtaactcgcctgcgcg

SEQ ID NO: 666 - DNA, 170 bp, Artificial Sequence
  Synthetic oligonucleotide
tatctacacgggtctcaccaaaacaaaaaaacacttcgggaggatgaagaaatgggcttgactacgacgtctccaatcatgagtctgcaatgtccaatttcgtacacaagaatgacagactcatgatagtagatggttttagagtgagaccagcgtaactcagagcaatt

SEQ ID NO: 667 - DNA, 170 bp, Artificial Sequence
  Synthetic oligonucleotide
tatctacacgggtctcaccaaaacaaaaaaacacttcgggaggatgaagaaatgggcttgactacgacgtcttctatcatgagtctgcaatgtccaatttcgtacacaagaatgacagactcatgatagtagatggttttagagtgagaccagcgtaactcccaattgat

SEQ ID NO: 668 - DNA, 170 bp, Artificial Sequence
  Synthetic oligonucleotide
tatctacacgggtctcaccaaaacaaaaaaacacttcgggaggatgaagaaatgggcttgactacgacgtctacaatcatgagtctgcaatgtccaatttcgtacacaagaatgacagactcatgatagtagatggttttagagtgagaccagcgtaactctcacaaatg

SEQ ID NO: 669 - DNA, 170 bp, Artificial Sequence
  Synthetic oligonucleotide
tatctacacgggtctcaccaaaacaaaaaaacacttcgggaggatgaagaaatgggcttgactacgacgtcttggatcatgagtctgcaatgtccaatttcgtacacaagaatgacagactcatgatagtagatggttttagagtgagaccagcgtaactccaacccttt

SEQ ID NO: 670 - DNA, 170 bp, Artificial Sequence
  Synthetic oligonucleotide
tatctacacgggtctcaccaaaacaaaaaaacacttcgggaggatgaagaaatgggcttgactacgacgtcttatatcatgagtctgcaatgtccaatttcgtacacaagaatgacagactcatgatagtagatggttttagagtgagaccagcgtaactcctcgtagga

SEQ ID NO: 671 - DNA, 170 bp, Artificial Sequence
  Synthetic oligonucleotide
tatctacacgggtctcaccaaaacaaaaaaacacttcgggaggatgaagaaatgggcttgactacgacgtctgttatcatgagtctgcaatgtccaatttcgtacacaagaatgacagactcatgatagtagatggttttagagtgagaccagcgtaactcggctgtcaa

SEQ ID NO: 672 - DNA, 170 bp, Artificial Sequence
  Synthetic oligonucleotide
tatctacacgggtctcaccaaaacaaaaaaacacttcgggaggatgaagaaatgggcttgactacgacgtctactgctatgagtctgcaatgtccaatttcgtacacaagaatgaaatcagactcatgatagtagatggttttagagtgagaccagcgtaactcggttgt

SEQ ID NO: 673 - DNA, 170 bp, Artificial Sequence
  Synthetic oligonucleotide
tatctacacgggtctcaccaaaacaaaaaaacacttcgggaggatgaagaaatgggcttgactacgacgtctactagaatgagtctgcaatgtccaatttcgtacacaagaatgaaatcagactcatgatagtagatggttttagagtgagaccagcgtaactccaggaa

SEQ ID NO: 674 - DNA, 170 bp, Artificial Sequence
  Synthetic oligonucleotide
tatctacacgggtctcaccaaaacaaaaaaacacttcgggaggatgaagaaatgggcttgactacgacgtctactaatatgagtctgcaatgtccaatttcgtacacaagaatgaaatcagactcatgatagtagatggttttagagtgagaccagcgtaactcagacta

SEQ ID NO: 675 - DNA, 170 bp, Artificial Sequence
  Synthetic oligonucleotide
tatctacacgggtctcaccaaaacaaaaaaacacttcgggaggatgaagaaatgggcttgactacgacgtctactgatatgagtctgcaatgtccaatttcgtacacaagaatgaaatcagactcatgatagtagatggttttagagtgagaccagcgtaactctctacc

SEQ ID NO: 676 - DNA, 170 bp, Artificial Sequence
  Synthetic oligonucleotide
tatctacacgggtctcaccaaaacaaaaaaacacttcgggaggatgaagaaatgggcttgactacgacgtctacttgtatgagtctgcaatgtccaatttcgtacacaagaatgaaatcagactcatgatagtagatggttttagagtgagaccagcgtaactccgagct

SEQ ID NO: 677 - DNA, 170 bp, Artificial Sequence
  Synthetic oligonucleotide
tatctacacgggtctcaccaaaacaaaaaaacacttcgggaggatgaagaaatgggcttgactacgacgtctactcaaatgagtctgcaatgtccaatttcgtacacaagaatgaaatcagactcatgatagtagatggttttagagtgagaccagcgtaactcctgtcg

SEQ ID NO: 678 - DNA, 170 bp, Artificial Sequence
  Synthetic oligonucleotide
tatctacacgggtctcaccaaaacaaaaaaacacttcgggaggatgaagaaatgggcttgactacgacgtctactgaaatgagtctgcaatgtccaatttcgtacacaagaatgaaatcagactcatgatagtagatggttttagagtgagaccagcgtaactcataggc

SEQ ID NO: 679 - DNA, 170 bp, Artificial Sequence
  Synthetic oligonucleotide
tatctacacgggtctcaccaaaacaaaaaaacacttcgggaggatgaagaaatgggcttgactacgacgtctactggtatgagtctgcaatgtccaatttcgtacacaagaatgaaatcagactcatgatagtagatggttttagagtgagaccagcgtaactcaagtga

SEQ ID NO: 680 - DNA, 170 bp, Artificial Sequence
  Synthetic oligonucleotide
tatctacacgggtctcaccaaaacaaaaaaacacttcgggaggatgaagaaatgggcttgactacgacgtctactcatatgagtctgcaatgtccaatttcgtacacaagaatgaaatcagactcatgatagtagatggttttagagtgagaccagcgtaactctcgggc

SEQ ID NO: 681 - DNA, 170 bp, Artificial Sequence
  Synthetic oligonucleotide
tatctacacgggtctcaccaaaacaaaaaaacacttcgggaggatgaagaaatgggcttgactacgacgtctactattatgagtctgcaatgtccaatttcgtacacaagaatgaaatcagactcatgatagtagatggttttagagtgagaccagcgtaactcccctcg

SEQ ID NO: 682 - DNA, 170 bp, Artificial Sequence
  Synthetic oligonucleotide
tatctacacgggtctcaccaaaacaaaaaaacacttcgggaggatgaagaaatgggcttgactacgacgtctactttgatgagtctgcaatgtccaatttcgtacacaagaatgaaatcagactcatgatagtagatggttttagagtgagaccagcgtaactctagcct

SEQ ID NO: 683 - DNA, 170 bp, Artificial Sequence
  Synthetic oligonucleotide
tatctacacgggtctcaccaaaacaaaaaaacacttcgggaggatgaagaaatgggcttgactacgacgtctactaaaatgagtctgcaatgtccaatttcgtacacaagaatgaaatcagactcatgatagtagatggttttagagtgagaccagcgtaactcatggag

SEQ ID NO: 684 - DNA, 170 bp, Artificial Sequence
  Synthetic oligonucleotide
tatctacacgggtctcaccaaaacaaaaaaacacttcgggaggatgaagaaatgggcttgactacgacgtctactatgatgagtctgcaatgtccaatttcgtacacaagaatgaaatcagactcatgatagtagatggttttagagtgagaccagcgtaactccgagtt

SEQ ID NO: 685 - DNA, 170 bp, Artificial Sequence
  Synthetic oligonucleotide
tatctacacgggtctcaccaaaacaaaaaaacacttcgggaggatgaagaaatgggcttgactacgacgtctacttttatgagtctgcaatgtccaatttcgtacacaagaatgaaatcagactcatgatagtagatggttttagagtgagaccagcgtaactcactgga

SEQ ID NO: 686 - DNA, 170 bp, Artificial Sequence
  Synthetic oligonucleotide
tatctacacgggtctcaccaaaacaaaaaaacacttcgggaggatgaagaaatgggcttgactacgacgtctactccaatgagtctgcaatgtccaatttcgtacacaagaatgaaatcagactcatgatagtagatggttttagagtgagaccagcgtaactctggttc

SEQ ID NO: 687 - DNA, 170 bp, Artificial Sequence
  Synthetic oligonucleotide
tatctacacgggtctcaccaaaacaaaaaaacacttcgggaggatgaagaaatgggcttgactacgacgtctacttctatgagtctgcaatgtccaatttcgtacacaagaatgaaatcagactcatgatagtagatggttttagagtgagaccagcgtaactcaccgct

SEQ ID NO: 688 - DNA, 170 bp, Artificial Sequence
  Synthetic oligonucleotide
tatctacacgggtctcaccaaaacaaaaaaacacttcgggaggatgaagaaatgggcttgactacgacgtctactactatgagtctgcaatgtccaatttcgtacacaagaatgaaatcagactcatgatagtagatggttttagagtgagaccagcgtaactcctcaag

SEQ ID NO: 689 - DNA, 170 bp, Artificial Sequence
  Synthetic oligonucleotide
tatctacacgggtctcaccaaaacaaaaaaacacttcgggaggatgaagaaatgggcttgactacgacgtctacttggatgagtctgcaatgtccaatttcgtacacaagaatgaaatcagactcatgatagtagatggttttagagtgagaccagcgtaactcgcttga

SEQ ID NO: 690 - DNA, 170 bp, Artificial Sequence
  Synthetic oligonucleotide
tatctacacgggtctcaccaaaacaaaaaaacacttcgggaggatgaagaaatgggcttgactacgacgtctacttatatgagtctgcaatgtccaatttcgtacacaagaatgaaatcagactcatgatagtagatggttttagagtgagaccagcgtaactcgccatg

SEQ ID NO: 691 - DNA, 170 bp, Artificial Sequence
  Synthetic oligonucleotide
tatctacacgggtctcaccaaaacaaaaaaacacttcgggaggatgaagaaatgggcttgactacgacgtctactgttatgagtctgcaatgtccaatttcgtacacaagaatgaaatcagactcatgatagtagatggttttagagtgagaccagcgtaactcagcgcc

SEQ ID NO: 692 - DNA, 170 bp, Artificial Sequence
  Synthetic oligonucleotide
tatctacacgggtctcaccaaaacaaaaaaacacttcgggaggatgaagaaatgggcttgactacgacgtctactatcgctagtctgcaatgtccaatttcgtacacaagaatgaaatacccagactcatgatagtagatggttttagagtgagaccagcgtaactctcg

SEQ ID NO: 693 - DNA, 170 bp, Artificial Sequence
  Synthetic oligonucleotide
tatctacacgggtctcaccaaaacaaaaaaacacttcgggaggatgaagaaatgggcttgactacgacgtctactatcagaagtctgcaatgtccaatttcgtacacaagaatgaaatacccagactcatgatagtagatggttttagagtgagaccagcgtaactcgta

SEQ ID NO: 694 - DNA, 170 bp, Artificial Sequence
  Synthetic oligonucleotide
tatctacacgggtctcaccaaaacaaaaaaacacttcgggaggatgaagaaatgggcttgactacgacgtctactatcaatagtctgcaatgtccaatttcgtacacaagaatgaaatacccagactcatgatagtagatggttttagagtgagaccagcgtaactccag

SEQ ID NO: 695 - DNA, 170 bp, Artificial Sequence
  Synthetic oligonucleotide
tatctacacgggtctcaccaaaacaaaaaaacacttcgggaggatgaagaaatgggcttgactacgacgtctactatcgatagtctgcaatgtccaatttcgtacacaagaatgaaatacccagactcatgatagtagatggttttagagtgagaccagcgtaactctca

SEQ ID NO: 696 - DNA, 170 bp, Artificial Sequence
  Synthetic oligonucleotide
tatctacacgggtctcaccaaaacaaaaaaacacttcgggaggatgaagaaatgggcttgactacgacgtctactatctgtagtctgcaatgtccaatttcgtacacaagaatgaaatacccagactcatgatagtagatggttttagagtgagaccagcgtaactctag

SEQ ID NO: 697 - DNA, 170 bp, Artificial Sequence
  Synthetic oligonucleotide
tatctacacgggtctcaccaaaacaaaaaaacacttcgggaggatgaagaaatgggcttgactacgacgtctactatccaaagtctgcaatgtccaatttcgtacacaagaatgaaatacccagactcatgatagtagatggttttagagtgagaccagcgtaactcacc

SEQ ID NO: 698 - DNA, 170 bp, Artificial Sequence
  Synthetic oligonucleotide
tatctacacgggtctcaccaaaacaaaaaaacacttcgggaggatgaagaaatgggcttgactacgacgtctactatcgaaagtctgcaatgtccaatttcgtacacaagaatgaaatacccagactcatgatagtagatggttttagagtgagaccagcgtaactctta

SEQ ID NO: 699 - DNA, 170 bp, Artificial Sequence
  Synthetic oligonucleotide
tatctacacgggtctcaccaaaacaaaaaaacacttcgggaggatgaagaaatgggcttgactacgacgtctactatcggtagtctgcaatgtccaatttcgtacacaagaatgaaatacccagactcatgatagtagatggttttagagtgagaccagcgtaactcgtg

SEQ ID NO: 700 - DNA, 170 bp, Artificial Sequence
  Synthetic oligonucleotide
tatctacacgggtctcaccaaaacaaaaaaacacttcgggaggatgaagaaatgggcttgactacgacgtctactatccatagtctgcaatgtccaatttcgtacacaagaatgaaatacccagactcatgatagtagatggttttagagtgagaccagcgtaactccca

SEQ ID NO: 701 - DNA, 170 bp, Artificial Sequence
  Synthetic oligonucleotide
tatctacacgggtctcaccaaaacaaaaaaacacttcgggaggatgaagaaatgggcttgactacgacgtctactatcattagtctgcaatgtccaatttcgtacacaagaatgaaatacccagactcatgatagtagatggttttagagtgagaccagcgtaactcaag

SEQ ID NO: 702 - DNA, 170 bp, Artificial Sequence
  Synthetic oligonucleotide
tatctacacgggtctcaccaaaacaaaaaaacacttcgggaggatgaagaaatgggcttgactacgacgtctactatcttgagtctgcaatgtccaatttcgtacacaagaatgaaatacccagactcatgatagtagatggttttagagtgagaccagcgtaactcgat

SEQ ID NO: 703 - DNA, 170 bp, Artificial Sequence
  Synthetic oligonucleotide
tatctacacgggtctcaccaaaacaaaaaaacacttcgggaggatgaagaaatgggcttgactacgacgtctactatcaaaagtctgcaatgtccaatttcgtacacaagaatgaaatacccagactcatgatagtagatggttttagagtgagaccagcgtaactctta

SEQ ID NO: 704 - DNA, 170 bp, Artificial Sequence
  Synthetic oligonucleotide
tatctacacgggtctcaccaaaacaaaaaaacacttcgggaggatgaagaaatgggcttgactacgacttctactatcatgagtctgcaatgtccaatttcgtacacaagaatgaaatacccagactcatgatagtagatggttttagagtgagaccagcgtaactccta

SEQ ID NO: 705 - DNA, 170 bp, Artificial Sequence
  Synthetic oligonucleotide
tatctacacgggtctcaccaaaacaaaaaaacacttcgggaggatgaagaaatgggcttgactacgacgtctactatctttagtctgcaatgtccaatttcgtacacaagaatgaaatacccagactcatgatagtagatggttttagagtgagaccagcgtaactcgag

SEQ ID NO: 706 - DNA, 170 bp, Artificial Sequence
  Synthetic oligonucleotide
tatctacacgggtctcaccaaaacaaaaaaacacttcgggaggatgaagaaatgggcttgactacgacgtctactatcccaagtctgcaatgtccaatttcgtacacaagaatgaaatacccagactcatgatagtagatggttttagagtgagaccagcgtaactcgtc

SEQ ID NO: 707 - DNA, 170 bp, Artificial Sequence
  Synthetic oligonucleotide
tatctacacgggtctcaccaaaacaaaaaaacacttcgggaggatgaagaaatgggcttgactacgacgtctactatctctagtctgcaatgtccaatttcgtacacaagaatgaaatacccagactcatgatagtagatggttttagagtgagaccagcgtaactcagt

SEQ ID NO: 708 - DNA, 170 bp, Artificial Sequence
  Synthetic oligonucleotide
tatctacacgggtctcaccaaaacaaaaaaacacttcgggaggatgaagaaatgggcttgactacgacgtctactatcactagtctgcaatgtccaatttcgtacacaagaatgaaatacccagactcatgatagtagatggttttagagtgagaccagcgtaactccac

SEQ ID NO: 709 - DNA, 170 bp, Artificial Sequence
  Synthetic oligonucleotide
tatctacacgggtctcaccaaaacaaaaaaacacttcgggaggatgaagaaatgggcttgactacgacgtctactatctggagtctgcaatgtccaatttcgtacacaagaatgaaatacccagactcatgatagtagatggttttagagtgagaccagcgtaactcgta

SEQ ID NO: 710 - DNA, 170 bp, Artificial Sequence
  Synthetic oligonucleotide
tatctacacgggtctcaccaaaacaaaaaaacacttcgggaggatgaagaaatgggcttgactacgacgtctactatctatagtctgcaatgtccaatttcgtacacaagaatgaaatacccagactcatgatagtagatggttttagagtgagaccagcgtaactcaga

SEQ ID NO: 711 - DNA, 170 bp, Artificial Sequence
  Synthetic oligonucleotide
tatctacacgggtctcaccaaaacaaaaaaacacttcgggaggatgaagaaatgggcttgactacgacgtctactatcgttagtctgcaatgtccaatttcgtacacaagaatgaaatacccagactcatgatagtagatggttttagagtgagaccagcgtaactctgg

SEQ ID NO: 712 - DNA, 170 bp, Artificial Sequence
  Synthetic oligonucleotide
tatctacacgggtctcaccaaaacttattgattttgaagggtatttcattcttgtgtacgagatcggacattgcagagccatgatagtagatgtggtagtcaagcccatttcttcatccttcattcttgtgtacgaaatgttttagagtgagaccagcgtaactcttgcc

SEQ ID NO: 713 - DNA, 170 bp, Artificial Sequence
  Synthetic oligonucleotide
tatctacacgggtctcaccaaaacttattgattttgaagggtatttcattcttgtgtacgagatcggacattgcagtctcatgatagtagatgtggtagtcaagcccatttcttcatccttcattcttgtgtacgaaatgttttagagtgagaccagcgtaactctgccc

SEQ ID NO: 714 - DNA, 170 bp, Artificial Sequence
  Synthetic oligonucleotide
tatctacacgggtctcaccaaaacttattgattttgaagggtatttcattcttgtgtacgagatcggacattgcagattcatgatagtagatgtggtagtcaagcccatttcttcatccttcattcttgtgtacgaaatgttttagagtgagaccagcgtaactcgcgct

SEQ ID NO: 715 - DNA, 170 bp, Artificial Sequence
  Synthetic oligonucleotide
tatctacacgggtctcaccaaaacttattgattttgaagggtatttcattcttgtgtacgagatcggacattgcagatccatgatagtagatgtggtagtcaagcccatttcttcatccttcattcttgtgtacgaaatgttttagagtgagaccagcgtaactcgggtg

SEQ ID NO: 716 - DNA, 170 bp, Artificial Sequence
  Synthetic oligonucleotide
tatctacacgggtctcaccaaaacttattgattttgaagggtatttcattcttgtgtacgagatcggacattgcagacacatgatagtagatgtggtagtcaagcccatttcttcatccttcattcttgtgtacgaaatgttttagagtgagaccagcgtaactccggcc

SEQ ID NO: 717 - DNA, 170 bp, Artificial Sequence
  Synthetic oligonucleotide
tatctacacgggtctcaccaaaacttattgattttgaagggtatttcattcttgtgtacgagatcggacattgcagttgcatgatagtagatgtggtagtcaagcccatttcttcatccttcattcttgtgtacgaaatgttttagagtgagaccagcgtaactcgttcc

SEQ ID NO: 718 - DNA, 170 bp, Artificial Sequence
  Synthetic oligonucleotide
tatctacacgggtctcaccaaaacttattgattttgaagggtatttcattcttgtgtacgagatcggacattgcagttccatgatagtagatgtggtagtcaagcccatttcttcatccttcattcttgtgtacgaaatgttttagagtgagaccagcgtaactctgcgg

SEQ ID NO: 719 - DNA, 170 bp, Artificial Sequence
  Synthetic oligonucleotide
tatctacacgggtctcaccaaaacttattgattttgaagggtatttcattcttgtgtacgagatcggacattgcagacccatgatagtagatgtggtagtcaagcccatttcttcatccttcattcttgtgtacgaaatgttttagagtgagaccagcgtaactcataga

SEQ ID NO: 720 - DNA, 170 bp, Artificial Sequence
  Synthetic oligonucleotide
tatctacacgggtctcaccaaaacttattgattttgaagggtatttcattcttgtgtacgagatcggacattgcagatgcatgatagtagatgtggtagtcaagcccatttcttcatccttcattcttgtgtacgaaatgttttagagtgagaccagcgtaactcgagga

SEQ ID NO: 721 - DNA, 170 bp, Artificial Sequence
  Synthetic oligonucleotide
tatctacacgggtctcaccaaaacttattgattttgaagggtatttcattcttgtgtacgagatcggacattgcagaatcatgatagtagatgtggtagtcaagcccatttcttcatccttcattcttgtgtacgaaatgttttagagtgagaccagcgtaactccgcgg

SEQ ID NO: 722 - DNA, 170 bp, Artificial Sequence
  Synthetic oligonucleotide
tatctacacgggtctcaccaaaacttattgattttgaagggtatttcattcttgtgtacgagatcggacattgcagcaacatgatagtagatgtggtagtcaagcccatttcttcatccttcattcttgtgtacgaaatgttttagagtgagaccagcgtaactctccgc

SEQ ID NO: 723 - DNA, 170 bp, Artificial Sequence
  Synthetic oligonucleotide
tatctacacgggtctcaccaaaacttattgattttgaagggtatttcattcttgtgtacgagatcggacattgcagtttcatgatagtagatgtggtagtcaagcccatttcttcatccttcattcttgtgtacgaaatgttttagagtgagaccagcgtaactcgcacg

SEQ ID NO: 724 - DNA, 170 bp, Artificial Sequence
  Synthetic oligonucleotide
tatctacacgggtctcaccaaaacttattgattttgaagggtatttcattcttgtgtacgagatcggacattgcagcatcatgatagtagatgtggtagtcaagcccatttcttcatccttcattcttgtgtacgaaatgttttagagtgagaccagcgtaactcatata

SEQ ID NO: 725 - DNA, 170 bp, Artificial Sequence
  Synthetic oligonucleotide
tatctacacgggtctcaccaaaacttattgattttgaagggtatttcattcttgtgtacgagatcggacattgcagaaacatgatagtagatgtggtagtcaagcccatttcttcatccttcattcttgtgtacgaaatgttttagagtgagaccagcgtaactcgtgac

SEQ ID NO: 726 - DNA, 170 bp, Artificial Sequence
  Synthetic oligonucleotide
tatctacacgggtctcaccaaaacttattgattttgaagggtatttcattcttgtgtacgagatcggacattgcagtggcatgatagtagatgtggtagtcaagcccatttcttcatccttcattcttgtgtacgaaatgttttagagtgagaccagcgtaactcgaacc

SEQ ID NO: 727 - DNA, 170 bp, Artificial Sequence
  Synthetic oligonucleotide
tatctacacgggtctcaccaaaacttattgattttgaagggtatttcattcttgtgtacgagatcggacattgcagagacatgatagtagatgtggtagtcaagcccatttcttcatccttcattcttgtgtacgaaatgttttagagtgagaccagcgtaactccgcat

SEQ ID NO: 728 - DNA, 170 bp, Artificial Sequence
  Synthetic oligonucleotide
tatctacacgggtctcaccaaaacttattgattttgaagggtatttcattcttgtgtacgagatcggacattgcagagtcatgatagtagatgtggtagtcaagcccatttcttcatccttcattcttgtgtacgaaatgttttagagtgagaccagcgtaactcttacg

SEQ ID NO: 729 - DNA, 170 bp, Artificial Sequence
  Synthetic oligonucleotide
tatctacacgggtctcaccaaaacttattgattttgaagggtatttcattcttgtgtacgagatcggacattgcagccacatgatagtagatgtggtagtcaagcccatttcttcatccttcattcttgtgtacgaaatgttttagagtgagaccagcgtaactcaggag

SEQ ID NO: 730 - DNA, 170 bp, Artificial Sequence
  Synthetic oligonucleotide
tatctacacgggtctcaccaaaacttattgattttgaagggtatttcattcttgtgtacgagatcggacattgcagatacatgatagtagatgtggtagtcaagcccatttcttcatccttcattcttgtgtacgaaatgttttagagtgagaccagcgtaactcgggtg

SEQ ID NO: 731 - DNA, 170 bp, Artificial Sequence
  Synthetic oligonucleotide
tatctacacgggtctcaccaaaacttattgattttgaagggtatttcattcttgtgtacgagatcggacattgcagaaccatgatagtagatgtggtagtcaagcccatttcttcatccttcattcttgtgtacgaaatgttttagagtgagaccagcgtaactccaagc

SEQ ID NO: 732 - protein, 201 aa, Saccharomyces cerevisiae
MARPVNTNAETESRGRPTQGGGYASNNNGSCNNNNGSNNNNNNNNNNNNNSNNSNNNNGPTSSGRTNGKQRLTAAQQQYIKNLIETHITDNHPDLRPKSHPMDFEEYTDAFLRRYKDHFQLDVPDNLTLQGYLLGSKLGAKTYSYKRNTQGQHDKRIHKRDLANVVRRHFDEHSIKETDCIPQFIYKVKNQKKKFKMEFRG

SEQ ID NO: 733 - protein, 148 aa, Saccharomyces cerevisiae
MSSSKRIAKELSDLERDPPTSCSAGPVGDDLYHWQASIMGPADSPYAGGVFFLSIHFPTDYPFKPPKISFTTKIYHPNINANGNICLDILKDQWSPALTLSKVLLSICSLLTDANPDDPLVPEIAHIYKTDRPKYEATAREWTKKYAV

SEQ ID NO: 734 - protein, 976 aa, Saccharomyces cerevisiae
MAKDLNDSGFPPKRKPLLRPQRSDFTANSSTTMNVNANTRGRGRQKQEGGKGSSRSPSLHSPKSWIRSASATGILGLRRPELAHSHSHAPSTGTPAGGNRSPLRRSTANATPVETGRSLTDGDINNVVDVLPSFEMYNTLHRHIPQGNVDPDRHDFPPSYQEANNSTATGAAGSSADLSHQSLSTDALGATRSSSTSNLENLIPLRTEHHSIAAHQSTAVDEDSLDIPPILDDLNDTDNIFIDKLYTLPKMSTPIEITIKTTKHAPIPHVKPEEESILKEYTSGDLIHGFITIENKSQANLKFEMFYVTLESYISIIDKVKSKRTIKRFLRMVDLSASWSYSKIALGSGVDFIPADVDYDGSVFGLNNSRVLEPGVKYKKFFIFKLPLQLLDVTCKQEHFSHCLLPPSFGIDKYRNNCKYSGIKVNRVLGCGHLGTKGSPILTNDMSDDNLSINYTIDARIVGKDQKASKLYIMKEREYNLRVIPFGFDANVVGERTTMSQLNDITKLVQERLDALRKIFQRLEKKEPITNRDIHGADLSGTIDDSIESDSQEILQRKLDQLHIKNRNNYLVNYNDLKLGHDLDNGRSGNSGHNTDTSRAWGPFVESELKYKLKNKSNSSSFLNFSHFLNSSSSSMSSSSNAGKNNHDLTGNKERTGLILVKAKIPKQGLPYWAPSLLRKTNVFESKSKHDQENWVRLSELIPEDVKKPLEKLDLQLTCIESDNSLPHDPPEIQSITTELICITAKSDNSIPIKLNSELLMNKEKLTSIKALYDDFHSKICEYETKFNKNFLELNELYNMNRGDRRPKELKFTDFITSQLFNDIESICNLKVSVHNLSNIFKKQVSTLKQHSKHALSEDSISHTGNGSSSSPSSASLTPVTSSSKSSLFLPSGSSSTSLKFTDQIVHKWVRIAPLQYKRDINVNLEFNKDIKETLIPSFESCLCCRFYCVRVMIKFENHLGVAKIDIPISVRQVTK

SEQ ID NO: 735 - protein, 382 aa, Saccharomyces cerevisiae
MRKELKYLICFNILLLLSIIYYTFDLLTLCIDDTVKDAILEEDLNPDAPPKPQLIPKIIHQTYKTEDIPEHWKEGRQKCLDLHPDYKYILWTDEMAYEFIKEEYPWFLDTFENYKYPIERADAIRYFILSHYGGVYIDLDDGCERKLDPLLAFPAFLRKTSPLGVSNDVMGSVPRHPFFLKALKSLKHYDKYWFIPYMTIMGSTGPLFLSVIWKQYKRWRIPKNGTVRILQPAYYKMHSYSFFSITKGSSWHLDDAKLMKALENHILSCVVTGFIFGFFILYGEFTFYCWLCSKNFSNLTKNWKLNAIKVRFVTILNSLGLRLKLSKSTSDTASATLLARQQKRLRKDSNTNIVLLKSSRKSDVYDLEKNDSSKYSLGNNSS

SEQ ID NO: 736 - protein, 904 aa, Saccharomyces cerevisiae
MINLEDYWEDETPGPDREPTNELRNEVEETITLMELLKVSELKDICRSVSFPVSGRKAVLQDLIRNFLQNALVVGKSDPYRVQAVKFLIERIRKNEPLPVYKDLWNALRKGTPLSAITVRSMEGPPTVQQQSPSVIRQSPTQRRKTSTTSSTSRAPPPTNPDASSSSSSFAVPTIHFKESPFYKIQRLIPELVMNVEVTGGRGMCSAKFKLSKADYNLLSNPNSKHRLYLFSGMINPLGSRGNEPIQFPFPNELRCNNVQIKDNIRGFKSKPGTAKPADLTPHLKPYTQQNNVELIYAFTTKEYKLFGYIVEMITPEQLLEKVLQHPKIIKQATLLYLKKTLREDEEMGLTTTSTIMSLQCPISYTRMKYPSKSINCKHLQCFDALWFLHSQLQIPTWQCPVCQIDIALENLAISEFVDDILQNCQKNVEQVELTSDGKWTAILEDDDDSDSDSNDGSRSPEKGTSVSDHHCSSSHPSEPIIINLDSDDDEPNGNNPHVTNNHDDSNRHSNDNNNNSIKNNDSHNKNNNNNNNNNNNNNDNNNSIENNDSNSNNKHDHGSRSNTPSHNHTKNLMNDNDDDDDDRLMAEITSNHLKSTNTDILTEKGSSAPSRTLDPKSYNIVASETTTPVTNRVIPEYLGNSSSYIGKQLPNILGKTPLNVTAVDNSSHLISPDVSVSSPTPRNTASNASSSALSTPPLIRMSSLDPRGSTVPDKTIRPPINSNSYTASISDSFVQPQESSVFPPREQNMDMSFPSTVNSRFNDPRLNTTRFPDSTLRGATILSNNGLDQRNNSLPTTEAITRNDVGRQNSTPVLPTLPQNVPIRTNSNKSGLPLINNENSVPNPPNTATIPLQKSRLIVNPFIPRRPYSNVLPQKRQLSNTSSTSPIMGTWKTQDYGKKYNSG

SEQ ID NO: 737 - protein, 409 aa, Saccharomyces cerevisiae
MVDGLNTSNIRKRARTLSNPNDFQEPNYLLDPGNHPSDHFRTRMSKFRFNIREKLLVFTNNQSFTLSRWQKKYRSAFNDLYFTYTSLMGSHTFYVLCLPMPVWFGYFETTKDMVYILGYSIYLSGFFKDYWCLPRPRAPPLHRITLSEYTTKEYGAPSSHTANATGVSLLFLYNIWRMQESSVMVQLLLSCVVLFYYMTLVFGRIYCGMHGILDLVSGGLIGIVCFIVRMYFKYRFPGLRIEEHWWFPLFSVGWGLLLLFKHVKPVDECPCFQDSVAFMGVVSGIECCDWLGKVFGVTLVYNLEPNCGWRLTLARLLVGLPCVVIWKYVISKPMIYTLLIKVFHLKDDRNVAARKRLEATHKEGASKYECPLYIGEPKIDILGRFIIYAGVPFTVVMCSPVLFSLLNIA

(19) United States
(12) Patent Application Publication
(10) Pub. No.: US 2019/0218533 A1
(43) Pub. Date: Jul. 18, 2019

(54) Genome-Scale Engineering of Cells with Single Nucleotide Precision

(71) Applicant: The Board of Trustees of the University of Illinois, Urbana, IL (US)

(72) Inventors: Huimin Zhao, Champaign, IL (US); Zehua Bao, Urbana, IL (US)

(21) Appl. No.: 16/248,899

(22) Filed: Jan. 16, 2019

Related U.S. Application Data
(60) Provisional application No. 62/617,890, filed on Jan. 16, 2018.

(51) Int. Cl. C12N 9/22; C12N 15/11; C12Q 1/686; C12N 15/10

(52) U.S. Cl. CPC C12N 9/22; C12N 15/11; C12N 2310/20; C12N 15/1093; C12N 2800/80; C12Q 1/686

(57) ABSTRACT

Provided herein are methods and compositions for a CRISPR and homology-directed-repair assisted genome-scale engineering that can rapidly output tens of thousands of specific genetic variants in host cells. More than 98% of target sequences can be efficiently edited with a high average frequency.

### CROSS-REFERENCE TO RELATED APPLICATIONS

This application claims the benefit of U.S. Provisional Patent Application No. 62/617,890, filed on Jan. 16, 2018, the disclosure of which is hereby incorporated by cross-reference in its entirety.

### STATEMENT REGARDING FEDERALLY SPONSORED RESEARCH OR DEVELOPMENT

This application was made with United States government support awarded by U.S. Department of Energy (DE-SC0018260). The United States government has certain rights in this invention.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 275 kilobytes in size, and titled “18-1869-US_SequenceListing_ST25.txt.”

### BACKGROUND

High-throughput genome-wide engineering of eukaryotic cells has not previously been accomplished. One problem with some existing genome-scale methods is that because Escherichia coli cannot readily repair double stranded breaks there is substantial selection pressure during mutagenesis for cells that have undergone homology-directed-repair. The same is not true in yeast and high-throughput approaches have thus far not been proven to work efficiently on a genome-wide scale.

### BRIEF SUMMARY

An embodiment provides a vector comprising a first promoter upstream of an insertion site and downstream of the insertion site: a terminator, a second promoter, a nucleic acid molecule encoding an RNA-guided DNA endonuclease protein, a third promoter, and a tracrRNA sequence, and in the insertion site a genetic engineering cassette comprising from a 5′ end to a 3′ end: a first direct repeat sequence;
(i) a homologous recombination editing template comprising two homology arms with a deletion portion, a substitution portion, or an insertion portion between the two homology arms;
(ii) a guide sequence; and
(iii) a second direct repeat sequence.

The homologous recombination editing template can comprise a deletion portion that removes a protospacer adjacent motif (PAM) sequence and causes a gene disruption. The genetic engineering cassette can further comprise a first priming site at a 5′ end of the cassette and a second priming site at a 3′ end of the cassette. The first priming site and the second priming site can each comprise a restriction enzyme cleavage site.

Another embodiment provides a pool of vectors comprising 20 or more of the vectors described above, wherein the vectors comprise genetic engineering cassettes specific for 20 or more target nucleic acid molecules.

Yet another embodiment provides a pool of host cells comprising two or more vectors.

Even another embodiment provides a method of homology directed repair-assisted engineering comprising delivering the pool of vectors to host cells to generate a pool of unique transformed genetic variant host cells. The pool of unique transformed variant host cells comprises host cells that have mutations throughout the host cell genome. The method can further comprise isolating transformed genetic variant host cells with one or more phenotypes; and determining a genomic locus of a nucleic acid molecule that causes one or more phenotypes. Determining the genomic locus can comprise using a genetic bar code or a sequence of the homologous recombination editing template. More than about 1,000 unique transformed genetic variant host cells can be generated using the method.

Another embodiment provides a method of saturation mutagenesis of a target nucleic acid molecule in host cells. The method can comprise making a plurality of genetic engineering cassettes that target a target nucleic acid molecule at a plurality of positions, wherein the genetic engineering cassettes comprise from a 5′ end to a 3′ end:
(i) a first direct repeat sequence;
(ii) a homologous recombination editing template comprising two homology arms with a deletion portion, a substitution portion, or an insertion portion between the two homology arms;
(iii) a guide sequence; and
(iv) a second direct repeat sequence;
   inserting the plurality of genetic engineering cassettes into insertions sites of vectors to create a vector pool; wherein the vectors comprise a first promoter upstream of the insertion sites and downstream of the insertion sites: a terminator, a second promoter, a nucleic acid molecule encoding an RNA-guided DNA endonuclease protein, a third promoter, and a tracrRNA sequence; delivering the pool of vectors to the host cells; isolating transformed host cells with one or more phenotypes; and determining the genomic locus of a nucleic acid molecule that causes one or more phenotypes.

Even another embodiment provides a method of engineering a desired phenotype of host cells. The method comprises constructing a vector library, wherein the vector library comprises two or more vectors each comprising a genetic engineering cassette in an insertion site of the vector that target one or more target sequences of the host cells at one or more positions, wherein the genetic engineering cassettes comprise from a 5′ end to a 3′ end:
(i) a first direct repeat sequence;
(ii) a homologous recombination editing template comprising two homology arms with a deletion portion, a substitution portion, or an insertion portion between the two homology arms;
(iii) a guide sequence; and
(iv) a second direct repeat sequence;
   The vectors comprise a first promoter upstream of the insertion site and downstream of the insertion site: a terminator, a second promoter, a nucleic acid molecule encoding an RNA-guided DNA endonuclease protein, a third promoter, and a tracrRNA sequence. The host cells are transformed with the vector library to form a transformed host cell pool and host cells with a desired phenotype are selected.

The transformed host cell pool can be enriched for the desired phenotype prior to selecting host cells with a desired phenotype. The vectors can be extracted from the transformed host cell pool and sequenced.

Yet another embodiment provides a genetic engineering cassette comprising from a 5′ end to a 3′ end:
(i) a first direct repeat sequence;
(ii) a first homologous recombination editing template comprising two homology arms with a deletion portion, a substitution portion, or an insertion portion between the two homology arms;
(iii) a first guide sequence;
(iv) a second direct repeat sequence;
(v) a second homologous recombination editing template comprising two homology arms with a deletion portion, a substitution portion, or an insertion portion between the two homology arms;
(vi) a second guide sequence; and
(vii) a third direct repeat sequence.

The genetic engineering cassette can further comprise a first priming site at a 5′ end of the cassette and a second priming site at a 3′ end of the cassette. The first priming site and the second priming site can each comprise a restriction enzyme cleavage site. The first homologous recombination editing template and the second homologous recombination editing template can each provide for a first substitution, first insertion, or first deletion, and a second substitution, second insertion, or second deletion in different locations of the same target polynucleotide. The first substitution, first insertion, or first deletion and the second substitution, second insertion, or second deletion site, can occur in any two loci across the whole genome of the host cell. The first substitution can be a substitution of 1 to 6 nucleic acids, the first insertion can be an insertion of 1 to 6 nucleic acids, the first deletion can be a deletion of 1 to 6 nucleic acids, the second substitution can be a substitution of 1 to 6 nucleic acids, the second insertion can be an insertion of 1 to 6 nucleic acids, and the second deletion can be a deletion of 1 to 6 nucleic acids.

An embodiment provides a vector comprising the genetic engineering cassette as described herein. The vector can comprise a first promoter upstream of the genetic engineering cassette and downstream of the genetic engineering cassette: a terminator, a second promoter, a nucleic acid molecule encoding an RNA-guided DNA endonuclease protein, a third promoter, and a tracrRNA sequence.

Another embodiment provides a pool of vectors comprising two or more of the vectors of described herein, wherein each of the genetic engineering cassettes is unique.

Even another embodiment provides a method of homology directed repair-assisted engineering comprising delivering the pool of vectors as described herein to host cells and isolating transformed host cells.

Yet another embodiment provides a genetically engineered yeast having attenuated expression of a polynucleotide encoding a SAP30 polypeptide, a UBC4 polypeptide, a BUL1 polypeptide, a SUR1 polypeptide, a SIZ1 polypeptide, a LCB3 polypeptide, or combination thereof. The SAP30 polypeptide can have at least 90% identity to SEQ ID N0:732, the UBC4 polypeptide can have at least 90% identity to SEQ ID NO:733, the BUL1 polypeptide can have at least 90% identity to SEQ ID NO:734, the SUR1 polypeptide can have at least 90% identity to SEQ ID NO:735, the SIZ1 polypeptide can have at least 90% sequence identity to SEQ ID NO:736, and the LCB3 polypeptide can have at least 90% sequence identity to SEQ ID NO:737.

An embodiment provides a genetically engineered yeast having improved furfural tolerance as compared to a wild-type yeast or control yeast, wherein the biological activity of an endogenous protein having at least 90% sequence identity to an amino acid sequence set forth in SEQ ID NO:732, SEQ ID NO:733, or SEQ ID NO:736, or a combination thereof is reduced or eliminated as compared to a wild-type or control yeast.

Another embodiment provides a genetically engineered yeast having improved acetic acid tolerance as compared to a wild-type yeast or control, wherein the biological activity of an endogenous protein having at least 90% sequence identity to an amino acid sequence set forth in SEQ ID NO:734 and SEQ ID NO:735, or SEQ ID NO:734 is reduced or eliminated as compared to a wild-type or control yeast. The attenuated expression can be caused by at least one gene disruption of a SAP30 gene, a UBC4 gene, a BUL1 gene, a SUR1 gene, a SIZ1 gene, a LCB3 gene, or combinations thereof which results in attenuated expression of the SAP30 gene, the UBC4 gene, the BUL1 gene, the SUR1 gene, the SIZ1 gene, the LCB3 gene, or combinations thereof. The yeast can express a SAP30 polypeptide, a UBC4 polypeptide, a BUL1 polypeptide, a SUR1 polypeptide, a SIZ1 polypeptide, a LCB3 polypeptide, or a combination thereof at a level of about 10%, 20%, 30%, 40%, 50%, 60%, 70%, 80%, 90% 95%, or 100% less than a wild-type or control yeast. The yeast can have improved furfural tolerance, improved acetic acid tolerance, or both as compared to a wild-type or control yeast. The yeast can be selected from Saccharomyces cerevisiae, Saccharomyces fermentati, Saccharomyces paradoxus, Saccharomyces uvarum, Saccharomyces bay anus, Schizosaccharomyces pombe, Schizosaccharomyces japonicus, Schizosaccharomyces octosporus, Schizosaccharomyces cryophilus, Torulaspora delbrueckii, Kluyveromyces marxianus, Pichia stipitis, Pichia pastoris, Pichia angusta, Zygosaccharomyces bailii, Brettanomyces inter medius, Brettanomyces bruxellensis, Brettanomyces anomalus, Brettanomyces custersianus, Brettanomyces naardenensis, Brettanomyces nanus, Dekkera bruxellensis, Dekkera anomala, Issatchenkia orientalis, Kloeckera apiculata; and Aureobasidium pullulans.

One or more of the regulatory elements controlling expression of the polynucleotides encoding a SAP30 polypeptide, a UBC4 polypeptide, a SUR1 polypeptide, a BUL1 polypeptide, a SIZ1 polypeptide, a LCB3 polypeptide, or a combination thereof can be mutated to prevent or attenuate expression of the SAP30 polypeptide, the UBC4 polypeptide, the SUR1 polypeptide, the BUL1 polypeptide, the SIZ1 polypeptide, the LCB3 polypeptide or a combination thereof as compared to a wild-type or control yeast. The regulatory elements controlling expression of the polynucleotides encoding SAP30, UBC4, SUR1, BUL1, SIZ1, LCB3 polypeptides or combinations thereof can be replaced with recombinant regulatory elements that prevent or attenuate the expression of the SAP30 polypeptide, the UBC4 polypeptide, the SUR1 polypeptide, the BUL1 polypeptide, the SIZ1 polypeptides, LCB3 polypeptides, or combinations thereof as compared to wild-type yeast or a control yeast.

Even another embodiment provides a method of making a genetically engineered yeast having improved tolerance of furfural or improved tolerance of acetic acid. The method comprises deleting or mutating a polynucleotide encoding at least one polypeptide selected from a SAP30 polypeptide, a UBC4 polypeptide, a SUR1 polypeptide, a BUL1 polypeptide, a SIZ1 polypeptide, a LCB3 polypeptide, or combinations thereof such that the SAP30 polypeptide, the UBC4 polypeptide, the SUR1 polypeptide, the UCB4 polypeptide, the SIZ1 polypeptide, the LCB3 polypeptide, or combinations thereof are expressed with an attenuated rate as compared to a wild-type or control yeast.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. CHAnGE enables rapid generation of genome-wide yeast disruption mutants and directed evolution of complex phenotypes. (a) Design of the CHAnGE cassette. DR, direct repeat. (b) The CHAnGE workflow. (c) Distribution of guide sequences by predicted scores. (d) Editing efficiencies of CHAnGE cassettes with varying predicted scores. The box extends from the 25^{th} to 75^{th} percentiles. The line in the middle of the box is plotted at the median. The plus symbol denotes the mean. The whiskers go down to the smallest value and up to the largest. n=12 for the group with scores over 60. n=18 for the group with scores less than 60. (e) Genetic screening of CAN1 disruption mutants in the presence of canavanine. Volcano plot is shown for canavanine stressed libraries versus untreated libraries. The X-axis represents enrichment levels of each guide sequence. The Y-axis represents log 10 transformed P values. Significantly enriched guides (p<0.05, fold change >1.5) are denoted by black dots, all others by gray dots. Dotted lines indicate 1.5-fold ratio (X-axis) and P value of 0.05 (Y-axis). n=2 independent experiments. (f) Enrichment of guide sequences during the first round and second round directed evolution of furfural tolerance. (g) Biomass accumulation of the wild type and mutant strains in the presence of furfural. n=3 independent experiments. Error bars represent standard error of the mean. Two-tailed t-tests were performed to determine significance levels against the wild type strain. *, P<0.05. ****, P<0.0001. ns, not significant.

FIG. 2. CHAnGE enables genome editing with a single-nucleotide resolution. (a) A representative figure showing the designed mutations in the Siz1 D345A CHAnGE cassette. The designed mutations in the HR template and the amino acid substitution were colored in red. A Sanger sequencing trace file of a representative edited colony was shown at the bottom. The wild-type nucleic acid is SEQ ID NO:83. The wild-type amino acid is SEQ ID NO:84. The template nucleic acid is SEQ ID NO:85. The template amino acid is SEQ ID NO:86. The edited nucleic acid is SEQ ID NO:85. The edited amino acid is SEQ ID NO:86. (b) A summary of SIZ1 precise editing efficiencies. For each mutagenesis, 5 randomly picked colonies were examined. (c) Spotting assay of SIZ1 mutants in the presence of furfural. Black triangles denote serial dilutions. (d) Design of a modified CHAnGE cassette for single-nucleotide resolution editing. Blue rectangles denote the target codon and the PAM. Red stars denote mutations for codon substitution and PAM elimination. (e) Editing efficiencies of modified CHAnGE cassettes with varying PAM-codon distances. The box extends from the 25^{th} to 75^{th} percentiles. The line in the middle of the box is plotted at the median. The plus symbol denotes the mean. The whiskers go down to the smallest value and up to the largest. n=10 for the group with distances less than 20 bp. n=20 for the group with distances over 20 bp. (f) Crystal structure of Siz1 SP-CTD forming a complex with SUMO. Black dashed lines denote hydrogen bonds. PDB code SJNE. (g) Heatmap showing the enrichment of 580 CHAnGE cassettes after selection with 5 mM furfural. Original and substitute amino acid residues are denoted on the top and at the left, respectively, and are colored according to the Lesk color scheme. Synonymous CHAnGE cassettes are denoted by green boxes. Cassette D345A is denoted by a blue box.

FIG. 3 shows a design of a sample oligonucleotide from 5′ to 3′ (SEQ ID No.:87).

FIG. 4 shows DNA sequencing analysis of the CHAnGE plasmid library.

FIG. 5 shows genome-scale engineering of furfural tolerance. Volcano plot is shown for furfural stressed libraries versus untreated libraries. The X-axis represents enrichment levels of each guide sequence. The Y-axis represents log 10 transformed P values. Significantly enriched guides (p<0.05, fold change >1.5) are denoted by black dots, all others by gray dots. Dotted lines indicate 1.5-fold ratio (X-axis) and P value of 0.05 (Y-axis). The red dots represent SIZ1 targeting guide sequences. The orange dots represent SAP30 targeting guide sequences. The blue dots represent UBC4 targeting guide sequences. The green dots represent non-editing control guide sequences. n=2 independent experiments.

FIG. 6 shows biomass accumulation of furfural tolerant mutants and the wild type strain in the presence of 5 mM furfural. The Y-axis represents optical density measured at 600 nm 24 hours after inoculation. SC, synthetic complete media. n=3 independent experiments. Error bars represent standard error of the mean. ***, P<0.001. ****, P<0.0001. ns, not significant.

FIG. 7 shows biomass accumulation of furfural tolerant single and double mutants and the wild type strain in the presence of 5 mM furfural. The Y-axis represents optical density measured at 600 nm 24 hours after inoculation. SC, synthetic complete media. n=3 independent experiments. Error bars represent standard error of the mean. **, P<0.01. ***, P<0.001.

FIG. 8 shows genome-scale engineering of yeast strains with higher HAc tolerance. Volcano plot is shown for HAc stressed libraries versus untreated libraries. The X-axis represents enrichment levels of each guide sequence. The Y-axis represents log 10 transformed P values. Significantly enriched guides (p<0.05, fold change >1.5) are denoted by black dots, all others by gray dots. Dotted lines indicate 1.5-fold ratio (X-axis) and P value of 0.05 (Y-axis). The red dots represent BUL1 targeting guide sequences. The green dots represent non-editing control guide sequences. n=2 independent experiments.

FIG. 9 shows biomass accumulation of BUL1A1 mutants and the wild type strain in the presence of 0.5% HAc. “BUL1Δ1 Screened” was the mutant recovered from the HAc stressed library. The Y-axis represents optical density measured at 600 nm 48 hours after inoculation. SC, synthetic complete media. n=3 independent experiments. Error bars represent standard error of the mean. ns, not significant.

FIG. 10 shows directed evolution of HAc tolerance. (a) Enrichment of guide sequences during the first round and second round directed evolution of HAc tolerance. (b) Biomass accumulation of the wild type and mutant strains in the presence of HAc. n=3 independent experiments. Error bars represent standard error of the mean. Two-tailed t-tests were performed to determine significance levels against the wild type strain. *, P<0.05. ***, P<0.001. ns, not significant.

FIG. 11 shows (a) design of F268A mutations and the sequence of a representative edited colony. The genomic nucleic acid sequence is SEQ ID NO:88. The genomic amino acid sequence is SEQ ID NO:89. The HR template nucleic acid sequence is SEQ ID NO:90. The HR template amino acid sequence is SEQ ID NO:91. The representative colony nucleic acid sequence is SEQ ID NO:90. The representative colony amino acid sequence is SEQ ID NO:91. (b) Design of I363A mutations and the sequence of a representative non-edited colony. The genomic nucleic acid sequence is SEQ ID NO:92. The genomic amino acid sequence is SEQ ID NO:93. The HR template nucleic acid sequence is SEQ ID NO:94. The HR template amino acid sequence is SEQ ID NO:95. The representative colony nucleic acid sequence is SEQ ID NO:92. The representative colony amino acid sequence is SEQ ID NO:93. (c) Design of S391D mutations and the sequence of a representative edited colony. The genomic nucleic acid sequence is SEQ ID NO:96. The genomic amino acid sequence is SEQ ID NO:97. The HR template nucleic acid sequence is SEQ ID NO:98. The HR template amino acid sequence is SEQ ID NO:99. The representative colony nucleic acid sequence is SEQ ID NO:98. The representative colony amino acid sequence is SEQ ID NO:99.

FIG. 12 shows (a) a bicistronic crRNA expression cassette for simultaneous introduction of two aa substitutions. Black diamonds denote direct repeats. (b) Design of F250A F299A mutations and the sequence of a representative edited colony. The genomic nucleic acid sequence for the F250A mutation is SEQ ID NO:100. The genomic amino acid sequence for the F250 mutationA is SEQ ID NO:101. The HR template nucleic acid sequence for the F250A mutation is SEQ ID NO:102. The HR template amino acid sequence for the F250A mutation is SEQ ID NO:103. The representative colony nucleic acid sequence for the F250A mutation is SEQ ID NO:102. The representative colony amino acid sequence for the F250A mutation is SEQ ID NO:103. The genomic nucleic acid sequence for the F299A mutation is SEQ ID NO:104. The genomic amino acid sequence for the F299A mutation is SEQ ID NO:105. The HR template nucleic acid sequence for the F299A mutation is SEQ ID NO:106. The HR template amino acid sequence for the F299A mutation is SEQ ID NO:107. The representative colony nucleic acid sequence for the F299A mutation is SEQ ID NO:106. The representative colony amino acid sequence for the F299A mutation is SEQ ID NO:107.

FIG. 13 shows design of FKSΔ mutations and the sequence of a representative edited colony. The genomic nucleic acid sequence is SEQ ID NO:108. The genomic amino acid sequence is SEQ ID NO:109. The HR template nucleic acid sequence is SEQ ID NO:110. The HR template amino acid sequence is SEQ ID NO:111. The representative colony nucleic acid sequence is SEQ ID NO:110. The representative colony amino acid sequence is SEQ ID NO:111.

FIG. 14 shows design of AAA insertional mutations and the sequence of a representative edited colony. The genomic nucleic acid sequence is SEQ ID NO:112. The genomic amino acid sequence is SEQ ID NO:113. The HR template nucleic acid sequence is SEQ ID NO:114. The HR template amino acid sequence is SEQ ID NO:115. The representative colony nucleic acid sequence is SEQ ID NO:114. The representative colony amino acid sequence is SEQ ID NO:115.

FIG. 15 shows (a) design of E184A#1 mutations and the sequence of a representative edited colony. The genomic nucleic acid sequence is SEQ ID NO:116. The genomic amino acid sequence is SEQ ID NO:117. The HR template nucleic acid sequence is SEQ ID NO:118. The HR template amino acid sequence is SEQ ID NO:119. The representative colony nucleic acid sequence is SEQ ID NO:118. The representative colony amino acid sequence is SEQ ID NO:119. (b) Design of E184A#2 mutations and the sequence of a representative edited colony. The genomic nucleic acid sequence is SEQ ID NO:120. The genomic amino acid sequence is SEQ ID NO:117. The HR template nucleic acid sequence is SEQ ID NO:121. The HR template amino acid sequence is SEQ ID NO:119. The representative colony nucleic acid sequence is SEQ ID NO:121. The representative colony amino acid sequence is SEQ ID NO:119. (c) Design of E184A#3 mutations and the sequence of a representative non-edited colony. The genomic nucleic acid sequence is SEQ ID NO:122. The genomic amino acid sequence is SEQ ID NO:123. The HR template nucleic acid sequence is SEQ ID NO:124. The HR template amino acid sequence is SEQ ID NO:125. The representative colony nucleic acid sequence is SEQ ID NO:122. The representative colony amino acid sequence is SEQ ID NO:123.

FIG. 16 shows (a) a summary of efficiencies of CAN1 precise editing. For each mutagenesis, 4 or 5 randomly picked colonies were examined. (b) Growth assay of CAN1 mutants in the presence of canavanine. SC, synthetic complete media. SC-R, synthetic complete media minus arginine. CAN1Δ::URA3, BY4741 strain with the CAN1 ORF replaced by a URA3 selection marker.

FIG. 17 shows (a) enrichment of UBC4 targeting guide sequences in the presence of HAc or furfural. (b) Crystal structure of Ubc4 showing the C86 residue. PDB code 1QCQ.

FIG. 18 shows (a) Design of C86A#1 mutations and the sequence of a representative edited colony. The genomic nucleic acid sequence is SEQ ID NO:126. The genomic amino acid sequence is SEQ ID NO:127. The HR template nucleic acid sequence is SEQ ID NO:128. The HR template amino acid sequence is SEQ ID NO:129. The representative colony nucleic acid sequence is SEQ ID NO:130. The representative colony amino acid sequence is SEQ ID NO:129. (b) Design of C86A#2 mutations and the sequence of a representative edited colony. The genomic nucleic acid sequence is SEQ ID NO:131. The genomic amino acid sequence is SEQ ID NO:132. The HR template nucleic acid sequence is SEQ ID NO:133. The HR template amino acid sequence is SEQ ID NO:134. The representative colony nucleic acid sequence is SEQ ID NO:135. The representative colony amino acid sequence is SEQ ID NO:134. (c) Design of C86A#3 mutations and the sequence of a representative edited colony. The genomic nucleic acid sequence is SEQ ID NO:136. The genomic amino acid sequence is SEQ ID NO:137. The HR template nucleic acid sequence is SEQ ID NO:138. The HR template amino acid sequence is SEQ ID NO:139. The representative colony nucleic acid sequence is SEQ ID NO:140. The representative colony amino acid sequence is SEQ ID NO:139. (d) Design of C86A#4 mutations and the sequence of a representative edited colony. The genomic nucleic acid sequence is SEQ ID NO:141. The genomic amino acid sequence is SEQ ID NO:142. The HR template nucleic acid sequence is SEQ ID NO:143. The HR template amino acid sequence is SEQ ID NO:144. The representative colony nucleic acid sequence is SEQ ID NO:145. The representative colony amino acid sequence is SEQ ID NO:144. (e) Design of C86A#5 mutations and the sequence of a representative edited colony. The genomic nucleic acid sequence is SEQ ID NO:146. The genomic amino acid sequence is SEQ ID NO:147. The HR template nucleic acid sequence is SEQ ID NO:148. The HR template amino acid sequence is SEQ ID NO:149. The representative colony nucleic acid sequence is SEQ ID NO:148. The representative colony amino acid sequence is SEQ ID NO:149.

FIG. 19 shows (a) a summary of efficiencies of UBC4 precise editing. For each mutagenesis, 4 or 5 randomly picked colonies were examined. (b) Spotting assay of UBC4 mutants in the presence of HAc or furfural.

FIG. 20 shows Sanger sequencing result showing precise editing of human EMX1 locus using a CHAnGE cassette. Arrows indicate primers for selective amplification of edited genomes. The forward primer anneals to a region 421 bp upstream of the protospacer and outside of the left homology arm, while the reverse primer anneals to the edited sequence. Expected edits are highlighted with red boxes. The genomic nucleic acid sequence is SEQ ID NO:150. The HR template nucleic acid sequence is SEQ ID NO:151. The Sanger sequencing nucleic acid is SEQ ID NO:151.

### DETAILED DESCRIPTION

Methods and compositions now will be described more fully hereinafter with reference to the accompanying drawings, in which some, but not all embodiments of the methods and compositions are shown. Indeed, the methods and compositions can be embodied in many different forms and should not be construed as limited to the embodiments set forth herein; rather, these embodiments are provided so that this disclosure will satisfy applicable legal requirements.

Likewise, many modifications and other embodiments of the methods and compositions described herein will come to mind to one of skill in the art to which the methods and compositions pertain having the benefit of the teachings presented in the foregoing descriptions and the associated drawings. Therefore, it is to be understood that the methods and compositions are not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the systems and methods pertain.

As used herein, the term “and/or” includes any and all combinations of one or more of the associated listed items. As used herein, the singular forms “a,” “an,” and “the” are intended to include the plural forms as well as the singular forms, unless the context clearly indicates otherwise.

The embodiments illustratively described herein suitably can be practiced in the absence of any element or elements, limitation or limitations that are not specifically disclosed herein. Thus, for example, in each instance herein any of the terms “comprising,” “consisting essentially of,” and “consisting of” may be replaced with either of the other two terms, while retaining their ordinary meanings.

The term “about” in association with a numerical value means that the numerical value can vary plus or minus by 5% or less of the numerical value. All patents, patent applications, and other scientific or technical writings referred to anywhere herein are incorporated by reference herein in their entirety.

Polynucleotides

The terms “polynucleotide,” “nucleotides,” “nucleic acid molecule” and “oligonucleotide” are used interchangeably. They refer to a polymeric form of nucleotides of any length, either deoxyribonucleotides or ribonucleotides, or analogs thereof. Polynucleotides can have any three dimensional structure, and can perform any function, known or unknown. Nucleic acid molecule means a single- or double-stranded linear polynucleotide containing either deoxyribonucleotides or ribonucleotides that are linked by 3′-5′-phosphodiester bonds. A nucleic acid construct is a nucleic acid molecule that is isolated from a naturally occurring gene or that has been modified to contain segments of nucleic acids that are combined and juxtaposed in a manner that would not otherwise exist in nature. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), single guide RNA (sgRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide can comprise one or more modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component.

A recombinant nucleic acid molecule, for instance a recombinant DNA molecule, is a nucleic acid molecule formed in vitro through the ligation of two or more nonhomologous DNA molecules (for example a recombinant plasmid containing one or more inserts of foreign DNA cloned into at least one cloning site).

A gene is any polynucleotide molecule that encodes a polypeptide, protein, or fragments thereof, optionally including one or more regulatory elements preceding (5′ non-coding sequences) and following (3′ non-coding sequences) the coding sequence. In one embodiment, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source, but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99% or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein (e.g., SIZ1, SAP30, UBC4, BUL1, SUR1, LCB3 and mutants or variants thereof).

Polynucleotides can comprise additional heterologous nucleotides that do not naturally occur contiguously with the polynucleotides. As used herein the term “heterologous” refers to a combination of elements that are not naturally occurring or that are obtained from different sources.

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can be obtained from nucleic acid sequences present in, for example, a microorganism such as a yeast or bacterium. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can comprise coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as rRNA genes or tRNA genes, the product is a functional RNA. The process of gene expression is used by all known life forms, i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

Homology refers to the similarity between two nucleic acid sequences. Homology among DNA, RNA, or proteins is typically inferred from their nucleotide or amino acid sequence similarity. Significant similarity is strong evidence that two sequences are related by evolutionary changes from a common ancestral sequence. Alignments of multiple sequences are used to indicate which regions of each sequence are homologous. The term “percent homology” is used herein to mean “sequence similarity.” The percentage of identical nucleic acids or residues (percent identity) or the percentage of nucleic acids residues conserved with similar physicochemical properties (percent similarity), e.g. leucine and isoleucine, is used to quantify the homology.

Complement or complementary sequence means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′. Downstream refers to a relative position in DNA or RNA and is the region towards the 3′ end of a strand. Upstream means on the 5′ side of any site in DNA or RNA.

As described herein, “sequence identity” is related to sequence homology. Homology comparisons can be conducted by eye or using sequence comparison programs. These commercially available computer programs can calculate percent (%) homology between two or more sequences and can also calculate the sequence identity shared by two or more amino acid or nucleic acid sequences. Sequence homologies may be generated by any of a number of computer programs known in the art, for example BLAST or FASTA.

Percentage (%) sequence identity can be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Ungapped alignments are performed only over a relatively short number of residues. Although this is a very simple and consistent method, it fails to take into consideration that, for example, in an otherwise identical pair of sequences, one insertion or deletion can cause the following amino acid residues to be put out of alignment, thus potentially resulting in a large reduction in percent homology when a global alignment is performed. Therefore, most sequence comparison methods are designed to produce optimal alignments that take into consideration possible insertions and deletions without unduly penalizing the overall homology or identity score. This is achieved by inserting “gaps” in the sequence alignment to try to maximize local homology or identity.

CRISPR Systems

A Clustered Regularly Interspersed Short Palindromic Repeats/CRISPR-associated (CRISPR/Cas) system comprise components of a prokaryotic adaptive immune system that is functionally analogous to eukaryotic RNA interference, and that uses RNA base pairing to direct DNA or RNA cleavage. Directing DNA double stranded breaks requires an RNA-guided DNA endonuclease (e.g., Cas9 protein or the equivalent) and CRISPR RNA (crRNA) and tracer RNA (tracrRNA) sequences that aid in directing the RNA-guided DNA endonuclease/RNA complex to target nucleic acid sequence. The modification of a single targeting RNA can be sufficient to alter the nucleotide target of an RNA-guided DNA endonuclease protein. crRNA and tracrRNA can be engineered as a single cr/tracrRNA hybrid to direct the RNA-guided DNA endonuclease cleavage activity. A CRISPR/Cas system can be used in vivo in bacteria, yeast, fungi, plants, animals, mammals, humans, and in in vitro systems.

A CRISPR system can comprise transcripts and other elements involved in the expression of or directing the activity of CRISPR-associated (“Cas”) genes, including sequences encoding an RNA-guided DNA endonuclease gene (i.e. Cas), a tracr (trans-activating CRISPR) sequence (e.g. tracrRNA or an active partial tracrRNA), a tracr-mate sequence (encompassing a “direct repeat” and a tracrRNA-processed partial direct repeat), a guide sequence, or other sequences and transcripts from a CRISPR locus. One or more elements of a CRISPR system can be derived from a type I, type II, type III, type IV, and type V CRISPR system. A CRISPR system comprises elements that promote the formation of a CRISPR complex at the site of a target sequence (also called a protospacer).

Typically, a CRISPR system can comprise a CRISPR complex (comprising a guide sequence hybridized to a target sequence and complexed with one or more RNA-guided DNA endonucleases) that results in cleavage of DNA in or near (e.g. within 1, 2, 3, 4, 5, 6, 7, 8, 9, 10, 20, 50, or more base pairs from) the target sequence.

The elements of CRISPR systems (e.g., direct repeats, homologous recombination editing templates, guide sequences, tracrRNA sequences, target sequences, priming sites, regulatory elements, and RNA-guided DNA endonucleases) are well known to those of skill in the art. That is, given a target sequence one of skill in the art can design functional CRISPR elements specific for a particular target sequence. The methods described herein are not limited to the use of specific CRISPR elements, but rather are intended to provide unique arrangements, compilations, and uses of the CRISPR elements.

Direct Repeats

A CRISPR direct repeat region contains sequences required for processing pre-crRNA into mature crRNA and tracrRNA binding. CRISPR direct repeat regions are about 23, 25, 28, 29, 30, 31, 32, 33, 34, 35, 36, 37, 40, 45, 50, 55 or more base pairs. Direct repeat regions can have dyad symmetry, which can result in the formation of a secondary structure such as a stem-loop (“hairpin”) in the RNA. A genetic engineering cassette can comprise 2 or 3 CRISPR direct repeats, which can have the same or different sequence.

A genetic engineering cassette described herein can have direct repeats flanking a spacer region, wherein the spacer region comprises a homologous recombination template and a guide sequence. The most commonly used type II CRISPR/Cas9 direct repeat can be found in the following references: Jinek et al. A programmable dual-RNA guided DNA endonuclease in adaptive bacterial immunity. Science. 337:816 (2012); Bao et al., ACS Synth Biol 4:585 (2015); Bao et al. Nat Biotechnol 36:505 (2018). Other direct repeats are described in, for example, Makarova et al., An updated evolutionary classification of CRISPR-Cas systems. Nat Rev Microbiol. 13:722 (2015). One of ordinary skill in the art can select appropriate direct repeat sequences.

Homologous Recombination Editing Template

A template that can be used for recombination into a targeted locus comprising a target sequence is an “editing template” or “homologous recombination editing template.” Guide RNA is coupled with an RNA-guided DNA endonuclease (e.g. Cas9) to create a DNA double-stranded break near a genomic region to be edited. A homologous recombination editing template is used to introduce desired mutations (e.g. deletion of nucleic acids, substitution of nucleic acids, insertion of nucleic acids) into a cell's genome. The cell can repair the double-stranded break with homology directed repair (HDR) via homologous recombination (HR) mechanism. To design a homologous recombination template a guide RNA is selected so the double-stranded cut site is within about 5, 10, 15, 20, 30, 40 or more base pairs from the targeted genomic region. The length of HR arms on both sides of the mutation is selected (e.g., about 20, 30, 40, 50, 60 or more nucleic acids or about 60, 50, 40, 30, 20 or less nucleic acids). A target genome, target gene or sequence, and PAM sequence is selected. Mutations to be made to the target sequence and/or the PAM sequence are incorporated into the homologous recombination editing template. More than one homologous recombination editing templates (e.g., 2, 3, 4, 5 or more) can be present in a genetic engineering cassette.

Homologous recombination editing templates used to create specific mutations or insert new elements into a target sequence require a certain amount of homology surrounding the target sequence that will be modified. In an embodiment each of the HR arms has about 70, 80, 90, 95, 99 or 100% homology to the target sequence.

RNA-guided DNA endonucleases can continue to cleave DNA once a double stranded break is introduced and repaired. As long as the gRNA target site/PAM site remains intact, the RNA-guided DNA endonuclease may keep cutting and repairing the DNA. A homologous recombination editing template can be designed to block further endonuclease targeting after the initial double stranded break is repaired. For example, the homologous recombination editing template can be designed to mutate the PAM sequence.

A homologous recombination editing template repairs a cleaved target polynucleotide by homologous recombination such that the repair results in a mutation comprising an insertion, deletion, or substitution of one or more nucleotides of the target polynucleotide. The mutation can result in one or more (e.g., 1, 2, 3, 4, or more) amino acid changes in a protein expressed from a gene comprising the target sequence.

A homologous recombination editing template can be provided in a vector, or provided as a separate polynucleotide. A homologous recombination editing template is designed to serve as a template in homologous recombination, such as within or near a target sequence cleaved by an RNA-guided DNA endonuclease as a part of a CRISPR complex. A homologous recombination editing template polynucleotide can be about 50, 60, 70, 80, 85, 90, 100, 105, 110, 120, 130, 150, 160, 175, 200, or more nucleotides in length. A homologous recombination editing template polynucleotide can be 200, 175, 160, 150, 130, 120, 110, 105, 100, 90, 85, 80, 70, 60 50 or less nucleotides in length. A homologous recombination editing template polynucleotide is complementary to a portion of a polynucleotide comprising the target sequence. When optimally aligned, an editing template polynucleotide will overlap with one or more nucleotides of a target sequence (e.g. about 1, 5, 10, 15, 20, 25, 30, 35, 40, 45, 50, 60, 70, 80, 90, 100 or more nucleotides).

In one embodiment, the methods provide for modification of a target polynucleotide in a host cell such as a eukaryotic cell or a prokaryotic cell. In some embodiments, the method comprises allowing an RNA-guided DNA endonuclease complex to bind to the target polynucleotide to effect cleavage of the target polynucleotide thereby modifying the target polynucleotide, wherein the RNA-guided DNA endonuclease comprises an RNA-guided DNA endonuclease complexed with a guide sequence hybridized to a target sequence within the target polynucleotide.

A homologous recombination editing template provides for the specific modification of a target polynucleotide. A deletion portion of a homologous recombination editing template comprises nucleotides that direct the deletion of 1, 2, 3, 4, 5, 6, 7, 8, 9, 10, or more nucleic acids from a targeted gene. A deletion of a certain amount of nucleic acids from a targeted gene can result in an inoperative gene product or no expression of the gene product. A gene deletion or knockout refers to a genetic technique in which a gene is made inoperative. That is, a gene product is no longer expressed. Knocking out two genes simultaneously results in a double knockout. Similarly, triple knockout (TKO) and quadruple knockouts (QKO) are used to describe three or four knocked out genes, respectively. Heterozygous knockouts refer to when only one of the two gene copies (alleles) is knocked out, and homozygous knockouts refer to when both gene copies are knocked out.

A substitution portion of a homologous recombination template comprises nucleotides that direct the substitution of 1, 2, 3, 4, 5, 6, 7, 8, 9, 10, or more nucleic acids with different nucleic acids in a targeted gene. A substitution of one or more nucleic acids in a targeted gene can result in the substitution of an amino acid (i.e., a different amino acid at a specific position) in protein expressed by the targeted gene.

An insertion portion of a homologous recombination template comprises nucleotides that direct the insertion of 1, 2, 3, 4, 5, 6, 7, 8, 9, 10, or more nucleic acids into a targeted gene. An insertion of a certain amount of nucleic acids into a targeted gene can result in an inoperative gene product, no expression of the gene product, or a gene product with new or additional biological functions.

Guide Sequences

As used herein, “single guide RNA,” “guide RNA (gRNA),” “guide sequence” and “sgRNA” can be used interchangeably herein and refer to a single RNA species capable of directing RNA-guided DNA endonuclease mediated double stranded cleavage of target DNA. Single-stranded gRNA sequences are transcribed from double-stranded DNA sequences inside the cell.

A guide RNA is a specific RNA sequence that recognizes a target DNA region of interest and directs an RNA-guided DNA endonuclease there for editing. A gRNA has at least two regions. First, a CRISPR RNA (crRNA) or spacer sequence, which is a nucleotide sequence complementary to the target nucleic acid, and second a tracr RNA, which serves as a binding scaffold for the RNA-guided DNA endonuclease. The target sequence that is complementary to the guide sequence is known as the protospacer. The crRNA and tracr RNA can exist as one molecule or as two separate molecules, as they are in nature. gRNA and sgRNA as used herein refer to a single molecule comprising at least a crRNA region and a tracr RNA region or two separate molecules wherein the first comprises the crRNA region and the second comprises a tracr RNA region. The crRNA region of the gRNA is a customizable component that enables specificity in every CRISPR reaction. A guide RNA used in the systems and methods can also comprise an endoribonuclease recognition site (e.g., Csy4) for multiplex processing of gRNAs. If an endoribonuclease recognition site is introduced between neighboring gRNA sequences, more than one gRNA can be transcribed in a single expression cassette. Direct repeats can also serve as endoribonuclease recognition sites for multiplex processing.

A guide RNA used in the systems and methods described herein are short, single-stranded polynucleotide molecules about 20 nucleotides to about 300 nucleotides in length. The spacer sequence (targeting sequence) that hybridizes to a complementary region of the target DNA of interest can be about 14, 15, 16, 17, 18, 19, 20, 25, 30, 35 or more nucleotides in length.

A sgRNA capable of directing RNA-guided DNA endonuclease mediated substitution of, insertion at, or deletion of target sequence can be about 10, 11, 12, 13, 14, 15, 16, 17, 18, 19, 20, 30, 40, 50 or more nucleotides in length. A sgRNA capable of directing RNA-guided DNA endonuclease mediated substitution of, insertion at, or deletion of target sequence can be about 50, 40, 30, 20, 19, 18, 17, 16, 15, 14, 13, 12, 11 or less nucleotides in length. The sgRNA used to direct insertion, substitution, or deletion can include HR sequences for homology-directed repair.

sgRNAs can be synthetically generated or by making the sgRNA in vivo or in vitro, starting from a DNA template.

A sgRNA can target a regulatory element (e.g., a promoter, enhancer, or other regulatory element) in the target genome. A sgRNA can also target a coding sequence in the target genome.

sgRNA that is capable of binding a target nucleic acid sequence and binding a RNA-guided DNA endonuclease protein can be expressed from a vector comprising a type II promoter or a type III promoter.

Target Sequences

In the context of formation of a CRISPR complex, a target sequence or target nucleic acid molecule is a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence can comprise any polynucleotide, such as DNA or RNA polynucleotides. In some embodiments, a target sequence is located in the nucleus or cytoplasm of a cell. In some embodiments, the target sequence can be within an organelle of a eukaryotic cell, for example, mitochondrion or chloroplast.

The degree of complementarity between a guide sequence and its corresponding target sequence, when optimally aligned using a suitable alignment algorithm, is about 50%, 60%, 75%, 80%, 85%, 90%, 95%, 97.5%, 99%, or more. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at m aq. sou rceforge. net).

The target polynucleotide of a CRISPR complex can be any polynucleotide endogenous or exogenous to a host cell, such as a eukaryotic cell. For example, the target polynucleotide can be a polynucleotide residing in the nucleus of the host cell. The target polynucleotide can be a sequence coding a gene product (e.g., a protein) or a non-coding sequence (e.g., a regulatory polynucleotide). The target sequence can be associated with a PAM (protospacer adjacent motif); that is, a short sequence recognized by the CRISPR complex. The precise sequence and length requirements for the PAM differ depending on the RNA-guided DNA endonuclease used, but PAMs are typically 2-5 base pair sequences adjacent to the protospacer (that is, the target sequence). Those of ordinary skill in the art skilled can identify PAM sequences for use with a given RNA-guided DNA endonuclease enzyme.

TracrRNA Sequence

A tracrRNA sequence, which can comprise all or a portion of a wild-type tracrRNA sequence (e.g. about 20, 26, 32, 45, 48, 54, 63, 67, 85, or more nucleotides of a wild-type tracrRNA sequence), can also form part of a CRISPR complex. A tracrRNA sequence can hybridize along at least a portion of a tracrRNA sequence to all or a portion of a direct repeat sequence.

The degree of complementarity between a tracrRNA sequence and a tracr mate sequence along the length of the shorter of the two when optimally aligned is about 25%, 30%, 40%, 50%, 60%, 70%, 80%, 90%, 95%, 97.5%, 99%, or higher. In some embodiments, the tracrRNA sequence is about or more than about 5, 6, 7, 8, 9, 10, 11, 12, 13, 14, 15, 16, 17, 18, 19, 20, 25, 30, 40, 50, or more nucleotides in length.

Markers

One or more vectors that express sgRNA and/or RNA-guided DNA endonuclease proteins can further comprise a polynucleotide encoding for a marker protein.

A polynucleotide encoding a marker protein can be expressed on a separate vector from a vector that expresses sgRNA and/or RNA-guided DNA endonuclease proteins.

A marker protein is a protein encoded by a gene that when introduced into a cell confers a trait suitable for artificial selection. Marker proteins are used in laboratory, molecular biology, and genetic engineering applications to indicate the success of a transformation, a transfection or other procedure meant to introduce foreign nucleic acids into a cell. Marker proteins include, but are not limited to, fluorescent proteins and proteins that confer resistance to antibiotics, herbicides, or other compounds, which would be lethal to cells, organelles or tissues not expressing the resistance gene or allele. Selection of transformants is accomplished by growing the cells or tissues under selective pressure, i.e., on media containing the antibiotic, herbicide or other compound. If the marker protein is a “lethal” marker, cells which express the marker protein will live, while cells lacking the marker protein will die. If the marker protein is “non-lethal,” transformants (i.e., cells expressing the selectable marker) will be identifiable by some means from non-transformants, but both transformants and non-transformants will live in the presence of the selection pressure.

Selective pressure refers to the influence exerted by some factor (such as an antibiotic, heat, light, pressure, or a marker protein) on natural selection to promote one group of organisms or cells over another. In the case of antibiotic resistance, applying antibiotics cause a selective pressure by killing susceptible cells, allowing antibiotic-resistant cells to survive and multiply.

Selective pressure can be applied by contacting the cells with an antibiotic and selecting the cells that survive. The antibiotic can be, for example, kanamycin, puromycin, spectinomycin, streptomycin, ampicillin, carbenicillin, bleomycin, erythromycin, polymyxin B, tetracycline, or chloramphenicol.

In an embodiment, the methods described herein can function without the use of a protein marker encoded by a genetic engineering cassette or by the vector.

Genetic Bar Codes

In an embodiment, a genetic engineering cassette or homologous recombination editing template, or guide sequence functions as a genetic barcode due to its unique sequence. The unique sequence can be used with next generation sequencing to quickly identify the mutation or mutations present in a transformed host cell. In an embodiment a genetic barcode is a unique sequence within a genetic engineering cassette that can be used in the same way. A genetic barcode can be present anywhere in the genetic engineering cassette, for example, between the homology arms.

Priming Site

A primer site is a region of a nucleic acid sequence where an RNA or DNA single-stranded primer binds to start replication. The primer site is on one of the two complementary strands of a double-stranded nucleotide polymer, in the strand which is to be copied, or is within a single-stranded nucleotide polymer sequence.

Genetic Engineering Cassettes

Targeted genome engineering is genetic engineering where nucleic acid molecules are inserted, deleted, modified, modulated, or replaced in the genome of a living organism or cell. Targeted genome engineering can involve substituting nucleic acids, integrating nucleic acids into, or deleting nucleic acids from genomic DNA at a target site of interest to manipulate (e.g., increase, decrease, knockout, activate, interfere with) the expression of one or more genes.

A genetic engineering cassette is a component of DNA, which can comprise several elements. In an embodiment a genetic engineering cassette can comprise from the 5′ to the 3′ end a first direct repeat sequence; a homologous recombination template comprising two homology arms with a deletion portion, a substitution portion, or an insertion portion between the two homology arms; a guide sequence; and a second direct repeat sequence. A genetic engineering cassette can comprise a first priming site at a 5′ end of the cassette and a second priming site at a 3′ end of the cassette. The priming sites can be the same or different. The first priming site and the second priming site can each comprise a restriction enzyme cleavage site. The priming sites can be operably linked to the genetic engineering cassette components. In an embodiment a genetic engineering cassette does not comprise a promoter. Instead a promoter is present on the vector backbone.

RNA-Guided DNA Endonucleases

An RNA-guided DNA endonuclease protein is directed to a specific DNA target by a gRNA, where it causes a double-strand break. There are many versions of RNA-guided DNA endonucleases isolated from different bacteria.

Each RNA-guided DNA endonuclease binds to its target sequence in the presence of a protospacer adjacent motif (PAM), on the non-targeted DNA strand. Therefore, the locations in a genome that can be targeted by different RNA-guided DNA endonuclease can be dictated by locations of PAM sequences. An RNA-guided DNA endonuclease cuts 3-4 nucleotides upstream of the PAM sequence. Recognition of the PAM sequence by an RNA-guided DNA endonuclease protein is thought to destabilize the adjacent DNA sequence, allowing interrogation of the sequence by the sgRNA, and allowing the sgRNA-DNA pairing when a matching sequence is present.

RNA-guided DNA endonucleases isolated from different bacterial species recognize different PAM sequences. For example, the SpCas9 nuclease cuts upstream of the PAM sequence 5′-NGG-3′ (where “N” can be any nucleotide base), while the PAM sequence 5′-NNGRR(N)-3′ is required for SaCas9 (from Staphylococcus aureus) to target a DNA region for editing. While the PAM sequence itself is necessary for cleavage, it is not included in the single guide RNA sequence.

RNA-guided DNA endonuclease proteins include, for example, Cas9 from Streptococcus pyogenes (SpCas9), Neisseria meningitides (NmCas9), Streptococcus thermophiles (St1Cas9), and Staphylococcus aureus (SaCas9) and Cpf1 from Lachnospiraceae bacterium ND2006 (LbCpf1) and Acidaminococcus sp. BV3L6 (AsCpf1).

Non-limiting examples of RNA-guided DNA endonuclease proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the RNA-guided DNA endonuclease directs cleavage of both strands of target DNA within about 1, 2, 3, 4, 5, 6, 7, 8, 9, 10, 15, 20, 25, 50, 100, 200, 500, or more base pairs from the first or last nucleotide of a target sequence.

In an embodiment, a coding sequence encoding an RNA-guided DNA endonuclease is codon optimized for expression in particular cells, such as eukaryotic cells. The eukaryotic cells can be those of or derived from a particular organism, such as a yeast or a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence.

A system described herein can comprise one or more sgRNA molecules that are capable of binding a target nucleic acid and an RNA-guided DNA endonuclease protein that causes a double-stranded nucleic acid break of one or more additional target nucleic acid molecules. In this aspect, the genome can be cut at several different sites (e.g., 1, 2, 3, 4, 5, 6, 7, 8, 9, or 10 sites) at or near the same time, and the homology directed repair donor included in the genetic engineering cassette can be inserted into those one or more sites (Bao et al., 2015, ACS Synth. Biol., 5:585-594).

An RNA-guided DNA endonuclease can be expressed from a nucleic acid molecule that is present in a vector. A vector can comprise an RNA-guided DNA endonuclease and regulatory elements to be expressed by a transformed or transfected cell, whereby the RNA-guided DNA endonuclease and regulatory elements direct the cell to make RNA and protein. Different types of RNA-guided DNA endonucleases and regulatory elements can be transformed or transfected into different organisms including yeast, plants, and mammalian cells as long as the proper regulatory element sequences are used.

Once a target sequence and RNA-guided DNA endonuclease have been selected, the next step is to design specific guide RNA sequences. Several software tools exist for designing an optimal guide with minimum off-target effects and maximum on-target efficiency. Examples include Synthego Design Tool, Desktop Genetics, Benchling, and MIT CRISPR Designer.

In some embodiments, the RNA-guided DNA endonuclease is part of a fusion protein comprising one or more heterologous protein domains (e.g. about or more than about 1, 2, 3, 4, 5, 6, 7, 8, 9, 10, or more domains in addition to the RNA-guided DNA endonuclease). A CRISPR enzyme fusion protein can comprise any additional protein sequences, and optionally a linker sequence between any two domains. Examples of protein domains that may be fused to an RNA-guided DNA endonuclease include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). An RNA-guided DNA endonuclease can be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

Vectors

In an embodiment, a vector comprises a genetic engineering cassette as described herein. Also provided herein are pools of vectors comprising two or more (e.g., 2, 5, 10, 50, 100, 1,000, 5,000, 10,000 or more) of the vectors described herein wherein each of the genetic engineering cassettes is unique.

A vector can comprise one or more insertion sites (e.g. about or more than about 1, 2, 3, 4, 5, 6, 7, 8, 9, 10, or more insertion sites), such as a restriction endonuclease recognition site. An insertion site can be present between a (i) first promoter and (ii) a terminator, a second promoter, a nucleic acid sequence encoding an RNA-guided DNA endonuclease protein, a third promoter, and a tracrRNA sequence. The first promoter can be upstream of the genetic expression cassette and can be operably linked to the genetic expression cassette. The terminator can be downstream of the genetic expression cassette and can be operably linked to the genetic engineering cassette. The second promoter can be operably linked to a nucleic acid sequence encoding an RNA-guided DNA endonuclease protein. The third promoter can be operably linked to the tracrRNA sequence.

Several aspects of the disclosure relate to vector systems comprising one or more vectors. Vectors can be designed for expression of RNA-guided DNA endonucleases, and polynucleotides (e.g. nucleic acid transcripts, proteins, or enzymes) in host cell such as eukaryotic cells. For example, RNA-guided DNA endonucleases or polynucleotides can be expressed in insect cells (using baculovirus expression vectors), bacterial cells, yeast cells, or mammalian cells. Suitable cells are discussed further in Goeddel, GENE EXPRESSION TECHNOLOGY: METHODS IN ENZYMOLOGY 185, Academic Press, San Diego, Calif. (1990). Alternatively, a recombinant expression vector can be transcribed and translated in vitro, for example using T7 promoter regulatory sequences and T7 polymerase.

A vector or expression vector is a replicon, such as a plasmid, phage, or cosmid, to which another nucleic acid segment can be attached so as to bring about the replication of the attached segment. A vector is capable of transferring polynucleotides (e.g. gene sequences) to target cells.

Expression refers to the process by which a polynucleotide is transcribed from a nucleic acid template (such as into a sgRNA, tRNA or mRNA) and/or the process by which a transcribed mRNA is subsequently translated into peptides, polypeptides, or proteins. Transcripts and encoded polypeptides can be collectively referred to as “gene product.” A polypeptide is a linear polymer of amino acids that are linked by peptide bonds. If the polynucleotide is derived from genomic DNA, expression may include splicing of the mRNA in a eukaryotic cell.

Many suitable vectors and features thereof are known in the art. Vectors can contain, without limitation, a centromeric (CEN) sequence, an autonomous replication sequence (ARS), a promoter, an origin of replication, and a marker gene (e.g., auxotrophic, antibiotic, or other selectable markers). Examples of expression vectors include plasmids, yeast artificial chromosomes, 2μττκ plasmids, yeast integrative plasmids, yeast replicative plasmids, shuttle vectors, episomal plasmids, and viral vectors. In an embodiment, the viral vector is a lentivirus vector, an adenovirus vector, or an adeno-associated vector (AAV).

In some embodiments, a vector is a yeast expression vector. Examples of vectors for expression in yeast Saccharomyces cerevisiae include pYepSecl (Baldari et al., 1987. EMBO J. 6: 229-234), pMFa (Kuijan & Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow & Summers, 1989. Virology 170: 31-39).

In some embodiments, a vector is capable of driving expression of one or more sequences in mammalian cells using a mammalian expression vector. Examples of mammalian expression vectors include, but are not limited to, pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, a recombinant mammalian expression vector is capable of directing expression of a nucleic acid in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987. Genes Dev. 1: 268-277), lymphoid-specific promoters (Calame & Eaton, 1988. Adv. Immunol. 43: 235-275), in particular promoters of T cell receptors (Winoto and Baltimore, 1989. EMBO J. 8: 729-733) and immunoglobulins (Baneiji et al., 1983. Cell 33: 729-740; Queen & Baltimore, 1983. Cell 33: 741-748), neuron-specific promoters (e.g., the neurofilament promoter; Byrne & Ruddle, 1989. Proc. Natl. Acad. Sci. USA 86: 5473-5477), pancreas-specific promoters (Edlund et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel & Gruss, 1990. Science 249: 374-379) and the α-fetoprotein promoter (Campes & Tilghman, 1989. Genes Dev. 3: 537-546).

Vectors can be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc.; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

Promoters and Other Regulatory Elements

Genetic engineering cassettes and vectors can comprise 1, 2, 3, 4, 5, or more promoters. The promoters can be the same or different promoters. A promoter is any nucleic acid sequence that regulates the initiation of transcription for a particular polypeptide-encoding nucleic acid under its control. A promoter minimally includes the genetic elements necessary for the initiation of transcription (e.g., RNA polymerase III-mediated transcription), and can further include one or more genetic regulatory elements that serve to specify the prerequisite conditions for transcriptional initiation. A promoter can be a cis-acting DNA sequence, about 10, 20, 30, 40, 50, 60, 70, 80, 90, 100, 110, 120, or more base pairs long and located upstream of the initiation site of a gene, to which RNA polymerase can bind and initiate correct transcription. There can be associated additional transcription regulatory sequences that provide on/off regulation of transcription and/or which enhance (increase) expression of the downstream coding sequence. A coding sequence is the part of a gene or cDNA that codes for the amino acid sequence of a protein, or for a functional RNA such as a tRNA or rRNA.

A promoter can be encoded by an endogenous genome of a cell, or it can be introduced as part of a recombinantly engineered polynucleotide. A promoter sequence can be taken from one species and used to drive expression of a gene in a cell of a different species. A promoter sequence can also be artificially designed for a particular mode of expression in a particular species, through random mutation or rational design. In recombinant engineering applications, specific promoters are used to express a recombinant gene under a desired set of physiological or temporal conditions or to modulate the amount of expression of a recombinant nucleic acid.

As discussed above, a tissue-specific promoter can direct expression primarily in a desired tissue of interest, such as muscle, neuron, bone, skin, blood, specific organs (e.g. liver, pancreas), or particular cell types (e.g. lymphocytes).

Promoters used in the systems described herein include, for example, type II promoters (e.g., TEF1p, GPDp, PGK1p, and HXT7p) and type III promoters (SNR52p, PROp, U6, H1, RPR1p, and TYRp).

Other regulatory elements include enhancers, internal ribosomal entry sites (IRES), and other expression control elements (e.g. transcription termination signals (i.e., terminators), such as polyadenylation signals and poly-U sequences). Vectors and genetic engineering cassettes described herein can additionally comprise one or more regulatory elements. Regulatory elements include those that direct constitutive expression of a nucleotide sequence in many types of host cell and those that direct expression of the nucleotide sequence only in certain host cells (e.g., tissue-specific regulatory sequences). Regulatory elements can also direct expression in a temporal-dependent manner, such as in a cell-cycle dependent or developmental stage-dependent manner, which may or may not also be tissue or cell-type specific.

Regulatory elements include enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

Two DNA sequences are operably linked if the nature of the linkage does not interfere with the ability of the sequences to affect their normal functions relative to each other. For instance, a promoter region would be operably linked to a coding sequence of the protein if the promoter were capable of effecting transcription of that coding sequence.

In an embodiment, a genetic engineering cassette does not comprise a promoter. Instead, one or more (e.g., about 1, 2, 3, 4, 5, or more) promoters are located on the vector at a position to act on the genetic engineering cassette (i.e., operably linked), which is placed into the vector.

A polynucleotide can comprise a nucleotide sequence encoding a nuclear localization sequence (NLS). A NLS is an amino acid sequence that tags a protein for import into the cell nucleus by nuclear transport. Typically, this signal consists of one or more short sequences of positively charged lysines or arginines exposed on the protein surface. Different nuclear localized proteins can share the same NLS. A NLS can be added to the C-terminus, N-terminus, or both termini of an RNA-guided DNA endonuclease protein (e.g., NLS-protein, protein-NLS, or NLS-protein-NLS) to ensure nuclease activity in the cell.

A polynucleotide can also comprise a nucleotide sequence encoding a polypeptide linker sequence. Linkers are short (e.g., about 3 to 20 amino acids) polypeptide sequences that can be used to operably link protein domains. Linkers can comprise flexible amino acid residues (e.g., glycine or serine) to permit adjacent protein domains to move freely related to one another.

Delivery of Polynucleotides and Vectors to Host Cells

Methods are provided herein for delivering one or more polynucleotides, such as one or more vectors as described herein, one or more transcripts thereof, and/or one or more proteins transcribed therefrom, to a host cell. Also provided herein are cells produced by such methods, and organisms (such as animals, plants, or fungi) comprising or produced from such cells. Viral and non-viral based gene transfer methods can be used to introduce nucleic acids and vectors into host cells (e.g., eukaryotic cells, prokaryotic cells, bacteria, yeast, fungi, mammalian cells, plant cells, or target tissues). Such methods can be used to administer nucleic acids encoding components of the systems described herein to cells in culture or in a host organism. Non-viral vector delivery systems include DNA plasm ids, RNA (e.g. a transcript of a vector described herein), naked nucleic acid, and nucleic acid complexed with a delivery vehicle, such as a liposome. Viral vector delivery systems include DNA and RNA viruses, which can have either episomal or integrated genomes after delivery to the cell.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA.

Viral vectors can be administered directly to host cells in vivo or they can be administered to cells in vitro, and the modified cells can optionally be administered to host organisms (ex vivo). Viral based vector systems include, for example retroviral, lentivirus, adenoviral, adeno-associated and herpes simplex virus vectors for gene transfer. Integration in the host genome is possible with retrovirus, lentivirus, and adeno-associated virus gene transfer methods, often resulting in long term expression of the inserted transgene. Additionally, high transduction efficiencies have been observed in many different cell types and target tissues.

Following insertion of a genetic expression cassette into an insertion site of a vector and upon expression in a host cell the guide sequence(s) direct(s) sequence-specific binding of a CRISPR complex to a target sequence in the host cell.

Genetic Engineering Cassettes

In an embodiment a genetic engineering cassette can comprise from the 5′ to the 3′ end a first direct repeat sequence; a homologous recombination template comprising two homology arms with a deletion portion, a substitution portion, or an insertion portion; a guide sequence; and a second direct repeat sequence. A cassette can also comprise a first priming site at a 5′ end of the cassette and a second priming site at a 3′ end of the cassette. The priming sites can be the same or different. The first priming site and the second priming site can each comprise a restriction enzyme cleavage site. The priming sites can be operably linked to the genetic engineering cassette components. In an embodiment a genetic engineering cassette does not comprise a promoter. Instead a promoter is present on the vector in which the cassette is present. The deletion portions, substitution portions, or insertion portions are present between two homology arms of the homologous recombination template.

A genetic engineering cassette can be put into the insertion site of a vector comprising a first promoter upstream of the insertion site. Downstream of the insertion site the vector can comprise a terminator, a second promoter, a nucleic acid sequence encoding an RNA-guided DNA endonuclease protein, a third promoter, and a tracrRNA sequence.

The homologous recombination editing template can comprises a deletion portion that removes a protospacer adjacent motif (PAM) sequence and causes a gene disruption through deletion of part or all of the nucleic acids of the target nucleic acid molecule.

The genetic engineering cassette can further comprise a first priming site at a 5′ end of the cassette and a second priming site at a 3′ end of the cassette. The first priming site and the second priming site can comprise a restriction enzyme cleavage site. The priming sites can be operably linked to the genetic engineering cassette components. The priming sites can be the same or different.

An embodiment provides a pool of vectors comprising two or more (e.g., 2, 10, 50, 100, 200, 300, 400, 500, 1,000, 2,000, 3,000, 4,000, 5,000, 10,000, 15,000, 20,000, 25,000, 30,000 or more) of the vectors, wherein each of the genetic engineering cassettes is unique. Each genetic engineering cassette can be specific for (i.e. target) a different target nucleic acid. Several genetic engineering cassettes can be designed to target a single target sequence at several positions (e.g., about 2, 3, 4, 5, 10, 20, 50, 100, 1,000, or more) of the target sequence.

Another type of genetic engineering cassette can be used for single-nucleotide resolution editing. A genetic engineering cassette can comprise from a 5′ end to a 3′ end: a first direct repeat sequence; a first homologous recombination template comprising two homology arms with a deletion portion, a substitution portion, or an insertion portion; a first guide sequence; a second direct repeat sequence; a second homologous recombination template comprising two homology arms with a deletion portion, a substitution portion, or an insertion portion; a second guide sequence; and a third direct repeat sequence. The deletion portions, substitution portions, or insertion portions are present between two homology arms of the homologous recombination template.

The genetic engineering cassette can further comprise a first priming site at a 5′ end of the cassette and a second priming site at a 3′ end of the cassette. The first priming site and the second priming site comprise a restriction enzyme cleavage site. The priming sites can be operably linked to the genetic engineering cassette components. The priming sites can be the same or different.

In an embodiment the first homologous recombination editing template and the second homologous recombination editing template each provide for a first substitution, first insertion, or first deletion, and a second substitution, second insertion, or second deletion in the same target polynucleotide. For example, the two homologous recombination editing templates can target the same gene or same non-coding sequence for two deletions, substitutions, or insertions.

The first substitution, first insertion, or first deletion can occur within about 10, 20, 30, 40, 50, 60, 70, 80, 90, 100, 125, 150, 175, 200, 225, 250, 300, 400, 500, 1,000, 5,000, 10,000, or more nucleic acids of the second substitution, second insertion, or second deletion. Therefore, the system can be used to simultaneously introduce two distal mutations in the same target sequence.

The first substitution can be a substitution of 1, 2, 3, 4, 5, 6, 7, 8, 9, 10, 11, 12, 15, 20 or more nucleic acids (in one example, about 1 to about 6 nucleic acids), the first insertion can be an insertion of 1, 2, 3, 4, 5, 6, 7, 8, 9, 10, 11, 12, 15, 20 or more nucleic acids (in one example, about 1 to about 6 nucleic acids), the first deletion can be a deletion of 1, 2, 3, 4, 5, 6, 7, 8, 9, 10, 11, 12, 15, 20 or more nucleic acids (in one example, about 1 to about 6 nucleic acids), the second substitution can be a substitution of 1, 2, 3, 4, 5, 6, 7, 8, 9, 10, 11, 12, 15, 20 or more nucleic acids (in one example, about 1 to about 6 nucleic acids), the second insertion can be an insertion of 1, 2, 3, 4, 5, 6, 7, 8, 9, 10, 11, 12, 15, 20 or more nucleic acids (in one example, about 1 to about 6 nucleic acids), the second deletion can be a deletion of 1, 2, 3, 4, 5, 6, 7, 8, 9, 10, 11, 12, 15, 20 or more nucleic acids (in one example, about 1 to about 6 nucleic acids). Therefore, mutations that are not likely to occur spontaneously (e.g., those that require 2 or 3 bases within a codon to be altered) can be introduced.

A genetic engineering cassette can be present in a vector. The vector can comprise a first promoter upstream of the genetic engineering cassette. Downstream of the genetic engineering cassette the vector can comprise a terminator, a second promoter, a nucleic acid sequence encoding an RNA-guided DNA endonuclease protein, a third promoter, and a tracrRNA sequence. An embodiment provides a pool of these vectors comprising two or more of the vectors (e.g., 2, 10, 50, 100, 200, 300, 400, 500, 1,000, 2,000, 3,000, 4,000, 5,000, 10,000, 15,000, 20,000, 25,000, 30,000 or more) wherein each of the genetic engineering cassettes is unique.

Methods of Use of Libraries

In one embodiment methods of modifying a target polynucleotide in a host cell (e.g. a eukaryotic cell or a prokaryotic cell), which may be in vivo, ex vivo or in vitro, are provided. Culturing can occur at any stage ex vivo. The cell or cells can be re-introduced into a non-human animal or organism. The homology-directed-repair engineering methods described herein can be used at a genome scale to provide about 500, 1,000, 2,000, 3,000, 5,000, 10,000, 15,000, 20,000 or more specific genetic variants in host cells. In an embodiment, more than about 80, 85, 90, 95, 96, 97, 98, 99% or more target sequences can be efficiently edited with an average frequency (i.e., editing efficiency) of about 70, 75, 80, 82, 85, 90, 95% or more.

An embodiment provides methods for using one or more elements of a CRISPR system. The CRISPR complexes and methods describes herein provide effective means for modifying target polynucleotides. CRISPR complexes and methods described herein have a wide variety of utility including modifying (e.g., deleting, inserting, translocating, inactivating, activating) a target polynucleotide in a multiplicity of cell types.

CRISPR complexes and methods described herein have a broad spectrum of applications in, e.g., gene therapy, drug screening, disease diagnosis, and prognosis.

A method of homology directed repair-assisted engineering is provided herein. The method comprises delivering a pool of vectors to host cells. Host cells can be prokaryotic or eukaryotic cells (e.g., bacterial, yeast, or mammalian cells). The vectors can comprise, as described in more detail above, a first promoter upstream of an insertion site and downstream of the insertion site: a terminator, a second promoter, a nucleic acid sequence encoding an RNA-guided DNA endonuclease protein, a third promoter, and a tracrRNA sequence, and in the insertion site a genetic engineering cassette comprising from a 5′ end to a 3′ end: a first direct repeat sequence; a homologous recombination editing template comprising two homology arms with a deletion portion, a substitution portion, or an insertion portion between the two homology arms; a guide sequence; and a second direct repeat sequence. The homologous recombination editing template can comprise, for example, a deletion portion that removes a protospacer adjacent motif (PAM) sequence and causes a gene disruption. A gene disruption means that an insertion, deletion, or substitution causes a gene product to not be expressed or to be expressed such that the gene product has lost most or all of its function. Transformed genetic variant host cells can be isolated having one or more phenotypes. The phenotype can be the same or different from that of the original host cells. More than about 20, 100, 500, 750, 1,000, 2,000, 5,000, 10,000 or more specific unique transformed genetic variant host cells can be generated.

A phenotype is a set of observable characteristics of a cell or population of cells resulting from the interaction of the genotype of the cells with the environment. Examples include antibiotic resistance, tolerance to certain chemicals, antigenic changes, morphological characteristics, metabolic activities such as increased or decreased ability to utilize some nutrients, lost or gained ability to synthesize particular enzyme, pigments, toxins etc., growth properties, motility, loss or gain of ability to use certain energy sources.

In an embodiment methods of homology directed repair-assisted engineering are used to identify cells with new or improved desirable phenotypes.

The genomic loci of the nucleic acid molecule that causes a new or improved phenotype can be identified by sequencing portions of the cell's nucleic acid molecules.

The unique genetic engineering cassette in each plasmid serves as a genetic barcode for mutant tracking or phenotype tracking by sequencing, such as next-generation sequencing (NGS). Furthermore, a unique barcode present in a genetic engineering cassette can be used for mutant tracking.

Saturation Mutagenesis

Methods are provided for methods of saturation mutagenesis. Saturation mutagenesis means mutating a specific target sequence, such as non-coding region or coding region of a protein at many if not all nucleic acids (e.g. about 5, 10, 25, 50, 75, 100, 500, 1,000, 2,000, 3,000, or more nucleic acids) within a pool of host cells. In general, each host cell will comprise 1 nucleic acid mutation (e.g. a deletion, substitution, or insertion), of the target sequence, but each host cell can comprise 2, 3, 4, 5, or more mutations of the target sequence. In an embodiment 2, 3, 4, 5, 6, 7, 8, 9, 10, or more target sequences are targeted in saturation mutagenesis.

In an embodiment, a method of saturation mutagenesis of a target nucleic acid molecule in host cells comprises designing and making a plurality of genetic engineering cassettes specific for (i.e., target) the target nucleic acid at a plurality of positions (i.e. changes, deletes, or causes an insertion at a particular nucleic acid position of the target molecule). A plurality can be 2, 5, 10, 20, 50, 100, 500, 1,000, or more. The genetic engineering cassettes can comprise from a 5′ end to a 3′ end a first direct repeat sequence; a homologous recombination editing template comprising two homology arms with a deletion portion, a substitution portion, or an insertion portion; a guide sequence; and a second direct repeat sequence. The deletion portion, substitution portion, or insertion portion is between the homology arms. The plurality of genetic engineering cassettes is inserted into vectors to create a vector pool. The vector can comprise a first promoter upstream of the insertion sites and downstream of the insertion sites: a terminator, a second promoter, a nucleic acid molecule encoding an RNA-guided DNA endonuclease protein, a third promoter, and a tracrRNA sequence. The pool of vectors is delivered to host cells. Transformed genetic variant host cells are isolated with one or more phenotypes. More than about 10, 20, 100, 500, 750, 1,000, 2,000, 5,000, 10,000 or more specific unique transformed genetic variant host cells can be generated. The genetic bar code, the specific sequence of the genetic engineering cassette, or specific sequence of the guide RNA can be used to ensure proper sequencing of the genetic variant host cells at the mutation site.

A transformed genetic variant host cell is a cell that has at least one nucleic acid modification (insertion, deletion, substitution) as the result of the methods described herein. A pool of unique transformed variant host cells comprises a group of host cells that have mutations throughout the host cell genome. Each host cell in the pool will have 1, 2, 3, or more nucleic acid modifications. In an embodiment, the pool of unique transformed variant host cells have about 10, 20, 50, 100, 500, 1,000, 5,000, 10,000, 20,000 or more different nucleic acid modifications throughout the genome.

The genomic loci of the nucleic acid molecule that causes one or more phenotypes can be determined through, e.g., sequencing.

Saturation mutagenesis can be useful for many applications including, for example, directed evolution and structure-function studies.

Engineering of Specific Phenotypes

Compositions and methods described herein can be used to engineer a desired phenotype of host cells. For example, a vector library can be constructed, wherein the vector library comprises two or more vectors comprising a genetic engineering cassette in an insertion site of the vectors that target one or more target sequences of the host cells at one or more nucleic acid positions (i.e. changes, deletes, or causes an insertion at a particular nucleic acid position of the target molecule). Genetic engineering cassettes can comprise from a 5′ end to a 3′ end: (i) a first direct repeat sequence; (ii) a homologous recombination editing template comprising two homology arms with a deletion portion, a substitution portion, or an insertion portion; (iii) a guide sequence; and (iv) a second direct repeat sequence. The deletion portion, substitution portion, or insertion portion are between the homology arms. The host cells can be transformed with the vector library to form a transformed genetic variant host cell pool. The vectors can comprise a first promoter upstream of the insertion site and downstream of the insertion site: a terminator, a second promoter, a nucleic acid molecule encoding an RNA-guided DNA endonuclease protein, a third promoter, and a tracrRNA sequence.

More than about 20, 100, 500, 750, 1,000, 2,000, 5,000, 10,000 or more specific unique transformed genetic variant host cells can be generated. Transformed host cells with a desired phenotype can be selected.

The transformed host cell pool (i.e., genetic variant host cell mutants) can be enriched for the desired phenotype prior to selecting host cells with a desired phenotype. Enrichment means exposing the genetic variant host cell mutants to conditions that will select for the desired phenotype. Methods of enrichment include, for example, exposing the genetic variant host cells to an antibiotic, certain chemicals, nutrients, enzymes, pigments, toxins, certain energy sources, certain pHs, or certain temperatures.

Plasmids can be extracted from the library of host cell mutants and sequenced.

In another method of homology directed repair-assisted engineering a pool of vectors each containing a unique genetic engineering cassette is delivered to host cells. A genetic engineering cassette can comprise from a 5′ end to a 3′ end: (i) a first direct repeat sequence; (ii) a first homologous recombination editing template comprising two homology arms with a deletion portion, a substitution portion, or an insertion portion; (iii) a first guide sequence; (iv) a second direct repeat sequence; (v) a second homologous recombination editing template comprising two homology arms with a deletion portion, a substitution portion, or an insertion portion; (vi) a second guide sequence; and (vii) a third direct repeat sequence. The deletion portion, substitution portion, or insertion portion can be between the homology arms. The genetic engineering cassette can further comprise a first priming site at a 5′ end of the cassette and a second priming site at a 3′ end of the cassette. The first priming site, the second priming site, or both the first and second priming site can comprise a restriction enzyme cleavage site. The priming sites can be the same or different. The priming sites can be operably linked to the genetic engineering cassette components.

The first homologous recombination editing template and the second homologous recombination editing template of the genetic engineering editing cassette can each provide for a first substitution, first insertion, or first deletion, and a second substitution, second insertion, or second deletion in different locations of the same target polynucleotide. That is, the genetic engineering editing cassette can provide for 2 different changes to the same target polynucleotide. The first substitution, first insertion, or first deletion can occurs within about 5, 10, 15, 20, 25, 30, 40, 50, 60, 70, 80, 90, 100, 200, 300, 400, 500, 1,000, 5,000, 10,000, or more nucleic acids of the second substitution, second insertion, or second deletion site. In an embodiment the first substitution, first insertion, or first deletion and the second substitution, second insertion, or second deletion site, can occur in any two distal loci across the whole genome of the host cell.

The first substitution can be a substitution of about 1, 2, 3, 4, 5, 10, 15, 20, or more nucleic acids, the first insertion can be an insertion of about 1, 2, 3, 4, 5, 10, 15, 20, or more nucleic acids, the first deletion can be a deletion of about 1, 2, 3, 4, 5, 10, 15, 20, or more nucleic acids, the second substitution can be a substitution of about 1, 2, 3, 4, 5, 10, 15, 20, or more nucleic acids, the second insertion can be an insertion of about 1, 2, 3, 4, 5, 10, 15, 20, or more nucleic acids, and the second deletion can be a deletion of about 1, 2, 3, 4, 5, 10, 15, 20, or more nucleic acids.

In an embodiment, the genetic engineering cassette is present in a vector. The vector can comprise a first promoter upstream of the genetic engineering cassette and downstream of the genetic engineering cassette the vector can comprise: a terminator, a second promoter, a nucleic acid molecule encoding an RNA-guided DNA endonuclease protein, a third promoter, and a tracrRNA sequence.

In an embodiment, a pool of vectors is provided wherein each of the genetic engineering cassettes within each vector is unique. A pool of vectors is provided comprising two or more (e.g., 2, 10, 50, 100, 200, 300, 400, 500, 1,000, 2,000, 3,000, 4,000, 5,000, 10,000, 15,000, 20,000, 25,000, 30,000 or more) of the vectors, wherein each of the genetic engineering cassettes is unique. Each genetic engineering cassette can be specific for (i.e. target) a different set of target nucleic acids. Genetic engineering cassettes can target different target nucleic acids or can target one particular target nucleic acid at several different positions.

The pool of vectors can be delivered to host cells to generate a pool of genetic variant host cells. More than about 20, 100, 500, 750, 1,000, 2,000, 5,000, 10,000 or more specific unique transformed genetic variant host cells can be generated. Each host cell can comprise a unique vector.

Kits

In an embodiment kits are provided that contain any one or more of the elements disclosed in the above methods and compositions. In some embodiments, the kit comprises a pool of vectors each comprising a unique genetic engineering cassette and instructions for using the kit. Elements can be provided individually or in combinations, and can be provided in any suitable container, such as a vial, a bottle, or a tube.

A kit can comprise one or more reagents for use in a process utilizing one or more of the elements described herein. Reagents can be provided in any suitable container. For example, a kit can provide one or more reaction or storage buffers. Reagents can be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof in some embodiments, the buffer is alkaline. In some embodiments, a buffer has a pH from about 7 to about 10.

Yeast Mutants

Genetically Engineered Microorganisms

Genetically engineered microorganisms of the disclosure comprise one or more gene disruptions of one or more polynucleotides encoding SAP30, UBC4, BUL1, SUR1, SIZ1, LCB3 or any combination thereof. In an embodiment the polynucleotides encoding SAP30, UBC4, BUL1, SUR1, SIZ1, or LCB3 can be endogenous and one or more gene disruptions can be genetically engineered into the SAP30, UBC4, BUL1, SUR1, SIZ1, or LCB3 polynucleotides. In another embodiment polynucleotides encoding SAP30, UBC4, BUL1, SIZ1, LCB3, or SUR1 polypeptides and having one or more gene disruptions can be genetically engineered into microorganisms that do not endogenously produce SAP30, UBC4, BUL1, SIZ1, LCB3, or SUR1. In an embodiment a genetically engineered microorganism comprises one or more gene disruptions of polynucleotides encoding SAP30, UBC4, BUL1, SUR1, SIZ1, or LCB3.

A heterologous or exogenous polypeptide or polynucleotide refers to any polynucleotide or polypeptide that does not naturally occur or that is not present in the starting target microorganism. For example, a polynucleotide from bacteria that is transformed into a yeast cell that does not naturally or otherwise comprise the bacterial polynucleotide, is a heterologous or exogenous polynucleotide. A heterologous or exogenous polypeptide or polynucleotide can be a wild-type, synthetic, or mutated polypeptide or polynucleotide. In an embodiment, a heterologous or exogenous polypeptide or polynucleotide is not naturally present in a starting target microorganism and is from a different genus or species than the starting target microorganism.

A homologous or endogenous polypeptide or polynucleotide refers to any polynucleotide or polypeptide that naturally occurs or that is otherwise present in a starting target microorganism. For example, a polynucleotide that is naturally present in a yeast cell is a homologous or endogenous polynucleotide. In an embodiment, a homologous or endogenous polypeptide or polynucleotide is naturally present in a starting target microorganism.

Improved Furfural and Acetic Acid Tolerance

Improved tolerance to furfural or acetic acid refers to a genetically modified microorganism that has a reduced lag time, an improved growth rate, increased biomass, or combinations thereof, in the presence of furfural or acetic acid than the parent microorganism from which it was derived, a wild-type microorganism, or a control microorganism. Furfural can be present at about 2, 3, 4, 5, 10 mM or more. Acetic acid can be present in about 0.1, 0.5, 0.75, 1.0, 2.0, 3.0% or more. An improved growth rate is at least 5%, such as at least 10%, such as at least 20%, such as at least 50%, such as at least 75% higher than that of a control, typically the parent cell or strain. A reduced lag time is at least 10%, such as at least 20%, such as at least 50%, such as at least 75%, such as at least 90% shorter than that of a control, typically the parent cell or strain. Improved biomass accumulation is at least 5%, such as at least 10%, such as at least 20%, such as at least 50%, such as at least 75% higher than that of a control, typically the parent cell or strain. A control or wild-type microorganism is an otherwise identical microorganism strain that has not been recombinantly modified as described herein.

Recombinant Microorganisms

A recombinant, transgenic, or genetically engineered microorganism is a microorganism, e.g., bacteria, fungus, or yeast that has been genetically modified from its native state. Thus, a “recombinant yeast” or “recombinant yeast cell” refers to a yeast cell (i.e., Ascomycota and Basidiomycota) that has been genetically modified from the native state. A recombinant yeast cell can have, for example, nucleotide insertions, nucleotide deletions, nucleotide rearrangements, gene disruptions, recombinant polynucleotides, heterologous polynucleotides, deleted polynucleotides, nucleotide modifications, or combinations thereof introduced into its DNA. These genetic modifications can be present in the chromosome of the yeast or yeast cell, or on a plasmid in the yeast or yeast cell. Recombinant cells disclosed herein can comprise exogenous nucleotide sequences on plasmids. Alternatively, recombinant cells can comprise exogenous nucleotide sequences stably incorporated into their chromosome.

A recombinant microorganism can comprise one or more polynucleotides not present in a corresponding wild-type cell, wherein the polynucleotides have been introduced into that microorganism using recombinant DNA techniques, or which polynucleotides are not present in a wild-type microorganism and is the result of one or more mutations.

A genetically modified or recombinant microorganism can be yeast (i.e., (i.e., Ascomycota and Basidiomycota). Examples include: Saccharomyceraceae, such as Saccharomyces cerevisiae, Saccharomyces cerevisiae strain S8, Saccharomyces pastorianus, Saccharomyces beticus, Saccharomyces fermentati, Saccharomyces paradoxus, Saccharomyces uvarum and Saccharomyces bayanus; Schizosaccharomyces such as Schizosaccharomyces pombe, Schizosaccharomyces japonicus, Schizosaccharomyces octosporus and Schizosaccharomyces cryophilus; Torulaspora such as Torulaspora delbrueckii; Kluyveromyces such as Kluyveromyces marxianus; Pichia such as Pichia stipitis, Pichia pastoris or pichia angusta, Zygosaccharomyces such as Zygosaccharomyces bailii; Brettanomyces such as Brettanomyces inter medius, Brettanomyces bruxellensis, Brettanomyces anomalus, Brettanomyces custersianus, Brettanomyces naardenensis, Brettanomyces nanus, Dekkera bruxellensis and Dekkera anomala; Metschmkowia, Issatchenkia, such as Issatchenkia orientalis, Kloeckera such as Kloeckera apiculata; Aureobasidium such as Aureobasidium pullulans.

In an embodiment, a genetically engineered or recombinant microorganism has attenuated expression of a polynucleotide encoding a SIZ1 polypeptide (SEQ ID NO:736), a SAP30 (SEQ ID NO:732) polypeptide, a UBC4 polypeptide (SEQ ID NO:733), a BUL1 polypeptide (SEQ ID NO:734), a SUR1 (SEQ ID NO:735) polypeptide, a LCB3 polypeptide (SEQ ID NO:737), or combinations thereof. Attenuated means reduced in amount, degree, intensity, or strength. Attenuated gene or polynucleotide expression can refer to a reduced amount and/or rate of transcription of the gene or polynucleotide in question. As nonlimiting examples, an attenuated gene or polynucleotide can be a mutated or disrupted gene or polynucleotide (e.g., a gene or polynucleotide disrupted by partial or total deletion, truncation, frameshifting, or insertional mutation) or that has decreased expression due to alteration or disruption of gene regulatory elements. An attenuated gene may also be a gene targeted by a construct that reduces expression of the gene or polynucleotide, such as, for example, an antisense RNA, microRNA, RNAi molecule, or ribozyme.

Attenuate also means to weaken, reduce, or diminish the biological activity of a gene product or the amount of a gene product expressed (e.g., SIZ1, SAP30, UBC4, BUL1, SUR1, LCB3 proteins) via, for example a decrease in translation, folding, or assembly of the protein. In an embodiment attenuation of a gene product (a SIZ1, SAP30, UBC4, BUL1, SUR1, LCB3 protein) means that the gene product is expressed at a rate or amount about 5, 10, 20, 30, 40, 50, 60, 70, 80, 90, 95, or 99% less (or any range between about 5 and 99% less; about 5 and 95% less; about 20 and 50% less, about 10 and 40% less, or about 10 and 90% less) than occurs in a wild-type or control organism. In an embodiment, attenuation of a gene product (e.g., SIZ1, SAP30, UBC4, BUL1, SUR1, LCB3) means that the biological activity of the gene product is about 5, 10, 20, 30, 40, 50, 60, 70, 80, 90, 95, or 99% less (or any range between about 5 and 99% less; about 5 and 95% less, about 10 and 90% less) than occurs in a wild-type or control organism. SIZ1 is a SUMO E3 ligase that promotes attachment of small ubiquitin-related modifier sumo (Smt3p) to primarily cytoplasmic proteins and regulates Rsp5p ubiquitin ligase activity. SAP30 is Sin3-Associated polypeptide, which is a component of Rpd3L histone deacetylase complex and is involved in silencing at telomeres, rDNA, and silent mating-type loci and in telomere maintenance. UBC4 is ubiquitin-conjugating enzyme (E2), which is a key E2 partner with Ubc1p for the anaphase-promoting complex (APC). UBC4 mediates degradation of abnormal or excess proteins, including calmodulin and histone H3, regulates levels of DNA polymerase-a to promote efficient and accurate DNA replication, interacts with many SCF ubiquitin protein ligases, and is a component of the cellular stress response. BUL1 is a ligase (Binds Ubiquitin Ligase) that is a ubiquitin-binding component of the Rsp5p E3-ubiquitin ligase complex. SUR1 is suppressor of Rvs161 and rvs167 mutations. SUR1 is a mannosylinositol phosphorylceramide (MIPC) synthase catalytic subunit and forms a complex with regulatory subunit Csg2p. LCB3 is long-chain base-1-phosphate phosphatase. LCB3 is specific for dihydrosphingosine-1-phosphate, regulates ceramide and long-chain base phosphates levels, and is involved in incorporation of exogenous long chain bases in sphingolipids.

In an embodiment a genetically engineered or recombinant microorganism expresses a polynucleotide encoding a SIZ1 polypeptide, a SAP30 polypeptide, a UBC4 polypeptide, a BUL1 polypeptide, a SUR1 polypeptide, a LCB polypeptide, or combinations thereof at an attenuated rate or amount (e.g., amount and/or rate of transcription of the gene or polynucleotide). An attenuated rate or amount is about 5, 10, 20, 30, 40, 50, 60, 70, 80, 90, 95, 99% less than the rate of a wild-type or control microorganism. The result of attenuated expression of polynucleotide encoding a SIZ1 polypeptide, a SAP30 polypeptide, a UBC4 polypeptide, a BUL1 polypeptide, a SUR1 polypeptide, a LCB3 polypeptide, or combinations thereof is attenuated expression of a SIZ1 polypeptide, a SAP30 polypeptide, a UBC4 polypeptide, a BUL1 polypeptide, a LCB3 polypeptide, and/or a SUR1 polypeptide.

Attenuated expression requires at least some expression of a biologically active wild-type or mutated SIZ1 polypeptide, wild-type or mutated SAP30 polypeptide, wild-type or mutated UBC4 polypeptide, wild-type or mutated BUL1 polypeptide, wild-type or mutated SUR1 polypeptide, wild-type or mutated LCB3 polypeptide, or combinations thereof.

Deleted or null gene or polynucleotide expression can be gene or polynucleotide expression that is eliminated, for example, reduced to an amount that is insignificant or undetectable. Deleted or null gene or polynucleotide expression can also be gene or polynucleotide expression that results in an RNA or protein that is nonfunctional, for example, deleted gene or polynucleotide expression can be gene or polynucleotide expression that results in a truncated RNA and/or polypeptide that has substantially no biological activity.

In an embodiment, a genetically engineered or recombinant microorganism has no expression of a polynucleotide encoding a SIZ1 polypeptide, a SAP30 polypeptide, a UBC4 polypeptide, a BUL1 polypeptide, a SUR1 polypeptide, a LCB3 polypeptide, or combination thereof. The result is that substantially no SIZ1 polypeptides, SAP30 polypeptides, UBC4 polypeptides, BUL1 polypeptides, SUR1 polypeptides, a LCB3 polypeptides, or combinations are present in the cell.

The lack of expression can be caused by at least one gene disruption or mutation of a SIZ1 gene, a SAP30 gene, a UBC4 gene, a BUL1 gene, a SUR1 gene, a LCB3 gene or combinations thereof which results in no expression of the SIZ1 gene, the SAP30 gene, the UBC4 gene, the BUL1 gene, the SUR1 gene, the LCB3 gene, or combinations thereof. For example, the lack of expression can be caused by a gene disruption in a SIZ1 gene, a SAP30 gene, a UBC4 gene, a BUL1 gene, a LCB3 gene, or a SUR1 gene which results in attenuated expression of the SIZ1 gene, the SAP30 gene, the UBC4 gene, the BUL1 gene, the LCB3 gene, or the SUR1 gene. Alternatively, a SIZ1 gene, a SAP30 gene, a UBC4 gene, a BUL1 gene, a SUR1 gene, a LCB3 gene or combinations thereof can be transcribed but not translated, or the genes can be transcribed and translated, but the resulting SIZ1 polypeptide, SAP30 polypeptide, UBC4 polypeptide, BUL1 polypeptide, SUR1 polypeptide, LCB3 polypeptide, or combinations thereof have substantially no biological activity.

In an embodiment, a recombinant microorganism is mutated or otherwise genetically altered such that there is substantially no expression of SAP30 and/or UBC4 polypeptides in the cell. In an embodiment, a recombinant microorganism is mutated or otherwise genetically altered such that there is substantially no expression of SIZ1, SAP30, LCB3, and/or UBC4 polypeptides in the cell. In an embodiment, a recombinant microorganism is mutated or otherwise genetically altered such that there is substantially no expression of SIZ1 and LCB3 polypeptides in the cell. In an embodiment, a recombinant microorganism is mutated or otherwise genetically altered such that there is substantially no expression of BUL1 and SUR1 polypeptides in the cell or substantially no expression of BUL1 polypeptides in a cell. In an embodiment, a recombinant microorganism is mutated or otherwise genetically altered such that there is substantially no expression of SIZ1, SAP30, UBC4, BUL1, SUR1, LCB3 polypeptides, or combinations thereof in the cell.

In an embodiment a SIZ1 polypeptide has at least 90% sequence identity to SEQ ID NO:736. In an embodiment a SAP30 polypeptide has at least 90% sequence identity to SEQ ID NO:732. In an embodiment a UBC4 polypeptide has at least 90% sequence identity to SEQ ID NO:733. In an embodiment a BUL1 polypeptide has at least 90% sequence identity to SEQ ID NO:734. In an embodiment a SUR1 polypeptide has at least 90% sequence identity to SEQ ID NO:735. In an embodiment a LCB3 polypeptide has at least 90% sequence identity to SEQ ID NO:737.

In an embodiment a genetically engineered yeast has improved furfural tolerance, wherein the biological activity of an endogenous protein having at least 90% sequence identity to an amino acid sequence set forth in SEQ ID NO:736, set forth in SEQ ID NO:737, set forth in SEQ ID NO:732, SEQ ID NO:733, or combinations thereof is reduced or eliminated as compared to a control yeast.

In an embodiment a genetically engineered yeast has improved acetic acid tolerance, wherein the biological activity of an endogenous protein having at least 90% sequence identity to an amino acid sequence set forth in SEQ ID NO:734, SEQ ID NO:735, or both is reduced or eliminated as compared to a control yeast.

A genetically engineered or recombinant microorganism can have improved furfural tolerance or improved acetic acid tolerance or both improved furfural tolerance and improved acetic acid tolerance as compared to a control or wild-type microorganism.

The polynucleotides encoding a SIZ1 polypeptide, a SAP30 polypeptide, a UBC4 polypeptide, a BUL1 polypeptide, a SUR1 polypeptide, a LCB3 polypeptide can be deleted or mutated using a genetic manipulation technique selected from, for example, TALEN, Zinc Finger Nucleases, and CRSPR-Cas9.

One or more regulatory elements controlling expression of the polynucleotides encoding a SIZ1 polypeptide, a SAP30 polypeptide, a UBC4 polypeptide, a BUL1 polypeptide, a SUR1 polypeptide, a LCB3 polypeptide, or combinations thereof can be mutated or replaced to prevent or attenuate expression of a SIZ1 polypeptide, a SAP30 polypeptide, a UBC4 polypeptide, a BUL1 polypeptide, a SUR1 polypeptide, a LCB3 polypeptide, or combinations thereof as compared to a control or wild-type microorganism. For example, a promoter can be mutated or replaced such that the gene expression or polypeptide expression is attenuated or such that the SIZ1, SAP30, UBC4, BUL1, LCB3, or SUR1 polynucleotides are not transcribed. In one embodiment, one or more promoters for SIZ1, SAP30, UBC4, BUL1, SUR1, LCB3, or combinations thereof are replaced with a promoter that has weaker activity (e.g., TEF1p, CYC1p, ADH1p, ACT1p, HXT7p, PGI1p, TDH2p, PGK1p) than the wild-type promoter. A promoter with weaker activity transcribes the polynucleotide at a rate about 5, 10, 20, 30, 40, 50, 60, 70, 80, or 90% less than the wild-type promoter for that polynucleotide. In another embodiment, one or more promoters for SIZ1, SAP30, UBC4, BUL1, SUR1, LCB3, or combinations thereof are replaced with a inducible promoter (e.g., TetO promoters such as TetO3, TetO7, and CUP1p) that can be controlled to attenuate expression of SIZ1, SAP30, UBC4, BUL1, LCB3, or SUR1 or combinations thereof.

The present disclosure provides genetically engineered microorganisms lacking expression or having attenuated or reduced expression of SIZ1, SAP30, UBC4, BUL1, LCB3, or SUR1 polypeptides or combinations thereof, or expression of mutant SIZ1, SAP30, UBC4, BUL1, LCB3, or SUR1 polypeptides or combinations thereof that have reduced activity.

The reduced expression, non-expression, or expression of mutated, inactive, or reduced activity polypeptides can be affected by deletion of the polynucleotide or gene encoding SIZ1, SAP30, UBC4, BUL1, LCB3, or SUR1, replacement of the wild-type polynucleotide or gene with mutated forms, deletion of a portion of a SIZ1, SAP30, UBC4, BUL1, LCB3, or SUR1 polynucleotide or gene or combinations thereof to cause expression of an inactive form of the polypeptides, or manipulation of the regulatory elements (e.g. promoter) to prevent or reduce expression of wild-type SIZ1, SAP30, UBC4, BUL1, LCB3, or SUR1 polypeptides. The promoter could also be replaced with a weaker promoter or an inducible promoter that leads to reduced expression of the polypeptides. Any method of genetic manipulation that leads to a lack of, or reduced expression and/or activity of SIZ1, SAP30, UBC4, BUL1, LCB3, or SUR1 polypeptides and can be used in the present methods, including expression of inhibitor RNAs (e.g. shRNA, siRNA, and the like).

Wild-type refers to a microorganism that is naturally occurring or which has not been recombinantly modified to increase furfural or acetic acid tolerance. A control microorganism is a microorganism (e.g. yeast) that lacks genetic modifications of a test microorganism (e.g., yeast) and that can be used to test altered biological activity of genetically modified microorganisms (e.g., yeast).

Gene Disruptions and Mutations

A genetic mutation comprises a change or changes in a nucleotide sequence of a gene or related regulatory region or polynucleotide that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be 2, 3, 4, 5, 6, 7, 8, 9, 10, or more nucleotide changes. Mutations can occur within the coding region of the gene or polynucleotide as well as within the non-coding and regulatory elements of a gene. A genetic mutation can also include silent and conservative mutations within a coding region as well as changes which alter the amino acid sequence of the polypeptide encoded by the gene or polynucleotide. A genetic mutation can, for example, increase, decrease, or otherwise alter the activity (e.g., biological activity) of the polypeptide product. A genetic mutation in a regulatory element can increase, decrease, or otherwise alter the expression of sequences operably linked to the altered regulatory element.

A gene disruption is a genetic alteration in a polynucleotide or gene that renders an encoded gene product (e.g., SIZ1, SAP30, UBC4, BUL1, LCB3, or SUR1) inactive or attenuated (e.g., produced at a lower amount or having lower biological activity). A gene disruption can include a disruption in a polynucleotide or gene that results in no expression of an encoded gene product, reduced expression of an encoded gene product, or expression of a gene product with reduced or attenuated biological activity. The genetic alteration can be, for example, deletion of the entire gene or polynucleotide, deletion of a regulatory element required for transcription or translation of the polynucleotide or gene, deletion of a regulatory element required for transcription or translation or the polynucleotide or gene, addition of a different regulatory element required for transcription or translation or the gene or polynucleotide, deletion of a portion (e.g. 1, 2, 3, 6, 9, 21, 30, 60, 90, 120 or more nucleic acids) of the gene or polynucleotide, which results in an inactive or partially active gene product, replacement of a gene's promoter with a weaker promoter, replacement or insertion of one or more amino acids of the encoded protein to reduce its activity, stability, or concentration, or inactivation of a gene's transactivating factor such as a regulatory protein. A gene disruption can include a null mutation, which is a mutation within a gene or a region containing a gene that results in the gene not being transcribed into RNA and/or translated into a functional gene product. An inactive gene product has no biological activity.

Zinc-finger nucleases (ZFNs), Talens, and CRSPR-Cas9 allow double strand DNA cleavage at specific sites in yeast chromosomes such that targeted gene insertion or deletion can be performed (Shukla et al., 2009, Nature 459:437-441; Townsend et al., 2009, Nature 459:442-445). This approach can be used to modify the promoter of endogenous genes or the endogenous genes themselves to modify expression of SIZ1, SAP30, UBC4, BUL1, LCB3, or SUR1, which can be present in the genome of yeast of interest. ZFNs, Talens or CRSPR/Cas9 can be used to change the sequences regulating the expression of the polypeptides to increase or decrease the expression or alter the timing of expression beyond that found in a non-engineered or wild-type yeast, or to delete the wild-type polynucleotide, or replace it with a deleted or mutated form to alter the expression and/or activity of SIZ1, SAP30, UBC4, BUL1, LCB3, or SUR1.

Polypeptides

A polypeptide is a polymer of two or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide, etc., has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure. A purified polypeptide does not include unpurified or semi-purified cell extracts or mixtures of polypeptides that are less than 70% pure.

The term “polypeptides” can refer to one or more of one type of polypeptide (a set of polypeptides). “Polypeptides” can also refer to mixtures of two or more different types of polypeptides (a mixture of polypeptides). The terms “polypeptides” or “polypeptide” can each also mean “one or more polypeptides.”

As used herein, the term “polypeptide of interest” or “polypeptides of interest”, “protein of interest”, “proteins of interest” includes any or a plurality of any of the SIZ1, SAP30, UBC4, BUL1 SUR1, LCB3 polypeptides or other polypeptides described herein.

A mutated protein or polypeptide comprises at least one deleted, inserted, and/or substituted amino acid, which can be accomplished via mutagenesis of polynucleotides encoding these amino acids. Mutagenesis includes well-known methods in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989).

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Variants will be sufficiently similar to the amino acid sequence of the polypeptides described herein. Such variants generally retain the functional activity of the polypeptides described herein. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

Polypeptides and polynucleotides that are sufficiently similar to polypeptides and polynucleotides described herein (e.g., SIZ1, SAP30, UBC4, BUL1, SUR1, LCB3) can be used herein. Polypeptides and polynucleotides that about 85, 90, 95, 96, 97, 98, 99% or more homology or identity to polypeptides and polynucleotides described herein (e.g., SIZ1, SAP30, UBC4, BUL1, SUR1, LCB3) can also be used herein.

Conditions

Fermentation conditions, such as temperature, cell density, selection of substrate(s), selection of nutrients, can be determined by those of skill in the art. Temperatures of the medium during each of the growth phase and the production phase can range from above about 1° C. to about 50° C. The optimal temperature can depend on the particular microorganism used. In an embodiment, the temperature is about 30, 35, 40, 45, 50° C.

During a production phase, the concentration of cells in the fermentation medium can be in the range of about 1 to about 150, about 3 to about 10, or about 3 to about 6 g dry cells/liter of fermentation medium.

A fermentation can be conducted aerobically, microaerobically or anaerobically. Fermentation medium can be buffered during the fermentation so that the pH is maintained in a range of about 5.0 to about 9.0, or about 5.5 to about 7.0. Suitable buffering agents include, for example, calcium hydroxide, calcium carbonate, sodium hydroxide, potassium hydroxide, potassium carbonate, sodium carbonate, ammonium carbonate, ammonia, ammonium hydroxide and the like.

The fermentation methods can be conducted continuously, batch-wise, or some combination thereof. A fermentation reaction can be conducted over about 1, 2, 5, 10, 15, 20, 24, 25, 30, 36, 48, or more or hours.

The following are provided for exemplification purposes only and are not intended to limit the scope of the invention described in broad terms above.

### EXAMPLES

### Example 1. Efficient Genome-Scale Precision Editing in Yeast Using CRISPR/Cas9 and Homology-Directed-Repair

A CRISPR/Cas9 and homology-directed-repair assisted genome-scale engineering method named CHAnGE is described that can rapidly output tens of thousands of specific genetic variants in host cells such as yeast. The system has single-nucleotide resolution genome-editing capability and creates a genome-wide gene disruption collection, which can be used to, for example, improve tolerance of cells to growth inhibitors.

Eukaryotic MAGE (eMAGE) enables genome engineering in yeast but the editing efficiency of eMAGE relies on close proximity (e.g., about 1.5 kb) of target sequences to a replication origin and co-selection of a URA3 marker. Barbieri, E. M., Muir, P., Akhuetie-Oni, B. O., Yellman, C. M. & Isaacs, F. J. Cell 171, 1453-1467 (2017). Additionally, eMAGE has not been shown to work on a genome scale. Described herein is a CRISPR/Cas9 and homology-directed-repair (HDR) assisted genome-scale engineering (CHAnGE) method that enables rapid engineering of Saccharomyces cerevisiae on a genome-scale with precise and trackable edits. Furthermore, co-selection with a protein marker like URA3 and close proximity (about 1.5 Kb) of target sequences to a replication origin is not required. Genome-scale means that target sequences throughout the entire genome can be engineered.

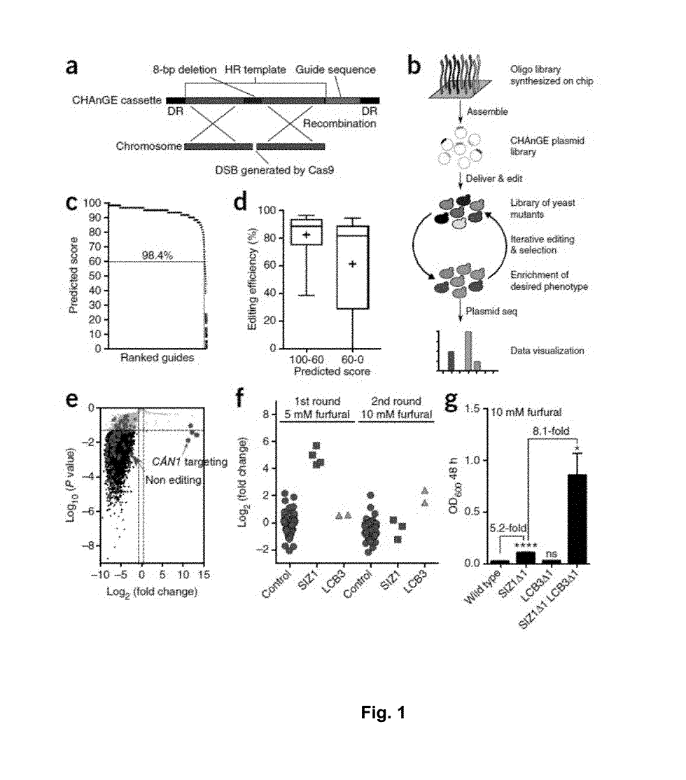

To enable large-scale engineering using HDR, a CRISPR guide sequence and a homologous recombination (HR) template is provided in a single oligonucleotide (a CHAnGE cassette, FIG. 1a). Unlike other cassettes, the long eukaryotic RNA promoter is located on the plasmid backbone to reduce oligonucleotide length. Cloning and delivering a pooled CHAnGE plasmid library into a yeast strain and subsequent editing generates a yeast mutant library (FIG. 1b). The unique CHAnGE cassette in each plasmid serves as a genetic barcode for mutant tracking by next generation sequencing (NGS).

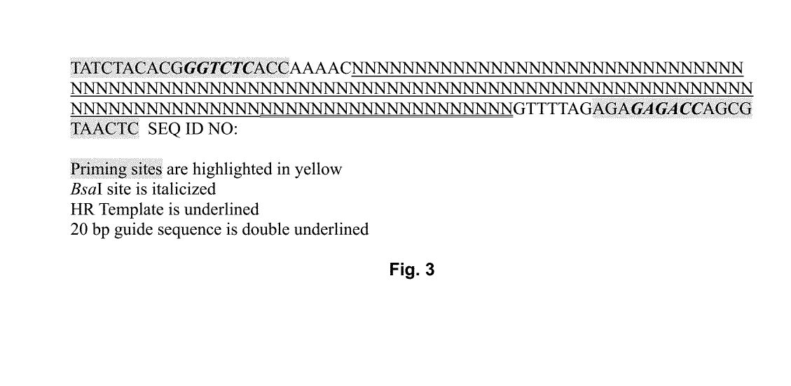

CHAnGE was applied for genome-wide gene disruption. To do this, previously described criteria (Bao, Z. et al. ACS Synth. Biol. 4, 585-594 (2015); Cong, L. et al. Science 339, 819-823 (2013); Wang, T., Wei, J. J., Sabatini, D. M. & Lander, E. S. Science 343, 80-84 (2014)) were used to maximize the efficacy and specificity of guide sequences were applied to design guides targeting each open reading frame (ORF) in the S. cerevisiae genome. Arbitrary weights were assigned to each criterion to derive a score for each guide (Table 1). For each ORF, four top-rank guides were selected. For some ORFs, less guides were selected due to short or repetitive ORF sequences. In total 24765 unique guide sequences were used targeting 6459 ORFs (˜97.8% of ORFs annotated in SGD, Table 2). Also included were 100 non-editing guide sequences as controls. For each ORF-targeting guide, a 100 bp HR template with 50 bp homology arms and a centered 8 bp deletion was used. The deletion removes the PAM sequence and causes a frame shift mutation for gene disruption (FIG. 1a). Adapters containing priming and BsaI sites were added to both ends of the oligonucleotide to facilitate cloning (FIG. 3). CHAnGE cassettes are listed in Table 3.

TABLE 1
Criteria for scoring each 20 bp guide sequence. The hit_12mer is the
number of target sites within the genome that share the same 12 bp seed sequence.

| | | Weight | | |
|---|---|---|---|---|
| | Criterion | (W) | Condition | Multiplier (M) |
| Efficacy | GC number | ⅓ | 7 to 15 (including 7 and | 1 |
| score | | | 15) | |
| | | | Less than 7 or more than | 0 |
| | | | 15 | |
| | Composition of the last four | ⅓ | | 0.25 × (#G) + 0.2 × (#A) + 0.15 × (#C) |
| | nucleotides | | | |
| | PAM position | ⅓ | Within the first 60% of | 1 |
| | | | the ORF | |
| | | | Between 60% and 80% | 0.85 |
| | | | of the ORF | |
| | | | Within the last 20% of | 0 |
| | | | the ORF | |

| | |
|---|---|
| Specificity | 1/(hit_12mer)² |
| score | |
| Total score | 100 × Σ/(Wi × Mi)/(hit_12mer)² |

TABLE 2
Guide sequence distribution within the designed oligonucleotide library.

| | ORF targeting | | | | | | | |
|---|---|---|---|---|---|---|---|---|
| | Guide # | | | | | | Control | Total |
| | 1 | 2 | 3 | 4 | 5 | 6 | 100 | 24765 |
| ORF # | 261 | 100 | 92 | 6003 | 2 | 1 | NA | 6459 |

TABLE 3
gBlock Sequences

| gBlocks | Sequences (5′ to 3′) |
|---|---|
| SIZ1 F268A | CTTTGGTCTCACCAAAACCAAATGAATTAAGGTGCAATAATGTTCAAATCA |
| | AAGATAATATAAGAGGTGCCAAGAGTAAGCCTGGCACAGCTAAGCCGGCG |
| | GATTTAACGCCTCATCTCAAACCTTATACTCAAAGAGGTTTCAAGAGTAAG |
| | CGTTTTAGAGAGAGACCTTTC SEQ ID NO: 01 |
| SIZ1 D345A | CTTTGGTCTCACCAAAACATCCAAAAATTATTAAACAAGCCACGTTACTTT |
| | ACTTGAAAAAAACACTTAGAGAAGCTGAAGAAATGGGCTTGACTACCACA |
| | TCTACTATCATGAGTCTGCAATGTCCTTGAAAAAAACACTTCGGGGTTTTA |
| | GAGAGAGACCTTTC SEQ ID NO: 02 |
| SIZ1I363A | CTTTGGTCTCACCAAAACAGATGCTTACAATTTATTGATTTTGAAGGGTAT |
| | TTCATTCTTGTGTACGATGCTGGACATTGCAGACTCATGATAGTAGATGT |
| | GGTAGTCAAGCCCATTTCTTTTCATTCTTGTGTACGAAATGTTTTAGAGAGA |
| | GACCTTTC SEQ ID NO: 03 |
| SIZ1 S391D | CTTTGGTCTCACCAAAACCTATATCAATTTGACATACTGGGCATTGCCACG |
| | TAGGAATTTGTAGTTGGTCGTGTAGAAACCATAATGCATCAAAACATTGCA |
| | GATGCTTACAATTTATTGATTTTGAGAATTTGTAGTTGGGAGTGTGTTTTAG |
| | AGAGAGACCTTTC SEQ ID NO: 04 |
| SIZ1 F250A | CTTTGGTCTCACCAAAACCCTCTTATATTATCTTTGATTTGAACATTATTGC |
| F299A | ACCTTAATTCATTTGGAGCTGGAAATTGGATGGGTTCATTACCTCGGGAT |
| | CCTAATGGATTAATCATCCCACCTTAATTCATTTGGGAAGTTTTAGAGCTATG |
| | CTGTTTTGAATGGTCCCAAAACGGAGTGATCATTTCTACAATGTACCCAAA |
| | TAGCTTGTATTCCTTCGTGGTAGCTGCATATATCAGCTCCACATTGTTTTG |
| | TTGAGTATAAGGTTTGAGATGAGGCTTGTATTCCTTCGTGGTGAGTTTTAGA |
| | GAGAGACCTTTC SEQ ID NO: 05 |
| SIZ1 FKS | CTTTGGTCTCACCAAAACCAAATGAATTAAGGTGCAATAATGTTCAAATCA |
| deletion | AAGATAATATAAGAGGTAAGCCTGGCACAGCTAAGCCGGCGGATTTAACG |
| | CCTCATCTCAAACCTTATACTCAAAGAGGTTTCAAGAGTAAGCGTTTTAGA |
| | GAGAGACCTTTC SEQ ID NO: 06 |
| SIZ1 AAA | CTTTGGTCTCACCAAAACCAAATGAATTAAGGTGCAATAATGTTCAAATCA |
| insertion | AAGATAATATAAGAGGTGCTGCTGCTTTCAAGAGTAAGCCTGGCACAGCTA |
| | AGCCGGCGGATTTAACGCCTCATCTCAAACCTTATACTCAAAGAGGTTTC |
| | AAGAGTAAGCGTTTTAGAGAGAGACCTTTC SEQ ID NO: 07 |
| CAN1 E184A#1 | CTTTGGTCTCACCAAAACAGTGGAACTTTGTACGTCCAAAATTGAATGAC |
| | TTGGCCAACTACACTAAGAGCTAAGGCAAAAGTGATTGCCCAAGAAAAC |
| | CAATACATGTAACCATTGGCCGCACGGCCAACTACACTAAGTTCCGTTTTA |
| | GAGAGAGACCTTTC SEQ ID NO: 08 |
| | CTTTGGTCTCACCAAAACGGTGCGGCCAATGGTTACATGTATTGGTTTTCT |
| CAN1 E184A#2 | TGGGCAATCACTTTTGCACTGGCTCTTAGTGTAGTTGGCCAAGTCATTCA |
| | ATTTTGGACGTACAAAGTTCCACTGCCAACTACACTAAGTTCCAGTTTTAG |
| | AGAGAGACCTTTC SEQ ID NO: 09 |
| | CTTTGGTCTCACCAAAACTGGTGCGGCCAATGGTTACATGTATTGGTTTTC |
| CAN1 E184A#3 | TTGGGCAATCACTTTTGCCCTTGCTCTTAGTGTAGTTGGCCAAGTCATTC |
| | AATTTTGGACGTACAAAGTTCCACTTTGGGCAATCACTTTTGCCCGTTTTAG |
| | AGAGAGACCTTTC SEQ ID NO: 10 |
| UBC4 C86A#1 | CTTTGGTCTCACCAAAACCAAGATATATCATCCAAATATCAATGCCAATGG |
| | TAACATCGCTCTTGACATCCTAAAGGATCAATGGTCACCAGCTCTAACTCTA |
| | TCGAAGGTCCTATTATCCATCTGTTTGCCAATGGTAACATCTGTCGTTTTAG |
| | AGAGAGACCTTTC SEQ ID NO: 11 |
| UBC4 C86A#2 | CTTTGGTCTCACCAAAACTCTCCTTCACAACCAAGATATATCATCCAAATA |
| | TCAATGCTAATGGTAACATCGCTCTGGACATCCTAAAGGATCAATGGTCACC |
| | AGCTCTAACTCTATCGAAGGTCCTATTATCCATCTGTTCATCCAAATATCAA |
| | TGCCAAGTTTTAGAGAGAGACCTTTC SEQ ID NO: 12 |
| UBC4 C86A#3 | CTTTGGTCTCACCAAAACCAAGATATATCATCCAAATATCAATGCCAATGG |
| | TAACATCGCTCTGGACATCTTGAAAGATCAATGGTCACCAGCTCTAACTCTA |
| | TCGAAGGTCCTATTATCCATCTGTTCATCTGTCTGGACATCCTAAGTTTTAG |
| | AGAGAGACCTTTC SEQ ID NO: 13 |
| UBC4 C86A#4 | CTTTGGTCTCACCAAAACTCTCCTTCACAACCAAGATATATCATCCAAATA |
| | TCAATGCAAATGGTAACATCGCTCTGGACATCCTAAAGGATCAATGGTCACC |
| | AGCTCTAACTCTATCGAAGGTCCTATTATCCATCTGTTGTCCAGACAGATG |
| | TTACCATGTTTTAGAGAGAGACCTTTC SEQ ID NO: 14 |
| UBC4 C86A#5 | CTTTGGTCTCACCAAAACCAAGATATATCATCCAAATATCAATGCCAATGG |
| | TAACATCGCTCTGGACATACTAAAGGATCAATGGTCACCAGCTCTAACTCTA |
| | TCGAAGGTCCTATTATCCATCTGTTCTGGAGACCATTGATCCTTTGTTTTAG |
| | AGAGAGACCTTTC SEQ ID NO: 15 |
| EMX1 | CTTTGAAGACGTCACCGAGTACAAACGGCAGAAGCTGGAGGAGGAAGGG |
| | CCTGAGTCCGAGCAGAAGCTTAAGGGCAGTGTAGTGATCAACCGGTGGCG |
| | CATTGCCACGAAGCAGGCCAATGGGGAGGACATCGAGAGTCCGAGCAGA |
| | AGAAGAAGTTTGGGTCTTCTTTC SEQ ID NO: 16 |
| CAN1-E184A-1 | CTTTGGTCTCACCAAAACTGTACGTCCAAAATTGAATGACTTGGCCAACTA |
| | CACTAAGAGCTAAGGCAAAAGTGATTGCCCAAGAAAACCAATACATGTAA |
| | CCATGGCCAACTACACTAAGTTCCGTTTTAGAGAGAGACCTTTC SEQ ID |
| | NO: 17 |
| CAN1-E184A-2 | CTTTGGTCTCACCAAAACTGTACGTCCAAAATTGAATGACTTGGCCAACTA |
| | CACTAAGAGCCAGTGCAAAAGTGATTGCCCAAGAAAACCAATACATGTAA |
| | CCATTGCCAACTACACTAAGTTCCAGTTTTAGAGAGAGACCTTTC SEQ ID |
| | NO: 18 |
| CAN1-E184A-3 | CTTTGGTCTCACCAAAACGGTTACATGTATTGGTTTTCTTGGGCAATCACT |
| | TTTGCCCTTGCTCTTAGTGTAGTTGGCCAAGTCATTCAATTTTGGACGTA |
| | CATTGGGCAATCACTTTTGCCCGTTTTAGAGAGAGACCTTTC SEQ ID NO: 19 |
| CAN1-E184A-4 | CTTTGGTCTCACCAAAACTTACATGTATTGGTTTTCTTGGGCAATCACTTT |
| | TGCCCTGGCTCTTTCAGTTGTTGGCCAAGTCATTCAATTTTGGACGTACAA |
| | AGTTCCACTGGCGGCCCTGGAACTTAGTGTAGTGTTTTAGAGAGAGACCTT |
| | TC SEQ ID NO: 20 |
| CAN1-E184A-5 | CTTTGGTCTCACCAAAACTGCCGCCAGTGGAACTTTGTACGTCCAAAATT |
| | GAATGACTTGACCAACTACACTAAGAGCCAGGGCAAAAGTGATTGCCCAA |
| | GAAAACCAATACATGTAAACGTCCAAAATTGAATGACTGTTTTAGAGAGAG |
| | ACCTTTC SEQ ID NO: 21 |
| CAN1-E184A-6 | CTTTGGTCTCACCAAAACTCCAGCATTTGGTGCGGCCAATGGTTACATGTA |
| | TTGGTTTAGCTGGGCAATCACTTTTGCCCTGGCTCTTAGTGTAGTTGGCCA |
| | AGTCATTCAATTTTGGACGTACATTACATGTATTGGTTTTCTTGTTTTAGAGA |
| | GAGACCTTTC SEQ ID NO: 22 |
| CAN1-E184A-7 | CTTTGGTCTCACCAAAACTCCAGCATTTGGTGCGGCCAATGGTTACATGTA |
| | TTGGTTTAGCTGGGCAATCACTTTTGCCCTGGCTCTTAGTGTAGTTGGCCA |
| | AGTCATTCAATTTTGGACGTACAGTTACATGTATTGGTTTTCTGTTTTAGAG |
| | AGAGACCTTTC SEQ ID NO: 23 |
| CAN1-E184A-8 | CTTTGGTCTCACCAAAACAAAAAATACTAATCCATGCCGCCAGTGGAACTT |
| | TGTACGTCCAGAACTGAATGACTTGGCCAACTACACTAAGAGCCAGGGCAA |
| | AAGTGATTGCCCAAGAAAACCAATACATGTAATTGGCCAAGTCATTCAATT |
| | TGTTTTAGAGAGAGACCTTTC SEQ ID NO: 24 |
| CAN1-E184A-9 | CTTTGGTCTCACCAAAACTCCTTTCTCCAGCATTTGGTGCGGCCAATGGT |
| | TACATGTACTGGTTTTCTTGGGCAATCACTTTTGCCCTGGCTCTTAGTGTAGT |
| | TGGCCAAGTCATTCAATTTTGGACGTACACGGCCAATGGTTACATGTATGT |
| | TTTAGAGAGAGACCTTTC SEQ ID NO: 25 |
| CAN1-E184A-10 | CTTTGGTCTCACCAAAACTGTACGTCCAAAATTGAATGACTTGGCCAACTA |
| | CACTAAGAGCCAGGGCAAAAGTGATTGCCCAAGAAAACCAATACATGTAAC |
| | CATTTGCCGCACCAAATGCTGGAGAAAGGAATCTTTGTGAGAAAACAAA |
| | CCAATACATGTAACCATGTTTTAGAGAGAGACCTTTC SEQ ID NO: 26 |
| ADE2-G158*-1 | CTTTGGTCTCACCAAAACCATTCGTCTTGAAGTCGAGGACTTTGGCATAC |
| | GATGGAAGATAAAACTTCGTTGTAAAGAATAAGGAAATGATTCCGGAAGC |
| | TTACTTTGGCATACGATGGAAGGTTTTAGAGAGAGACCTTTC SEQ ID NO: 27 |
| ADE2-G158*-2 | CTTTGGTCTCACCAAAACTGGGTTTTCCATTCGTCTTGAAGTCGAGGACT |
| | TTGGCATATGATGGAAGATAAAACTTCGTTGTAAAGAATAAGGAAATGATT |
| | CCGGAAGCTTTCGAGGACTTTGGCATACGAGTTTTAGAGAGAGACCTTTC |
| | SEQ ID NO: 28 |
| ADE2-G158*-3 | CTTTGGTCTCACCAAAACAAGAGATTTGGGTTTTCCATTCGTCTTGAAGT |
| | CGAGGACTCTTGCATACGATGGAAGATAAAACTTCGTTGTAAAGAATAAGG |
| | AAATGATTCCGGAAGCTTCGTCTTGAAGTCGAGGACTTGTTTTAGAGAGAG |
| | ACCTTTC SEQ ID NO: 29 |
| ADE2-G158*-4 | CTTTGGTCTCACCAAAACATTCGTCTTGAAGTCGAGGACTTTGGCATACG |
| | ATGGAAGATAAAACTTCGTTGTAAAGAACAAAGAAATGATTCCGGAAGCTT |
| | TGGAAGTACTGAAGGATCGTCCTAACTTCGTTGTAAAGAATAGTTTTAGAG |
| | AGAGACCTTTC SEQ ID NO: 30 |
| ADE2-G158*-5 | CTTTGGTCTCACCAAAACTGTTGGAAGAGATTTGGGTTTTCCATTCGTCTT |
| | GAAGTCGAGAACTTTGGCATACGATGGAAGATAAAACTTCGTTGTAAAGAA |
| | TAAGGAAATGATTCCGGAAGCTTTTCCATTCGTCTTGAAGTCGGTTTTAGA |
| | GAGAGACCTTTC SEQ ID NO: 31 |
| ADE2-G158*-6 | CTTTGGTCTCACCAAAACTTTCGGCGTACAAAGGACGATCCTTCAGTACT |
| | TCCAAAGCCTCCGGAATCATTTCCTTATTCTTTACAACGAAGTTTATCTTCC |
| | ATCGTATGCCAAAGTCCTCGACTTCAAGACGAATTTCAGTACTTCCAAAG |
| | CTTCGTTTTAGAGAGAGACCTTTC SEQ ID NO: 32 |
| ADE2-G158*-7 | CTTTGGTCTCACCAAAACATTCGTCTTGAAGTCGAGGACTTTGGCATACG |
| | ATGGAAGATAAAACTTCGTTGTAAAGAATAAGGAAATGATTCCTGAAGCTTT |
| | GGAAGTACTGAAGGATCGTCCTTTGTACGCCGAAAAGAATAAGGAAATGA |
| | TTCGTTTTAGAGAGAGACCTTTC SEQ ID NO: 33 |
| ADE2-G158*-8 | CTTTGGTCTCACCAAAACATTCGTCTTGAAGTCGAGGACTTTGGCATACG |
| | ATGGAAGATAAAACTTCGTTGTAAAGAATAAGGAAATGATTCCGGAAGCTCT |
| | TGAAGTACTGAAGGATCGTCCTTTGTACGCCGAAAAATGGGCGGAAATGA |
| | TTCCGGAAGCTTGTTTTAGAGAGAGACCTTTC SEQ ID NO: 34 |
| ADE2-G158*-9 | CTTTGGTCTCACCAAAACAAGCTTCCGGAATCATTTCCTTATTCTTTACAA |
| | CGAAGTTTTATCTTCCATCGTATGCCAAAGTCCTCGACTTCAAGACAAATGG |
| | AAAACCCAAATCTCTTCCAACATTCAATAGGGACGTCTCAGTCCTCGACT |
| | TCAAGACGAAGTTTTAGAGAGAGACCTTTC SEQ ID NO: 35 |
| ADE2-G158*-10 | CTTTGGTCTCACCAAAACACAAGCCAGTGAGACGTCCCTATTGAATGTTG |
| | GAAGAGATCTAGGTTTTCCATTCGTCTTGAAGTCGAGGACTTTGGCATACGA |
| | TGGAAGATAAAACTTCGTTGTAAAGAATAAGGAAATGATTCCGGAAGCTT |
| | TTGAATGTTGGAAGAGATTTGTTTTAGAGAGAGACCTTTC SEQ ID NO: 36 |
| LYP1-R181*-1 | CTTTGGTCTCACCAAAACGTTTATCCCCGTGACATCATCTATCACTGTCTT |
| | TTCGAAGTAATTCTTATCACCTGCATTCGGTGTTTCTAACGGCTACATGTC |
| | TATCACTGTCTTTTCGAAGGTTTTAGAGAGAGACCTTTC SEQ ID NO: 37 |
| LYP1-R181*-2 | CTTTGGTCTCACCAAAACCCCAATTGAACCAGTACATGTAGCCGTTAGAA |
| | ACACCGAAAGCAGGTGATAAGAATTACTTCGAAAAGACAGTGATAGATGA |
| | TGTCACGGGGATAAACCCGTTAGAAACACCGAATGCGTTTTAGAGAGAGAC |
| | CTTTC |
| | SEQ ID NO: 38 |
| LYP1-R181*-3 | CTTTGGTCTCACCAAAACGTTTATCCCCGTGACATCATCTATCACTGTCTT |
| | TTCGAAGTAATTCTTATCACCTGCTTTCGGTGTTTCTAACGGCTACATGTAC |
| | TGGTTCAATTGGGCTATTAGGTTCTTATCACCTGCATTGTTTTAGAGAGAGA |
| | CCTTTC |
| | SEQ ID NO: 39 |
| LYP1-R181*-4 | CTTTGGTCTCACCAAAACGTTTATCCCCGTGACATCATCTATCACTGTCTT |
| | TTCGAAGTAATTCTTATCACCTGCATTCGGTGTTAGCAACGGCTACATGTACT |
| | GGTTCAATTGGGCTATTACTTATGCTGTGCCTGCATTCGGTGTTTCTAAGTT |
| | TTAGAGAGAGACCTTTC SEQ ID NO: 40 |
| LYP1-R181*-5 | CTTTGGTCTCACCAAAACGTTTATCCCCGTGACATCATCTATCACTGTCTT |
| | TTCGAAGTAATTCTTATCACCTGCATTCGGTGTTTCTAACGGCTACATGTATTG |
| | GTTCAATTGGGCTATTACTTATGCTGTGGAGGTTTCTGTCATTTCTAACGG |
| | CTACATGTACGTTTTAGAGAGAGACCTTTC SEQ ID NO: 41 |
| LYP1-R181*-6 | CTTTGGTCTCACCAAAACACATGTAGCCGTTAGAAACACCGAATGCAGGT |
| | GATAAGAATTACTTCGAAAAGACAGTGATAGATGATGTCACTGGGATAAACG |
| | TAGCCATCTCACCAAGTGACTGGGTAACGAAGACAGTGATAGATGATGTC |
| | AGTTTTAGAGAGAGACCTTTC SEQ ID NO: 42 |
| LYP1-R181*-7 | CTTTGGTCTCACCAAAACACATGTAGCCGTTAGAAACACCGAATGCAGGT |
| | GATAAGAATTACTTCGAAAAGACAGTGATAGATGATGTAACGGGGATAAACG |
| | TAGCCATCTCACCAAGTGACTGGGTAACGAAGACAGTGATAGATGATGTC |
| | ACGTTTTAGAGAGAGACCTTTC SEQ ID NO: 43 |
| LYP1-R181*-8 | CTTTGGTCTCACCAAAACACATGTAGCCGTTAGAAACACCGAATGCAGGT |
| | GATAAGAATTACTTCGAAAAGACAGTGATAGATGATGTCACGGGAATAAACG |
| | TAGCCATCTCACCAAGTGACTGGGTAACGAAGTCAGTGATAGATGATGTC |
| | ACGGTTTTAGAGAGAGACCTTTC SEQ ID NO: 44 |
| LYP1-R181*-9 | CTTTGGTCTCACCAAAACTGGGCACCATTGTCTACTTCGTTACCCAGTCA |
| | CTTGGTGAAATGGCTACGTTTATCCCCGTGACATCATCTATCACTGTCTTTTCG |
| | AAGTAATTCTTATCACCTGCATTCGGTGTTTCTAACGGCTACATGTTACCC |
| | AGTCACTTGGTGAGAGTTTTAGAGAGAGACCTTTC SEQ ID NO: 45 |
| LYP1-R181*-10 | CTTTGGTCTCACCAAAACGTTTATCCCCGTGACATCATCTATCACTGTCTT |
| | TTCGAAGTAATTCTTATCACCTGCATTCGGTGTTTCTAACGGCTACATGTACTG |
| | GTTCAACTGGGCTATTACTTATGCTGTGGAGGTTTCTGTCATTGGCCAAG |
| | GCTACATGTACTGGTTCAATGTTTTAGAGAGAGACCTTTC SEQ ID NO: 46 |
| CAN1-score-1 | CTTTGGTCTCACCAAAACGAAACCCAGGTGCCTGGGGTCCAGGTATAATA |
| | TCTAAGGATAAAAACGAACTTAGGTTGGGTTTCCTCTTTGATTAACGCTG |
| | CCTTCACATTTCAAGGTACTAAGGATAAAAACGAAGGGGTTTTAGAGAGAG |
| | ACCTTTC |
| | SEQ ID NO: 47 |
| CAN1-score-2 | CTTTGGTCTCACCAAAACCTGGGGTCCAGGTATAATATCTAAGGATAAAAA |
| | CGAAGGGAGGTTCTTAGTCCTCTTTGATTAACGCTGCCTTCACATTTCAA |
| | GGTACTGAACTAGTTGGCGAAGGGAGGTTCTTAGGTTGTTTTAGAGAGAGA |
| | CCTTTC |
| | SEQ ID NO: 48 |
| CAN1-score-3 | CTTTGGTCTCACCAAAACGGGAGGTTCTTAGGTTGGGTTTCCTCTTTGAT |
| | TAACGCTGCCTTCACATTCTGAACTAGTTGGTATCACTGCTGGTGAAGCT |
| | GCAAACCCCAGAAAATCCAACGCTGCCTTCACATTTCAGTTTTAGAGAGAG |
| | ACCTTTC |
| | SEQ ID NO: 49 |
| CAN1-score-4 | CTTTGGTCTCACCAAAACACCTTGAATAATGATAATGATCGTCATAAATGT |
| | GGCCGCATAATAAGCCAATTAATTTAGCTTTAAATGGTAACTCGTCACGA |
| | GAGATGCCACGGTATTTGGCCGCATAATAAGCCAAGCGTTTTAGAGAGAGA |
| | CCTTTC |
| | SEQ ID NO: 50 |
| CAN1-score-5 | CTTTGGTCTCACCAAAACATGACGATCATTATCATTATTCAAGGTTTCACG |
| | GCTTTTGCACCAAAATTTTAGCTTTGCTGCCGCCTATATCTCTATTTTCCT |
| | GTTCTTAGCTGTTTGGGCTTTTGCACCAAAATTCAAGTTTTAGAGAGAGAC |
| | CTTTC |
| | SEQ ID NO: 51 |
| CAN1-score-6 | CTTTGGTCTCACCAAAACATGGTGTTAGCTTTGCTGCCGCCTATATCTCTA |
| | TTTTCCTGTTCTTAGCTCTTATTTCAATGCATATTCAGATGCAGATTTATT |
| | TGGAAGATTGGAGATGTTTTCCTGTTCTTAGCTGTTGTTTTAGAGAGAGACC |
| | TTTC SEQ ID NO: 52 |
| CAN1-score-7 | CTTTGGTCTCACCAAAACGTAAATGGCGAGGATACGTTCTCTATGGAGGA |
| | TGGCATAGGTGATGAAGAAAGTACAGAACGCTGAAGTGAAGAGAGAGC |
| | TTAAGCAAAGACATATTGGTGGCATAGGTGATGAAGATGAGTTTTAGAGAG |
| | AGACCTTT SEQ ID NO: 53 |
| CAN1-score-8 | CTTTGGTCTCACCAAAACTTTTGGTGCAAAAGCCGTGAAACCTTGAATAA |
| | TGATAATGATCGTCATAAGCATAATAAGCCAAGCCGGGCATTAATTTAGC |
| | TTTAAATGGTAACTCGTCGATAATGATCGTCATAAATGGTTTTAGAGAGAGA |
| | CCTTTC |
| | SEQ ID NO: 54 |
| CAN1-score-9 | CTTTGGTCTCACCAAAACTCGTGACGAGTTACCATTTAAAGCTAAATTAAT |
| | GCCCGGCTTGGCTTATTACATTTATGACGATCATTATCATTATTCAAGGTT |
| | TCACGGCTTTTGCACCGCCCGGCTTGGCTTATTATGGTTTTAGAGAGAGACC |
| | TTTC SEQ ID NO: 55 |
| CAN1-score-10 | CTTTGGTCTCACCAAAACACACCTCTGACCAACGCCGGCCCAGTGGGCG |
| | CTCTTATATCATATTTATTCTTTGGCATATTCTGTCACGCAGTCCTTGGGT |
| | GAAATGGCTACATTCATCCTTATATCATATTTATTTATGTTTTAGAGAGAGACC |
| | TTTC |
| | SEQ ID NO: 56 |
| ADE2-score-1 | CTTTGGTCTCACCAAAACGATTTGGGTTTTCCATTCGTCTTGAAGTCGAG |
| | GACTTTGGCATACGATGGACTTCGTTGTAAAGAATAAGGAAATGATTCCG |
| | GAAGCTTTGGAAGTACTGACTTTGGCATACGATGGAAGGTTTTAGAGAGAG |
| | ACCTTTC |
| | SEQ ID NO: 57 |
| ADE2-score-2 | CTTTGGTCTCACCAAAACTTTTGTATGTTTGTCTCCAAGAACATTTAGCAT |
| | AATGGCGTTCGTTGTAAAAAGATGTGAAATTCTTTGGCATTGGCAAATCC |
| | AATATTGATCTCAAATGAATGGCGTTCGTTGTAATGGGTTTTAGAGAGAGAC |
| | CTTTC |
| | SEQ ID NO: 58 |
| ADE2-score-3 | CTTTGGTCTCACCAAAACAATATCAGTTCTACCTGTAATGTAGTTCAGCCT |
| | TTGTTCACATTCCGCCAGCAATAATATTTATGTGACCTACTTTTCTGTTAG |
| | GTCTAGACTCTTTTCCTTGTTCACATTCCGCCATACGTTTTAGAGAGAGACC |
| | TTTC |
| | SEQ ID NO: 59 |
| ADE2-score-4 | CTTTGGTCTCACCAAAACAATTTCACATCTTTCTCCACCATTACAACGAAC |
| | GCCATTATGCTAAATGTACAAACATACAAAAGATAAAGAGCTAGAAACTT |
| | GCGAAAGAGCATTGGCGGCCATTATGCTAAATGTTCTGTTTTAGAGAGAGA |
| | CCTTTC |
| | SEQ ID NO: 60 |
| ADE2-score-5 | CTTTGGTCTCACCAAAACACAATCAGATTGATACAAGACAAATATATTCAA |
| | AAAGAGCATTTAATCAATAGCAGTTACCCAAAGTGTTCCTGTGGAACAA |
| | GCCAGTGAGACGTCCCTAAAAGAGCATTTAATCAAAAAGTTTTAGAGAGA |
| | GACCTTTC SEQ ID NO: 61 |
| ADE2-score-6 | CTTTGGTCTCACCAAAACCCTTTTACGGGCACACCGATGACAGGAAGTGG |
| | TGTCATTGCAGCCACCATAGTGAGCAGCCCCACCAGCTCCAGCGATAAT |
| | TGTTTTAATTCCACGCTTGGTCATTGCAGCCACCATACCGTTTTAGAGAGAG |
| | ACCTTTC |
| | SEQ ID NO: 62 |
| ADE2-score-7 | CTTTGGTCTCACCAAAACACATTTAGCATAATGGCGTTCGTTGTAATGGTG |
| | GAGAAAGATGTGAAATTTTGGCAAATCCAATATTGATCTCAAATGAGCTT |
| | CAAATTGAGAAGTGACGGAGAAAGATGTGAAATTCTTGTTTTAGAGAGAGA |
| | CCTTTC |
| | SEQ ID NO: 63 |
| ADE2-score-8 | CTTTGGTCTCACCAAAACGCCAAGCAGTCTGACAGCCAACAGCGCAGCG |
| | TTCGTACTATTATTAATAGGCTACTGGAACACCTCTAGGCATTTGCACAAT |
| | TGAATGTAAAGAATCTACCGTACTATTATTAATAGCGAGTTTTAGAGAGAGA |
| | CCTTTC |
| | SEQ ID NO: 64 |
| ADE2-score-9 | CTTTGGTCTCACCAAAACAAAATCTCTGTCGCTCAAAAGTTGGACTTGGA |
| | AGCAATGGTCAAACCATTTCATCATGGGATCAGACTCTGACTTGCCGGT |
| | AATGTCTGCCGCATGTGCGGCAATGGTCAAACCATTGGTGTTTTAGAGAGA |
| | GACCTTTC SEQ ID NO: 65 |
| ADE2-score-10 | CTTTGGTCTCACCAAAACAGCGCAGCGTTCGTACTATTATTAATAGCGACG |
| | GTAGCTACTGGAACACCTTTGCACAATTGAATGTAAAGAATCTACTCCAT |
| | CTAGACAAGAACCTTTTGTAGCTACTGGAACACCTCTGTTTTAGAGAGAGA |
| | CCTTTC |
| | SEQ ID NO: 66 |
| LYP1-score-1 | CTTTGGTCTCACCAAAACGTGAGATGGCTACGTTTATCCCCGTGACATCAT |
| | CTATCACTGTCTTTTCGCTTATCACCTGCATTCGGTGTTTCTAACGGCTA |
| | CATGTACTGGTTCAATTCTATCACTGTCTTTTCGAAGGTTTTAGAGAGAGAC |
| | CTTTC |
| | SEQ ID NO: 67 |
| LYP1-score-2 | CTTTGGTCTCACCAAAACCCATCCGAGAAAACGGCCTTCACTTTTATCACT |
| | GGAGATGATGCCTGGCCCCTGGATTTCTCCAGTACCTGAAACCGATAGG |
| | GCCCTGGTGGGATCCACCGGAGATGATGCCTGGCCCCCGTTTTAGAGAGAG |
| | ACCTTTC SEQ ID NO: 68 |
| LYP1-score-3 | CTTTGGTCTCACCAAAACGTCGTCTTATTACTTGGATCTATTGCTTCCATC |
| | TCATGTTCTATCTGGTCATTCCTGCATGCTCTGTTCGCCAATGTTGTTTT |
| | GTTTCTCGTCCCATTTATCATGTTCTATCTGGTCTTCGTTTTAGAGAGAGACC |
| | TTTC |
| | SEQ ID NO: 69 |
| LYP1-score-4 | CTTTGGTCTCACCAAAACAATAGTACGATTCTAAAGACGACTTTATTGATA |
| | GCTCTTGGAACGGTCTTTAGCCGCTTCACCAGCGGTGATCCCAACCAGT |
| | TCAGTACCTTGGTACGTAGCTCTTGGAACGGTCTTTCTGTTTTAGAGAGAG |
| | ACCTTTC |
| | SEQ ID NO: 70 |
| LYP1-score-5 | CTTTGGTCTCACCAAAACACGGTGCTTTAAAGCTTGCATGAACCTAATATG |
| | TGCCAAAGAGATGAATACATAACCCAGCCAAAGTGGAAATGTTGATCAA |
| | CCAGTTAAATGCAGTGTTTGCCAAAGAGATGAATAACCGTTTTAGAGAGAG |
| | ACCTTTC SEQ ID NO: 71 |
| LYP1-score-6 | CTTTGGTCTCACCAAAACGTTAAAGTTTTAGCCATTATGGGTTACTTGATAT |
| | ATGCTTTGATTATTGTGATCCCACCAGGGCCCTATCGGTTTCAGGTACTG |
| | GAGAAATCCAGGAGCCTATGCTTTGATTATTGTCTGGTTTTAGAGAGAGACC |
| | TTTC |
| | SEQ ID NO: 72 |
| LYP1-score-7 | CTTTGGTCTCACCAAAACCATGAAAATGTAAGCAATCAGGGACCCCACAG |
| | GGCCAGCATTACTCAAGGATACCAACGAAAAGACCAGTACCGATTGTAC |
| | CACCTAGTGCAATCATACCGCCAGCATTACTCAAGGGAGGTTTTAGAGAGA |
| | GACCTTTC SEQ ID NO: 73 |
| LYP1-score-8 | CTTTGGTCTCACCAAAACGTGGATCCCACCAGGGCCCTATCGGTTTCAGG |
| | TACTGGAGAAATCCAGGAGCCAGGCATCATCTCCAGTGATAAAAGTGAA |
| | GGCCGTTTTCTCGGATGGGACTGGAGAAATCCAGGAGCCGTTTTAGAGAG |
| | AGACCTTTC SEQ ID NO: 74 |
| LYP1-score-9 | CTTTGGTCTCACCAAAACCATAATATAGAATAGTACGATTCTAAAGACGAC |
| | TTTATTGATAGCTCTTGTTTCTTGGGTTAGCCGCTTCACCAGCGGTGATC |
| | CCAACCAGTTCAGTACCTTTATTGATAGCTCTTGGAAGTTTTAGAGAGAGAC |
| | CTTTC |
| | SEQ ID NO: 75 |
| LYP1-score-10 | CTTTGGTCTCACCAAAACAGCTAGAAGATATTGACATCGATTCCGACAGA |
| | AGAGAAATCGAAGCAATTAGACGACGAGCCTAAGAATTTATGGGAGAAA |
| | TTCTGGGCTGCTGTTGCATGAGAAATCGAAGCAATTATTGTTTTAGAGAGA |
| | GACCTTTC SEQ ID NO: 76 |

Homology arm: Bold; Mutations: italics; Guide sequence: underline; Direct repeat: double underline.

The editing efficiencies of CHAnGE cassettes were measured with varying scores. In the designed library, 98.4% of the cassettes have a score of more than 60 (FIG. 1c). 30 cassettes were tested targeting CAN1, ADE2, and LYP1 (Table 4). Cassettes with a score >60 have median and average editing efficiencies of 88% and 82%, respectively. Cassettes with a score <60 have median and average editing efficiencies of 81% and 61% (FIG. 1d). Considering that there are only 1.6% low score cassettes in the library, these results suggest that CHAnGE cassettes enable efficient editing. Compared with eMAGE (from ˜1.0% at a distance of 20 kb to >40% next to a replication origin), editing efficiency using CHAnGE was superior, independent of target site.

TABLE 4
A summary of library coverage.

| | | Yeast | | | Control | Enriched |
|---|---|---|---|---|---|---|
| | E. coli CFU/fold | CFU/fold | | Cassettes | cassettes | control |
| Experiment | coverage | coverage | Reads/cassette* | observed | observed | cassettes** |
| Canavanine | 1.2 - | 9.8 × 10⁶/395 | 97.5 | 13992 | 89 | 0 |
| | 4 × 10⁷/480-1600 | | | (56.3%) | | |
| HAc | 1.2 - | 9.8 × 10⁶/395 | 49.3 | 14678 | 84 | 0 |
| 1^{st} round | 4 × 10⁷/480-1600 | | | (59.0%) | | |
| HAc | 1.2 - | 3.2 × 10⁶/129 | 72.8 | 9266 | 58 | 0 |
| 2^{nd} round | 4 × 10⁷/480-1600 | | | (37.3%) | | |
| Furfural | 1.2 - | 9.8 × 10⁶/395 | 95.1 | 18082 | 92 | 2 |
| 1^{st} round | 4 × 10⁷/480-1600 | | | (72.7%) | | |
| Furfural | 1.2 - | 1.2 × 10⁷/499 | 67.3 | 16509 | 91 | 0 |
| 2^{nd} round | 4 × 10⁷/480-1600 | | | (66.4%) | | |
| SIZ1 tiling | 3.8 - | 1.9 × 10⁶/3200 | 744.3 | 580 | 29 | 3 |
| mutagenesis | 8 × 10⁵/655-1379 | | | (100%) | | |

*total mapped read counts divided by library size
**P value <0.05, fold change >1.5

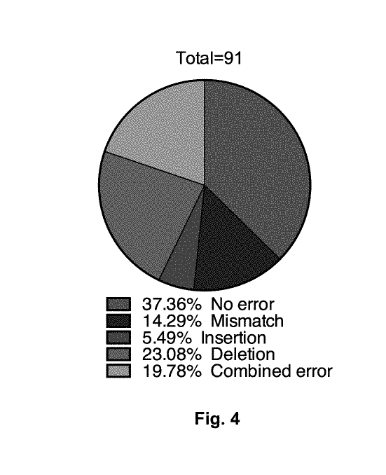

To generate a pooled plasmid library, designed oligonucleotides were synthesized on chip and then assembled into pCRCT Bao, Z. et al. ACS Synth. Biol. 4, 585-594 (2015). (FIG. 1b). Sequencing of 91 assembled plasmids revealed that 37.36% were correct (FIG. 4), reflecting a 0.58% synthesis error rate per base. NGS of the plasmid library captured 95.5% of the designed guide sequences, which cover 99.5% of the targeted ORFs. The plasmid library was heat-shock transformed into S. cerevisiae, to yield pooled single mutants, each containing an 8 nucleotide deletion in a single gene. A 395-fold coverage was achieved (Table 5), ensuring the completeness of a collection of genome-wide gene deletions. The number of transformations can be scaled up to obtain efficiencies required for even larger library sizes. The mutant library was screened for CAN1 mutants in the presence of L-(+)-(S)-canavanine and identified all four CAN1-targeting guides, with depletion of non-edited controls since wild-type yeast cells are killed by canavanine (FIG. 1e). Some cassettes were not observed due to the low NGS read depth (Table 5). Reducing the synthesis error rate or assigning more reads to each sample could alleviate this problem.

TABLE 5

| Primers | Sequences (5′ to 3′) |
|---|---|
| Bsal-LIB-for | TATCTACACGGGTCTCACC SEQ ID NO: 77 |
| Bsal-LIB-rev | GAGTTACGCTGGTCTCTCT SEQ ID NO: 78 |
| HiSeq-CHAnGE- | GTCTCGTGGGCTCGGAGTGAAAGATAAATGATC |
| for | GG SEQ ID NO: 79 |
| HiSeq-CHAnGE- | TCGTCGGCAGCGTCATTTTGAAGCTATGCAGAC |
| rev | SEQ ID NO: 80 |
| EMX1-selective- | AAGAAGCGATTATGATCTCTCCTCTAGAAACTC |
| for | SEQ ID NO: 81 |
| EMX1-selective- | GCCACCGGTTGATCACTACAC SEQ ID NO: |
| rev | 82 |

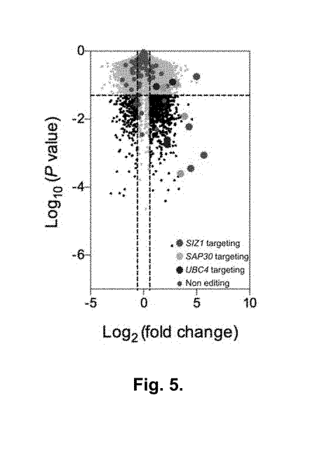

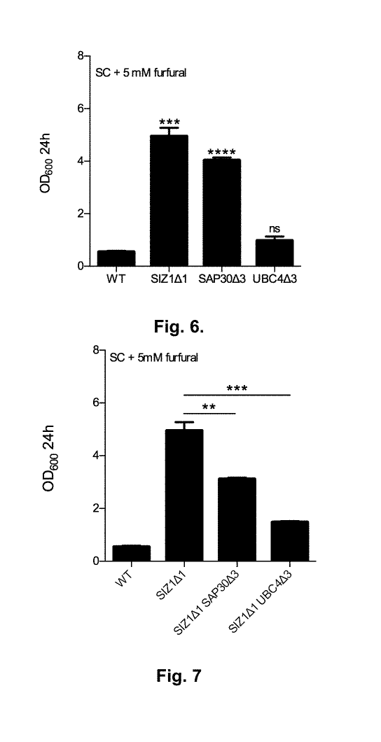

CHAnGE was then used to engineer furfural tolerance. Selection with 5 mM furfural enriched SIZ1 targeting guides (FIG. 1f and FIG. 5). Guide sequences targeting newly identified genes SAP30 and UBC4, were also enriched. All three disruption mutants grew faster in the presence of furfural compared with the wild-type parent (FIG. 6).

SIZ1 DAA12251.1
SEQ ID NO: 736

| | |
|---|---|
| 1 | minledywed etpgpdrept nelrneveet itlmellkvs |
| | elkdicrsvs fpvsgrkavl |
| 61 | qdlirnflqn alvvgksdpy rvqavkflie rirkneplpv |
| | ykdlwnalrk gtplsaitvr |
| 121 | smegpptvqq qspsvirqsp tqrrktstts stsrappptn |
| | pdassssssf avptihfkes |
| 181 | pfykiqrlip elvmnvevtg grgmcsakfk lskadynlls |
| | npnskhrlyl fsgminplgs |
| 241 | rgnepiqfpf pnelrcnnvq ikdnirgfks kpgtakpadl |
| | tphlkpytqq nnveliyaft |
| 301 | tkeyklfgyi vemitpeqll ekvlqhpkii kqatllylkk |
| | tlredeemgl tttstimslq |
| 361 | cpisytrmky psksinckhl qcfdalwflh sqlqiptwqc |
| | pvcqidiale nlaisefvdd |
| 421 | ilqncqknve qveltsdgkw tailedddds dsdsndgsrs |
| | pekgtsvsdh hcssshpsep |
| 481 | iiinldsddd epngnnphvt nnhddsnrhs ndnnnnsikn |
| | ndshnknnnn nnnnnnnnnd |
| 541 | nnnsiennds nsnnkhdhgs rsntpshnht knlmndnddd |
| | dddrlmaeit snhlkstntd |
| 601 | iltekgssap srtldpksyn ivasetttpv tnrvipeylg |
| | nsssyigkql pnilgktpln |
| 661 | vtavdnsshl ispdvsvssp tprntasnas ssalstppli |
| | rmssldprgs tvpdktirpp |
| 721 | insnsytasi sdsfvqpqes svfppreqnm dmsfpstvns |
| | rfndprlntt rfpdstlrga |
| 781 | tilsnngldq rnnslpttea itrndvgrqn stpvlptlpq |
| | nvpirtnsnk sglplinnen |
| 841 | svpnppntat iplqksrliv npfiprrpys nvlpqkrqls |
| | ntsstspimg twktqdygkk |
| 901 | ynsg |

SAP30 DAA410163.1
SEQ ID NO: 732

| | |
|---|---|
| 1 | marpvntnae tesrgrptqg ggyasnnngs cnnnngsnnn |
| | nnnnnnnnnn snnsnnnngp |
| 61 | tssgrtngkq rltaaqqqyi knliethitd nhpdlrpksh |
| | pmdfeeytda flrrykdhfq |
| 121 | ldvpdnltlq gyllgsklga ktysykrntq gqhdkrihkr |
| | dlanvvrrhf dehsiketdc |
| 181 | ipqfiykvkn qkkkfkmefr g |

UBC? 24 DAA07201.1
SEQ ID NO: 733

| | |
|---|---|
| 1 | mssskriake lsdlerdppt scsagpvgdd lyhwqasimg |
| | padspyaggv fflsihfptd |
| 61 | ypfkppkisf ttkiyhpnin angnicldil kdqwspaltl |
| | skvllsicsl ltdanpddpl |
| 121 | vpeiahiykt drpkyeatar ewtkkyav |

LCB3 DAA08666.1
SEQ ID NO: 737

| | |
|---|---|
| 1 | mvdglntsni rkrartlsnp ndfqepnyll dpgnhpsdhf |
| | rtrmskfrfn irekllvftn |
| 61 | nqsftlsrwq kkyrsafndl yftytslmgs htfyvlclpm |
| | pvwfgyfett kdmvyilgys |
| 121 | iylsgffkdy wclprprapp lhritlseyt tkeygapssh |
| | tanatgvsll flyniwrmqe |
| 181 | ssvmvqllls cvvlfyymtl vfgriycgmh gildlvsggl |
| | igivcfivrm yfkyrfpglr |
| 241 | ieehwwfplf svgwgllllf khvkpvdecp cfqdsvafmg |
| | vvsgieccdw lgkvfgvtlv |
| 301 | ynlepncgwr ltlarllvgl pcvviwkyvi skpmiytlli |
| | kvfhlkddrn vaarkrleat |
| 361 | hkegaskyec plyigepkid ilgrfiiyag vpftvvmcsp |
| | vlfsllnia |

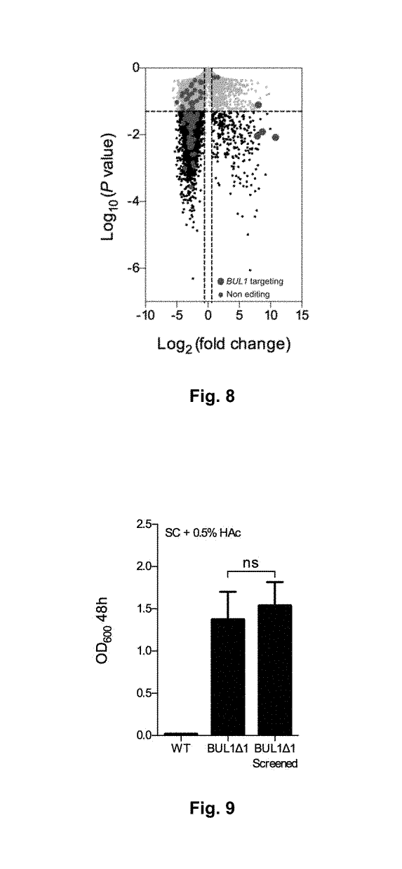

However, combining the individual gene disruptions into a single strain did not improve tolerance further (FIG. 7), suggesting that these beneficial mutations are neither additive nor synergistic. SIZ1Δ1 (edited by CHAnGE cassette SIZ1_1) was selected as the parental strain and iterated the CHAnGE workflow a second time. LCB3 targeting guides were enriched in 10 mM furfural during the second round of evolution (FIG. 1f). Increased tolerance was confirmed by measuring growth of wild-type, single, and double mutants in 10 mM furfural stress (FIG. 1g). Interestingly, the phenotype of the LCB3 mutant was dependent on SIZ1 disruption; LCB3 targeting guides were not enriched in the first round of evolution, and the single LCB3 disruption mutant LCB3Δ1 showed similar growth as wild-type (FIG. 1f,g), showing epistasis. CHAnGE was also applied for directed evolution of acetic acid tolerance and achieved 20-fold improvement (FIG. 8-10).

### Example 2. Directed Evolution of Acetic Acid (HAc) Tolerance

The single mutant library was screened in the presence of 0.5% (v/v) HAc and observed many enriched guide sequences as compared to non-editing controls (FIG. 8). Among these guides, BUL1 targeting guides were the most enriched. From the HAc stressed library, a BUL1 disruption mutant was recovered with an 8 bp deletion introduced by CHAnGE cassette BUL1_1 (Table 3). This mutant was named BUL1Δ1. To confirm that the mutant is indeed resistant to HAc and this resistance is not due to adaptive mutagenesis, the BUL1Δ1 mutant was independently constructed using the HI-CRISPR method and biomass accumulation of both mutants and the wild type strain was measured in the presence of HAc. Indeed, both the recovered and reconstructed BUL1Δ1 mutants exhibited faster biomass accumulation than the wild type strain (FIG. 9). No significant difference was observed between the two BUL1Δ1 mutants, indicating that the obtained HAc tolerance was a result of the designed genotype.

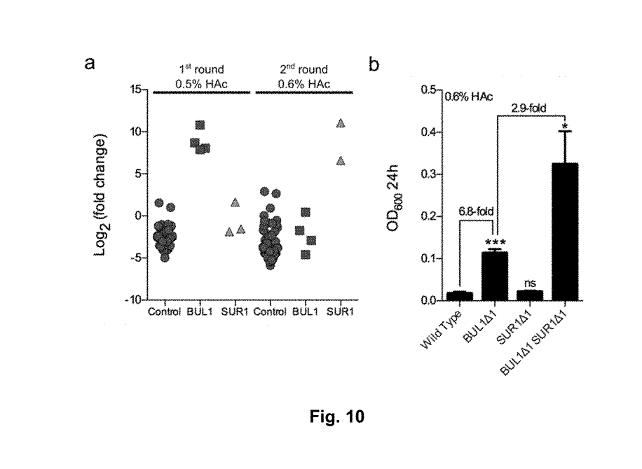

BUL1Δ1 was selected as the parental strain for the second round evolution of HAc tolerance. When screened under 0.6% (v/v) HAc, SUR1 targeting guide sequences were identified as significantly enriched as compared to non-editing controls (FIG. 10a). The BUL1 targeting guide sequences were not enriched in the second round of evolution (FIG. 10a), which is expected since the BUL1 gene was already disrupted in the parental strain BUL1Δ1. Notably, SUR1 targeting guide sequences were not enriched during the first round of evolution (FIG. 10a), suggesting that BUL1 disruption is a prerequisite for improved HAc tolerance conferred by SUR1 disruption. Mutants SUR1Δ1 and BUL1Δ1 SUR1Δ1 were constructed, and biomass accumulation was compared with the wild type and parental BUL1Δ1 strains under 0.6% HAc. As expected, the double mutant BUL1Δ1 SUR1Δ1 showed faster biomass accumulation than the parental strain BUL1Δ1, while the single mutant SUR1Δ1 showed little HAc tolerance (FIG. 10b).

BUL1 DAA10176.1
SEQ ID NO: 734

| | |
|---|---|
| 1 | makdlndsgf ppkrkpllrp qrsdftanss ttmnvnantr |
| | grgrqkqegg kgssrspslh |
| 61 | spkswirsas atgilglrrp elahshshap stgtpaggnr |
| | splrrstana tpvetgrslt |
| 121 | dgdinnvvdv lpsfemyntl hrhipqgnvd pdrhdfppsy |
| | qeannstatg aagssadlsh |
| 181 | qslstdalga trssstsnle nliplrtehh siaahqstav |
| | dedsldippi lddlndtdni |
| 241 | fidklytlpk mstpieitik ttkhapiphv kpeeesilke |
| | ytsgdlihgf itienksqan |
| 301 | lkfemfyvtl esyisiidkv kskrtikrfl rmvdlsasws |
| | yskialgsgv dfipadvdyd |
| 361 | gsvfglnnsr vlepgvkykk ffifklplql ldvtckqehf |
| | shcllppsfg idkyrnncky |
| 421 | sgikvnrvlg cghlgtkgsp iltndmsddn lsinytidar |
| | ivgkdqkask lyimkereyn |
| 481 | lrvipfgfda nvvgerttms qlnditklvg erldalrkif |
| | qrlekkepit nrdihgadls |
| 541 | gtiddsiesd sqeilqrkld qlhiknrnny lvnyndlklg |
| | hdldngrsgn sghntdtsra |
| 601 | wgpfveselk yklknksnss sflnfshfln sssssmssss |
| | nagknnhdlt gnkertglil |
| 661 | vkakipkqgl pywapsllrk tnvfeskskh dqenwvrlse |
| | lipedvkkpl ekldlqltci |
| 721 | esdnslphdp peiqsittel icitaksdns ipiklnsell |
| | mnkekltsik alyddfhski |
| 781 | ceyetkfnkn flelnelynm nrgdrrpkel kftdfitsql |
| | fndiesicnl kvsvhnlsni |
| 841 | fkkqvstlkq hskhalseds ishtgngsss spssasltpv |
| | tsssksslfl psgssstslk |
| 901 | ftdqivhkwv riaplqykrd invnlefnkd iketlipsfe |
| | scilcrfycv rvmikfenhl |
| 961 | gvakidipis vrqvtk |

SUR1 DAA11373.1
SEQ ID NO: 735

| | |
|---|---|
| 1 | mrkelkylic fnillllsii yytfdlltlc iddtvkdail |
| | eedlnpdapp kpqlipkiih |
| 61 | qtyktedipe hwkegrqkcl dlhpdykyil wtdemayefi |
| | keeypwfldt fenykypier |
| 121 | adairyfils hyggvyidld dgcerkldpl lafpaflrkt |
| | splgvsndvm gsvprhpffl |
| 181 | kalkslkhyd kywfipymti mgstgplfls viwkqykrwr |
| | ipkngtvril qpayykmhsy |
| 241 | sffsitkgss whlddaklmk alenhilscv vtgfifgffi |
| | lygeftfycw lcsknfsnlt |
| 301 | knwklnaikv rfvtilnslg lrlklsksts dtasatllar |
| | qqkrlrkdsn tnivllkssr |
| 361 | ksdvydlekn dsskyslgnn ss |

### Example 3. Precision Editing of SIZ1

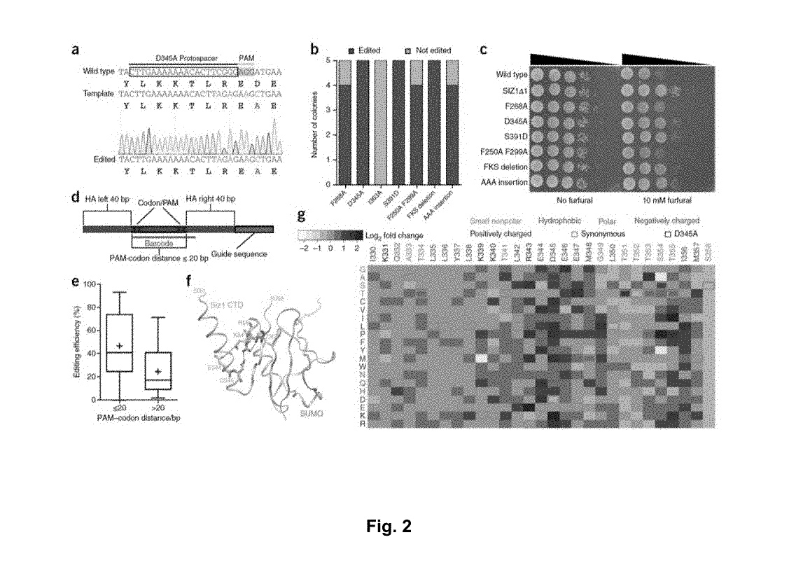

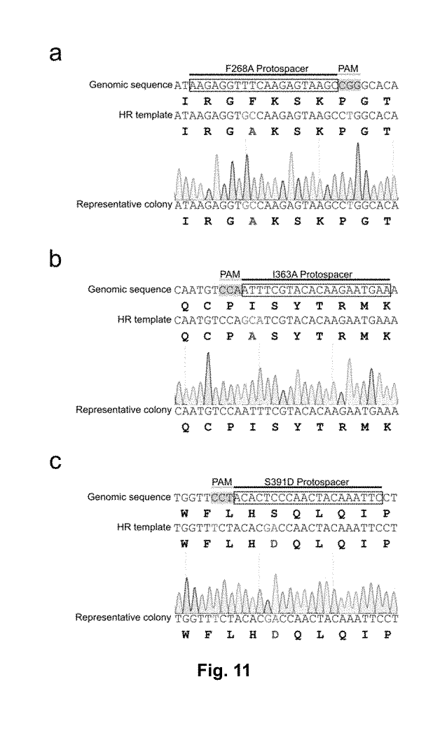

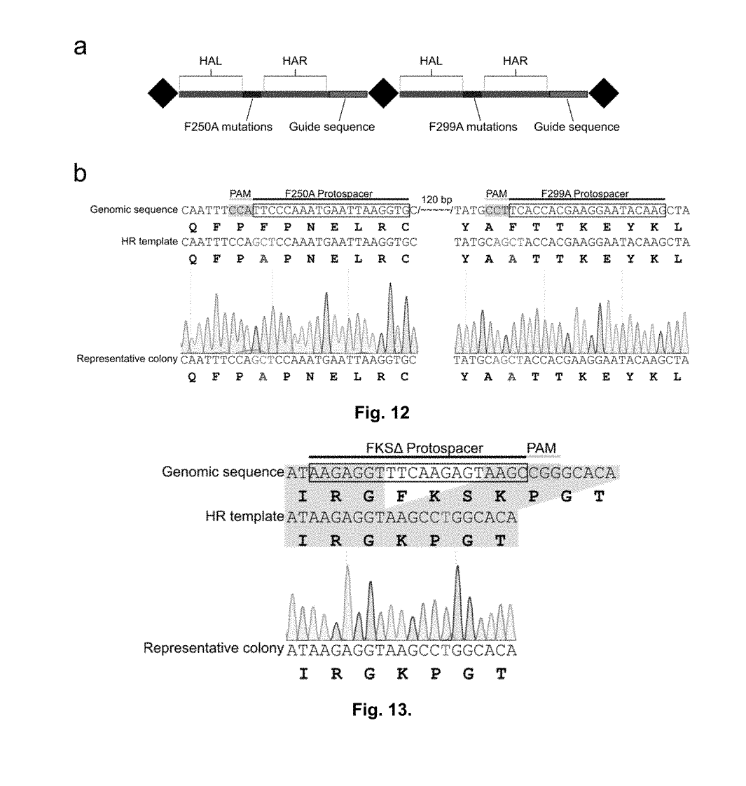

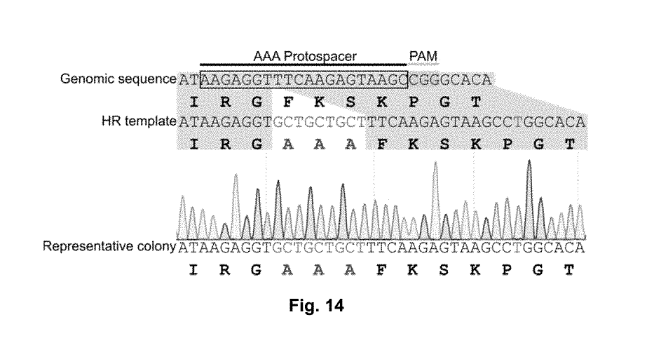

Next, CHAnGE was applied for single-nucleotide resolution editing. Exogenous Siz1 mutations (F268A, D345A, I363A, S391D, F250A/F299A, FKSΔ) are known to diminish SUMO conjugation to PCNA. Seven CHAnGE cassettes were designed to introduce these seven mutations and an insertion mutation (FIG. 2a and FIG. 11-14). In each cassette, codon substitutions were placed between the homology arms. To compare with CREATE, CHAnGE cassette F250A F299A was designed to simultaneously introduce two distal codon substitutions (147 bp apart, FIG. 12). Except for I363A, we observed all other designed Siz1 mutations with efficiencies from 80% to 100% (FIG. 2b). These results highlight the capability of CHAnGE to introduce mutations that are unlikely to occur spontaneously, such as those requiring two or three bases within a codon to be altered (e.g., F268A and S391D). F268A, D345A, S391D, FKSΔ, and AAA all showed improved furfural tolerance (FIG. 2c), suggesting that reducing PCNA sumoylation has a role in acquired furfural tolerance. An increased growth rate was not observed for F250A F299A, which may represent a difference between endogenously and episomally expressed mutants. 8 CHAnGE cassettes were designed targeting CAN1 and UBC4, and achieved an average editing efficiency of 90% for 7/8 cassettes which provides evidence that the method is generalizable to different loci.

### Example 4. Precision Editing of CAN1 and UBC4

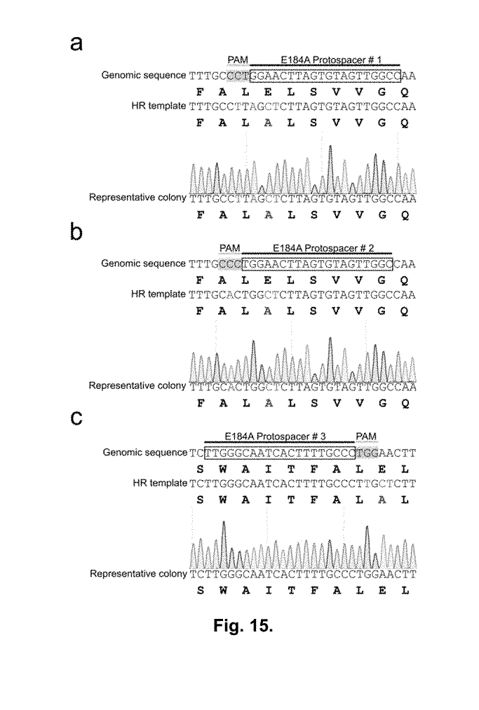

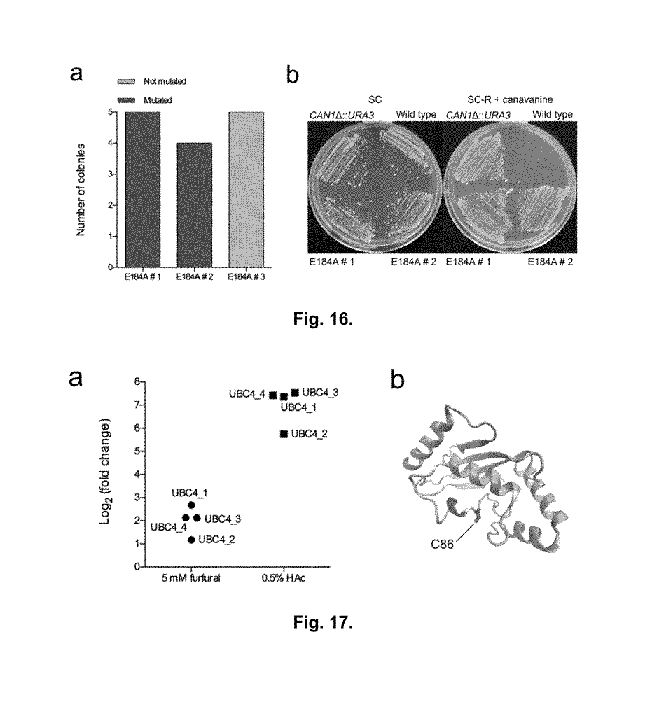

Three CHAnGE cassettes (FIG. 15 and Table 4) were designed for mutating the E184 residue of Can1 to an alanine residue. E184 is a critical residue for transporting arginine into S. cerevisiae. It was hypothesized that it is also critical for transporting the arginine analog canavanine. As a result, mutating E184 should abolish the ability of Can1 to transport canavanine, thus rescuing the cell in the presence of canavanine. Two of the three designed CHAnGE cassettes (E184A#1 and 2, FIG. 15a,b) successfully mutated E184 to alanine, with a 100% efficiency for both designs (FIG. 16a). However, E184A#3 (FIG. 15c) did not mutate any of the five colonies examined (FIG. 16a). The E184A mutants were able to grow in the presence of canavanine (FIG. 16b), which validated the hypothesis.

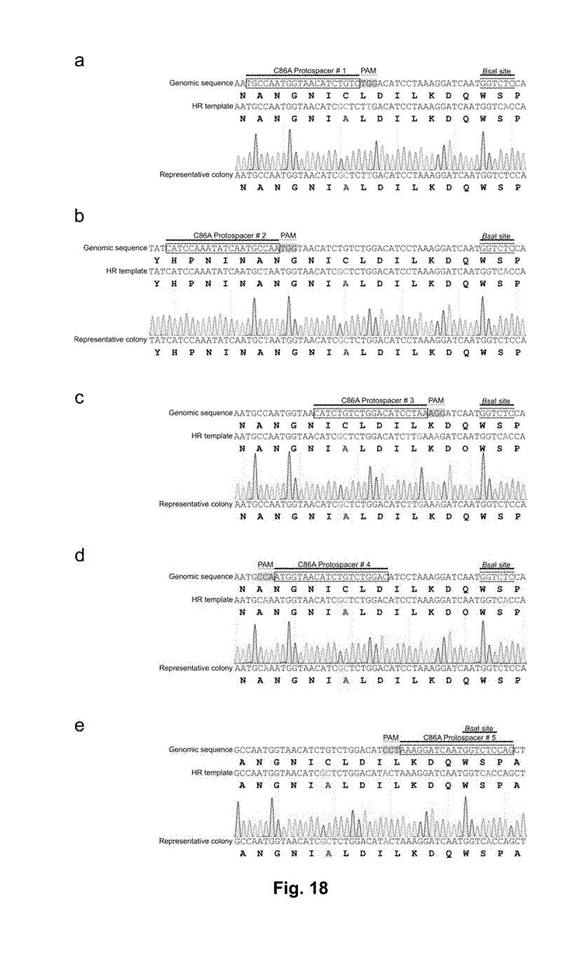

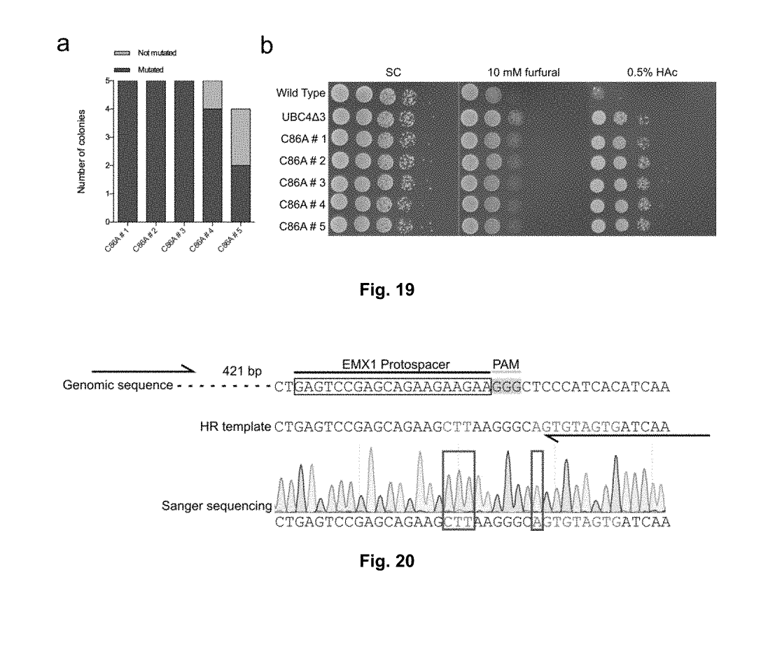

Protein Ubc4 was targeted next. UBC4 targeting guide sequences were enriched in both HAc and furfural screening experiments (FIG. 17a). Ubc4 is a class 1 ubiquitin conjugating enzyme. Amino acid C86 acts as the ubiquitin accepting residue in the enzymatic catalysis of ubiquitin conjugation (FIG. 17b). Five different CHAnGE cassettes were designed to mutate C86 to an alanine residue (FIG. 18 and Table 4). Since there is a BsaI restriction site 23 bp downstream of the C86 codon, a silent mutation was also designed to remove the BsaI site to enable Golden Gate assembly (FIG. 18). All five cassettes mutated C86 to alanine with efficiencies ranging from 50% to 100% (FIG. 19a). Interestingly, mutation of the BsaI site was only observed once with CHAnGE cassette C86A#5 (FIG. 18e). Spotting assay showed that the C86A mutants were both HAc and furfural tolerant (FIG. 19b), suggesting that the abolishment of Ubc4 mediated ubiquitin conjugation of substrate proteins plays a role in both HAc and furfural tolerance.

### Example 5. Single-Nucleotide Resolution Editing

Tiling mutagenesis of the Siz1 SP-CTD domain was carried out. The CHAnGE cassette was modified to reduce the length of homology arms to 40 bp, so that the sequence between the target codon and the PAM could be accommodated (FIG. 2d). Five CHAnGE cassettes were designed with 40 bp homology arms targeting UBC4, and achieved an average editing efficiency of 86% (FIG. 19a). To minimize the length of CHAnGE cassettes, the PAM-codon distance was restricted to 20 bp or less. Given that the density of NGG PAMs is one per 8 bp, there is a 93% chance of a PAM for any given codon. A genetic barcode was also used within the donor to enable NGS tracking because 20 bp guides may not be unique (FIG. 2d). To evaluate editing efficiencies of CHAnGE cassettes with varying PAM-codon distances, 30 CHAnGE cassettes were designed to disrupt CAN1, ADE2, and LYP1 (Table 4). Cassettes with a PAM-codon up to 20 bp have 41% (median) and 47% (average) editing efficiencies respectively. Cassettes with a PAM-codon of more than 20 bp have less than 25% editing efficiencies (FIG. 2e). 580 CHAnGE cassettes were designed (Table 6; SEQ ID NOs:152-731) for saturation mutagenesis of the 29 amino acid residues of the SP-CTD domain, which consists of an α-helix and a β-strand. Amino acid residues from the C-terminal of the α-helix and the entire β-strand interact extensively with SUMO (FIG. 2f). For example, E344 and D345 from the α-helix form hydrogen bonds with SUMO K54 and R55, respectively. T355 from the β-strand form a hydrogen bond with SUMO R55. When the yeast Siz1 mutant library was subject to furfural selection, enrichment of the validated D345A was observed, but no enrichment of most of the synonymous cassettes (FIG. 2g and Table 5) was observed. Using this method two enrichment hot spots were identified centered around D345 and T355, consistent with molecular interactions between SP-CTD and SUMO.

SUPPLEMENTARY TABLE 6
A summary of 580 SIZ1 CHAnGE cassette sequences.

| CHAnGE | | |
|---|---|---|
| cassette | | SEQ ID |
| name | Oligonucleotide sequence | NO: |
| I330A | TATCTACACGGGTCTCACCAAAACGGAGCAACTCCTGGAAAAAGTATTACAGCATCCAAAA | 152 |
| | ATTGCTAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCGACGTGT | |
| I330R | TATCTACACGGGTCTCACCAAAACGGAGCAACTCCTGGAAAAAGTATTACAGCATCCAAAA | 153 |
| | ATTAGAAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCTTGGTTA | |
| I330N | TATCTACACGGGTCTCACCAAAACGGAGCAACTCCTGGAAAAAGTATTACAGCATCCAAAA | 154 |
| | ATTAATAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCGGGTGTA | |
| I330D | TATCTACACGGGTCTCACCAAAACGGAGCAACTCCTGGAAAAAGTATTACAGCATCCAAAA | 155 |
| | ATTGATAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCCACAATG | |
| I330C | TATCTACACGGGTCTCACCAAAACGGAGCAACTCCTGGAAAAAGTATTACAGCATCCAAAA | 156 |
| | ATTTGTAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCCGTCGCT | |
| I330Q | TATCTACACGGGTCTCACCAAAACGGAGCAACTCCTGGAAAAAGTATTACAGCATCCAAAA | 157 |
| | ATTCAAAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCCTCGGGG | |
| I330E | TATCTACACGGGTCTCACCAAAACGGAGCAACTCCTGGAAAAAGTATTACAGCATCCAAAA | 158 |
| | ATTGAAAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCTGGCTGC | |
| I330G | TATCTACACGGGTCTCACCAAAACGGAGCAACTCCTGGAAAAAGTATTACAGCATCCAAAA | 159 |
| | ATTGGTAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCACTCCTG | |
| I330H | TATCTACACGGGTCTCACCAAAACGGAGCAACTCCTGGAAAAAGTATTACAGCATCCAAAA | 160 |
| | ATTCATAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCAGAGGAC | |
| I330I | TATCTACACGGGTCTCACCAAAACGGAGCAACTCCTGGAAAAAGTATTACAGCATCCAAAA | 161 |
| | ATTATTAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCACATTGG | |
| I330L | TATCTACACGGGTCTCACCAAAACGGAGCAACTCCTGGAAAAAGTATTACAGCATCCAAAA | 162 |
| | ATTTTGAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCCATCCTA | |
| I330K | TATCTACACGGGTCTCACCAAAACGGAGCAACTCCTGGAAAAAGTATTACAGCATCCAAAA | 163 |
| | ATTAAAAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCGTTAAAT | |
| I330M | TATCTACACGGGTCTCACCAAAACGGAGCAACTCCTGGAAAAAGTATTACAGCATCCAAAA | 164 |
| | ATTATGAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCCTTATAA | |
| I330F | TATCTACACGGGTCTCACCAAAACGGAGCAACTCCTGGAAAAAGTATTACAGCATCCAAAA | 165 |
| | ATTTTCAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCAGTGACA | |
| I330P | TATCTACACGGGTCTCACCAAAACGGAGCAACTCCTGGAAAAAGTATTACAGCATCCAAAA | 166 |
| | ATTCCAAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCTAGTCCC | |
| I330S | TATCTACACGGGTCTCACCAAAACGGAGCAACTCCTGGAAAAAGTATTACAGCATCCAAAA | 167 |
| | ATTTCTAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCGTTTCTA | |
| I330T | TATCTACACGGGTCTCACCAAAACGGAGCAACTCCTGGAAAAAGTATTACAGCATCCAAAA | 168 |
| | ATTACTAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCGGATCCG | |
| I330W | TATCTACACGGGTCTCACCAAAACGGAGCAACTCCTGGAAAAAGTATTACAGCATCCAAAA | 169 |
| | ATTTGGAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCTCCGCCT | |
| I330Y | TATCTACACGGGTCTCACCAAAACGGAGCAACTCCTGGAAAAAGTATTACAGCATCCAAAA | 170 |
| | ATTTATAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCGATTCTG | |
| I330V | TATCTACACGGGTCTCACCAAAACGGAGCAACTCCTGGAAAAAGTATTACAGCATCCAAAA | 171 |
| | ATTGTTAAACAAGCGACCTTACTTTACTTGAAAAAAACACTTCGGGAGGATGAAGAAATTT | |
| | TCAAGTAAAGTAACGGTTTTAGAGTGAGACCAGCGTAACTCCACGCCA | |
| K331A | TATCTACACGGGTCTCACCAAAACGCAACTCCTGGAAAAAGTATTACAGCATCCAAAAATT | 172 |
| | ATTGCTCAAGCGACCTTACTTTACTTGAAAAAAACACTTCGGGAGGATGAAGAAATTTTCA | |
| | AGTAAAGTAACGGTTTTAGAGTGAGACCAGCGTAACTCCCATTATCAA | |
| K331R | TATCTACACGGGTCTCACCAAAACGCAACTCCTGGAAAAAGTATTACAGCATCCAAAAATT | 173 |
| | ATTAGACAAGCGACCTTACTTTACTTGAAAAAAACACTTCGGGAGGATGAAGAAATTTTCA | |
| | AGTAAAGTAACGGTTTTAGAGTGAGACCAGCGTAACTCATTCGCAAAG | |
| K331N | TATCTACACGGGTCTCACCAAAACGCAACTCCTGGAAAAAGTATTACAGCATCCAAAAATT | 174 |
| | ATTAATCAAGCGACCTTACTTTACTTGAAAAAAACACTTCGGGAGGATGAAGAAATTTTCA | |
| | AGTAAAGTAACGGTTTTAGAGTGAGACCAGCGTAACTCCTACCGACAG | |
| K331D | TATCTACACGGGTCTCACCAAAACGCAACTCCTGGAAAAAGTATTACAGCATCCAAAAATT | 175 |
| | ATTGATCAAGCGACCTTACTTTACTTGAAAAAAACACTTCGGGAGGATGAAGAAATTTTCA | |
| | AGTAAAGTAACGGTTTTAGAGTGAGACCAGCGTAACTCTCCATGCATG | |
| K331C | TATCTACACGGGTCTCACCAAAACGCAACTCCTGGAAAAAGTATTACAGCATCCAAAAATT | 176 |
| | ATTTGTCAAGCGACCTTACTTTACTTGAAAAAAACACTTCGGGAGGATGAAGAAATTTTCA | |
| | AGTAAAGTAACGGTTTTAGAGTGAGACCAGCGTAACTCCCCTTCATGA | |
| K331Q | TATCTACACGGGTCTCACCAAAACGCAACTCCTGGAAAAAGTATTACAGCATCCAAAAATT | 177 |
| | ATTCAACAAGCGACCTTACTTTACTTGAAAAAAACACTTCGGGAGGATGAAGAAATTTTCA | |
| | AGTAAAGTAACGGTTTTAGAGTGAGACCAGCGTAACTCGATTACGTCC | |
| K331E | TATCTACACGGGTCTCACCAAAACGCAACTCCTGGAAAAAGTATTACAGCATCCAAAAATT | 178 |
| | ATTGAACAAGCGACCTTACTTTACTTGAAAAAAACACTTCGGGAGGATGAAGAAATTTTCA | |
| | AGTAAAGTAACGGTTTTAGAGTGAGACCAGCGTAACTCCTATGCTTTT | |
| K331G | TATCTACACGGGTCTCACCAAAACGCAACTCCTGGAAAAAGTATTACAGCATCCAAAAATT | 179 |
| | ATTGGTCAAGCGACCTTACTTTACTTGAAAAAAACACTTCGGGAGGATGAAGAAATTTTCA | |
| | AGTAAAGTAACGGTTTTAGAGTGAGACCAGCGTAACTCTTTCTAATTT | |
| K331H | TATCTACACGGGTCTCACCAAAACGCAACTCCTGGAAAAAGTATTACAGCATCCAAAAATT | 180 |
| | ATTCATCAAGCGACCTTACTTTACTTGAAAAAAACACTTCGGGAGGATGAAGAAATTTTCA | |
| | AGTAAAGTAACGGTTTTAGAGTGAGACCAGCGTAACTCAAGCGCGACG | |
| K331I | TATCTACACGGGTCTCACCAAAACGCAACTCCTGGAAAAAGTATTACAGCATCCAAAAATT | 181 |
| | ATTATTCAAGCGACCTTACTTTACTTGAAAAAAACACTTCGGGAGGATGAAGAAATTTTCA | |
| | AGTAAAGTAACGGTTTTAGAGTGAGACCAGCGTAACTCGACAATTTCG | |
| K331L | TATCTACACGGGTCTCACCAAAACGCAACTCCTGGAAAAAGTATTACAGCATCCAAAAATT | 182 |
| | ATTTTGCAAGCGACCTTACTTTACTTGAAAAAAACACTTCGGGAGGATGAAGAAATTTTCA | |
| | AGTAAAGTAACGGTTTTAGAGTGAGACCAGCGTAACTCTCGGAATTCC | |
| K331K | TATCTACACGGGTCTCACCAAAACGCAACTCCTGGAAAAAGTATTACAGCATCCAAAAATT | 183 |
| | ATTAAACAAGCGACCTTACTTTACTTGAAAAAAACACTTCGGGAGGATGAAGAAATTTTCA | |
| | AGTAAAGTAACGGTTTTAGAGTGAGACCAGCGTAACTCGCCACATACA | |
| K331M | TATCTACACGGGTCTCACCAAAACGCAACTCCTGGAAAAAGTATTACAGCATCCAAAAATT | 184 |
| | ATTATGCAAGCGACCTTACTTTACTTGAAAAAAACACTTCGGGAGGATGAAGAAATTTTCA | |
| | AGTAAAGTAACGGTTTTAGAGTGAGACCAGCGTAACTCATTGCGTCTC | |
| K331F | TATCTACACGGGTCTCACCAAAACGCAACTCCTGGAAAAAGTATTACAGCATCCAAAAATT | 185 |
| | ATTTTCCAAGCGACCTTACTTTACTTGAAAAAAACACTTCGGGAGGATGAAGAAATTTTCA | |
| | AGTAAAGTAACGGTTTTAGAGTGAGACCAGCGTAACTCCGCTTCTTGT | |
| K331P | TATCTACACGGGTCTCACCAAAACGCAACTCCTGGAAAAAGTATTACAGCATCCAAAAATT | 186 |
| | ATTCCACAAGCGACCTTACTTTACTTGAAAAAAACACTTCGGGAGGATGAAGAAATTTTCA | |
| | AGTAAAGTAACGGTTTTAGAGTGAGACCAGCGTAACTCAATCGATCGA | |
| K331S | TATCTACACGGGTCTCACCAAAACGCAACTCCTGGAAAAAGTATTACAGCATCCAAAAATT | 187 |
| | ATTTCTCAAGCGACCTTACTTTACTTGAAAAAAACACTTCGGGAGGATGAAGAAATTTTCA | |
| | AGTAAAGTAACGGTTTTAGAGTGAGACCAGCGTAACTCTGGTCTAAAT | |
| K331T | TATCTACACGGGTCTCACCAAAACGCAACTCCTGGAAAAAGTATTACAGCATCCAAAAATT | 188 |
| | ATTACTCAAGCGACCTTACTTTACTTGAAAAAAACACTTCGGGAGGATGAAGAAATTTTCA | |
| | AGTAAAGTAACGGTTTTAGAGTGAGACCAGCGTAACTCATCTCATTAG | |
| K331W | TATCTACACGGGTCTCACCAAAACGCAACTCCTGGAAAAAGTATTACAGCATCCAAAAATT | 189 |
| | ATTTGGCAAGCGACCTTACTTTACTTGAAAAAAACACTTCGGGAGGATGAAGAAATTTTCA | |
| | AGTAAAGTAACGGTTTTAGAGTGAGACCAGCGTAACTCACAGAACCAA | |
| K331Y | TATCTACACGGGTCTCACCAAAACGCAACTCCTGGAAAAAGTATTACAGCATCCAAAAATT | 190 |
| | ATTTATCAAGCGACCTTACTTTACTTGAAAAAAACACTTCGGGAGGATGAAGAAATTTTCA | |
| | AGTAAAGTAACGGTTTTAGAGTGAGACCAGCGTAACTCGCAGGAGCAA | |
| K331V | TATCTACACGGGTCTCACCAAAACGCAACTCCTGGAAAAAGTATTACAGCATCCAAAAATT | 191 |
| | ATTGTTCAAGCGACCTTACTTTACTTGAAAAAAACACTTCGGGAGGATGAAGAAATTTTCA | |
| | AGTAAAGTAACGGTTTTAGAGTGAGACCAGCGTAACTCCCACTTTTGG | |
| Q332A | TATCTACACGGGTCTCACCAAAACACTCCTGGAAAAAGTATTACAGCATCCAAAAATTATT | 192 |
| | AAAGCTGCGACCTTACTTTACTTGAAAAAAACACTTCGGGAGGATGAAGAAATTTTCAAGT | |
| | AAAGTAACGGTTTTAGAGTGAGACCAGCGTAACTCTTAGCTCTGGCTC | |
| Q332R | TATCTACACGGGTCTCACCAAAACACTCCTGGAAAAAGTATTACAGCATCCAAAAATTATT | 193 |
| | AAAAGAGCGACCTTACTTTACTTGAAAAAAACACTTCGGGAGGATGAAGAAATTTTCAAGT | |
| | AAAGTAACGGTTTTAGAGTGAGACCAGCGTAACTCAAGAAGTTCAGCT | |
| Q332N | TATCTACACGGGTCTCACCAAAACACTCCTGGAAAAAGTATTACAGCATCCAAAAATTATT | 194 |
| | AAAAATGCGACCTTACTTTACTTGAAAAAAACACTTCGGGAGGATGAAGAAATTTTCAAGT | |
| | AAAGTAACGGTTTTAGAGTGAGACCAGCGTAACTCCGAACGGATCGGT | |
| Q332D | TATCTACACGGGTCTCACCAAAACACTCCTGGAAAAAGTATTACAGCATCCAAAAATTATT | 195 |
| | AAAGATGCGACCTTACTTTACTTGAAAAAAACACTTCGGGAGGATGAAGAAATTTTCAAGT | |
| | AAAGTAACGGTTTTAGAGTGAGACCAGCGTAACTCCGACCCTATCAAC | |
| Q332C | TATCTACACGGGTCTCACCAAAACACTCCTGGAAAAAGTATTACAGCATCCAAAAATTATT | 196 |
| | AAATGTGCGACCTTACTTTACTTGAAAAAAACACTTCGGGAGGATGAAGAAATTTTCAAGT | |
| | AAAGTAACGGTTTTAGAGTGAGACCAGCGTAACTCGGATCACATGCAC | |
| Q332Q | TATCTACACGGGTCTCACCAAAACACTCCTGGAAAAAGTATTACAGCATCCAAAAATTATT | 197 |
| | AAACAAGCGACCTTACTTTACTTGAAAAAAACACTTCGGGAGGATGAAGAAATTTTCAAGT | |
| | AAAGTAACGGTTTTAGAGTGAGACCAGCGTAACTCCAACAGGCCTGGA | |
| Q332E | TATCTACACGGGTCTCACCAAAACACTCCTGGAAAAAGTATTACAGCATCCAAAAATTATT | 198 |
| | AAAGAAGCGACCTTACTTTACTTGAAAAAAACACTTCGGGAGGATGAAGAAATTTTCAAGT | |
| | AAAGTAACGGTTTTAGAGTGAGACCAGCGTAACTCTTGACGTAGCAGG | |
| Q332G | TATCTACACGGGTCTCACCAAAACACTCCTGGAAAAAGTATTACAGCATCCAAAAATTATT | 199 |
| | AAAGGTGCGACCTTACTTTACTTGAAAAAAACACTTCGGGAGGATGAAGAAATTTTCAAGT | |
| | AAAGTAACGGTTTTAGAGTGAGACCAGCGTAACTCCACGCGGTCATGA | |
| Q332H | TATCTACACGGGTCTCACCAAAACACTCCTGGAAAAAGTATTACAGCATCCAAAAATTATT | 200 |
| | AAACATGCGACCTTACTTTACTTGAAAAAAACACTTCGGGAGGATGAAGAAATTTTCAAGT | |
| | AAAGTAACGGTTTTAGAGTGAGACCAGCGTAACTCACATTTTCGTGAA | |
| Q332I | TATCTACACGGGTCTCACCAAAACACTCCTGGAAAAAGTATTACAGCATCCAAAAATTATT | 201 |
| | AAAATTGCGACCTTACTTTACTTGAAAAAAACACTTCGGGAGGATGAAGAAATTTTCAAGT | |
| | AAAGTAACGGTTTTAGAGTGAGACCAGCGTAACTCCCTGTAGATTCCC | |
| Q332L | TATCTACACGGGTCTCACCAAAACACTCCTGGAAAAAGTATTACAGCATCCAAAAATTATT | 202 |
| | AAATTGGCGACCTTACTTTACTTGAAAAAAACACTTCGGGAGGATGAAGAAATTTTCAAGT | |
| | AAAGTAACGGTTTTAGAGTGAGACCAGCGTAACTCGTGAGGAAGGGCT | |
| Q332K | TATCTACACGGGTCTCACCAAAACACTCCTGGAAAAAGTATTACAGCATCCAAAAATTATT | 203 |
| | AAAAAAGCGACCTTACTTTACTTGAAAAAAACACTTCGGGAGGATGAAGAAATTTTCAAGT | |
| | AAAGTAACGGTTTTAGAGTGAGACCAGCGTAACTCACGGACAGCCGCA | |
| Q332M | TATCTACACGGGTCTCACCAAAACACTCCTGGAAAAAGTATTACAGCATCCAAAAATTATT | 204 |
| | AAAATGGCGACCTTACTTTACTTGAAAAAAACACTTCGGGAGGATGAAGAAATTTTCAAGT | |
| | AAAGTAACGGTTTTAGAGTGAGACCAGCGTAACTCGGGCACATCCACT | |
| Q332F | TATCTACACGGGTCTCACCAAAACACTCCTGGAAAAAGTATTACAGCATCCAAAAATTATT | 205 |
| | AAATTTGCGACCTTACTTTACTTGAAAAAAACACTTCGGGAGGATGAAGAAATTTTCAAGT | |
| | AAAGTAACGGTTTTAGAGTGAGACCAGCGTAACTCCCTCCTGCCCTTT | |
| Q332P | TATCTACACGGGTCTCACCAAAACACTCCTGGAAAAAGTATTACAGCATCCAAAAATTATT | 206 |
| | AAACCAGCGACCTTACTTTACTTGAAAAAAACACTTCGGGAGGATGAAGAAATTTTCAAGT | |
| | AAAGTAACGGTTTTAGAGTGAGACCAGCGTAACTCGTCTCGGGTTTAG | |
| Q332S | TATCTACACGGGTCTCACCAAAACACTCCTGGAAAAAGTATTACAGCATCCAAAAATTATT | 207 |
| | AAATCTGCGACCTTACTTTACTTGAAAAAAACACTTCGGGAGGATGAAGAAATTTTCAAGT | |
| | AAAGTAACGGTTTTAGAGTGAGACCAGCGTAACTCAGAGTGTTCTACG | |
| Q332T | TATCTACACGGGTCTCACCAAAACACTCCTGGAAAAAGTATTACAGCATCCAAAAATTATT | 208 |
| | AAAACTGCGACCTTACTTTACTTGAAAAAAACACTTCGGGAGGATGAAGAAATTTTCAAGT | |
| | AAAGTAACGGTTTTAGAGTGAGACCAGCGTAACTCGCGTCCTTAACAT | |
| Q332W | TATCTACACGGGTCTCACCAAAACACTCCTGGAAAAAGTATTACAGCATCCAAAAATTATT | 209 |
| | AAATGGGCGACCTTACTTTACTTGAAAAAAACACTTCGGGAGGATGAAGAAATTTTCAAGT | |
| | AAAGTAACGGTTTTAGAGTGAGACCAGCGTAACTCAGAACGAAGGACG | |
| Q332Y | TATCTACACGGGTCTCACCAAAACACTCCTGGAAAAAGTATTACAGCATCCAAAAATTATT | 210 |
| | AAATATGCGACCTTACTTTACTTGAAAAAAACACTTCGGGAGGATGAAGAAATTTTCAAGT | |
| | AAAGTAACGGTTTTAGAGTGAGACCAGCGTAACTCACCGCGGCCGTGC | |
| Q332V | TATCTACACGGGTCTCACCAAAACACTCCTGGAAAAAGTATTACAGCATCCAAAAATTATT | 211 |
| | AAAGTTGCGACCTTACTTTACTTGAAAAAAACACTTCGGGAGGATGAAGAAATTTTCAAGT | |
| | AAAGTAACGGTTTTAGAGTGAGACCAGCGTAACTCAGGTTACAAAAGC | |
| A333A | TATCTACACGGGTCTCACCAAAACCCTGGAAAAAGTATTACAGCATCCAAAAATTATTAAA | 212 |
| | CAAGCGACCTTACTTTACTTGAAAAAAACACTTCGGGAGGATGAAGAAATTTTCAAGTAAA | |
| | GTAACGGTTTTAGAGTGAGACCAGCGTAACTCGAGTGACTCAAGATCC | |
| A333R | TATCTACACGGGTCTCACCAAAACCCTGGAAAAAGTATTACAGCATCCAAAAATTATTAAA | 213 |
| | CAACGGACCTTACTTTACTTGAAAAAAACACTTCGGGAGGATGAAGAAATTTTCAAGTAAA | |
| | GTAACGGTTTTAGAGTGAGACCAGCGTAACTCCTCTTATCACACTGAC | |
| A333N | TATCTACACGGGTCTCACCAAAACCCTGGAAAAAGTATTACAGCATCCAAAAATTATTAAA | 214 |
| | CAAAACACCTTACTTTACTTGAAAAAAACACTTCGGGAGGATGAAGAAATTTTCAAGTAAA | |
| | GTAACGGTTTTAGAGTGAGACCAGCGTAACTCAATATTGACGTAACAT | |
| A333D | TATCTACACGGGTCTCACCAAAACCCTGGAAAAAGTATTACAGCATCCAAAAATTATTAAA | 215 |
| | CAAGACACCTTACTTTACTTGAAAAAAACACTTCGGGAGGATGAAGAAATTTTCAAGTAAA | |
| | GTAACGGTTTTAGAGTGAGACCAGCGTAACTCCATCGCTGCTTCCCGC | |
| A333C | TATCTACACGGGTCTCACCAAAACCCTGGAAAAAGTATTACAGCATCCAAAAATTATTAAA | 216 |
| | CAATGCACCTTACTTTACTTGAAAAAAACACTTCGGGAGGATGAAGAAATTTTCAAGTAAA | |
| | GTAACGGTTTTAGAGTGAGACCAGCGTAACTCAATATAAAGCTTAGCG | |
| A333Q | TATCTACACGGGTCTCACCAAAACCCTGGAAAAAGTATTACAGCATCCAAAAATTATTAAA | 217 |
| | CAACAGACCTTACTTTACTTGAAAAAAACACTTCGGGAGGATGAAGAAATTTTCAAGTAAA | |
| | GTAACGGTTTTAGAGTGAGACCAGCGTAACTCTTTAGGAGTGGGTTAG | |
| A333E | TATCTACACGGGTCTCACCAAAACCCTGGAAAAAGTATTACAGCATCCAAAAATTATTAAA | 218 |
| | CAAGAGATCTTACTTTACTTGAAAAAAACACTTCGGGAGGATGAAGAAATTTTCAAGTAAA | |
| | GTAACGGTTTTAGAGTGAGACCAGCGTAACTCTAAAATTTTATATACA | |
| A333G | TATCTACACGGGTCTCACCAAAACCCTGGAAAAAGTATTACAGCATCCAAAAATTATTAAA | 219 |
| | CAAGGGACCTTACTTTACTTGAAAAAAACACTTCGGGAGGATGAAGAAATTTTCAAGTAAA | |
| | GTAACGGTTTTAGAGTGAGACCAGCGTAACTCCATCATGGAATTAGAA | |
| A333H | TATCTACACGGGTCTCACCAAAACCCTGGAAAAAGTATTACAGCATCCAAAAATTATTAAA | 220 |
| | CAACACACCTTACTTTACTTGAAAAAAACACTTCGGGAGGATGAAGAAATTTTCAAGTAAA | |
| | GTAACGGTTTTAGAGTGAGACCAGCGTAACTCGGTTACTCGGAAAGAC | |
| A333I | TATCTACACGGGTCTCACCAAAACCCTGGAAAAAGTATTACAGCATCCAAAAATTATTAAA | 221 |
| | CAAATCACCTTACTTTACTTGAAAAAAACACTTCGGGAGGATGAAGAAATTTTCAAGTAAA | |
| | GTAACGGTTTTAGAGTGAGACCAGCGTAACTCTCGACGACAGCCCATG | |
| A333L | TATCTACACGGGTCTCACCAAAACCCTGGAAAAAGTATTACAGCATCCAAAAATTATTAAA | 222 |
| | CAACTCACCTTACTTTACTTGAAAAAAACACTTCGGGAGGATGAAGAAATTTTCAAGTAAA | |
| | GTAACGGTTTTAGAGTGAGACCAGCGTAACTCGGATGCTACACTCTCC | |
| A333K | TATCTACACGGGTCTCACCAAAACCCTGGAAAAAGTATTACAGCATCCAAAAATTATTAAA | 223 |
| | CAAAAGACCTTACTTTACTTGAAAAAAACACTTCGGGAGGATGAAGAAATTTTCAAGTAAA | |
| | GTAACGGTTTTAGAGTGAGACCAGCGTAACTCTCTCAACGGTGAGTTG | |
| A333M | TATCTACACGGGTCTCACCAAAACCCTGGAAAAAGTATTACAGCATCCAAAAATTATTAAA | 224 |
| | CAAATGACCTTACTTTACTTGAAAAAAACACTTCGGGAGGATGAAGAAATTTTCAAGTAAA | |
| | GTAACGGTTTTAGAGTGAGACCAGCGTAACTCTGGGATTGTGACCTCC | |
| A333F | TATCTACACGGGTCTCACCAAAACCCTGGAAAAAGTATTACAGCATCCAAAAATTATTAAA | 225 |
| | CAATTCACCTTACTTTACTTGAAAAAAACACTTCGGGAGGATGAAGAAATTTTCAAGTAAA | |
| | GTAACGGTTTTAGAGTGAGACCAGCGTAACTCCTAACCGTTTTGATGC | |
| A333P | TATCTACACGGGTCTCACCAAAACCCTGGAAAAAGTATTACAGCATCCAAAAATTATTAAA | 226 |
| | CAACCGACCTTACTTTACTTGAAAAAAACACTTCGGGAGGATGAAGAAATTTTCAAGTAAA | |
| | GTAACGGTTTTAGAGTGAGACCAGCGTAACTCTGAATTTTGATTCAAC | |
| A333S | TATCTACACGGGTCTCACCAAAACCCTGGAAAAAGTATTACAGCATCCAAAAATTATTAAA | 227 |
| | CAAAGCACCTTACTTTACTTGAAAAAAACACTTCGGGAGGATGAAGAAATTTTCAAGTAAA | |
| | GTAACGGTTTTAGAGTGAGACCAGCGTAACTCAAGTAATAGGTGGGTC | |
| A333T | TATCTACACGGGTCTCACCAAAACCCTGGAAAAAGTATTACAGCATCCAAAAATTATTAAA | 228 |
| | CAAACGACCTTACTTTACTTGAAAAAAACACTTCGGGAGGATGAAGAAATTTTCAAGTAAA | |
| | GTAACGGTTTTAGAGTGAGACCAGCGTAACTCGGTCTGGCCTGTTCGA | |
| A333W | TATCTACACGGGTCTCACCAAAACCCTGGAAAAAGTATTACAGCATCCAAAAATTATTAAA | 229 |
| | CAATGGACCTTACTTTACTTGAAAAAAACACTTCGGGAGGATGAAGAAATTTTCAAGTAAA | |
| | GTAACGGTTTTAGAGTGAGACCAGCGTAACTCGCACAAATTGAGTTTG | |
| A333Y | TATCTACACGGGTCTCACCAAAACCCTGGAAAAAGTATTACAGCATCCAAAAATTATTAAA | 230 |
| | CAATACACCTTACTTTACTTGAAAAAAACACTTCGGGAGGATGAAGAAATTTTCAAGTAAA | |
| | GTAACGGTTTTAGAGTGAGACCAGCGTAACTCTTCGATCCTGGTAACA | |
| A333V | TATCTACACGGGTCTCACCAAAACCCTGGAAAAAGTATTACAGCATCCAAAAATTATTAAA | 231 |
| | CAAGTCACCTTACTTTACTTGAAAAAAACACTTCGGGAGGATGAAGAAATTTTCAAGTAAA | |
| | GTAACGGTTTTAGAGTGAGACCAGCGTAACTCACCGCCCGTGGCATAC | |
| T334A | TATCTACACGGGTCTCACCAAAACCTGGAAAAAGTATTACAGCATCCAAAAATTATTAAAC | 232 |
| | AAGCGGCGTTACTTTACTTGAAAAAAACACTTCGGGAGGATGAAGAAAAATTTTCAAGTAA | |
| | AGTAACGGTTTTAGAGTGAGACCAGCGTAACTCACTATGGTGGTTTTC | |
| T334R | TATCTACACGGGTCTCACCAAAACCTGGAAAAAGTATTACAGCATCCAAAAATTATTAAAC | 233 |
| | AAGCGCGGTTACTTTACTTGAAAAAAACACTTCGGGAGGATGAAGAAAAATTTTCAAGTAA | |
| | AGTAACGGTTTTAGAGTGAGACCAGCGTAACTCTCTCCAACTCCATAC | |
| T334N | TATCTACACGGGTCTCACCAAAACCTGGAAAAAGTATTACAGCATCCAAAAATTATTAAAC | 234 |
| | AAGCGAACTTACTTTACTTGAAAAAAACACTTCGGGAGGATGAAGAAAAATTTTCAAGTAA | |
| | AGTAACGGTTTTAGAGTGAGACCAGCGTAACTCGAAGATGCCAGTGAC | |
| T334D | TATCTACACGGGTCTCACCAAAACCTGGAAAAAGTATTACAGCATCCAAAAATTATTAAAC | 235 |
| | AAGCGGACTTACTTTACTTGAAAAAAACACTTCGGGAGGATGAAGAAAAATTTTCAAGTAA | |
| | AGTAACGGTTTTAGAGTGAGACCAGCGTAACTCGGAGACCGAGCGCCC | |
| T334C | TATCTACACGGGTCTCACCAAAACCTGGAAAAAGTATTACAGCATCCAAAAATTATTAAAC | 236 |
| | AAGCGTGCTTACTTTACTTGAAAAAAACACTTCGGGAGGATGAAGAAAAATTTTCAAGTAA | |
| | AGTAACGGTTTTAGAGTGAGACCAGCGTAACTCTTGATTCCGCGAGAG | |
| T334Q | TATCTACACGGGTCTCACCAAAACCTGGAAAAAGTATTACAGCATCCAAAAATTATTAAAC | 237 |
| | AAGCGCAGTTACTTTACTTGAAAAAAACACTTCGGGAGGATGAAGAAAAATTTTCAAGTAA | |
| | AGTAACGGTTTTAGAGTGAGACCAGCGTAACTCCTTGGTCGGAATGAT | |
| T334E | TATCTACACGGGTCTCACCAAAACCTGGAAAAAGTATTACAGCATCCAAAAATTATTAAAC | 238 |
| | AAGCGGAGTTACTTTACTTGAAAAAAACACTTCGGGAGGATGAAGAAAAATTTTCAAGTAA | |
| | AGTAACGGTTTTAGAGTGAGACCAGCGTAACTCACCAGAGTGAGTACC | |
| T334G | TATCTACACGGGTCTCACCAAAACCTGGAAAAAGTATTACAGCATCCAAAAATTATTAAAC | 239 |
| | AAGCGGGGTTACTTTACTTGAAAAAAACACTTCGGGAGGATGAAGAAAAATTTTCAAGTAA | |
| | AGTAACGGTTTTAGAGTGAGACCAGCGTAACTCACCATTGTATCAAGC | |
| T334H | TATCTACACGGGTCTCACCAAAACCTGGAAAAAGTATTACAGCATCCAAAAATTATTAAAC | 240 |
| | AAGCGCACTTACTTTACTTGAAAAAAACACTTCGGGAGGATGAAGAAAAATTTTCAAGTAA | |
| | AGTAACGGTTTTAGAGTGAGACCAGCGTAACTCTGTAGTTACCTATGT | |
| T334I | TATCTACACGGGTCTCACCAAAACCTGGAAAAAGTATTACAGCATCCAAAAATTATTAAAC | 241 |
| | AAGCGATCTTACTTTACTTGAAAAAAACACTTCGGGAGGATGAAGAAAAATTTTCAAGTAA | |
| | AGTAACGGTTTTAGAGTGAGACCAGCGTAACTCAAATCAATTTTCGCC | |
| T334L | TATCTACACGGGTCTCACCAAAACCTGGAAAAAGTATTACAGCATCCAAAAATTATTAAAC | 242 |
| | AAGCGCTCTTACTTTACTTGAAAAAAACACTTCGGGAGGATGAAGAAAAATTTTCAAGTAA | |
| | AGTAACGGTTTTAGAGTGAGACCAGCGTAACTCCACATAGGTGAGGTT | |
| T334K | TATCTACACGGGTCTCACCAAAACCTGGAAAAAGTATTACAGCATCCAAAAATTATTAAAC | 243 |
| | AAGCGAAGTTACTTTACTTGAAAAAAACACTTCGGGAGGATGAAGAAAAATTTTCAAGTAA | |
| | AGTAACGGTTTTAGAGTGAGACCAGCGTAACTCCTCGTTGTCTGGCCC | |
| T334M | TATCTACACGGGTCTCACCAAAACCTGGAAAAAGTATTACAGCATCCAAAAATTATTAAAC | 244 |
| | AAGCGATGTTACTTTACTTGAAAAAAACACTTCGGGAGGATGAAGAAAAATTTTCAAGTAA | |
| | AGTAACGGTTTTAGAGTGAGACCAGCGTAACTCTTCCGCCTAATAGGC | |
| T334F | TATCTACACGGGTCTCACCAAAACCTGGAAAAAGTATTACAGCATCCAAAAATTATTAAAC | 245 |
| | AAGCGTTCTTACTTTACTTGAAAAAAACACTTCGGGAGGATGAAGAAAAATTTTCAAGTAA | |
| | AGTAACGGTTTTAGAGTGAGACCAGCGTAACTCTTCGGATGAATCGCG | |
| T334P | TATCTACACGGGTCTCACCAAAACCTGGAAAAAGTATTACAGCATCCAAAAATTATTAAAC | 246 |
| | AAGCGCCGTTACTTTACTTGAAAAAAACACTTCGGGAGGATGAAGAAAAATTTTCAAGTAA | |
| | AGTAACGGTTTTAGAGTGAGACCAGCGTAACTCCATTGGAATGCGACC | |
| T334S | TATCTACACGGGTCTCACCAAAACCTGGAAAAAGTATTACAGCATCCAAAAATTATTAAAC | 247 |
| | AAGCGAGCTTACTTTACTTGAAAAAAACACTTCGGGAGGATGAAGAAAAATTTTCAAGTAA | |
| | AGTAACGGTTTTAGAGTGAGACCAGCGTAACTCGTACCCTGCTCCCCC | |
| T334T | TATCTACACGGGTCTCACCAAAACCTGGAAAAAGTATTACAGCATCCAAAAATTATTAAAC | 248 |
| | AAGCGACGTTACTTTACTTGAAAAAAACACTTCGGGAGGATGAAGAAAAATTTTCAAGTAA | |
| | AGTAACGGTTTTAGAGTGAGACCAGCGTAACTCGACACCTGCGAAGAC | |
| T334W | TATCTACACGGGTCTCACCAAAACCTGGAAAAAGTATTACAGCATCCAAAAATTATTAAAC | 249 |
| | AAGCGTGGTTACTTTACTTGAAAAAAACACTTCGGGAGGATGAAGAAAAATTTTCAAGTAA | |
| | AGTAACGGTTTTAGAGTGAGACCAGCGTAACTCTGAAACATTAAGAAG | |
| T334Y | TATCTACACGGGTCTCACCAAAACCTGGAAAAAGTATTACAGCATCCAAAAATTATTAAAC | 250 |
| | AAGCGTACTTACTTTACTTGAAAAAAACACTTCGGGAGGATGAAGAAAAATTTTCAAGTAA | |
| | AGTAACGGTTTTAGAGTGAGACCAGCGTAACTCATCTGTCACGTCGTG | |
| T334V | TATCTACACGGGTCTCACCAAAACCTGGAAAAAGTATTACAGCATCCAAAAATTATTAAAC | 251 |
| | AAGCGGTCTTACTTTACTTGAAAAAAACACTTCGGGAGGATGAAGAAAAATTTTCAAGTAA | |
| | AGTAACGGTTTTAGAGTGAGACCAGCGTAACTCAGAGGAAACTCTCAG | |
| L335A | TATCTACACGGGTCTCACCAAAACCTGGAAAAAGTATTACAGCATCCAAAAATTATTAAAC | 252 |
| | AAGCGACCGCTCTTTACTTGAAAAAAACACTTCGGGAGGATGAAGAAATGGAATTTTCAAG | |
| | TAAAGTAACGGTTTTAGAGTGAGACCAGCGTAACTCTGGACATATCAT | |
| L335R | TATCTACACGGGTCTCACCAAAACCTGGAAAAAGTATTACAGCATCCAAAAATTATTAAAC | 253 |
| | AAGCGACCAGACTTTACTTGAAAAAAACACTTCGGGAGGATGAAGAAATGGAATTTTCAAG | |
| | TAAAGTAACGGTTTTAGAGTGAGACCAGCGTAACTCGTGTGCGGGATA | |
| L335N | TATCTACACGGGTCTCACCAAAACCTGGAAAAAGTATTACAGCATCCAAAAATTATTAAAC | 254 |
| | AAGCGACCAATCTTTACTTGAAAAAAACACTTCGGGAGGATGAAGAAATGGAATTTTCAAG | |
| | TAAAGTAACGGTTTTAGAGTGAGACCAGCGTAACTCAACCTCCTAATG | |
| L335D | TATCTACACGGGTCTCACCAAAACCTGGAAAAAGTATTACAGCATCCAAAAATTATTAAAC | 255 |
| | AAGCGACCGATCTTTACTTGAAAAAAACACTTCGGGAGGATGAAGAAATGGAATTTTCAAG | |
| | TAAAGTAACGGTTTTAGAGTGAGACCAGCGTAACTCTTCCTCCTTCAT | |
| L335C | TATCTACACGGGTCTCACCAAAACCTGGAAAAAGTATTACAGCATCCAAAAATTATTAAAC | 256 |
| | AAGCGACCTGTCTTTACTTGAAAAAAACACTTCGGGAGGATGAAGAAATGGAATTTTCAAG | |
| | TAAAGTAACGGTTTTAGAGTGAGACCAGCGTAACTCGGTATGCGCGGT | |
| L335Q | TATCTACACGGGTCTCACCAAAACCTGGAAAAAGTATTACAGCATCCAAAAATTATTAAAC | 257 |
| | AAGCGACCCAACTTTACTTGAAAAAAACACTTCGGGAGGATGAAGAAATGGAATTTTCAAG | |
| | TAAAGTAACGGTTTTAGAGTGAGACCAGCGTAACTCAACCATCACGCG | |
| L335E | TATCTACACGGGTCTCACCAAAACCTGGAAAAAGTATTACAGCATCCAAAAATTATTAAAC | 258 |
| | AAGCGACCGAACTTTACTTGAAAAAAACACTTCGGGAGGATGAAGAAATGGAATTTTCAAG | |
| | TAAAGTAACGGTTTTAGAGTGAGACCAGCGTAACTCCCAGGCGGTCGG | |
| L335G | TATCTACACGGGTCTCACCAAAACCTGGAAAAAGTATTACAGCATCCAAAAATTATTAAAC | 259 |
| | AAGCGACCGGTCTTTACTTGAAAAAAACACTTCGGGAGGATGAAGAAATGGAATTTTCAAG | |
| | TAAAGTAACGGTTTTAGAGTGAGACCAGCGTAACTCTGGTTGTCAACG | |
| L335H | TATCTACACGGGTCTCACCAAAACCTGGAAAAAGTATTACAGCATCCAAAAATTATTAAAC | 260 |
| | AAGCGACCCATCTTTACTTGAAAAAAACACTTCGGGAGGATGAAGAAATGGAATTTTCAAG | |
| | TAAAGTAACGGTTTTAGAGTGAGACCAGCGTAACTCTAGATTGCCAGG | |
| L335I | TATCTACACGGGTCTCACCAAAACCTGGAAAAAGTATTACAGCATCCAAAAATTATTAAAC | 261 |
| | AAGCGACCATTCTTTACTTGAAAAAAACACTTCGGGAGGATGAAGAAATGGAATTTTCAAG | |
| | TAAAGTAACGGTTTTAGAGTGAGACCAGCGTAACTCGGCACACCAGTG | |
| L335L | TATCTACACGGGTCTCACCAAAACCTGGAAAAAGTATTACAGCATCCAAAAATTATTAAAC | 262 |
| | AAGCGACCTTGCTTTACTTGAAAAAAACACTTCGGGAGGATGAAGAAATGGAATTTTCAAG | |
| | TAAAGTAACGGTTTTAGAGTGAGACCAGCGTAACTCGCCAGGTTTTAG | |
| L335K | TATCTACACGGGTCTCACCAAAACCTGGAAAAAGTATTACAGCATCCAAAAATTATTAAAC | 263 |
| | AAGCGACCAAACTTTACTTGAAAAAAACACTTCGGGAGGATGAAGAAATGGAATTTTCAAG | |
| | TAAAGTAACGGTTTTAGAGTGAGACCAGCGTAACTCTACGTCTTGCCA | |
| L335M | TATCTACACGGGTCTCACCAAAACCTGGAAAAAGTATTACAGCATCCAAAAATTATTAAAC | 264 |
| | AAGCGACCATGCTTTACTTGAAAAAAACACTTCGGGAGGATGAAGAAATGGAATTTTCAAG | |
| | TAAAGTAACGGTTTTAGAGTGAGACCAGCGTAACTCGGACGAATGCGG | |
| L335F | TATCTACACGGGTCTCACCAAAACCTGGAAAAAGTATTACAGCATCCAAAAATTATTAAAC | 265 |
| | AAGCGACCTTTCTTTACTTGAAAAAAACACTTCGGGAGGATGAAGAAATGGAATTTTCAAG | |
| | TAAAGTAACGGTTTTAGAGTGAGACCAGCGTAACTCCTGACACATGGG | |
| L335P | TATCTACACGGGTCTCACCAAAACCTGGAAAAAGTATTACAGCATCCAAAAATTATTAAAC | 266 |
| | AAGCGACCCCACTTTACTTGAAAAAAACACTTCGGGAGGATGAAGAAATGGAATTTTCAAG | |
| | TAAAGTAACGGTTTTAGAGTGAGACCAGCGTAACTCGCCCCCGTAAAG | |
| L335S | TATCTACACGGGTCTCACCAAAACCTGGAAAAAGTATTACAGCATCCAAAAATTATTAAAC | 267 |
| | AAGCGACCTCTCTTTACTTGAAAAAAACACTTCGGGAGGATGAAGAAATGGAATTTTCAAG | |
| | TAAAGTAACGGTTTTAGAGTGAGACCAGCGTAACTCGAAGCAGCTACA | |
| L335T | TATCTACACGGGTCTCACCAAAACCTGGAAAAAGTATTACAGCATCCAAAAATTATTAAAC | 268 |
| | AAGCGACCACTCTTTACTTGAAAAAAACACTTCGGGAGGATGAAGAAATGGAATTTTCAAG | |
| | TAAAGTAACGGTTTTAGAGTGAGACCAGCGTAACTCTATCCACGGTCA | |
| L335W | TATCTACACGGGTCTCACCAAAACCTGGAAAAAGTATTACAGCATCCAAAAATTATTAAAC | 269 |
| | AAGCGACCTGGCTTTACTTGAAAAAAACACTTCGGGAGGATGAAGAAATGGAATTTTCAAG | |
| | TAAAGTAACGGTTTTAGAGTGAGACCAGCGTAACTCGTACACGTATGG | |
| L335Y | TATCTACACGGGTCTCACCAAAACCTGGAAAAAGTATTACAGCATCCAAAAATTATTAAAC | 270 |
| | AAGCGACCTATCTTTACTTGAAAAAAACACTTCGGGAGGATGAAGAAATGGAATTTTCAAG | |
| | TAAAGTAACGGTTTTAGAGTGAGACCAGCGTAACTCCGCCGAGCCTGC | |
| L335V | TATCTACACGGGTCTCACCAAAACCTGGAAAAAGTATTACAGCATCCAAAAATTATTAAAC | 271 |
| | AAGCGACCGTTCTTTACTTGAAAAAAACACTTCGGGAGGATGAAGAAATGGAATTTTCAAG | |
| | TAAAGTAACGGTTTTAGAGTGAGACCAGCGTAACTCCATAGCCCTTGA | |
| L336A | TATCTACACGGGTCTCACCAAAACCTGGAAAAAGTATTACAGCATCCAAAAATTATTAAAC | 272 |
| | AAGCGACCTTAGCTTACTTGAAAAAAACACTTCGGGAGGATGAAGAAATGGGCTAATTTTC | |
| | AAGTAAAGTAACGGTTTTAGAGTGAGACCAGCGTAACTCCCTATGGGA | |
| L336R | TATCTACACGGGTCTCACCAAAACCTGGAAAAAGTATTACAGCATCCAAAAATTATTAAAC | 273 |
| | AAGCGACCTTAAGATACTTGAAAAAAACACTTCGGGAGGATGAAGAAATGGGCTAATTTTC | |
| | AAGTAAAGTAACGGTTTTAGAGTGAGACCAGCGTAACTCAACCTAGAC | |
| L336N | TATCTACACGGGTCTCACCAAAACCTGGAAAAAGTATTACAGCATCCAAAAATTATTAAAC | 274 |
| | AAGCGACCTTAAATTACTTGAAAAAAACACTTCGGGAGGATGAAGAAATGGGCTAATTTTC | |
| | AAGTAAAGTAACGGTTTTAGAGTGAGACCAGCGTAACTCCACGCTAAA | |
| L336D | TATCTACACGGGTCTCACCAAAACCTGGAAAAAGTATTACAGCATCCAAAAATTATTAAAC | 275 |
| | AAGCGACCTTAGATTACTTGAAAAAAACACTTCGGGAGGATGAAGAAATGGGCTAATTTTC | |
| | AAGTAAAGTAACGGTTTTAGAGTGAGACCAGCGTAACTCGCCCAATCC | |
| L336C | TATCTACACGGGTCTCACCAAAACCTGGAAAAAGTATTACAGCATCCAAAAATTATTAAAC | 276 |
| | AAGCGACCTTATGTTACTTGAAAAAAACACTTCGGGAGGATGAAGAAATGGGCTAATTTTC | |
| | AAGTAAAGTAACGGTTTTAGAGTGAGACCAGCGTAACTCGTGAAGAAC | |
| L336Q | TATCTACACGGGTCTCACCAAAACCTGGAAAAAGTATTACAGCATCCAAAAATTATTAAAC | 277 |
| | AAGCGACCTTACAATACTTGAAAAAAACACTTCGGGAGGATGAAGAAATGGGCTAATTTTC | |
| | AAGTAAAGTAACGGTTTTAGAGTGAGACCAGCGTAACTCCCATTGGTC | |
| L336E | TATCTACACGGGTCTCACCAAAACCTGGAAAAAGTATTACAGCATCCAAAAATTATTAAAC | 278 |
| | AAGCGACCTTAGAATACTTGAAAAAAACACTTCGGGAGGATGAAGAAATGGGCTAATTTTC | |
| | AAGTAAAGTAACGGTTTTAGAGTGAGACCAGCGTAACTCAAGTAGGGA | |
| L336G | TATCTACACGGGTCTCACCAAAACCTGGAAAAAGTATTACAGCATCCAAAAATTATTAAAC | 279 |
| | AAGCGACCTTAGGTTACTTGAAAAAAACACTTCGGGAGGATGAAGAAATGGGCTAATTTTC | |
| | AAGTAAAGTAACGGTTTTAGAGTGAGACCAGCGTAACTCATGTCCGCA | |
| L336H | TATCTACACGGGTCTCACCAAAACCTGGAAAAAGTATTACAGCATCCAAAAATTATTAAAC | 280 |
| | AAGCGACCTTACATTACTTGAAAAAAACACTTCGGGAGGATGAAGAAATGGGCTAATTTTC | |
| | AAGTAAAGTAACGGTTTTAGAGTGAGACCAGCGTAACTCAACTCGCAG | |
| L336I | TATCTACACGGGTCTCACCAAAACCTGGAAAAAGTATTACAGCATCCAAAAATTATTAAAC | 281 |
| | AAGCGACCTTAATTTACTTGAAAAAAACACTTCGGGAGGATGAAGAAATGGGCTAATTTTC | |
| | AAGTAAAGTAACGGTTTTAGAGTGAGACCAGCGTAACTCATATTCCTC | |
| L336L | TATCTACACGGGTCTCACCAAAACCTGGAAAAAGTATTACAGCATCCAAAAATTATTAAAC | 282 |
| | AAGCGACCTTATTGTACTTGAAAAAAACACTTCGGGAGGATGAAGAAATGGGCTAATTTTC | |
| | AAGTAAAGTAACGGTTTTAGAGTGAGACCAGCGTAACTCATCCGTGAA | |
| L336K | TATCTACACGGGTCTCACCAAAACCTGGAAAAAGTATTACAGCATCCAAAAATTATTAAAC | 283 |
| | AAGCGACCTTAAAATACTTGAAAAAAACACTTCGGGAGGATGAAGAAATGGGCTAATTTTC | |
| | AAGTAAAGTAACGGTTTTAGAGTGAGACCAGCGTAACTCGGTCCACAG | |
| L336M | TATCTACACGGGTCTCACCAAAACCTGGAAAAAGTATTACAGCATCCAAAAATTATTAAAC | 284 |
| | AAGCGACCTTAATGTACTTGAAAAAAACACTTCGGGAGGATGAAGAAATGGGCTAATTTTC | |
| | AAGTAAAGTAACGGTTTTAGAGTGAGACCAGCGTAACTCAGGTTACGC | |
| L336F | TATCTACACGGGTCTCACCAAAACCTGGAAAAAGTATTACAGCATCCAAAAATTATTAAAC | 285 |
| | AAGCGACCTTATTTTACTTGAAAAAAACACTTCGGGAGGATGAAGAAATGGGCTAATTTTC | |
| | AAGTAAAGTAACGGTTTTAGAGTGAGACCAGCGTAACTCAAGTGTTTA | |
| L336P | TATCTACACGGGTCTCACCAAAACCTGGAAAAAGTATTACAGCATCCAAAAATTATTAAAC | 286 |
| | AAGCGACCTTACCATACTTGAAAAAAACACTTCGGGAGGATGAAGAAATGGGCTAATTTTC | |
| | AAGTAAAGTAACGGTTTTAGAGTGAGACCAGCGTAACTCGGCGTCGTC | |
| L336S | TATCTACACGGGTCTCACCAAAACCTGGAAAAAGTATTACAGCATCCAAAAATTATTAAAC | 287 |
| | AAGCGACCTTATCTTACTTGAAAAAAACACTTCGGGAGGATGAAGAAATGGGCTAATTTTC | |
| | AAGTAAAGTAACGGTTTTAGAGTGAGACCAGCGTAACTCGACGTTCGA | |
| L336T | TATCTACACGGGTCTCACCAAAACCTGGAAAAAGTATTACAGCATCCAAAAATTATTAAAC | 288 |
| | AAGCGACCTTAACTTACTTGAAAAAAACACTTCGGGAGGATGAAGAAATGGGCTAATTTTC | |
| | AAGTAAAGTAACGGTTTTAGAGTGAGACCAGCGTAACTCTCAATGCTT | |
| L336W | TATCTACACGGGTCTCACCAAAACCTGGAAAAAGTATTACAGCATCCAAAAATTATTAAAC | 289 |
| | AAGCGACCTTATGGTACTTGAAAAAAACACTTCGGGAGGATGAAGAAATGGGCTAATTTTC | |
| | AAGTAAAGTAACGGTTTTAGAGTGAGACCAGCGTAACTCTGGAACTAT | |
| L336Y | TATCTACACGGGTCTCACCAAAACCTGGAAAAAGTATTACAGCATCCAAAAATTATTAAAC | 290 |
| | AAGCGACCTTATATTACTTGAAAAAAACACTTCGGGAGGATGAAGAAATGGGCTAATTTTC | |
| | AAGTAAAGTAACGGTTTTAGAGTGAGACCAGCGTAACTCAAGGCGGCA | |
| L336V | TATCTACACGGGTCTCACCAAAACCTGGAAAAAGTATTACAGCATCCAAAAATTATTAAAC | 291 |
| | AAGCGACCTTAGTTTACTTGAAAAAAACACTTCGGGAGGATGAAGAAATGGGCTAATTTTC | |
| | AAGTAAAGTAACGGTTTTAGAGTGAGACCAGCGTAACTCCTAGCACGC | |
| Y337A | TATCTACACGGGTCTCACCAAAACCTGGAAAAAGTATTACAGCATCCAAAAATTATTAAAC | 292 |
| | AAGCGACCTTACTTGCTTTGAAAAAAACACTTCGGGAGGATGAAGAAATGGGCTTGAAATT | |
| | TTCAAGTAAAGTAACGGTTTTAGAGTGAGACCAGCGTAACTCCACGGC | |
| Y337R | TATCTACACGGGTCTCACCAAAACCTGGAAAAAGTATTACAGCATCCAAAAATTATTAAAC | 293 |
| | AAGCGACCTTACTTAGATTGAAAAAAACACTTCGGGAGGATGAAGAAATGGGCTTGAAATT | |
| | TTCAAGTAAAGTAACGGTTTTAGAGTGAGACCAGCGTAACTCCCGTAT | |
| Y337N | TATCTACACGGGTCTCACCAAAACCTGGAAAAAGTATTACAGCATCCAAAAATTATTAAAC | 294 |
| | AAGCGACCTTACTTAATTTGAAAAAAACACTTCGGGAGGATGAAGAAATGGGCTTGAAATT | |
| | TTCAAGTAAAGTAACGGTTTTAGAGTGAGACCAGCGTAACTCAACTCG | |
| Y337D | TATCTACACGGGTCTCACCAAAACCTGGAAAAAGTATTACAGCATCCAAAAATTATTAAAC | 295 |
| | AAGCGACCTTACTTGATTTGAAAAAAACACTTCGGGAGGATGAAGAAATGGGCTTGAAATT | |
| | TTCAAGTAAAGTAACGGTTTTAGAGTGAGACCAGCGTAACTCCAGGTC | |
| Y337C | TATCTACACGGGTCTCACCAAAACCTGGAAAAAGTATTACAGCATCCAAAAATTATTAAAC | 296 |
| | AAGCGACCTTACTTTGTTTGAAAAAAACACTTCGGGAGGATGAAGAAATGGGCTTGAAATT | |
| | TTCAAGTAAAGTAACGGTTTTAGAGTGAGACCAGCGTAACTCCTCAGT | |
| Y337Q | TATCTACACGGGTCTCACCAAAACCTGGAAAAAGTATTACAGCATCCAAAAATTATTAAAC | 297 |
| | AAGCGACCTTACTTCAATTGAAAAAAACACTTCGGGAGGATGAAGAAATGGGCTTGAAATT | |
| | TTCAAGTAAAGTAACGGTTTTAGAGTGAGACCAGCGTAACTCACGGCT | |
| Y337E | TATCTACACGGGTCTCACCAAAACCTGGAAAAAGTATTACAGCATCCAAAAATTATTAAAC | 298 |
| | AAGCGACCTTACTTGAATTGAAAAAAACACTTCGGGAGGATGAAGAAATGGGCTTGAAATT | |
| | TTCAAGTAAAGTAACGGTTTTAGAGTGAGACCAGCGTAACTCCTCATT | |
| Y337G | TATCTACACGGGTCTCACCAAAACCTGGAAAAAGTATTACAGCATCCAAAAATTATTAAAC | 299 |
| | AAGCGACCTTACTTGGTTTGAAAAAAACACTTCGGGAGGATGAAGAAATGGGCTTGAAATT | |
| | TTCAAGTAAAGTAACGGTTTTAGAGTGAGACCAGCGTAACTCGCGGGG | |
| Y337H | TATCTACACGGGTCTCACCAAAACCTGGAAAAAGTATTACAGCATCCAAAAATTATTAAAC | 300 |
| | AAGCGACCTTACTTCATTTGAAAAAAACACTTCGGGAGGATGAAGAAATGGGCTTGAAATT | |
| | TTCAAGTAAAGTAACGGTTTTAGAGTGAGACCAGCGTAACTCGCACCA | |
| Y337I | TATCTACACGGGTCTCACCAAAACCTGGAAAAAGTATTACAGCATCCAAAAATTATTAAAC | 301 |
| | AAGCGACCTTACTTATTTTGAAAAAAACACTTCGGGAGGATGAAGAAATGGGCTTGAAATT | |
| | TTCAAGTAAAGTAACGGTTTTAGAGTGAGACCAGCGTAACTCCTAATT | |
| Y337L | TATCTACACGGGTCTCACCAAAACCTGGAAAAAGTATTACAGCATCCAAAAATTATTAAAC | 302 |
| | AAGCGACCTTACTTTTGTTGAAAAAAACACTTCGGGAGGATGAAGAAATGGGCTTGAAATT | |
| | TTCAAGTAAAGTAACGGTTTTAGAGTGAGACCAGCGTAACTCGCGTAG | |
| Y337K | TATCTACACGGGTCTCACCAAAACCTGGAAAAAGTATTACAGCATCCAAAAATTATTAAAC | 303 |
| | AAGCGACCTTACTTAAATTGAAAAAAACACTTCGGGAGGATGAAGAAATGGGCTTGAAATT | |
| | TTCAAGTAAAGTAACGGTTTTAGAGTGAGACCAGCGTAACTCTGTTTG | |
| Y337M | TATCTACACGGGTCTCACCAAAACCTGGAAAAAGTATTACAGCATCCAAAAATTATTAAAC | 304 |
| | AAGCGACCTTACTTATGTTGAAAAAAACACTTCGGGAGGATGAAGAAATGGGCTTGAAATT | |
| | TTCAAGTAAAGTAACGGTTTTAGAGTGAGACCAGCGTAACTCAAGTAT | |
| Y337F | TATCTACACGGGTCTCACCAAAACCTGGAAAAAGTATTACAGCATCCAAAAATTATTAAAC | 305 |
| | AAGCGACCTTACTTTTCTTGAAAAAAACACTTCGGGAGGATGAAGAAATGGGCTTGAAATT | |
| | TTCAAGTAAAGTAACGGTTTTAGAGTGAGACCAGCGTAACTCAATAAA | |
| Y337P | TATCTACACGGGTCTCACCAAAACCTGGAAAAAGTATTACAGCATCCAAAAATTATTAAAC | 306 |
| | AAGCGACCTTACTTCCATTGAAAAAAACACTTCGGGAGGATGAAGAAATGGGCTTGAAATT | |
| | TTCAAGTAAAGTAACGGTTTTAGAGTGAGACCAGCGTAACTCTGGTTG | |
| Y337S | TATCTACACGGGTCTCACCAAAACCTGGAAAAAGTATTACAGCATCCAAAAATTATTAAAC | 307 |
| | AAGCGACCTTACTTTCTTTGAAAAAAACACTTCGGGAGGATGAAGAAATGGGCTTGAAATT | |
| | TTCAAGTAAAGTAACGGTTTTAGAGTGAGACCAGCGTAACTCATAGCT | |
| Y337T | TATCTACACGGGTCTCACCAAAACCTGGAAAAAGTATTACAGCATCCAAAAATTATTAAAC | 308 |
| | AAGCGACCTTACTTACTTTGAAAAAAACACTTCGGGAGGATGAAGAAATGGGCTTGAAATT | |
| | TTCAAGTAAAGTAACGGTTTTAGAGTGAGACCAGCGTAACTCAGCTAA | |
| Y337W | TATCTACACGGGTCTCACCAAAACCTGGAAAAAGTATTACAGCATCCAAAAATTATTAAAC | 309 |
| | AAGCGACCTTACTTTGGTTGAAAAAAACACTTCGGGAGGATGAAGAAATGGGCTTGAAATT | |
| | TTCAAGTAAAGTAACGGTTTTAGAGTGAGACCAGCGTAACTCCGTAAC | |
| Y337Y | TATCTACACGGGTCTCACCAAAACCTGGAAAAAGTATTACAGCATCCAAAAATTATTAAAC | 310 |
| | AAGCGACCTTACTTTATTTGAAAAAAACACTTCGGGAGGATGAAGAAATGGGCTTGAAATT | |
| | TTCAAGTAAAGTAACGGTTTTAGAGTGAGACCAGCGTAACTCAACAAG | |
| Y337V | TATCTACACGGGTCTCACCAAAACCTGGAAAAAGTATTACAGCATCCAAAAATTATTAAAC | 311 |
| | AAGCGACCTTACTTGTTTTGAAAAAAACACTTCGGGAGGATGAAGAAATGGGCTTGAAATT | |
| | TTCAAGTAAAGTAACGGTTTTAGAGTGAGACCAGCGTAACTCTCTGAT | |
| L338A | TATCTACACGGGTCTCACCAAAACCTGGAAAAAGTATTACAGCATCCAAAAATTATTAAAC | 312 |
| | AAGCGACCTTACTTTACGCTAAAAAAACACTTCGGGAGGATGAAGAAATGGGCTTGACTAA | |
| | ATTTTCAAGTAAAGTAACGGTTTTAGAGTGAGACCAGCGTAACTCTGG | |
| L338R | TATCTACACGGGTCTCACCAAAACCTGGAAAAAGTATTACAGCATCCAAAAATTATTAAAC | 313 |
| | AAGCGACCTTACTTTACAGAAAAAAAACACTTCGGGAGGATGAAGAAATGGGCTTGACTAA | |
| | ATTTTCAAGTAAAGTAACGGTTTTAGAGTGAGACCAGCGTAACTCGTA | |
| L338N | TATCTACACGGGTCTCACCAAAACCTGGAAAAAGTATTACAGCATCCAAAAATTATTAAAC | 314 |
| | AAGCGACCTTACTTTACAATAAAAAAACACTTCGGGAGGATGAAGAAATGGGCTTGACTAA | |
| | ATTTTCAAGTAAAGTAACGGTTTTAGAGTGAGACCAGCGTAACTCGAC | |
| L338D | TATCTACACGGGTCTCACCAAAACCTGGAAAAAGTATTACAGCATCCAAAAATTATTAAAC | 315 |
| | AAGCGACCTTACTTTACGATAAAAAAACACTTCGGGAGGATGAAGAAATGGGCTTGACTAA | |
| | ATTTTCAAGTAAAGTAACGGTTTTAGAGTGAGACCAGCGTAACTCTTA | |
| L338C | TATCTACACGGGTCTCACCAAAACCTGGAAAAAGTATTACAGCATCCAAAAATTATTAAAC | 316 |
| | AAGCGACCTTACTTTACTGTAAAAAAACACTTCGGGAGGATGAAGAAATGGGCTTGACTAA | |
| | ATTTTCAAGTAAAGTAACGGTTTTAGAGTGAGACCAGCGTAACTCTGA | |
| L338Q | TATCTACACGGGTCTCACCAAAACCTGGAAAAAGTATTACAGCATCCAAAAATTATTAAAC | 317 |
| | AAGCGACCTTACTTTACCAAAAAAAAACACTTCGGGAGGATGAAGAAATGGGCTTGACTAA | |
| | ATTTTCAAGTAAAGTAACGGTTTTAGAGTGAGACCAGCGTAACTCAAA | |
| L338E | TATCTACACGGGTCTCACCAAAACCTGGAAAAAGTATTACAGCATCCAAAAATTATTAAAC | 318 |
| | AAGCGACCTTACTTTACGAAAAAAAAACACTTCGGGAGGATGAAGAAATGGGCTTGACTAA | |
| | ATTTTCAAGTAAAGTAACGGTTTTAGAGTGAGACCAGCGTAACTCCGT | |
| L338G | TATCTACACGGGTCTCACCAAAACCTGGAAAAAGTATTACAGCATCCAAAAATTATTAAAC | 319 |
| | AAGCGACCTTACTTTACGGTAAAAAAACACTTCGGGAGGATGAAGAAATGGGCTTGACTAA | |
| | ATTTTCAAGTAAAGTAACGGTTTTAGAGTGAGACCAGCGTAACTCTTC | |
| L338H | TATCTACACGGGTCTCACCAAAACCTGGAAAAAGTATTACAGCATCCAAAAATTATTAAAC | 320 |
| | AAGCGACCTTACTTTACCATAAAAAAACACTTCGGGAGGATGAAGAAATGGGCTTGACTAA | |
| | ATTTTCAAGTAAAGTAACGGTTTTAGAGTGAGACCAGCGTAACTCAGC | |
| L338I | TATCTACACGGGTCTCACCAAAACCTGGAAAAAGTATTACAGCATCCAAAAATTATTAAAC | 321 |
| | AAGCGACCTTACTTTACATTAAAAAAACACTTCGGGAGGATGAAGAAATGGGCTTGACTAA | |
| | ATTTTCAAGTAAAGTAACGGTTTTAGAGTGAGACCAGCGTAACTCGTG | |
| L338L | TATCTACACGGGTCTCACCAAAACCTGGAAAAAGTATTACAGCATCCAAAAATTATTAAAC | 322 |
| | AAGCGACCTTACTTTACTTGAAAAAAACACTTCGGGAGGATGAAGAAATGGGCTTGACTAA | |
| | ATTTTCAAGTAAAGTAACGGTTTTAGAGTGAGACCAGCGTAACTCCAC | |
| L338K | TATCTACACGGGTCTCACCAAAACCTGGAAAAAGTATTACAGCATCCAAAAATTATTAAAC | 323 |
| | AAGCGACCTTACTTTACAAAAAAAAAACACTTCGGGAGGATGAAGAAATGGGCTTGACTAA | |
| | ATTTTCAAGTAAAGTAACGGTTTTAGAGTGAGACCAGCGTAACTCATC | |
| L338M | TATCTACACGGGTCTCACCAAAACCTGGAAAAAGTATTACAGCATCCAAAAATTATTAAAC | 324 |
| | AAGCGACCTTACTTTACATGAAAAAAACACTTCGGGAGGATGAAGAAATGGGCTTGACTAA | |
| | ATTTTCAAGTAAAGTAACGGTTTTAGAGTGAGACCAGCGTAACTCCAA | |
| L338F | TATCTACACGGGTCTCACCAAAACCTGGAAAAAGTATTACAGCATCCAAAAATTATTAAAC | 325 |
| | AAGCGACCTTACTTTACTTTAAAAAAACACTTCGGGAGGATGAAGAAATGGGCTTGACTAA | |
| | ATTTTCAAGTAAAGTAACGGTTTTAGAGTGAGACCAGCGTAACTCACC | |
| L338P | TATCTACACGGGTCTCACCAAAACCTGGAAAAAGTATTACAGCATCCAAAAATTATTAAAC | 326 |
| | AAGCGACCTTACTTTACCCAAAAAAAACACTTCGGGAGGATGAAGAAATGGGCTTGACTAA | |
| | ATTTTCAAGTAAAGTAACGGTTTTAGAGTGAGACCAGCGTAACTCATT | |
| L338S | TATCTACACGGGTCTCACCAAAACCTGGAAAAAGTATTACAGCATCCAAAAATTATTAAAC | 327 |
| | AAGCGACCTTACTTTACTCTAAAAAAACACTTCGGGAGGATGAAGAAATGGGCTTGACTAA | |
| | ATTTTCAAGTAAAGTAACGGTTTTAGAGTGAGACCAGCGTAACTCAGA | |
| L338T | TATCTACACGGGTCTCACCAAAACCTGGAAAAAGTATTACAGCATCCAAAAATTATTAAAC | 328 |
| | AAGCGACCTTACTTTACACTAAAAAAACACTTCGGGAGGATGAAGAAATGGGCTTGACTAA | |
| | ATTTTCAAGTAAAGTAACGGTTTTAGAGTGAGACCAGCGTAACTCGTC | |
| L338W | TATCTACACGGGTCTCACCAAAACCTGGAAAAAGTATTACAGCATCCAAAAATTATTAAAC | 329 |
| | AAGCGACCTTACTTTACTGGAAAAAAACACTTCGGGAGGATGAAGAAATGGGCTTGACTAA | |
| | ATTTTCAAGTAAAGTAACGGTTTTAGAGTGAGACCAGCGTAACTCGAC | |
| L338Y | TATCTACACGGGTCTCACCAAAACCTGGAAAAAGTATTACAGCATCCAAAAATTATTAAAC | 330 |
| | AAGCGACCTTACTTTACTATAAAAAAACACTTCGGGAGGATGAAGAAATGGGCTTGACTAA | |
| | ATTTTCAAGTAAAGTAACGGTTTTAGAGTGAGACCAGCGTAACTCAAA | |
| L338V | TATCTACACGGGTCTCACCAAAACCTGGAAAAAGTATTACAGCATCCAAAAATTATTAAAC | 331 |
| | AAGCGACCTTACTTTACGTTAAAAAAACACTTCGGGAGGATGAAGAAATGGGCTTGACTAA | |
| | ATTTTCAAGTAAAGTAACGGTTTTAGAGTGAGACCAGCGTAACTCCGA | |
| K339A | TATCTACACGGGTCTCACCAAAACGCATCCAAAAATTATTAAACAAGCCACGTTACTTTAC | 332 |
| | TTGGCTAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCT | |
| K339R | TATCTACACGGGTCTCACCAAAACGCATCCAAAAATTATTAAACAAGCCACGTTACTTTAC | 333 |
| | TTGAGAAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCT | |
| K339N | TATCTACACGGGTCTCACCAAAACGCATCCAAAAATTATTAAACAAGCCACGTTACTTTAC | 334 |
| | TTGAATAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCT | |
| K339D | TATCTACACGGGTCTCACCAAAACGCATCCAAAAATTATTAAACAAGCCACGTTACTTTAC | 335 |
| | TTGGATAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCC | |
| K339C | TATCTACACGGGTCTCACCAAAACGCATCCAAAAATTATTAAACAAGCCACGTTACTTTAC | 336 |
| | TTGTGTAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCG | |
| K339Q | TATCTACACGGGTCTCACCAAAACGCATCCAAAAATTATTAAACAAGCCACGTTACTTTAC | 337 |
| | TTGCAAAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCT | |
| K339E | TATCTACACGGGTCTCACCAAAACGCATCCAAAAATTATTAAACAAGCCACGTTACTTTAC | 338 |
| | TTGGAAAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCG | |
| K339G | TATCTACACGGGTCTCACCAAAACGCATCCAAAAATTATTAAACAAGCCACGTTACTTTAC | 339 |
| | TTGGGTAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCA | |
| K339H | TATCTACACGGGTCTCACCAAAACGCATCCAAAAATTATTAAACAAGCCACGTTACTTTAC | 340 |
| | TTGCATAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCT | |
| K339I | TATCTACACGGGTCTCACCAAAACGCATCCAAAAATTATTAAACAAGCCACGTTACTTTAC | 341 |
| | TTGATTAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCG | |
| K339L | TATCTACACGGGTCTCACCAAAACGCATCCAAAAATTATTAAACAAGCCACGTTACTTTAC | 342 |
| | TTGTTGAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCC | |
| K339K | TATCTACACGGGTCTCACCAAAACGCATCCAAAAATTATTAAACAAGCCACGTTACTTTAC | 343 |
| | TTGAAAAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCT | |
| K339M | TATCTACACGGGTCTCACCAAAACGCATCCAAAAATTATTAAACAAGCCACGTTACTTTAC | 344 |
| | TTGATGAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCA | |
| K339F | TATCTACACGGGTCTCACCAAAACGCATCCAAAAATTATTAAACAAGCCACGTTACTTTAC | 345 |
| | TTGTTTAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCC | |
| K339P | TATCTACACGGGTCTCACCAAAACGCATCCAAAAATTATTAAACAAGCCACGTTACTTTAC | 346 |
| | TTGCCAAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCG | |
| K339S | TATCTACACGGGTCTCACCAAAACGCATCCAAAAATTATTAAACAAGCCACGTTACTTTAC | 347 |
| | TTGTCTAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCC | |
| K339T | TATCTACACGGGTCTCACCAAAACGCATCCAAAAATTATTAAACAAGCCACGTTACTTTAC | 348 |
| | TTGACTAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCA | |
| K339W | TATCTACACGGGTCTCACCAAAACGCATCCAAAAATTATTAAACAAGCCACGTTACTTTAC | 349 |
| | TTGTGGAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCT | |
| K339Y | TATCTACACGGGTCTCACCAAAACGCATCCAAAAATTATTAAACAAGCCACGTTACTTTAC | 350 |
| | TTGTATAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCC | |
| K339V | TATCTACACGGGTCTCACCAAAACGCATCCAAAAATTATTAAACAAGCCACGTTACTTTAC | 351 |
| | TTGGTTAAAACACTTAGAGAAGACGAAGAAATGGGCTTGACTACCACATCTACTATCATGA | |
| | GCTTGAAAAAAACACTTCGGGGTTTTAGAGTGAGACCAGCGTAACTCT | |
| K340A | TATCTACACGGGTCTCACCAAAACTCCAAAAATTATTAAACAAGCCACGTTACTTTACTTG | 352 |
| | AAAGCTACACTTAGAGAAGACGAAGAAATGGGCTTGACTACCACATCTACTATCATGAGCT | |
| | TGAAAAAAACACTTCGGGGTTTTAGAGTGAGACCAGCGTAACTCAAAA | |
| K340R | TATCTACACGGGTCTCACCAAAACTCCAAAAATTATTAAACAAGCCACGTTACTTTACTTG | 353 |
| | AAAAGAACACTTAGAGAAGACGAAGAAATGGGCTTGACTACCACATCTACTATCATGAGCT | |
| | TGAAAAAAACACTTCGGGGTTTTAGAGTGAGACCAGCGTAACTCACGT | |
| K340N | TATCTACACGGGTCTCACCAAAACTCCAAAAATTATTAAACAAGCCACGTTACTTTACTTG | 354 |
| | AAAAATACACTTAGAGAAGACGAAGAAATGGGCTTGACTACCACATCTACTATCATGAGCT | |
| | TGAAAAAAACACTTCGGGGTTTTAGAGTGAGACCAGCGTAACTCCAAG | |
| K340D | TATCTACACGGGTCTCACCAAAACTCCAAAAATTATTAAACAAGCCACGTTACTTTACTTG | 355 |
| | AAAGATACACTTAGAGAAGACGAAGAAATGGGCTTGACTACCACATCTACTATCATGAGCT | |
| | TGAAAAAAACACTTCGGGGTTTTAGAGTGAGACCAGCGTAACTCTGCA | |
| K340C | TATCTACACGGGTCTCACCAAAACTCCAAAAATTATTAAACAAGCCACGTTACTTTACTTG | 356 |
| | AAATGTACACTTAGAGAAGACGAAGAAATGGGCTTGACTACCACATCTACTATCATGAGCT | |
| | TGAAAAAAACACTTCGGGGTTTTAGAGTGAGACCAGCGTAACTCAAAG | |
| K340Q | TATCTACACGGGTCTCACCAAAACTCCAAAAATTATTAAACAAGCCACGTTACTTTACTTG | 357 |
| | AAACAAACACTTAGAGAAGACGAAGAAATGGGCTTGACTACCACATCTACTATCATGAGCT | |
| | TGAAAAAAACACTTCGGGGTTTTAGAGTGAGACCAGCGTAACTCAGTA | |
| K340E | TATCTACACGGGTCTCACCAAAACTCCAAAAATTATTAAACAAGCCACGTTACTTTACTTG | 358 |
| | AAAGAAACACTTAGAGAAGACGAAGAAATGGGCTTGACTACCACATCTACTATCATGAGCT | |
| | TGAAAAAAACACTTCGGGGTTTTAGAGTGAGACCAGCGTAACTCTCTC | |
| K340G | TATCTACACGGGTCTCACCAAAACTCCAAAAATTATTAAACAAGCCACGTTACTTTACTTG | 359 |
| | AAAGGTACACTTAGAGAAGACGAAGAAATGGGCTTGACTACCACATCTACTATCATGAGCT | |
| | TGAAAAAAACACTTCGGGGTTTTAGAGTGAGACCAGCGTAACTCGCTA | |
| K340H | TATCTACACGGGTCTCACCAAAACTCCAAAAATTATTAAACAAGCCACGTTACTTTACTTG | 360 |
| | AAACATACACTTAGAGAAGACGAAGAAATGGGCTTGACTACCACATCTACTATCATGAGCT | |
| | TGAAAAAAACACTTCGGGGTTTTAGAGTGAGACCAGCGTAACTCAAAT | |
| K340I | TATCTACACGGGTCTCACCAAAACTCCAAAAATTATTAAACAAGCCACGTTACTTTACTTG | 361 |
| | AAAATTACACTTAGAGAAGACGAAGAAATGGGCTTGACTACCACATCTACTATCATGAGCT | |
| | TGAAAAAAACACTTCGGGGTTTTAGAGTGAGACCAGCGTAACTCCTGT | |
| K340L | TATCTACACGGGTCTCACCAAAACTCCAAAAATTATTAAACAAGCCACGTTACTTTACTTG | 362 |
| | AAATTGACACTTAGAGAAGACGAAGAAATGGGCTTGACTACCACATCTACTATCATGAGCT | |
| | TGAAAAAAACACTTCGGGGTTTTAGAGTGAGACCAGCGTAACTCGGAT | |
| K340K | TATCTACACGGGTCTCACCAAAACTCCAAAAATTATTAAACAAGCCACGTTACTTTACTTG | 363 |
| | AAAAAAACACTTAGAGAAGACGAAGAAATGGGCTTGACTACCACATCTACTATCATGAGCT | |
| | TGAAAAAAACACTTCGGGGTTTTAGAGTGAGACCAGCGTAACTCTTAT | |
| K340M | TATCTACACGGGTCTCACCAAAACTCCAAAAATTATTAAACAAGCCACGTTACTTTACTTG | 364 |
| | AAAATGACACTTAGAGAAGACGAAGAAATGGGCTTGACTACCACATCTACTATCATGAGCT | |
| | TGAAAAAAACACTTCGGGGTTTTAGAGTGAGACCAGCGTAACTCTACA | |
| K340F | TATCTACACGGGTCTCACCAAAACTCCAAAAATTATTAAACAAGCCACGTTACTTTACTTG | 365 |
| | AAATTTACACTTAGAGAAGACGAAGAAATGGGCTTGACTACCACATCTACTATCATGAGCT | |
| | TGAAAAAAACACTTCGGGGTTTTAGAGTGAGACCAGCGTAACTCCCAT | |
| K340P | TATCTACACGGGTCTCACCAAAACTCCAAAAATTATTAAACAAGCCACGTTACTTTACTTG | 366 |
| | AAACCAACACTTAGAGAAGACGAAGAAATGGGCTTGACTACCACATCTACTATCATGAGCT | |
| | TGAAAAAAACACTTCGGGGTTTTAGAGTGAGACCAGCGTAACTCTTCT | |
| K340S | TATCTACACGGGTCTCACCAAAACTCCAAAAATTATTAAACAAGCCACGTTACTTTACTTG | 367 |
| | AAATCTACACTTAGAGAAGACGAAGAAATGGGCTTGACTACCACATCTACTATCATGAGCT | |
| | TGAAAAAAACACTTCGGGGTTTTAGAGTGAGACCAGCGTAACTCTTCG | |
| K340T | TATCTACACGGGTCTCACCAAAACTCCAAAAATTATTAAACAAGCCACGTTACTTTACTTG | 368 |
| | AAAACTACACTTAGAGAAGACGAAGAAATGGGCTTGACTACCACATCTACTATCATGAGCT | |
| | TGAAAAAAACACTTCGGGGTTTTAGAGTGAGACCAGCGTAACTCTAGC | |
| K340W | TATCTACACGGGTCTCACCAAAACTCCAAAAATTATTAAACAAGCCACGTTACTTTACTTG | 369 |
| | AAATGGACACTTAGAGAAGACGAAGAAATGGGCTTGACTACCACATCTACTATCATGAGCT | |
| | TGAAAAAAACACTTCGGGGTTTTAGAGTGAGACCAGCGTAACTCGGAT | |
| K340Y | TATCTACACGGGTCTCACCAAAACTCCAAAAATTATTAAACAAGCCACGTTACTTTACTTG | 370 |
| | AAATATACACTTAGAGAAGACGAAGAAATGGGCTTGACTACCACATCTACTATCATGAGCT | |
| | TGAAAAAAACACTTCGGGGTTTTAGAGTGAGACCAGCGTAACTCATAT | |
| K340V | TATCTACACGGGTCTCACCAAAACTCCAAAAATTATTAAACAAGCCACGTTACTTTACTTG | 371 |
| | AAAGTTACACTTAGAGAAGACGAAGAAATGGGCTTGACTACCACATCTACTATCATGAGCT | |
| | TGAAAAAAACACTTCGGGGTTTTAGAGTGAGACCAGCGTAACTCTAAA | |
| T341A | TATCTACACGGGTCTCACCAAAACAAAAATTATTAAACAAGCCACGTTACTTTACTTGAAA | 372 |
| | AAAGCTCTTAGAGAAGACGAAGAAATGGGCTTGACTACCACATCTACTATCATGAGCTTGA | |
| | AAAAAACACTTCGGGGTTTTAGAGTGAGACCAGCGTAACTCCTTATTT | |
| T341R | TATCTACACGGGTCTCACCAAAACAAAAATTATTAAACAAGCCACGTTACTTTACTTGAAA | 373 |
| | AAAAGACTTAGAGAAGACGAAGAAATGGGCTTGACTACCACATCTACTATCATGAGCTTGA | |
| | AAAAAACACTTCGGGGTTTTAGAGTGAGACCAGCGTAACTCTCCACGC | |
| T341N | TATCTACACGGGTCTCACCAAAACAAAAATTATTAAACAAGCCACGTTACTTTACTTGAAA | 374 |
| | AAAAATCTTAGAGAAGACGAAGAAATGGGCTTGACTACCACATCTACTATCATGAGCTTGA | |
| | AAAAAACACTTCGGGGTTTTAGAGTGAGACCAGCGTAACTCATTTGCG | |
| T341D | TATCTACACGGGTCTCACCAAAACAAAAATTATTAAACAAGCCACGTTACTTTACTTGAAA | 375 |
| | AAAGATCTTAGAGAAGACGAAGAAATGGGCTTGACTACCACATCTACTATCATGAGCTTGA | |
| | AAAAAACACTTCGGGGTTTTAGAGTGAGACCAGCGTAACTCTCAGCCT | |
| T341C | TATCTACACGGGTCTCACCAAAACAAAAATTATTAAACAAGCCACGTTACTTTACTTGAAA | 376 |
| | AAATGTCTTAGAGAAGACGAAGAAATGGGCTTGACTACCACATCTACTATCATGAGCTTGA | |
| | AAAAAACACTTCGGGGTTTTAGAGTGAGACCAGCGTAACTCTCCAGTG | |
| T341Q | TATCTACACGGGTCTCACCAAAACAAAAATTATTAAACAAGCCACGTTACTTTACTTGAAA | 377 |
| | AAACAACTTAGAGAAGACGAAGAAATGGGCTTGACTACCACATCTACTATCATGAGCTTGA | |
| | AAAAAACACTTCGGGGTTTTAGAGTGAGACCAGCGTAACTCAAGCTTT | |
| T341E | TATCTACACGGGTCTCACCAAAACAAAAATTATTAAACAAGCCACGTTACTTTACTTGAAA | 378 |
| | AAAGAACTTAGAGAAGACGAAGAAATGGGCTTGACTACCACATCTACTATCATGAGCTTGA | |
| | AAAAAACACTTCGGGGTTTTAGAGTGAGACCAGCGTAACTCATGTATC | |
| T341G | TATCTACACGGGTCTCACCAAAACAAAAATTATTAAACAAGCCACGTTACTTTACTTGAAA | 379 |
| | AAAGGTCTTAGAGAAGACGAAGAAATGGGCTTGACTACCACATCTACTATCATGAGCTTGA | |
| | AAAAAACACTTCGGGGTTTTAGAGTGAGACCAGCGTAACTCATGCTGG | |
| T341H | TATCTACACGGGTCTCACCAAAACAAAAATTATTAAACAAGCCACGTTACTTTACTTGAAA | 380 |
| | AAACATCTTAGAGAAGACGAAGAAATGGGCTTGACTACCACATCTACTATCATGAGCTTGA | |
| | AAAAAACACTTCGGGGTTTTAGAGTGAGACCAGCGTAACTCCTCGCGG | |
| T341I | TATCTACACGGGTCTCACCAAAACAAAAATTATTAAACAAGCCACGTTACTTTACTTGAAA | 381 |
| | AAAATTCTTAGAGAAGACGAAGAAATGGGCTTGACTACCACATCTACTATCATGAGCTTGA | |
| | AAAAAACACTTCGGGGTTTTAGAGTGAGACCAGCGTAACTCCTTCCGC | |
| T341L | TATCTACACGGGTCTCACCAAAACAAAAATTATTAAACAAGCCACGTTACTTTACTTGAAA | 382 |
| | AAATTGCTTAGAGAAGACGAAGAAATGGGCTTGACTACCACATCTACTATCATGAGCTTGA | |
| | AAAAAACACTTCGGGGTTTTAGAGTGAGACCAGCGTAACTCACGAACT | |
| T341K | TATCTACACGGGTCTCACCAAAACAAAAATTATTAAACAAGCCACGTTACTTTACTTGAAA | 383 |
| | AAAAAACTTAGAGAAGACGAAGAAATGGGCTTGACTACCACATCTACTATCATGAGCTTGA | |
| | AAAAAACACTTCGGGGTTTTAGAGTGAGACCAGCGTAACTCCCTCTTT | |
| T341M | TATCTACACGGGTCTCACCAAAACAAAAATTATTAAACAAGCCACGTTACTTTACTTGAAA | 384 |
| | AAAATGCTTAGAGAAGACGAAGAAATGGGCTTGACTACCACATCTACTATCATGAGCTTGA | |
| | AAAAAACACTTCGGGGTTTTAGAGTGAGACCAGCGTAACTCCCTAATC | |
| T341F | TATCTACACGGGTCTCACCAAAACAAAAATTATTAAACAAGCCACGTTACTTTACTTGAAA | 385 |
| | AAATTTCTTAGAGAAGACGAAGAAATGGGCTTGACTACCACATCTACTATCATGAGCTTGA | |
| | AAAAAACACTTCGGGGTTTTAGAGTGAGACCAGCGTAACTCTCGCCCC | |
| T341P | TATCTACACGGGTCTCACCAAAACAAAAATTATTAAACAAGCCACGTTACTTTACTTGAAA | 386 |
| | AAACCACTTAGAGAAGACGAAGAAATGGGCTTGACTACCACATCTACTATCATGAGCTTGA | |
| | AAAAAACACTTCGGGGTTTTAGAGTGAGACCAGCGTAACTCTATACGA | |
| T341s | TATCTACACGGGTCTCACCAAAACAAAAATTATTAAACAAGCCACGTTACTTTACTTGAAA | 387 |
| | AAATCTCTTAGAGAAGACGAAGAAATGGGCTTGACTACCACATCTACTATCATGAGCTTGA | |
| | AAAAAACACTTCGGGGTTTTAGAGTGAGACCAGCGTAACTCGTTCAGG | |
| T31T | TATCTACACGGGTCTCACCAAAACAAAAATTATTAAACAAGCCACGTTACTTTACTTGAAA | 388 |
| | AAAACTCTTAGAGAAGACGAAGAAATGGGCTTGACTACCACATCTACTATCATGAGCTTGA | |
| | AAAAAACACTTCGGGGTTTTAGAGTGAGACCAGCGTAACTCGTTTACA | |
| T341W | TATCTACACGGGTCTCACCAAAACAAAAATTATTAAACAAGCCACGTTACTTTACTTGAAA | 389 |
| | AAATGGCTTAGAGAAGACGAAGAAATGGGCTTGACTACCACATCTACTATCATGAGCTTGA | |
| | AAAAAACACTTCGGGGTTTTAGAGTGAGACCAGCGTAACTCTCCCGGC | |
| T341Y | TATCTACACGGGTCTCACCAAAACAAAAATTATTAAACAAGCCACGTTACTTTACTTGAAA | 390 |
| | AAATATCTTAGAGAAGACGAAGAAATGGGCTTGACTACCACATCTACTATCATGAGCTTGA | |
| | AAAAAACACTTCGGGGTTTTAGAGTGAGACCAGCGTAACTCGGATTGT | |
| T341V | TATCTACACGGGTCTCACCAAAACAAAAATTATTAAACAAGCCACGTTACTTTACTTGAAA | 391 |
| | AAAGTTCTTAGAGAAGACGAAGAAATGGGCTTGACTACCACATCTACTATCATGAGCTTGA | |
| | AAAAAACACTTCGGGGTTTTAGAGTGAGACCAGCGTAACTCGCGTCCG | |
| L342A | TATCTACACGGGTCTCACCAAAACAATTATTAAACAAGCCACGTTACTTTACTTGAAAAAA | 392 |
| | ACAGCTAGAGAAGACGAAGAAATGGGCTTGACTACCACATCTACTATCATGAGCTTGAAAA | |
| | AAACACTTCGGGGTTTTAGAGTGAGACCAGCGTAACTCTCCGCATGTC | |
| L342R | TATCTACACGGGTCTCACCAAAACAATTATTAAACAAGCCACGTTACTTTACTTGAAAAAA | 393 |
| | ACAAGAAGAGAAGACGAAGAAATGGGCTTGACTACCACATCTACTATCATGAGCTTGAAAA | |
| | AAACACTTCGGGGTTTTAGAGTGAGACCAGCGTAACTCCTATTCTCCG | |
| L342N | TATCTACACGGGTCTCACCAAAACAATTATTAAACAAGCCACGTTACTTTACTTGAAAAAA | 394 |
| | ACAAATAGAGAAGACGAAGAAATGGGCTTGACTACCACATCTACTATCATGAGCTTGAAAA | |
| | AAACACTTCGGGGTTTTAGAGTGAGACCAGCGTAACTCGGATGGGCCG | |
| L342D | TATCTACACGGGTCTCACCAAAACAATTATTAAACAAGCCACGTTACTTTACTTGAAAAAA | 395 |
| | ACAGATAGAGAAGACGAAGAAATGGGCTTGACTACCACATCTACTATCATGAGCTTGAAAA | |
| | AAACACTTCGGGGTTTTAGAGTGAGACCAGCGTAACTCGTTTCTCTAA | |
| L342C | TATCTACACGGGTCTCACCAAAACAATTATTAAACAAGCCACGTTACTTTACTTGAAAAAA | 396 |
| | ACATGTAGAGAAGACGAAGAAATGGGCTTGACTACCACATCTACTATCATGAGCTTGAAAA | |
| | AAACACTTCGGGGTTTTAGAGTGAGACCAGCGTAACTCACTTTTGGCG | |
| L342Q | TATCTACACGGGTCTCACCAAAACAATTATTAAACAAGCCACGTTACTTTACTTGAAAAAA | 397 |
| | ACACAAAGAGAAGACGAAGAAATGGGCTTGACTACCACATCTACTATCATGAGCTTGAAAA | |
| | AAACACTTCGGGGTTTTAGAGTGAGACCAGCGTAACTCTTGAGCTGGT | |
| L342E | TATCTACACGGGTCTCACCAAAACAATTATTAAACAAGCCACGTTACTTTACTTGAAAAAA | 398 |
| | ACAGAAAGAGAAGACGAAGAAATGGGCTTGACTACCACATCTACTATCATGAGCTTGAAAA | |
| | AAACACTTCGGGGTTTTAGAGTGAGACCAGCGTAACTCCGAGGTTATT | |
| L342G | TATCTACACGGGTCTCACCAAAACAATTATTAAACAAGCCACGTTACTTTACTTGAAAAAA | 399 |
| | ACAGGTAGAGAAGACGAAGAAATGGGCTTGACTACCACATCTACTATCATGAGCTTGAAAA | |
| | AAACACTTCGGGGTTTTAGAGTGAGACCAGCGTAACTCTAGGGGGTGT | |
| L342H | TATCTACACGGGTCTCACCAAAACAATTATTAAACAAGCCACGTTACTTTACTTGAAAAAA | 400 |
| | ACACATAGAGAAGACGAAGAAATGGGCTTGACTACCACATCTACTATCATGAGCTTGAAAA | |
| | AAACACTTCGGGGTTTTAGAGTGAGACCAGCGTAACTCTCCAACGTTC | |
| L342I | TATCTACACGGGTCTCACCAAAACAATTATTAAACAAGCCACGTTACTTTACTTGAAAAAA | 401 |
| | ACAATTAGAGAAGACGAAGAAATGGGCTTGACTACCACATCTACTATCATGAGCTTGAAAA | |
| | AAACACTTCGGGGTTTTAGAGTGAGACCAGCGTAACTCGTGAACACGG | |
| L342L | TATCTACACGGGTCTCACCAAAACAATTATTAAACAAGCCACGTTACTTTACTTGAAAAAA | 402 |
| | ACATTGAGAGAAGACGAAGAAATGGGCTTGACTACCACATCTACTATCATGAGCTTGAAAA | |
| | AAACACTTCGGGGTTTTAGAGTGAGACCAGCGTAACTCTCTAAAAGAT | |
| L342K | TATCTACACGGGTCTCACCAAAACAATTATTAAACAAGCCACGTTACTTTACTTGAAAAAA | 403 |
| | ACAAAAAGAGAAGACGAAGAAATGGGCTTGACTACCACATCTACTATCATGAGCTTGAAAA | |
| | AAACACTTCGGGGTTTTAGAGTGAGACCAGCGTAACTCGCCTCCGAGC | |
| L342M | TATCTACACGGGTCTCACCAAAACAATTATTAAACAAGCCACGTTACTTTACTTGAAAAAA | 404 |
| | ACAATGAGAGAAGACGAAGAAATGGGCTTGACTACCACATCTACTATCATGAGCTTGAAAA | |
| | AAACACTTCGGGGTTTTAGAGTGAGACCAGCGTAACTCCCTAAGGCGC | |
| L342F | TATCTACACGGGTCTCACCAAAACAATTATTAAACAAGCCACGTTACTTTACTTGAAAAAA | 405 |
| | ACATTTAGAGAAGACGAAGAAATGGGCTTGACTACCACATCTACTATCATGAGCTTGAAAA | |
| | AAACACTTCGGGGTTTTAGAGTGAGACCAGCGTAACTCGTCAACTGAC | |
| L342P | TATCTACACGGGTCTCACCAAAACAATTATTAAACAAGCCACGTTACTTTACTTGAAAAAA | 406 |
| | ACACCAAGAGAAGACGAAGAAATGGGCTTGACTACCACATCTACTATCATGAGCTTGAAAA | |
| | AAACACTTCGGGGTTTTAGAGTGAGACCAGCGTAACTCGGTATATCCC | |
| L342S | TATCTACACGGGTCTCACCAAAACAATTATTAAACAAGCCACGTTACTTTACTTGAAAAAA | 407 |
| | ACATCTAGAGAAGACGAAGAAATGGGCTTGACTACCACATCTACTATCATGAGCTTGAAAA | |
| | AAACACTTCGGGGTTTTAGAGTGAGACCAGCGTAACTCCCGTTGTGTC | |
| L342T | TATCTACACGGGTCTCACCAAAACAATTATTAAACAAGCCACGTTACTTTACTTGAAAAAA | 408 |
| | ACAACTAGAGAAGACGAAGAAATGGGCTTGACTACCACATCTACTATCATGAGCTTGAAAA | |
| | AAACACTTCGGGGTTTTAGAGTGAGACCAGCGTAACTCGGACCTTAAC | |
| L342W | TATCTACACGGGTCTCACCAAAACAATTATTAAACAAGCCACGTTACTTTACTTGAAAAAA | 409 |
| | ACATGGAGAGAAGACGAAGAAATGGGCTTGACTACCACATCTACTATCATGAGCTTGAAAA | |
| | AAACACTTCGGGGTTTTAGAGTGAGACCAGCGTAACTCTTATGCCTGC | |
| L342Y | TATCTACACGGGTCTCACCAAAACAATTATTAAACAAGCCACGTTACTTTACTTGAAAAAA | 410 |
| | ACATATAGAGAAGACGAAGAAATGGGCTTGACTACCACATCTACTATCATGAGCTTGAAAA | |
| | AAACACTTCGGGGTTTTAGAGTGAGACCAGCGTAACTCAGCGAGATAG | |
| L342V | TATCTACACGGGTCTCACCAAAACAATTATTAAACAAGCCACGTTACTTTACTTGAAAAAA | 411 |
| | ACAGTTAGAGAAGACGAAGAAATGGGCTTGACTACCACATCTACTATCATGAGCTTGAAAA | |
| | AAACACTTCGGGGTTTTAGAGTGAGACCAGCGTAACTCCTTCGATGGA | |
| R343A | TATCTACACGGGTCTCACCAAAACTATTAAACAAGCCACGTTACTTTACTTGAAAAAAACA | 412 |
| | CTTGCTGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCC | |
| R343R | TATCTACACGGGTCTCACCAAAACTATTAAACAAGCCACGTTACTTTACTTGAAAAAAACA | 413 |
| | CTTAGAGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCT | |
| R343N | TATCTACACGGGTCTCACCAAAACTATTAAACAAGCCACGTTACTTTACTTGAAAAAAACA | 414 |
| | CTTAATGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCC | |
| R343D | TATCTACACGGGTCTCACCAAAACTATTAAACAAGCCACGTTACTTTACTTGAAAAAAACA | 415 |
| | CTTGATGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCC | |
| R343C | TATCTACACGGGTCTCACCAAAACTATTAAACAAGCCACGTTACTTTACTTGAAAAAAACA | 416 |
| | CTTTGTGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCC | |
| R343Q | TATCTACACGGGTCTCACCAAAACTATTAAACAAGCCACGTTACTTTACTTGAAAAAAACA | 417 |
| | CTTCAAGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCT | |
| R343E | TATCTACACGGGTCTCACCAAAACTATTAAACAAGCCACGTTACTTTACTTGAAAAAAACA | 418 |
| | CTTGAAGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCT | |
| R343G | TATCTACACGGGTCTCACCAAAACTATTAAACAAGCCACGTTACTTTACTTGAAAAAAACA | 419 |
| | CTTGGTGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCA | |
| R343H | TATCTACACGGGTCTCACCAAAACTATTAAACAAGCCACGTTACTTTACTTGAAAAAAACA | 420 |
| | CTTCATGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCG | |
| R343I | TATCTACACGGGTCTCACCAAAACTATTAAACAAGCCACGTTACTTTACTTGAAAAAAACA | 421 |
| | CTTATTGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCG | |
| R343L | TATCTACACGGGTCTCACCAAAACTATTAAACAAGCCACGTTACTTTACTTGAAAAAAACA | 422 |
| | CTTTTGGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCT | |
| R343K | TATCTACACGGGTCTCACCAAAACTATTAAACAAGCCACGTTACTTTACTTGAAAAAAACA | 423 |
| | CTTAAAGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCC | |
| R343M | TATCTACACGGGTCTCACCAAAACTATTAAACAAGCCACGTTACTTTACTTGAAAAAAACA | 424 |
| | CTTATGGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCG | |
| R343F | TATCTACACGGGTCTCACCAAAACTATTAAACAAGCCACGTTACTTTACTTGAAAAAAACA | 425 |
| | CTTTTCGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCG | |
| R343P | TATCTACACGGGTCTCACCAAAACTATTAAACAAGCCACGTTACTTTACTTGAAAAAAACA | 426 |
| | CTTCCAGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCA | |
| R343S | TATCTACACGGGTCTCACCAAAACTATTAAACAAGCCACGTTACTTTACTTGAAAAAAACA | 427 |
| | CTTTCTGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCA | |
| R343T | TATCTACACGGGTCTCACCAAAACTATTAAACAAGCCACGTTACTTTACTTGAAAAAAACA | 428 |
| | CTTACTGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCA | |
| R343W | TATCTACACGGGTCTCACCAAAACTATTAAACAAGCCACGTTACTTTACTTGAAAAAAACA | 429 |
| | CTTTGGGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCA | |
| R343Y | TATCTACACGGGTCTCACCAAAACTATTAAACAAGCCACGTTACTTTACTTGAAAAAAACA | 430 |
| | CTTTATGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCC | |
| R343V | TATCTACACGGGTCTCACCAAAACTATTAAACAAGCCACGTTACTTTACTTGAAAAAAACA | 431 |
| | CTTGTTGAGGATGAAGAGATGGGGTTGACTACCACATCTACTATCATGAGTCTGCAATGTC | |
| | CACTTCGGGAGGATGAAGAAAGTTTTAGAGTGAGACCAGCGTAACTCT | |
| E344A | TATCTACACGGGTCTCACCAAAACTAAACAAGCCACGTTACTTTACTTGAAAAAAACACTT | 432 |
| | CGGGCTGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTCAA | |
| E344R | TATCTACACGGGTCTCACCAAAACTAAACAAGCCACGTTACTTTACTTGAAAAAAACACTT | 433 |
| | CGGAGAGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTGTA | |
| E344N | TATCTACACGGGTCTCACCAAAACTAAACAAGCCACGTTACTTTACTTGAAAAAAACACTT | 434 |
| | CGGAATGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCGTTC | |
| E344D | TATCTACACGGGTCTCACCAAAACTAAACAAGCCACGTTACTTTACTTGAAAAAAACACTT | 435 |
| | CGGGATGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTGTG | |
| E344C | TATCTACACGGGTCTCACCAAAACTAAACAAGCCACGTTACTTTACTTGAAAAAAACACTT | 436 |
| | CGGTGTGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCGTCT | |
| E344Q | TATCTACACGGGTCTCACCAAAACTAAACAAGCCACGTTACTTTACTTGAAAAAAACACTT | 437 |
| | CGGCAAGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTCTC | |
| E344E | TATCTACACGGGTCTCACCAAAACTAAACAAGCCACGTTACTTTACTTGAAAAAAACACTT | 438 |
| | CGGGAAGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCAACG | |
| E344G | TATCTACACGGGTCTCACCAAAACTAAACAAGCCACGTTACTTTACTTGAAAAAAACACTT | 439 |
| | CGGGGTGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCAACG | |
| E344H | TATCTACACGGGTCTCACCAAAACTAAACAAGCCACGTTACTTTACTTGAAAAAAACACTT | 440 |
| | CGGCATGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCCAGA | |
| E344I | TATCTACACGGGTCTCACCAAAACTAAACAAGCCACGTTACTTTACTTGAAAAAAACACTT | 441 |
| | CGGATTGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCACCT | |
| E344L | TATCTACACGGGTCTCACCAAAACTAAACAAGCCACGTTACTTTACTTGAAAAAAACACTT | 442 |
| | CGGTTGGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCATTG | |
| E344K | TATCTACACGGGTCTCACCAAAACTAAACAAGCCACGTTACTTTACTTGAAAAAAACACTT | 443 |
| | CGGAAAGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTCCT | |
| E344M | TATCTACACGGGTCTCACCAAAACTAAACAAGCCACGTTACTTTACTTGAAAAAAACACTT | 444 |
| | CGGATGGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTCCC | |
| E344F | TATCTACACGGGTCTCACCAAAACTAAACAAGCCACGTTACTTTACTTGAAAAAAACACTT | 445 |
| | CGGTTTGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTAGG | |
| E344P | TATCTACACGGGTCTCACCAAAACTAAACAAGCCACGTTACTTTACTTGAAAAAAACACTT | 446 |
| | CGGCCAGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTTTC | |
| E344S | TATCTACACGGGTCTCACCAAAACTAAACAAGCCACGTTACTTTACTTGAAAAAAACACTT | 447 |
| | CGGTCTGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCCCTG | |
| E344T | TATCTACACGGGTCTCACCAAAACTAAACAAGCCACGTTACTTTACTTGAAAAAAACACTT | 448 |
| | CGGACTGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCGTTT | |
| E344W | TATCTACACGGGTCTCACCAAAACTAAACAAGCCACGTTACTTTACTTGAAAAAAACACTT | 449 |
| | CGGTGGGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCAGGC | |
| E344Y | TATCTACACGGGTCTCACCAAAACTAAACAAGCCACGTTACTTTACTTGAAAAAAACACTT | 450 |
| | CGGTATGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCCATA | |
| E344V | TATCTACACGGGTCTCACCAAAACTAAACAAGCCACGTTACTTTACTTGAAAAAAACACTT | 451 |
| | CGGGTTGATGAAGAGATGGGGTTGACTACCACATCTACTATCATGAGTCTGCAATGTCCAC | |
| | TTCGGGAGGATGAAGAAAGTTTTAGAGTGAGACCAGCGTAACTCTTTT | |
| D345A | TATCTACACGGGTCTCACCAAAACACAAGCCACGTTACTTTACTTGAAAAAAACACTTCGG | 452 |
| | GAGGCTGAAGAGATGGGGTTGACTACCACATCTACTATCATGAGTCTGCAATGTCCACTTC | |
| | GGGAGGATGAAGAAAGTTTTAGAGTGAGACCAGCGTAACTCGGCGCTC | |
| D345R | TATCTACACGGGTCTCACCAAAACACAAGCCACGTTACTTTACTTGAAAAAAACACTTCGG | 453 |
| | GAGAGAGAAGAGATGGGGTTGACTACCACATCTACTATCATGAGTCTGCAATGTCCACTTC | |
| | GGGAGGATGAAGAAAGTTTTAGAGTGAGACCAGCGTAACTCCCAGCTC | |
| D345N | TATCTACACGGGTCTCACCAAAACACAAGCCACGTTACTTTACTTGAAAAAAACACTTCGG | 454 |
| | GAGAATGAAGAGATGGGGTTGACTACCACATCTACTATCATGAGTCTGCAATGTCCACTTC | |
| | GGGAGGATGAAGAAAGTTTTAGAGTGAGACCAGCGTAACTCGGAAAGC | |
| D345D | TATCTACACGGGTCTCACCAAAACACAAGCCACGTTACTTTACTTGAAAAAAACACTTCGG | 455 |
| | GAGGATGAAGAGATGGGGTTGACTACCACATCTACTATCATGAGTCTGCAATGTCCACTTC | |
| | GGGAGGATGAAGAAAGTTTTAGAGTGAGACCAGCGTAACTCCACATTC | |
| D345C | TATCTACACGGGTCTCACCAAAACACAAGCCACGTTACTTTACTTGAAAAAAACACTTCGG | 456 |
| | GAGTGTGAAGAGATGGGGTTGACTACCACATCTACTATCATGAGTCTGCAATGTCCACTTC | |
| | GGGAGGATGAAGAAAGTTTTAGAGTGAGACCAGCGTAACTCGTATGCT | |
| D345Q | TATCTACACGGGTCTCACCAAAACACAAGCCACGTTACTTTACTTGAAAAAAACACTTCGG | 457 |
| | GAGCAAGAAGAGATGGGGTTGACTACCACATCTACTATCATGAGTCTGCAATGTCCACTTC | |
| | GGGAGGATGAAGAAAGTTTTAGAGTGAGACCAGCGTAACTCCACTATC | |
| D345E | TATCTACACGGGTCTCACCAAAACACAAGCCACGTTACTTTACTTGAAAAAAACACTTCGG | 458 |
| | GAGGAAGAAGAGATGGGGTTGACTACCACATCTACTATCATGAGTCTGCAATGTCCACTTC | |
| | GGGAGGATGAAGAAAGTTTTAGAGTGAGACCAGCGTAACTCTGCGTAC | |
| D345G | TATCTACACGGGTCTCACCAAAACACAAGCCACGTTACTTTACTTGAAAAAAACACTTCGG | 459 |
| | GAGGGTGAAGAGATGGGGTTGACTACCACATCTACTATCATGAGTCTGCAATGTCCACTTC | |
| | GGGAGGATGAAGAAAGTTTTAGAGTGAGACCAGCGTAACTCTGAGGAG | |
| D345H | TATCTACACGGGTCTCACCAAAACACAAGCCACGTTACTTTACTTGAAAAAAACACTTCGG | 460 |
| | GAGCATGAAGAGATGGGGTTGACTACCACATCTACTATCATGAGTCTGCAATGTCCACTTC | |
| | GGGAGGATGAAGAAAGTTTTAGAGTGAGACCAGCGTAACTCGTGGTGG | |
| D345I | TATCTACACGGGTCTCACCAAAACACAAGCCACGTTACTTTACTTGAAAAAAACACTTCGG | 461 |
| | GAGATTGAAGAGATGGGGTTGACTACCACATCTACTATCATGAGTCTGCAATGTCCACTTC | |
| | GGGAGGATGAAGAAAGTTTTAGAGTGAGACCAGCGTAACTCCGTAGTA | |
| D345L | TATCTACACGGGTCTCACCAAAACACAAGCCACGTTACTTTACTTGAAAAAAACACTTCGG | 462 |
| | GAGTTGGAAGAGATGGGGTTGACTACCACATCTACTATCATGAGTCTGCAATGTCCACTTC | |
| | GGGAGGATGAAGAAAGTTTTAGAGTGAGACCAGCGTAACTCTCGACCT | |
| D345K | TATCTACACGGGTCTCACCAAAACACAAGCCACGTTACTTTACTTGAAAAAAACACTTCGG | 463 |
| | GAGAAAGAAGAGATGGGGTTGACTACCACATCTACTATCATGAGTCTGCAATGTCCACTTC | |
| | GGGAGGATGAAGAAAGTTTTAGAGTGAGACCAGCGTAACTCGTTATGG | |
| D345M | TATCTACACGGGTCTCACCAAAACACAAGCCACGTTACTTTACTTGAAAAAAACACTTCGG | 464 |
| | GAGATGGAAGAGATGGGGTTGACTACCACATCTACTATCATGAGTCTGCAATGTCCACTTC | |
| | GGGAGGATGAAGAAAGTTTTAGAGTGAGACCAGCGTAACTCATCATGA | |
| D345F | TATCTACACGGGTCTCACCAAAACACAAGCCACGTTACTTTACTTGAAAAAAACACTTCGG | 465 |
| | GAGTTTGAAGAGATGGGGTTGACTACCACATCTACTATCATGAGTCTGCAATGTCCACTTC | |
| | GGGAGGATGAAGAAAGTTTTAGAGTGAGACCAGCGTAACTCCATTCAT | |
| D345P | TATCTACACGGGTCTCACCAAAACACAAGCCACGTTACTTTACTTGAAAAAAACACTTCGG | 466 |
| | GAGCCAGAAGAGATGGGGTTGACTACCACATCTACTATCATGAGTCTGCAATGTCCACTTC | |
| | GGGAGGATGAAGAAAGTTTTAGAGTGAGACCAGCGTAACTCACAACAG | |
| D345S | TATCTACACGGGTCTCACCAAAACACAAGCCACGTTACTTTACTTGAAAAAAACACTTCGG | 467 |
| | GAGTCTGAAGAGATGGGGTTGACTACCACATCTACTATCATGAGTCTGCAATGTCCACTTC | |
| | GGGAGGATGAAGAAAGTTTTAGAGTGAGACCAGCGTAACTCATATCAT | |
| D345T | TATCTACACGGGTCTCACCAAAACACAAGCCACGTTACTTTACTTGAAAAAAACACTTCGG | 468 |
| | GAGACTGAAGAGATGGGGTTGACTACCACATCTACTATCATGAGTCTGCAATGTCCACTTC | |
| | GGGAGGATGAAGAAAGTTTTAGAGTGAGACCAGCGTAACTCAGACTCA | |
| D345W | TATCTACACGGGTCTCACCAAAACACAAGCCACGTTACTTTACTTGAAAAAAACACTTCGG | 469 |
| | GAGTGGGAAGAGATGGGGTTGACTACCACATCTACTATCATGAGTCTGCAATGTCCACTTC | |
| | GGGAGGATGAAGAAAGTTTTAGAGTGAGACCAGCGTAACTCGCGGAGA | |
| D345Y | TATCTACACGGGTCTCACCAAAACACAAGCCACGTTACTTTACTTGAAAAAAACACTTCGG | 470 |
| | GAGTATGAAGAGATGGGGTTGACTACCACATCTACTATCATGAGTCTGCAATGTCCACTTC | |
| | GGGAGGATGAAGAAAGTTTTAGAGTGAGACCAGCGTAACTCCTGGAGG | |
| D345V | TATCTACACGGGTCTCACCAAAACACAAGCCACGTTACTTTACTTGAAAAAAACACTTCGG | 471 |
| | GAGGTTGAAGAGATGGGGTTGACTACCACATCTACTATCATGAGTCTGCAATGTCCACTTC | |
| | GGGAGGATGAAGAAAGTTTTAGAGTGAGACCAGCGTAACTCCATGACA | |
| E346A | TATCTACACGGGTCTCACCAAAACAGCCACGTTACTTTACTTGAAAAAAACACTTCGGGAG | 472 |
| | GATGCTGAGATGGGGTTGACTACCACATCTACTATCATGAGTCTGCAATGTCCACTTCGGG | |
| | AGGATGAAGAAAGTTTTAGAGTGAGACCAGCGTAACTCACCCACCGGG | |
| E346R | TATCTACACGGGTCTCACCAAAACAGCCACGTTACTTTACTTGAAAAAAACACTTCGGGAG | 473 |
| | GATAGAGAGATGGGGTTGACTACCACATCTACTATCATGAGTCTGCAATGTCCACTTCGGG | |
| | AGGATGAAGAAAGTTTTAGAGTGAGACCAGCGTAACTCGCCCATGACT | |
| E346N | TATCTACACGGGTCTCACCAAAACAGCCACGTTACTTTACTTGAAAAAAACACTTCGGGAG | 474 |
| | GATAATGAGATGGGGTTGACTACCACATCTACTATCATGAGTCTGCAATGTCCACTTCGGG | |
| | AGGATGAAGAAAGTTTTAGAGTGAGACCAGCGTAACTCCCTCCTGCGT | |
| E346D | TATCTACACGGGTCTCACCAAAACAGCCACGTTACTTTACTTGAAAAAAACACTTCGGGAG | 475 |
| | GATGATGAGATGGGGTTGACTACCACATCTACTATCATGAGTCTGCAATGTCCACTTCGGG | |
| | AGGATGAAGAAAGTTTTAGAGTGAGACCAGCGTAACTCGTCCCTATGC | |
| E346C | TATCTACACGGGTCTCACCAAAACAGCCACGTTACTTTACTTGAAAAAAACACTTCGGGAG | 476 |
| | GATTGTGAGATGGGGTTGACTACCACATCTACTATCATGAGTCTGCAATGTCCACTTCGGG | |
| | AGGATGAAGAAAGTTTTAGAGTGAGACCAGCGTAACTCTGGTAGTCTA | |
| E346Q | TATCTACACGGGTCTCACCAAAACAGCCACGTTACTTTACTTGAAAAAAACACTTCGGGAG | 477 |
| | GATCAAGAGATGGGGTTGACTACCACATCTACTATCATGAGTCTGCAATGTCCACTTCGGG | |
| | AGGATGAAGAAAGTTTTAGAGTGAGACCAGCGTAACTCTAGAAAAGTC | |
| E346E | TATCTACACGGGTCTCACCAAAACAGCCACGTTACTTTACTTGAAAAAAACACTTCGGGAG | 478 |
| | GATGAAGAGATGGGGTTGACTACCACATCTACTATCATGAGTCTGCAATGTCCACTTCGGG | |
| | AGGATGAAGAAAGTTTTAGAGTGAGACCAGCGTAACTCGTACACAGAA | |
| E346G | TATCTACACGGGTCTCACCAAAACAGCCACGTTACTTTACTTGAAAAAAACACTTCGGGAG | 479 |
| | GATGGTGAGATGGGGTTGACTACCACATCTACTATCATGAGTCTGCAATGTCCACTTCGGG | |
| | AGGATGAAGAAAGTTTTAGAGTGAGACCAGCGTAACTCGACCTCCCTG | |
| E346H | TATCTACACGGGTCTCACCAAAACAGCCACGTTACTTTACTTGAAAAAAACACTTCGGGAG | 480 |
| | GATCATGAGATGGGGTTGACTACCACATCTACTATCATGAGTCTGCAATGTCCACTTCGGG | |
| | AGGATGAAGAAAGTTTTAGAGTGAGACCAGCGTAACTCACCACGTTAT | |
| E346I | TATCTACACGGGTCTCACCAAAACAGCCACGTTACTTTACTTGAAAAAAACACTTCGGGAG | 481 |
| | GATATTGAGATGGGGTTGACTACCACATCTACTATCATGAGTCTGCAATGTCCACTTCGGG | |
| | AGGATGAAGAAAGTTTTAGAGTGAGACCAGCGTAACTCATTGCGGGCC | |
| E346L | TATCTACACGGGTCTCACCAAAACAGCCACGTTACTTTACTTGAAAAAAACACTTCGGGAG | 482 |
| | GATTTGGAGATGGGGTTGACTACCACATCTACTATCATGAGTCTGCAATGTCCACTTCGGG | |
| | AGGATGAAGAAAGTTTTAGAGTGAGACCAGCGTAACTCTATAACCGAA | |
| E346K | TATCTACACGGGTCTCACCAAAACAGCCACGTTACTTTACTTGAAAAAAACACTTCGGGAG | 483 |
| | GATAAAGAGATGGGGTTGACTACCACATCTACTATCATGAGTCTGCAATGTCCACTTCGGG | |
| | AGGATGAAGAAAGTTTTAGAGTGAGACCAGCGTAACTCATCAGGGTCC | |
| E346M | TATCTACACGGGTCTCACCAAAACAGCCACGTTACTTTACTTGAAAAAAACACTTCGGGAG | 484 |
| | GATATGGAGATGGGGTTGACTACCACATCTACTATCATGAGTCTGCAATGTCCACTTCGGG | |
| | AGGATGAAGAAAGTTTTAGAGTGAGACCAGCGTAACTCCGAGAACGTA | |
| E346F | TATCTACACGGGTCTCACCAAAACAGCCACGTTACTTTACTTGAAAAAAACACTTCGGGAG | 485 |
| | GATTTTGAGATGGGGTTGACTACCACATCTACTATCATGAGTCTGCAATGTCCACTTCGGG | |
| | AGGATGAAGAAAGTTTTAGAGTGAGACCAGCGTAACTCTCCATCATTG | |
| E346P | TATCTACACGGGTCTCACCAAAACAGCCACGTTACTTTACTTGAAAAAAACACTTCGGGAG | 486 |
| | GATCCAGAGATGGGGTTGACTACCACATCTACTATCATGAGTCTGCAATGTCCACTTCGGG | |
| | AGGATGAAGAAAGTTTTAGAGTGAGACCAGCGTAACTCCGCACGGGGT | |
| E346S | TATCTACACGGGTCTCACCAAAACAGCCACGTTACTTTACTTGAAAAAAACACTTCGGGAG | 487 |
| | GATTCTGAGATGGGGTTGACTACCACATCTACTATCATGAGTCTGCAATGTCCACTTCGGG | |
| | AGGATGAAGAAAGTTTTAGAGTGAGACCAGCGTAACTCAAGTATCAAC | |
| E346T | TATCTACACGGGTCTCACCAAAACAGCCACGTTACTTTACTTGAAAAAAACACTTCGGGAG | 488 |
| | GATACTGAGATGGGGTTGACTACCACATCTACTATCATGAGTCTGCAATGTCCACTTCGGG | |
| | AGGATGAAGAAAGTTTTAGAGTGAGACCAGCGTAACTCGGGCTTACAA | |
| E346W | TATCTACACGGGTCTCACCAAAACAGCCACGTTACTTTACTTGAAAAAAACACTTCGGGAG | 489 |
| | GATTGGGAGATGGGGTTGACTACCACATCTACTATCATGAGTCTGCAATGTCCACTTCGGG | |
| | AGGATGAAGAAAGTTTTAGAGTGAGACCAGCGTAACTCAATTTGAGTA | |
| E346Y | TATCTACACGGGTCTCACCAAAACAGCCACGTTACTTTACTTGAAAAAAACACTTCGGGAG | 490 |
| | GATTATGAGATGGGGTTGACTACCACATCTACTATCATGAGTCTGCAATGTCCACTTCGGG | |
| | AGGATGAAGAAAGTTTTAGAGTGAGACCAGCGTAACTCTCAGTACATA | |
| E346V | TATCTACACGGGTCTCACCAAAACAGCCACGTTACTTTACTTGAAAAAAACACTTCGGGAG | 491 |
| | GATGTTGAGATGGGGTTGACTACCACATCTACTATCATGAGTCTGCAATGTCCACTTCGGG | |
| | AGGATGAAGAAAGTTTTAGAGTGAGACCAGCGTAACTCCACTCAGTCT | |
| E347A | TATCTACACGGGTCTCACCAAAACCACGTTACTTTACTTGAAAAAAACACTTCGGGAGGAT | 492 |
| | GAAGCGATGGGGTTGACTACCACATCTACTATCATGAGTCTGCAATGTCCACTTCGGGAGG | |
| | ATGAAGAAAGTTTTAGAGTGAGACCAGCGTAACTCTAGTGACTATGCT | |
| E347R | TATCTACACGGGTCTCACCAAAACCACGTTACTTTACTTGAAAAAAACACTTCGGGAGGAT | 493 |
| | GAACGGATGGGGTTGACTACCACATCTACTATCATGAGTCTGCAATGTCCACTTCGGGAGG | |
| | ATGAAGAAAGTTTTAGAGTGAGACCAGCGTAACTCCGTTTCCACCGTA | |
| E347N | TATCTACACGGGTCTCACCAAAACCACGTTACTTTACTTGAAAAAAACACTTCGGGAGGAT | 494 |
| | GAAAACATGGGGTTGACTACCACATCTACTATCATGAGTCTGCAATGTCCACTTCGGGAGG | |
| | ATGAAGAAAGTTTTAGAGTGAGACCAGCGTAACTCTTACCAACAACCA | |
| E347D | TATCTACACGGGTCTCACCAAAACCACGTTACTTTACTTGAAAAAAACACTTCGGGAGGAT | 495 |
| | GAAGACATGGGGTTGACTACCACATCTACTATCATGAGTCTGCAATGTCCACTTCGGGAGG | |
| | ATGAAGAAAGTTTTAGAGTGAGACCAGCGTAACTCGTTCAGAATTAAA | |
| E347C | TATCTACACGGGTCTCACCAAAACCACGTTACTTTACTTGAAAAAAACACTTCGGGAGGAT | 496 |
| | GAATGCATGGGGTTGACTACCACATCTACTATCATGAGTCTGCAATGTCCACTTCGGGAGG | |
| | ATGAAGAAAGTTTTAGAGTGAGACCAGCGTAACTCTAGGGACATTTCA | |
| E347Q | TATCTACACGGGTCTCACCAAAACCACGTTACTTTACTTGAAAAAAACACTTCGGGAGGAT | 497 |
| | GAACAGATGGGGTTGACTACCACATCTACTATCATGAGTCTGCAATGTCCACTTCGGGAGG | |
| | ATGAAGAAAGTTTTAGAGTGAGACCAGCGTAACTCTGATGGGTGACCA | |
| E347E | TATCTACACGGGTCTCACCAAAACCACGTTACTTTACTTGAAAAAAACACTTCGGGAGGAT | 498 |
| | GAAGAGATGGGGTTGACTACCACATCTACTATCATGAGTCTGCAATGTCCACTTCGGGAGG | |
| | ATGAAGAAAGTTTTAGAGTGAGACCAGCGTAACTCTTGGTCTACCTTG | |
| E347G | TATCTACACGGGTCTCACCAAAACCACGTTACTTTACTTGAAAAAAACACTTCGGGAGGAT | 499 |
| | GAAGGGATGGGGTTGACTACCACATCTACTATCATGAGTCTGCAATGTCCACTTCGGGAGG | |
| | ATGAAGAAAGTTTTAGAGTGAGACCAGCGTAACTCCTGTATGCTTTGC | |
| E347H | TATCTACACGGGTCTCACCAAAACCACGTTACTTTACTTGAAAAAAACACTTCGGGAGGAT | 500 |
| | GAACACATGGGGTTGACTACCACATCTACTATCATGAGTCTGCAATGTCCACTTCGGGAGG | |
| | ATGAAGAAAGTTTTAGAGTGAGACCAGCGTAACTCTTTTTCCTCGACT | |
| E347I | TATCTACACGGGTCTCACCAAAACCACGTTACTTTACTTGAAAAAAACACTTCGGGAGGAT | 501 |
| | GAAATCATGGGGTTGACTACCACATCTACTATCATGAGTCTGCAATGTCCACTTCGGGAGG | |
| | ATGAAGAAAGTTTTAGAGTGAGACCAGCGTAACTCACACAAATGGCGG | |
| E347L | TATCTACACGGGTCTCACCAAAACCACGTTACTTTACTTGAAAAAAACACTTCGGGAGGAT | 502 |
| | GAACTCATGGGGTTGACTACCACATCTACTATCATGAGTCTGCAATGTCCACTTCGGGAGG | |
| | ATGAAGAAAGTTTTAGAGTGAGACCAGCGTAACTCTTATACGCCATGG | |
| E347K | TATCTACACGGGTCTCACCAAAACCACGTTACTTTACTTGAAAAAAACACTTCGGGAGGAT | 503 |
| | GAAAAGATGGGGTTGACTACCACATCTACTATCATGAGTCTGCAATGTCCACTTCGGGAGG | |
| | ATGAAGAAAGTTTTAGAGTGAGACCAGCGTAACTCTCTTCCCTAGGCC | |
| E347M | TATCTACACGGGTCTCACCAAAACCACGTTACTTTACTTGAAAAAAACACTTCGGGAGGAT | 504 |
| | GAAATGATGGGGTTGACTACCACATCTACTATCATGAGTCTGCAATGTCCACTTCGGGAGG | |
| | ATGAAGAAAGTTTTAGAGTGAGACCAGCGTAACTCGAGTCTCATCCGC | |
| E347F | TATCTACACGGGTCTCACCAAAACCACGTTACTTTACTTGAAAAAAACACTTCGGGAGGAT | 505 |
| | GAATTCATGGGGTTGACTACCACATCTACTATCATGAGTCTGCAATGTCCACTTCGGGAGG | |
| | ATGAAGAAAGTTTTAGAGTGAGACCAGCGTAACTCGGTGGTAATATAA | |
| E347P | TATCTACACGGGTCTCACCAAAACCACGTTACTTTACTTGAAAAAAACACTTCGGGAGGAT | 506 |
| | GAACCGATGGGGTTGACTACCACATCTACTATCATGAGTCTGCAATGTCCACTTCGGGAGG | |
| | ATGAAGAAAGTTTTAGAGTGAGACCAGCGTAACTCGTGATAATAGGCA | |
| E347S | TATCTACACGGGTCTCACCAAAACCACGTTACTTTACTTGAAAAAAACACTTCGGGAGGAT | 507 |
| | GAAAGCATGGGGTTGACTACCACATCTACTATCATGAGTCTGCAATGTCCACTTCGGGAGG | |
| | ATGAAGAAAGTTTTAGAGTGAGACCAGCGTAACTCAGATACATATGAG | |
| E347T | TATCTACACGGGTCTCACCAAAACCACGTTACTTTACTTGAAAAAAACACTTCGGGAGGAT | 508 |
| | GAAACGATGGGGTTGACTACCACATCTACTATCATGAGTCTGCAATGTCCACTTCGGGAGG | |
| | ATGAAGAAAGTTTTAGAGTGAGACCAGCGTAACTCTGTTATTTATGCC | |
| E347W | TATCTACACGGGTCTCACCAAAACCACGTTACTTTACTTGAAAAAAACACTTCGGGAGGAT | 509 |
| | GAATGGATGGGGTTGACTACCACATCTACTATCATGAGTCTGCAATGTCCACTTCGGGAGG | |
| | ATGAAGAAAGTTTTAGAGTGAGACCAGCGTAACTCCGTGTAATCGCAC | |
| E347Y | TATCTACACGGGTCTCACCAAAACCACGTTACTTTACTTGAAAAAAACACTTCGGGAGGAT | 510 |
| | GAATACATGGGGTTGACTACCACATCTACTATCATGAGTCTGCAATGTCCACTTCGGGAGG | |
| | ATGAAGAAAGTTTTAGAGTGAGACCAGCGTAACTCCCGTGCTGGAAGA | |
| E347V | TATCTACACGGGTCTCACCAAAACCACGTTACTTTACTTGAAAAAAACACTTCGGGAGGAT | 511 |
| | GAAGTCATGGGGTTGACTACCACATCTACTATCATGAGTCTGCAATGTCCACTTCGGGAGG | |
| | ATGAAGAAAGTTTTAGAGTGAGACCAGCGTAACTCCCAGCTAGATAGA | |
| M348A | TATCTACACGGGTCTCACCAAAACGTTACTTTACTTGAAAAAAACACTTCGGGAGGATGAA | 512 |
| | GAGGCGGGGTTGACTACCACATCTACTATCATGAGTCTGCAATGTCCACTTCGGGAGGATG | |
| | AAGAAAGTTTTAGAGTGAGACCAGCGTAACTCCATACCACAAAATTAT | |
| M348R | TATCTACACGGGTCTCACCAAAACGTTACTTTACTTGAAAAAAACACTTCGGGAGGATGAA | 513 |
| | GAGCGGGGGTTGACTACCACATCTACTATCATGAGTCTGCAATGTCCACTTCGGGAGGATG | |
| | AAGAAAGTTTTAGAGTGAGACCAGCGTAACTCGCGGCTCAGTGCACCA | |
| M348N | TATCTACACGGGTCTCACCAAAACGTTACTTTACTTGAAAAAAACACTTCGGGAGGATGAA | 514 |
| | GAAAACGGGTTGACTACCACATCTACTATCATGAGTCTGCAATGTCCACTTCGGGAGGATG | |
| | AAGAAAGTTTTAGAGTGAGACCAGCGTAACTCGAAGCTATGGTAGCCA | |
| M348D | TATCTACACGGGTCTCACCAAAACGTTACTTTACTTGAAAAAAACACTTCGGGAGGATGAA | 515 |
| | GAAGACGGGTTGACTACCACATCTACTATCATGAGTCTGCAATGTCCACTTCGGGAGGATG | |
| | AAGAAAGTTTTAGAGTGAGACCAGCGTAACTCCGTCAGCTAGCAGCAC | |
| M348C | TATCTACACGGGTCTCACCAAAACGTTACTTTACTTGAAAAAAACACTTCGGGAGGATGAA | 516 |
| | GAATGCGGGTTGACTACCACATCTACTATCATGAGTCTGCAATGTCCACTTCGGGAGGATG | |
| | AAGAAAGTTTTAGAGTGAGACCAGCGTAACTCGGCGTGAAAAACCTTC | |
| M348Q | TATCTACACGGGTCTCACCAAAACGTTACTTTACTTGAAAAAAACACTTCGGGAGGATGAA | 517 |
| | GAGCAGGGGTTGACTACCACATCTACTATCATGAGTCTGCAATGTCCACTTCGGGAGGATG | |
| | AAGAAAGTTTTAGAGTGAGACCAGCGTAACTCTAGCCACCTGCCACTG | |
| M348E | TATCTACACGGGTCTCACCAAAACGTTACTTTACTTGAAAAAAACACTTCGGGAGGATGAA | 518 |
| | GAGGAGGGGTTGACTACCACATCTACTATCATGAGTCTGCAATGTCCACTTCGGGAGGATG | |
| | AAGAAAGTTTTAGAGTGAGACCAGCGTAACTCATACATTTAATAGCCA | |
| M348G | TATCTACACGGGTCTCACCAAAACGTTACTTTACTTGAAAAAAACACTTCGGGAGGATGAA | 519 |
| | GAGGGGGGGTTGACTACCACATCTACTATCATGAGTCTGCAATGTCCACTTCGGGAGGATG | |
| | AAGAAAGTTTTAGAGTGAGACCAGCGTAACTCCCCGCGGCCTATTAGC | |
| M348H | TATCTACACGGGTCTCACCAAAACGTTACTTTACTTGAAAAAAACACTTCGGGAGGATGAA | 520 |
| | GAACACGGGTTGACTACCACATCTACTATCATGAGTCTGCAATGTCCACTTCGGGAGGATG | |
| | AAGAAAGTTTTAGAGTGAGACCAGCGTAACTCGTAAAGTGACGAGGAT | |
| M348I | TATCTACACGGGTCTCACCAAAACGTTACTTTACTTGAAAAAAACACTTCGGGAGGATGAA | 521 |
| | GAAATCGGGTTGACTACCACATCTACTATCATGAGTCTGCAATGTCCACTTCGGGAGGATG | |
| | AAGAAAGTTTTAGAGTGAGACCAGCGTAACTCGTTTGTATCGCCACTG | |
| M348L | TATCTACACGGGTCTCACCAAAACGTTACTTTACTTGAAAAAAACACTTCGGGAGGATGAA | 522 |
| | GAACTCGGGTTGACTACCACATCTACTATCATGAGTCTGCAATGTCCACTTCGGGAGGATG | |
| | AAGAAAGTTTTAGAGTGAGACCAGCGTAACTCCCAGCCTCGCGACCAG | |
| M348K | TATCTACACGGGTCTCACCAAAACGTTACTTTACTTGAAAAAAACACTTCGGGAGGATGAA | 523 |
| | GAGAAGGGGTTGACTACCACATCTACTATCATGAGTCTGCAATGTCCACTTCGGGAGGATG | |
| | AAGAAAGTTTTAGAGTGAGACCAGCGTAACTCTAACGCCGAGAAGCTT | |
| M348M | TATCTACACGGGTCTCACCAAAACGTTACTTTACTTGAAAAAAACACTTCGGGAGGATGAA | 524 |
| | GAGATGGGGTTGACTACCACATCTACTATCATGAGTCTGCAATGTCCACTTCGGGAGGATG | |
| | AAGAAAGTTTTAGAGTGAGACCAGCGTAACTCCGTATGTGCCAGTTAT | |
| M348F | TATCTACACGGGTCTCACCAAAACGTTACTTTACTTGAAAAAAACACTTCGGGAGGATGAA | 525 |
| | GAATTCGGGTTGACTACCACATCTACTATCATGAGTCTGCAATGTCCACTTCGGGAGGATG | |
| | AAGAAAGTTTTAGAGTGAGACCAGCGTAACTCAAACATAAGAACGTCG | |
| M348P | TATCTACACGGGTCTCACCAAAACGTTACTTTACTTGAAAAAAACACTTCGGGAGGATGAA | 526 |
| | GAGCCGGGGTTGACTACCACATCTACTATCATGAGTCTGCAATGTCCACTTCGGGAGGATG | |
| | AAGAAAGTTTTAGAGTGAGACCAGCGTAACTCAGATACCCGATGGGAG | |
| M348S | TATCTACACGGGTCTCACCAAAACGTTACTTTACTTGAAAAAAACACTTCGGGAGGATGAA | 527 |
| | GAAAGCGGGTTGACTACCACATCTACTATCATGAGTCTGCAATGTCCACTTCGGGAGGATG | |
| | AAGAAAGTTTTAGAGTGAGACCAGCGTAACTCGCGCACATAGACCAAT | |
| M348T | TATCTACACGGGTCTCACCAAAACGTTACTTTACTTGAAAAAAACACTTCGGGAGGATGAA | 528 |
| | GAGACGGGGTTGACTACCACATCTACTATCATGAGTCTGCAATGTCCACTTCGGGAGGATG | |
| | AAGAAAGTTTTAGAGTGAGACCAGCGTAACTCGGGTCACCGATAAGAA | |
| M348W | TATCTACACGGGTCTCACCAAAACGTTACTTTACTTGAAAAAAACACTTCGGGAGGATGAA | 529 |
| | GAGTGGGGGTTGACTACCACATCTACTATCATGAGTCTGCAATGTCCACTTCGGGAGGATG | |
| | AAGAAAGTTTTAGAGTGAGACCAGCGTAACTCTGATACGTGTGTACAT | |
| M348Y | TATCTACACGGGTCTCACCAAAACGTTACTTTACTTGAAAAAAACACTTCGGGAGGATGAA | 530 |
| | GAATACGGGTTGACTACCACATCTACTATCATGAGTCTGCAATGTCCACTTCGGGAGGATG | |
| | AAGAAAGTTTTAGAGTGAGACCAGCGTAACTCTGAAACGCCAGGTCGG | |
| M348V | TATCTACACGGGTCTCACCAAAACGTTACTTTACTTGAAAAAAACACTTCGGGAGGATGAA | 531 |
| | GAAGTCGGGTTGACTACCACATCTACTATCATGAGTCTGCAATGTCCACTTCGGGAGGATG | |
| | AAGAAAGTTTTAGAGTGAGACCAGCGTAACTCGTTCCGTTACCACAGT | |
| G349A | TATCTACACGGGTCTCACCAAAACTTACTTTACTTGAAAAAAACACTTCGGGAGGATGAAG | 532 |
| | AGATGGCGTTGACTACCACATCTACTATCATGAGTCTGCAATGTCCAAACTTCGGGAGGAT | |
| | GAAGAAAGTTTTAGAGTGAGACCAGCGTAACTCGGACTGGAATAAAGA | |
| G349R | TATCTACACGGGTCTCACCAAAACTTACTTTACTTGAAAAAAACACTTCGGGAGGATGAAG | 533 |
| | AAATGCGGTTGACTACCACATCTACTATCATGAGTCTGCAATGTCCAAACTTCGGGAGGAT | |
| | GAAGAAAGTTTTAGAGTGAGACCAGCGTAACTCCAAGAAAGTAGCAAG | |
| G349N | TATCTACACGGGTCTCACCAAAACTTACTTTACTTGAAAAAAACACTTCGGGAGGATGAAG | 534 |
| | AAATGAACTTGACTACCACATCTACTATCATGAGTCTGCAATGTCCAAACTTCGGGAGGAT | |
| | GAAGAAAGTTTTAGAGTGAGACCAGCGTAACTCAACCTAGTTCAGTTC | |
| G349D | TATCTACACGGGTCTCACCAAAACTTACTTTACTTGAAAAAAACACTTCGGGAGGATGAAG | 535 |
| | AGATGGACTTGACTACCACATCTACTATCATGAGTCTGCAATGTCCAAACTTCGGGAGGAT | |
| | GAAGAAAGTTTTAGAGTGAGACCAGCGTAACTCATGCCGAGCTATGCC | |
| G349C | TATCTACACGGGTCTCACCAAAACTTACTTTACTTGAAAAAAACACTTCGGGAGGATGAAG | 536 |
| | AAATGTGCTTGACTACCACATCTACTATCATGAGTCTGCAATGTCCAAACTTCGGGAGGAT | |
| | GAAGAAAGTTTTAGAGTGAGACCAGCGTAACTCCGGGGAAGATAGCAA | |
| G349Q | TATCTACACGGGTCTCACCAAAACTTACTTTACTTGAAAAAAACACTTCGGGAGGATGAAG | 537 |
| | AAATGCAGTTGACTACCACATCTACTATCATGAGTCTGCAATGTCCAAACTTCGGGAGGAT | |
| | GAAGAAAGTTTTAGAGTGAGACCAGCGTAACTCACATGGGGGGGATGC | |
| G349E | TATCTACACGGGTCTCACCAAAACTTACTTTACTTGAAAAAAACACTTCGGGAGGATGAAG | 538 |
| | AGATGGAGTTGACTACCACATCTACTATCATGAGTCTGCAATGTCCAAACTTCGGGAGGAT | |
| | GAAGAAAGTTTTAGAGTGAGACCAGCGTAACTCCCGGGCCTCAGCCGT | |
| G349G | TATCTACACGGGTCTCACCAAAACTTACTTTACTTGAAAAAAACACTTCGGGAGGATGAAG | 539 |
| | AGATGGGTTTGACTACCACATCTACTATCATGAGTCTGCAATGTCCAAACTTCGGGAGGAT | |
| | GAAGAAAGTTTTAGAGTGAGACCAGCGTAACTCTGGGTCGGAGTGCTT | |
| G349H | TATCTACACGGGTCTCACCAAAACTTACTTTACTTGAAAAAAACACTTCGGGAGGATGAAG | 540 |
| | AAATGCACTTGACTACCACATCTACTATCATGAGTCTGCAATGTCCAAACTTCGGGAGGAT | |
| | GAAGAAAGTTTTAGAGTGAGACCAGCGTAACTCCAAGTGTTTCTCGCT | |
| G349I | TATCTACACGGGTCTCACCAAAACTTACTTTACTTGAAAAAAACACTTCGGGAGGATGAAG | 541 |
| | AAATGATCTTGACTACCACATCTACTATCATGAGTCTGCAATGTCCAAACTTCGGGAGGAT | |
| | GAAGAAAGTTTTAGAGTGAGACCAGCGTAACTCTGGCTGAATGCGTTC | |
| G349L | TATCTACACGGGTCTCACCAAAACTTACTTTACTTGAAAAAAACACTTCGGGAGGATGAAG | 542 |
| | AAATGCTCTTGACTACCACATCTACTATCATGAGTCTGCAATGTCCAAACTTCGGGAGGAT | |
| | GAAGAAAGTTTTAGAGTGAGACCAGCGTAACTCGCGACTCTTGCCCCA | |
| G349K | TATCTACACGGGTCTCACCAAAACTTACTTTACTTGAAAAAAACACTTCGGGAGGATGAAG | 543 |
| | AAATGAAGTTGACTACCACATCTACTATCATGAGTCTGCAATGTCCAAACTTCGGGAGGAT | |
| | GAAGAAAGTTTTAGAGTGAGACCAGCGTAACTCTCGTGATTAAGTTGT | |
| G349M | TATCTACACGGGTCTCACCAAAACTTACTTTACTTGAAAAAAACACTTCGGGAGGATGAAG | 544 |
| | AAATGATGTTGACTACCACATCTACTATCATGAGTCTGCAATGTCCAAACTTCGGGAGGAT | |
| | GAAGAAAGTTTTAGAGTGAGACCAGCGTAACTCGTCGTAGTAATGCAG | |
| G349F | TATCTACACGGGTCTCACCAAAACTTACTTTACTTGAAAAAAACACTTCGGGAGGATGAAG | 545 |
| | AAATGTTCTTGACTACCACATCTACTATCATGAGTCTGCAATGTCCAAACTTCGGGAGGAT | |
| | GAAGAAAGTTTTAGAGTGAGACCAGCGTAACTCGCGGCGTCAAAACGG | |
| G349P | TATCTACACGGGTCTCACCAAAACTTACTTTACTTGAAAAAAACACTTCGGGAGGATGAAG | 546 |
| | AAATGCCGTTGACTACCACATCTACTATCATGAGTCTGCAATGTCCAAACTTCGGGAGGAT | |
| | GAAGAAAGTTTTAGAGTGAGACCAGCGTAACTCCTTTACCTTAATTCG | |
| G349S | TATCTACACGGGTCTCACCAAAACTTACTTTACTTGAAAAAAACACTTCGGGAGGATGAAG | 547 |
| | AAATGAGCTTGACTACCACATCTACTATCATGAGTCTGCAATGTCCAAACTTCGGGAGGAT | |
| | GAAGAAAGTTTTAGAGTGAGACCAGCGTAACTCTGCTGAAGGCAGATG | |
| G349T | TATCTACACGGGTCTCACCAAAACTTACTTTACTTGAAAAAAACACTTCGGGAGGATGAAG | 548 |
| | AAATGACGTTGACTACCACATCTACTATCATGAGTCTGCAATGTCCAAACTTCGGGAGGAT | |
| | GAAGAAAGTTTTAGAGTGAGACCAGCGTAACTCGATCCACCCCTGTTT | |
| G349W | TATCTACACGGGTCTCACCAAAACTTACTTTACTTGAAAAAAACACTTCGGGAGGATGAAG | 549 |
| | AAATGTGGTTGACTACCACATCTACTATCATGAGTCTGCAATGTCCAAACTTCGGGAGGAT | |
| | GAAGAAAGTTTTAGAGTGAGACCAGCGTAACTCGGGAAACAAAAGGTG | |
| G349Y | TATCTACACGGGTCTCACCAAAACTTACTTTACTTGAAAAAAACACTTCGGGAGGATGAAG | 550 |
| | AAATGTACTTGACTACCACATCTACTATCATGAGTCTGCAATGTCCAAACTTCGGGAGGAT | |
| | GAAGAAAGTTTTAGAGTGAGACCAGCGTAACTCGTCTTATCGCAAATC | |
| G349V | TATCTACACGGGTCTCACCAAAACTTACTTTACTTGAAAAAAACACTTCGGGAGGATGAAG | 551 |
| | AGATGGTCTTGACTACCACATCTACTATCATGAGTCTGCAATGTCCAAACTTCGGGAGGAT | |
| | GAAGAAAGTTTTAGAGTGAGACCAGCGTAACTCAAGGTATGCCCGGAT | |
| L350A | TATCTACACGGGTCTCACCAAAACTTACTTTACTTGAAAAAAACACTTCGGGAGGATGAAG | 552 |
| | AGATGGGGGCTACTACCACATCTACTATCATGAGTCTGCAATGTCCAATTTACTTCGGGAG | |
| | GATGAAGAAAGTTTTAGAGTGAGACCAGCGTAACTCGATCCAGTCCGA | |
| L350R | TATCTACACGGGTCTCACCAAAACTTACTTTACTTGAAAAAAACACTTCGGGAGGATGAAG | 553 |
| | AGATGGGGAGAACTACCACATCTACTATCATGAGTCTGCAATGTCCAATTTACTTCGGGAG | |
| | GATGAAGAAAGTTTTAGAGTGAGACCAGCGTAACTCCAAAATTCAAAG | |
| L350N | TATCTACACGGGTCTCACCAAAACTTACTTTACTTGAAAAAAACACTTCGGGAGGATGAAG | 554 |
| | AGATGGGGAATACTACCACATCTACTATCATGAGTCTGCAATGTCCAATTTACTTCGGGAG | |
| | GATGAAGAAAGTTTTAGAGTGAGACCAGCGTAACTCTTCACGGCAGAC | |
| L350D | TATCTACACGGGTCTCACCAAAACTTACTTTACTTGAAAAAAACACTTCGGGAGGATGAAG | 555 |
| | AGATGGGGGATACTACCACATCTACTATCATGAGTCTGCAATGTCCAATTTACTTCGGGAG | |
| | GATGAAGAAAGTTTTAGAGTGAGACCAGCGTAACTCTAAGGCCCTGCC | |
| L350C | TATCTACACGGGTCTCACCAAAACTTACTTTACTTGAAAAAAACACTTCGGGAGGATGAAG | 556 |
| | AGATGGGGTGTACTACCACATCTACTATCATGAGTCTGCAATGTCCAATTTACTTCGGGAG | |
| | GATGAAGAAAGTTTTAGAGTGAGACCAGCGTAACTCGAAGCCCTCCAC | |
| L350Q | TATCTACACGGGTCTCACCAAAACTTACTTTACTTGAAAAAAACACTTCGGGAGGATGAAG | 557 |
| | AGATGGGGCAAACTACCACATCTACTATCATGAGTCTGCAATGTCCAATTTACTTCGGGAG | |
| | GATGAAGAAAGTTTTAGAGTGAGACCAGCGTAACTCCCCCAAAAATAG | |
| L350E | TATCTACACGGGTCTCACCAAAACTTACTTTACTTGAAAAAAACACTTCGGGAGGATGAAG | 558 |
| | AGATGGGGGAAACTACCACATCTACTATCATGAGTCTGCAATGTCCAATTTACTTCGGGAG | |
| | GATGAAGAAAGTTTTAGAGTGAGACCAGCGTAACTCTGGGATCGAGTG | |
| L350G | TATCTACACGGGTCTCACCAAAACTTACTTTACTTGAAAAAAACACTTCGGGAGGATGAAG | 559 |
| | AGATGGGGGGTACTACCACATCTACTATCATGAGTCTGCAATGTCCAATTTACTTCGGGAG | |
| | GATGAAGAAAGTTTTAGAGTGAGACCAGCGTAACTCCGTCGTAAGGAT | |
| L350H | TATCTACACGGGTCTCACCAAAACTTACTTTACTTGAAAAAAACACTTCGGGAGGATGAAG | 560 |
| | AGATGGGGCATACTACCACATCTACTATCATGAGTCTGCAATGTCCAATTTACTTCGGGAG | |
| | GATGAAGAAAGTTTTAGAGTGAGACCAGCGTAACTCCCGGCAGAGGGC | |
| L350I | TATCTACACGGGTCTCACCAAAACTTACTTTACTTGAAAAAAACACTTCGGGAGGATGAAG | 561 |
| | AGATGGGGATTACTACCACATCTACTATCATGAGTCTGCAATGTCCAATTTACTTCGGGAG | |
| | GATGAAGAAAGTTTTAGAGTGAGACCAGCGTAACTCGTGTCGACCAGT | |
| L350L | TATCTACACGGGTCTCACCAAAACTTACTTTACTTGAAAAAAACACTTCGGGAGGATGAAG | 562 |
| | AGATGGGGTTAACTACCACATCTACTATCATGAGTCTGCAATGTCCAATTTACTTCGGGAG | |
| | GATGAAGAAAGTTTTAGAGTGAGACCAGCGTAACTCGCGAACAACTCG | |
| L350K | TATCTACACGGGTCTCACCAAAACTTACTTTACTTGAAAAAAACACTTCGGGAGGATGAAG | 563 |
| | AGATGGGGAAAACTACCACATCTACTATCATGAGTCTGCAATGTCCAATTTACTTCGGGAG | |
| | GATGAAGAAAGTTTTAGAGTGAGACCAGCGTAACTCGGGGGTACACTT | |
| L350M | TATCTACACGGGTCTCACCAAAACTTACTTTACTTGAAAAAAACACTTCGGGAGGATGAAG | 564 |
| | AGATGGGGATGACTACCACATCTACTATCATGAGTCTGCAATGTCCAATTTACTTCGGGAG | |
| | GATGAAGAAAGTTTTAGAGTGAGACCAGCGTAACTCGCATACCAAATA | |
| L350F | TATCTACACGGGTCTCACCAAAACTTACTTTACTTGAAAAAAACACTTCGGGAGGATGAAG | 565 |
| | AGATGGGGTTTACTACCACATCTACTATCATGAGTCTGCAATGTCCAATTTACTTCGGGAG | |
| | GATGAAGAAAGTTTTAGAGTGAGACCAGCGTAACTCTAAACCACTCAG | |
| L350P | TATCTACACGGGTCTCACCAAAACTTACTTTACTTGAAAAAAACACTTCGGGAGGATGAAG | 566 |
| | AGATGGGGCCAACTACCACATCTACTATCATGAGTCTGCAATGTCCAATTTACTTCGGGAG | |
| | GATGAAGAAAGTTTTAGAGTGAGACCAGCGTAACTCTCGGACAATACG | |
| L350S | TATCTACACGGGTCTCACCAAAACTTACTTTACTTGAAAAAAACACTTCGGGAGGATGAAG | 567 |
| | AGATGGGGTCTACTACCACATCTACTATCATGAGTCTGCAATGTCCAATTTACTTCGGGAG | |
| | GATGAAGAAAGTTTTAGAGTGAGACCAGCGTAACTCGAGGTTGACCTC | |
| L350T | TATCTACACGGGTCTCACCAAAACTTACTTTACTTGAAAAAAACACTTCGGGAGGATGAAG | 568 |
| | AGATGGGGACTACTACCACATCTACTATCATGAGTCTGCAATGTCCAATTTACTTCGGGAG | |
| | GATGAAGAAAGTTTTAGAGTGAGACCAGCGTAACTCTTCCAGGTTGGA | |
| L350W | TATCTACACGGGTCTCACCAAAACTTACTTTACTTGAAAAAAACACTTCGGGAGGATGAAG | 569 |
| | AGATGGGGTGGACTACCACATCTACTATCATGAGTCTGCAATGTCCAATTTACTTCGGGAG | |
| | GATGAAGAAAGTTTTAGAGTGAGACCAGCGTAACTCACTGTACACCTG | |
| L350Y | TATCTACACGGGTCTCACCAAAACTTACTTTACTTGAAAAAAACACTTCGGGAGGATGAAG | 570 |
| | AGATGGGGTATACTACCACATCTACTATCATGAGTCTGCAATGTCCAATTTACTTCGGGAG | |
| | GATGAAGAAAGTTTTAGAGTGAGACCAGCGTAACTCGTGTGATTGCGC | |
| L350V | TATCTACACGGGTCTCACCAAAACTTACTTTACTTGAAAAAAACACTTCGGGAGGATGAAG | 571 |
| | AGATGGGGGTTACTACCACATCTACTATCATGAGTCTGCAATGTCCAATTTACTTCGGGAG | |
| | GATGAAGAAAGTTTTAGAGTGAGACCAGCGTAACTCACGTGGGGTCCC | |
| T351A | TATCTACACGGGTCTCACCAAAACTTACTTTACTTGAAAAAAACACTTCGGGAGGATGAAG | 572 |
| | AGATGGGGTTGGCTACCACATCTACTATCATGAGTCTGCAATGTCCAATTTCGTACTTCGG | |
| | GAGGATGAAGAAAGTTTTAGAGTGAGACCAGCGTAACTCGCGTGGATC | |
| T351R | TATCTACACGGGTCTCACCAAAACTTACTTTACTTGAAAAAAACACTTCGGGAGGATGAAG | 573 |
| | AGATGGGGTTGAGAACCACATCTACTATCATGAGTCTGCAATGTCCAATTTCGTACTTCGG | |
| | GAGGATGAAGAAAGTTTTAGAGTGAGACCAGCGTAACTCTACTGAGTA | |
| T351N | TATCTACACGGGTCTCACCAAAACTTACTTTACTTGAAAAAAACACTTCGGGAGGATGAAG | 574 |
| | AGATGGGGTTGAATACCACATCTACTATCATGAGTCTGCAATGTCCAATTTCGTACTTCGG | |
| | GAGGATGAAGAAAGTTTTAGAGTGAGACCAGCGTAACTCCTAAGAATG | |
| T351D | TATCTACACGGGTCTCACCAAAACTTACTTTACTTGAAAAAAACACTTCGGGAGGATGAAG | 575 |
| | AGATGGGGTTGGATACCACATCTACTATCATGAGTCTGCAATGTCCAATTTCGTACTTCGG | |
| | GAGGATGAAGAAAGTTTTAGAGTGAGACCAGCGTAACTCTGAAGAGTA | |
| T351C | TATCTACACGGGTCTCACCAAAACTTACTTTACTTGAAAAAAACACTTCGGGAGGATGAAG | 576 |
| | AGATGGGGTTGTGTACCACATCTACTATCATGAGTCTGCAATGTCCAATTTCGTACTTCGG | |
| | GAGGATGAAGAAAGTTTTAGAGTGAGACCAGCGTAACTCTATTTACGG | |
| T351Q | TATCTACACGGGTCTCACCAAAACTTACTTTACTTGAAAAAAACACTTCGGGAGGATGAAG | 577 |
| | AGATGGGGTTGCAAACCACATCTACTATCATGAGTCTGCAATGTCCAATTTCGTACTTCGG | |
| | GAGGATGAAGAAAGTTTTAGAGTGAGACCAGCGTAACTCATTAGCTAA | |
| T351E | TATCTACACGGGTCTCACCAAAACTTACTTTACTTGAAAAAAACACTTCGGGAGGATGAAG | 578 |
| | AGATGGGGTTGGAAACCACATCTACTATCATGAGTCTGCAATGTCCAATTTCGTACTTCGG | |
| | GAGGATGAAGAAAGTTTTAGAGTGAGACCAGCGTAACTCTTCCACATG | |
| T351G | TATCTACACGGGTCTCACCAAAACTTACTTTACTTGAAAAAAACACTTCGGGAGGATGAAG | 579 |
| | AGATGGGGTTGGGTACCACATCTACTATCATGAGTCTGCAATGTCCAATTTCGTACTTCGG | |
| | GAGGATGAAGAAAGTTTTAGAGTGAGACCAGCGTAACTCCCGACGTAC | |
| T351H | TATCTACACGGGTCTCACCAAAACTTACTTTACTTGAAAAAAACACTTCGGGAGGATGAAG | 580 |
| | AGATGGGGTTGCATACCACATCTACTATCATGAGTCTGCAATGTCCAATTTCGTACTTCGG | |
| | GAGGATGAAGAAAGTTTTAGAGTGAGACCAGCGTAACTCTCATAATCA | |
| T351I | TATCTACACGGGTCTCACCAAAACTTACTTTACTTGAAAAAAACACTTCGGGAGGATGAAG | 581 |
| | AGATGGGGTTGATTACCACATCTACTATCATGAGTCTGCAATGTCCAATTTCGTACTTCGG | |
| | GAGGATGAAGAAAGTTTTAGAGTGAGACCAGCGTAACTCTATAACACC | |
| T351L | TATCTACACGGGTCTCACCAAAACTTACTTTACTTGAAAAAAACACTTCGGGAGGATGAAG | 582 |
| | AGATGGGGTTGTTGACCACATCTACTATCATGAGTCTGCAATGTCCAATTTCGTACTTCGG | |
| | GAGGATGAAGAAAGTTTTAGAGTGAGACCAGCGTAACTCAATACTGAA | |
| T351K | TATCTACACGGGTCTCACCAAAACTTACTTTACTTGAAAAAAACACTTCGGGAGGATGAAG | 583 |
| | AGATGGGGTTGAAAACCACATCTACTATCATGAGTCTGCAATGTCCAATTTCGTACTTCGG | |
| | GAGGATGAAGAAAGTTTTAGAGTGAGACCAGCGTAACTCCCCGGTGAC | |
| T351M | TATCTACACGGGTCTCACCAAAACTTACTTTACTTGAAAAAAACACTTCGGGAGGATGAAG | 584 |
| | AGATGGGGTTGATGACCACATCTACTATCATGAGTCTGCAATGTCCAATTTCGTACTTCGG | |
| | GAGGATGAAGAAAGTTTTAGAGTGAGACCAGCGTAACTCCTTCTGACG | |
| T351F | TATCTACACGGGTCTCACCAAAACTTACTTTACTTGAAAAAAACACTTCGGGAGGATGAAG | 585 |
| | AGATGGGGTTGTTTACCACATCTACTATCATGAGTCTGCAATGTCCAATTTCGTACTTCGG | |
| | GAGGATGAAGAAAGTTTTAGAGTGAGACCAGCGTAACTCCAGCGTACG | |
| T351P | TATCTACACGGGTCTCACCAAAACTTACTTTACTTGAAAAAAACACTTCGGGAGGATGAAG | 586 |
| | AGATGGGGTTGCCAACCACATCTACTATCATGAGTCTGCAATGTCCAATTTCGTACTTCGG | |
| | GAGGATGAAGAAAGTTTTAGAGTGAGACCAGCGTAACTCGAGGATACG | |
| T351S | TATCTACACGGGTCTCACCAAAACTTACTTTACTTGAAAAAAACACTTCGGGAGGATGAAG | 587 |
| | AGATGGGGTTGTCTACCACATCTACTATCATGAGTCTGCAATGTCCAATTTCGTACTTCGG | |
| | GAGGATGAAGAAAGTTTTAGAGTGAGACCAGCGTAACTCAGAGCTTTA | |
| T351T | TATCTACACGGGTCTCACCAAAACTTACTTTACTTGAAAAAAACACTTCGGGAGGATGAAG | 588 |
| | AGATGGGGTTGACAACCACATCTACTATCATGAGTCTGCAATGTCCAATTTCGTACTTCGG | |
| | GAGGATGAAGAAAGTTTTAGAGTGAGACCAGCGTAACTCCCGTTTTGC | |
| T351W | TATCTACACGGGTCTCACCAAAACTTACTTTACTTGAAAAAAACACTTCGGGAGGATGAAG | 589 |
| | AGATGGGGTTGTGGACCACATCTACTATCATGAGTCTGCAATGTCCAATTTCGTACTTCGG | |
| | GAGGATGAAGAAAGTTTTAGAGTGAGACCAGCGTAACTCCGGAAATAC | |
| T351Y | TATCTACACGGGTCTCACCAAAACTTACTTTACTTGAAAAAAACACTTCGGGAGGATGAAG | 590 |
| | AGATGGGGTTGTATACCACATCTACTATCATGAGTCTGCAATGTCCAATTTCGTACTTCGG | |
| | GAGGATGAAGAAAGTTTTAGAGTGAGACCAGCGTAACTCCAAGTCTCT | |
| T351V | TATCTACACGGGTCTCACCAAAACTTACTTTACTTGAAAAAAACACTTCGGGAGGATGAAG | 591 |
| | AGATGGGGTTGGTTACCACATCTACTATCATGAGTCTGCAATGTCCAATTTCGTACTTCGG | |
| | GAGGATGAAGAAAGTTTTAGAGTGAGACCAGCGTAACTCGGTATGGTG | |
| T352A | TATCTACACGGGTCTCACCAAAACTTACTTTACTTGAAAAAAACACTTCGGGAGGATGAAG | 592 |
| | AGATGGGGTTGACTGCTACATCTACTATCATGAGTCTGCAATGTCCAATTTCGTACAACTT | |
| | CGGGAGGATGAAGAAAGTTTTAGAGTGAGACCAGCGTAACTCTAGGCA | |
| T352R | TATCTACACGGGTCTCACCAAAACTTACTTTACTTGAAAAAAACACTTCGGGAGGATGAAG | 593 |
| | AGATGGGGTTGACTAGAACATCTACTATCATGAGTCTGCAATGTCCAATTTCGTACAACTT | |
| | CGGGAGGATGAAGAAAGTTTTAGAGTGAGACCAGCGTAACTCCTCTAG | |
| T352N | TATCTACACGGGTCTCACCAAAACTTACTTTACTTGAAAAAAACACTTCGGGAGGATGAAG | 594 |
| | AGATGGGGTTGACTAATACATCTACTATCATGAGTCTGCAATGTCCAATTTCGTACAACTT | |
| | CGGGAGGATGAAGAAAGTTTTAGAGTGAGACCAGCGTAACTCTTTTCA | |
| T352D | TATCTACACGGGTCTCACCAAAACTTACTTTACTTGAAAAAAACACTTCGGGAGGATGAAG | 595 |
| | AGATGGGGTTGACTGATACATCTACTATCATGAGTCTGCAATGTCCAATTTCGTACAACTT | |
| | CGGGAGGATGAAGAAAGTTTTAGAGTGAGACCAGCGTAACTCGCCATA | |
| T352C | TATCTACACGGGTCTCACCAAAACTTACTTTACTTGAAAAAAACACTTCGGGAGGATGAAG | 596 |
| | AGATGGGGTTGACTTGTACATCTACTATCATGAGTCTGCAATGTCCAATTTCGTACAACTT | |
| | CGGGAGGATGAAGAAAGTTTTAGAGTGAGACCAGCGTAACTCAGTAGA | |
| T352Q | TATCTACACGGGTCTCACCAAAACTTACTTTACTTGAAAAAAACACTTCGGGAGGATGAAG | 597 |
| | AGATGGGGTTGACTCAAACATCTACTATCATGAGTCTGCAATGTCCAATTTCGTACAACTT | |
| | CGGGAGGATGAAGAAAGTTTTAGAGTGAGACCAGCGTAACTCCGCCAT | |
| T352E | TATCTACACGGGTCTCACCAAAACTTACTTTACTTGAAAAAAACACTTCGGGAGGATGAAG | 598 |
| | AGATGGGGTTGACTGAAACATCTACTATCATGAGTCTGCAATGTCCAATTTCGTACAACTT | |
| | CGGGAGGATGAAGAAAGTTTTAGAGTGAGACCAGCGTAACTCAGGCTC | |
| T352G | TATCTACACGGGTCTCACCAAAACTTACTTTACTTGAAAAAAACACTTCGGGAGGATGAAG | 599 |
| | AGATGGGGTTGACTGGTACATCTACTATCATGAGTCTGCAATGTCCAATTTCGTACAACTT | |
| | CGGGAGGATGAAGAAAGTTTTAGAGTGAGACCAGCGTAACTCATTTCT | |
| T352H | TATCTACACGGGTCTCACCAAAACTTACTTTACTTGAAAAAAACACTTCGGGAGGATGAAG | 600 |
| | AGATGGGGTTGACTCATACATCTACTATCATGAGTCTGCAATGTCCAATTTCGTACAACTT | |
| | CGGGAGGATGAAGAAAGTTTTAGAGTGAGACCAGCGTAACTCCAGTAG | |
| T352I | TATCTACACGGGTCTCACCAAAACTTACTTTACTTGAAAAAAACACTTCGGGAGGATGAAG | 601 |
| | AGATGGGGTTGACTATTACATCTACTATCATGAGTCTGCAATGTCCAATTTCGTACAACTT | |
| | CGGGAGGATGAAGAAAGTTTTAGAGTGAGACCAGCGTAACTCTCTTGT | |
| T352L | TATCTACACGGGTCTCACCAAAACTTACTTTACTTGAAAAAAACACTTCGGGAGGATGAAG | 602 |
| | AGATGGGGTTGACTTTGACATCTACTATCATGAGTCTGCAATGTCCAATTTCGTACAACTT | |
| | CGGGAGGATGAAGAAAGTTTTAGAGTGAGACCAGCGTAACTCGAGTAT | |
| T352K | TATCTACACGGGTCTCACCAAAACTTACTTTACTTGAAAAAAACACTTCGGGAGGATGAAG | 603 |
| | AGATGGGGTTGACTAAAACATCTACTATCATGAGTCTGCAATGTCCAATTTCGTACAACTT | |
| | CGGGAGGATGAAGAAAGTTTTAGAGTGAGACCAGCGTAACTCTCAGTG | |
| T352M | TATCTACACGGGTCTCACCAAAACTTACTTTACTTGAAAAAAACACTTCGGGAGGATGAAG | 604 |
| | AGATGGGGTTGACTATGACATCTACTATCATGAGTCTGCAATGTCCAATTTCGTACAACTT | |
| | CGGGAGGATGAAGAAAGTTTTAGAGTGAGACCAGCGTAACTCCAAGTA | |
| T352F | TATCTACACGGGTCTCACCAAAACTTACTTTACTTGAAAAAAACACTTCGGGAGGATGAAG | 605 |
| | AGATGGGGTTGACTTTTACATCTACTATCATGAGTCTGCAATGTCCAATTTCGTACAACTT | |
| | CGGGAGGATGAAGAAAGTTTTAGAGTGAGACCAGCGTAACTCTGTTGG | |
| T352P | TATCTACACGGGTCTCACCAAAACTTACTTTACTTGAAAAAAACACTTCGGGAGGATGAAG | 606 |
| | AGATGGGGTTGACTCCAACATCTACTATCATGAGTCTGCAATGTCCAATTTCGTACAACTT | |
| | CGGGAGGATGAAGAAAGTTTTAGAGTGAGACCAGCGTAACTCACTATC | |
| T352S | TATCTACACGGGTCTCACCAAAACTTACTTTACTTGAAAAAAACACTTCGGGAGGATGAAG | 607 |
| | AGATGGGGTTGACTTCTACATCTACTATCATGAGTCTGCAATGTCCAATTTCGTACAACTT | |
| | CGGGAGGATGAAGAAAGTTTTAGAGTGAGACCAGCGTAACTCACTTAG | |
| T352T | TATCTACACGGGTCTCACCAAAACTTACTTTACTTGAAAAAAACACTTCGGGAGGATGAAG | 608 |
| | AGATGGGGTTGACTACTACATCTACTATCATGAGTCTGCAATGTCCAATTTCGTACAACTT | |
| | CGGGAGGATGAAGAAAGTTTTAGAGTGAGACCAGCGTAACTCGCTATC | |
| T352W | TATCTACACGGGTCTCACCAAAACTTACTTTACTTGAAAAAAACACTTCGGGAGGATGAAG | 609 |
| | AGATGGGGTTGACTTGGACATCTACTATCATGAGTCTGCAATGTCCAATTTCGTACAACTT | |
| | CGGGAGGATGAAGAAAGTTTTAGAGTGAGACCAGCGTAACTCTGGCGC | |
| T352Y | TATCTACACGGGTCTCACCAAAACTTACTTTACTTGAAAAAAACACTTCGGGAGGATGAAG | 610 |
| | AGATGGGGTTGACTTATACATCTACTATCATGAGTCTGCAATGTCCAATTTCGTACAACTT | |
| | CGGGAGGATGAAGAAAGTTTTAGAGTGAGACCAGCGTAACTCCGTAGT | |
| T352V | TATCTACACGGGTCTCACCAAAACTTACTTTACTTGAAAAAAACACTTCGGGAGGATGAAG | 611 |
| | AGATGGGGTTGACTGTTACATCTACTATCATGAGTCTGCAATGTCCAATTTCGTACAACTT | |
| | CGGGAGGATGAAGAAAGTTTTAGAGTGAGACCAGCGTAACTCCTGATT | |
| T353A | TATCTACACGGGTCTCACCAAAACTTACTTTACTTGAAAAAAACACTTCGGGAGGATGAAG | 612 |
| | AGATGGGGTTGACTACCGCTTCTACTATCATGAGTCTGCAATGTCCAATTTCGTACACAAA | |
| | CTTCGGGAGGATGAAGAAAGTTTTAGAGTGAGACCAGCGTAACTCGAT | |
| T353R | TATCTACACGGGTCTCACCAAAACTTACTTTACTTGAAAAAAACACTTCGGGAGGATGAAG | 613 |
| | AGATGGGGTTGACTACCAGATCTACTATCATGAGTCTGCAATGTCCAATTTCGTACACAAA | |
| | CTTCGGGAGGATGAAGAAAGTTTTAGAGTGAGACCAGCGTAACTCTGT | |
| T353N | TATCTACACGGGTCTCACCAAAACTTACTTTACTTGAAAAAAACACTTCGGGAGGATGAAG | 614 |
| | AGATGGGGTTGACTACCAATTCTACTATCATGAGTCTGCAATGTCCAATTTCGTACACAAA | |
| | CTTCGGGAGGATGAAGAAAGTTTTAGAGTGAGACCAGCGTAACTCAAA | |
| T353D | TATCTACACGGGTCTCACCAAAACTTACTTTACTTGAAAAAAACACTTCGGGAGGATGAAG | 615 |
| | AGATGGGGTTGACTACCGATTCTACTATCATGAGTCTGCAATGTCCAATTTCGTACACAAA | |
| | CTTCGGGAGGATGAAGAAAGTTTTAGAGTGAGACCAGCGTAACTCACC | |
| T353C | TATCTACACGGGTCTCACCAAAACTTACTTTACTTGAAAAAAACACTTCGGGAGGATGAAG | 616 |
| | AGATGGGGTTGACTACCTGTTCTACTATCATGAGTCTGCAATGTCCAATTTCGTACACAAA | |
| | CTTCGGGAGGATGAAGAAAGTTTTAGAGTGAGACCAGCGTAACTCTCT | |
| T353Q | TATCTACACGGGTCTCACCAAAACTTACTTTACTTGAAAAAAACACTTCGGGAGGATGAAG | 617 |
| | AGATGGGGTTGACTACCCAATCTACTATCATGAGTCTGCAATGTCCAATTTCGTACACAAA | |
| | CTTCGGGAGGATGAAGAAAGTTTTAGAGTGAGACCAGCGTAACTCCTG | |
| T353E | TATCTACACGGGTCTCACCAAAACTTACTTTACTTGAAAAAAACACTTCGGGAGGATGAAG | 618 |
| | AGATGGGGTTGACTACCGAATCTACTATCATGAGTCTGCAATGTCCAATTTCGTACACAAA | |
| | CTTCGGGAGGATGAAGAAAGTTTTAGAGTGAGACCAGCGTAACTCGCT | |
| T353G | TATCTACACGGGTCTCACCAAAACTTACTTTACTTGAAAAAAACACTTCGGGAGGATGAAG | 619 |
| | AGATGGGGTTGACTACCGGTTCTACTATCATGAGTCTGCAATGTCCAATTTCGTACACAAA | |
| | CTTCGGGAGGATGAAGAAAGTTTTAGAGTGAGACCAGCGTAACTCAGT | |
| T353H | TATCTACACGGGTCTCACCAAAACTTACTTTACTTGAAAAAAACACTTCGGGAGGATGAAG | 620 |
| | AGATGGGGTTGACTACCCATTCTACTATCATGAGTCTGCAATGTCCAATTTCGTACACAAA | |
| | CTTCGGGAGGATGAAGAAAGTTTTAGAGTGAGACCAGCGTAACTCAAG | |
| T353I | TATCTACACGGGTCTCACCAAAACTTACTTTACTTGAAAAAAACACTTCGGGAGGATGAAG | 621 |
| | AGATGGGGTTGACTACCATTTCTACTATCATGAGTCTGCAATGTCCAATTTCGTACACAAA | |
| | CTTCGGGAGGATGAAGAAAGTTTTAGAGTGAGACCAGCGTAACTCCGC | |
| T353L | TATCTACACGGGTCTCACCAAAACTTACTTTACTTGAAAAAAACACTTCGGGAGGATGAAG | 622 |
| | AGATGGGGTTGACTACCTTGTCTACTATCATGAGTCTGCAATGTCCAATTTCGTACACAAA | |
| | CTTCGGGAGGATGAAGAAAGTTTTAGAGTGAGACCAGCGTAACTCACT | |
| T353K | TATCTACACGGGTCTCACCAAAACTTACTTTACTTGAAAAAAACACTTCGGGAGGATGAAG | 623 |
| | AGATGGGGTTGACTACCAAATCTACTATCATGAGTCTGCAATGTCCAATTTCGTACACAAA | |
| | CTTCGGGAGGATGAAGAAAGTTTTAGAGTGAGACCAGCGTAACTCCAG | |
| T353M | TATCTACACGGGTCTCACCAAAACTTACTTTACTTGAAAAAAACACTTCGGGAGGATGAAG | 624 |
| | AGATGGGGTTGACTACCATGTCTACTATCATGAGTCTGCAATGTCCAATTTCGTACACAAA | |
| | CTTCGGGAGGATGAAGAAAGTTTTAGAGTGAGACCAGCGTAACTCAGC | |
| T353F | TATCTACACGGGTCTCACCAAAACTTACTTTACTTGAAAAAAACACTTCGGGAGGATGAAG | 625 |
| | AGATGGGGTTGACTACCTTTTCTACTATCATGAGTCTGCAATGTCCAATTTCGTACACAAA | |
| | CTTCGGGAGGATGAAGAAAGTTTTAGAGTGAGACCAGCGTAACTCGGC | |
| T353P | TATCTACACGGGTCTCACCAAAACTTACTTTACTTGAAAAAAACACTTCGGGAGGATGAAG | 626 |
| | AGATGGGGTTGACTACCCCATCTACTATCATGAGTCTGCAATGTCCAATTTCGTACACAAA | |
| | CTTCGGGAGGATGAAGAAAGTTTTAGAGTGAGACCAGCGTAACTCATT | |
| T353S | TATCTACACGGGTCTCACCAAAACTTACTTTACTTGAAAAAAACACTTCGGGAGGATGAAG | 627 |
| | AGATGGGGTTGACTACCTCTTCTACTATCATGAGTCTGCAATGTCCAATTTCGTACACAAA | |
| | CTTCGGGAGGATGAAGAAAGTTTTAGAGTGAGACCAGCGTAACTCCGC | |
| T353T | TATCTACACGGGTCTCACCAAAACTTACTTTACTTGAAAAAAACACTTCGGGAGGATGAAG | 628 |
| | AGATGGGGTTGACTACCACTTCTACTATCATGAGTCTGCAATGTCCAATTTCGTACACAAA | |
| | CTTCGGGAGGATGAAGAAAGTTTTAGAGTGAGACCAGCGTAACTCAGT | |
| T353W | TATCTACACGGGTCTCACCAAAACTTACTTTACTTGAAAAAAACACTTCGGGAGGATGAAG | 629 |
| | AGATGGGGTTGACTACCTGGTCTACTATCATGAGTCTGCAATGTCCAATTTCGTACACAAA | |
| | CTTCGGGAGGATGAAGAAAGTTTTAGAGTGAGACCAGCGTAACTCGAC | |
| T353Y | TATCTACACGGGTCTCACCAAAACTTACTTTACTTGAAAAAAACACTTCGGGAGGATGAAG | 630 |
| | AGATGGGGTTGACTACCTATTCTACTATCATGAGTCTGCAATGTCCAATTTCGTACACAAA | |
| | CTTCGGGAGGATGAAGAAAGTTTTAGAGTGAGACCAGCGTAACTCAGC | |
| T353V | TATCTACACGGGTCTCACCAAAACTTACTTTACTTGAAAAAAACACTTCGGGAGGATGAAG | 631 |
| | AGATGGGGTTGACTACCGTTTCTACTATCATGAGTCTGCAATGTCCAATTTCGTACACAAA | |
| | CTTCGGGAGGATGAAGAAAGTTTTAGAGTGAGACCAGCGTAACTCTGT | |
| S354A | TATCTACACGGGTCTCACCAAAACAAAAAAACACTTCGGGAGGATGAAGAAATGGGCTTGA | 632 |
| | CTACGACGGCTACTATCATGAGTCTGCAATGTCCAATTTCGTACACAAGAACAGACTCATG | |
| | ATAGTAGATGGTTTTAGAGTGAGACCAGCGTAACTCTTAAAGGTGTTA | |
| S354R | TATCTACACGGGTCTCACCAAAACAAAAAAACACTTCGGGAGGATGAAGAAATGGGCTTGA | 633 |
| | CTACGACGAGAACTATCATGAGTCTGCAATGTCCAATTTCGTACACAAGAACAGACTCATG | |
| | ATAGTAGATGGTTTTAGAGTGAGACCAGCGTAACTCAAACACGGGGAT | |
| S354N | TATCTACACGGGTCTCACCAAAACAAAAAAACACTTCGGGAGGATGAAGAAATGGGCTTGA | 634 |
| | CTACGACGAATACTATCATGAGTCTGCAATGTCCAATTTCGTACACAAGAACAGACTCATG | |
| | ATAGTAGATGGTTTTAGAGTGAGACCAGCGTAACTCGTCTCTGGGAGC | |
| S354D | TATCTACACGGGTCTCACCAAAACAAAAAAACACTTCGGGAGGATGAAGAAATGGGCTTGA | 635 |
| | CTACGACGGATACTATCATGAGTCTGCAATGTCCAATTTCGTACACAAGAACAGACTCATG | |
| | ATAGTAGATGGTTTTAGAGTGAGACCAGCGTAACTCAAAGTATTTCAT | |
| S354C | TATCTACACGGGTCTCACCAAAACAAAAAAACACTTCGGGAGGATGAAGAAATGGGCTTGA | 636 |
| | CTACGACGTGTACTATCATGAGTCTGCAATGTCCAATTTCGTACACAAGAACAGACTCATG | |
| | ATAGTAGATGGTTTTAGAGTGAGACCAGCGTAACTCCTCGACTATCGA | |
| S354Q | TATCTACACGGGTCTCACCAAAACAAAAAAACACTTCGGGAGGATGAAGAAATGGGCTTGA | 637 |
| | CTACGACGCAAACTATCATGAGTCTGCAATGTCCAATTTCGTACACAAGAACAGACTCATG | |
| | ATAGTAGATGGTTTTAGAGTGAGACCAGCGTAACTCCCCTCGTGGTCG | |
| S354E | TATCTACACGGGTCTCACCAAAACAAAAAAACACTTCGGGAGGATGAAGAAATGGGCTTGA | 638 |
| | CTACGACGGAAACTATCATGAGTCTGCAATGTCCAATTTCGTACACAAGAACAGACTCATG | |
| | ATAGTAGATGGTTTTAGAGTGAGACCAGCGTAACTCAGGCGGCGTCAC | |
| S354G | TATCTACACGGGTCTCACCAAAACAAAAAAACACTTCGGGAGGATGAAGAAATGGGCTTGA | 639 |
| | CTACGACGGGTACTATCATGAGTCTGCAATGTCCAATTTCGTACACAAGAACAGACTCATG | |
| | ATAGTAGATGGTTTTAGAGTGAGACCAGCGTAACTCTCATCCTGTTAG | |
| S354H | TATCTACACGGGTCTCACCAAAACAAAAAAACACTTCGGGAGGATGAAGAAATGGGCTTGA | 640 |
| | CTACGACGCATACTATCATGAGTCTGCAATGTCCAATTTCGTACACAAGAACAGACTCATG | |
| | ATAGTAGATGGTTTTAGAGTGAGACCAGCGTAACTCGAGTGTAATTTA | |
| S354I | TATCTACACGGGTCTCACCAAAACAAAAAAACACTTCGGGAGGATGAAGAAATGGGCTTGA | 641 |
| | CTACGACGATTACTATCATGAGTCTGCAATGTCCAATTTCGTACACAAGAACAGACTCATG | |
| | ATAGTAGATGGTTTTAGAGTGAGACCAGCGTAACTCGACAAAGAAACC | |
| S354L | TATCTACACGGGTCTCACCAAAACAAAAAAACACTTCGGGAGGATGAAGAAATGGGCTTGA | 642 |
| | CTACGACGTTGACTATCATGAGTCTGCAATGTCCAATTTCGTACACAAGAACAGACTCATG | |
| | ATAGTAGATGGTTTTAGAGTGAGACCAGCGTAACTCGGCCAGGTGCGA | |
| S354K | TATCTACACGGGTCTCACCAAAACAAAAAAACACTTCGGGAGGATGAAGAAATGGGCTTGA | 643 |
| | CTACGACGAAAACTATCATGAGTCTGCAATGTCCAATTTCGTACACAAGAACAGACTCATG | |
| | ATAGTAGATGGTTTTAGAGTGAGACCAGCGTAACTCAGATGGGCGGGC | |
| S354M | TATCTACACGGGTCTCACCAAAACAAAAAAACACTTCGGGAGGATGAAGAAATGGGCTTGA | 644 |
| | CTACGACGATGACTATCATGAGTCTGCAATGTCCAATTTCGTACACAAGAACAGACTCATG | |
| | ATAGTAGATGGTTTTAGAGTGAGACCAGCGTAACTCTTCTTAAACCCT | |
| S354F | TATCTACACGGGTCTCACCAAAACAAAAAAACACTTCGGGAGGATGAAGAAATGGGCTTGA | 645 |
| | CTACGACGTTTACTATCATGAGTCTGCAATGTCCAATTTCGTACACAAGAACAGACTCATG | |
| | ATAGTAGATGGTTTTAGAGTGAGACCAGCGTAACTCGACTGGTAAGCA | |
| S354P | TATCTACACGGGTCTCACCAAAACAAAAAAACACTTCGGGAGGATGAAGAAATGGGCTTGA | 646 |
| | CTACGACGCCAACTATCATGAGTCTGCAATGTCCAATTTCGTACACAAGAACAGACTCATG | |
| | ATAGTAGATGGTTTTAGAGTGAGACCAGCGTAACTCATCTTCGTCTCT | |
| S354S | TATCTACACGGGTCTCACCAAAACAAAAAAACACTTCGGGAGGATGAAGAAATGGGCTTGA | 647 |
| | CTACGACGAGTACTATCATGAGTCTGCAATGTCCAATTTCGTACACAAGAACAGACTCATG | |
| | ATAGTAGATGGTTTTAGAGTGAGACCAGCGTAACTCGCGACCCCTTGA | |
| S354T | TATCTACACGGGTCTCACCAAAACAAAAAAACACTTCGGGAGGATGAAGAAATGGGCTTGA | 648 |
| | CTACGACGACTACTATCATGAGTCTGCAATGTCCAATTTCGTACACAAGAACAGACTCATG | |
| | ATAGTAGATGGTTTTAGAGTGAGACCAGCGTAACTCCTCATTGTCTCA | |
| S354W | TATCTACACGGGTCTCACCAAAACAAAAAAACACTTCGGGAGGATGAAGAAATGGGCTTGA | 649 |
| | CTACGACGTGGACTATCATGAGTCTGCAATGTCCAATTTCGTACACAAGAACAGACTCATG | |
| | ATAGTAGATGGTTTTAGAGTGAGACCAGCGTAACTCTCAGCGATCTTA | |
| S354Y | TATCTACACGGGTCTCACCAAAACAAAAAAACACTTCGGGAGGATGAAGAAATGGGCTTGA | 650 |
| | CTACGACGTATACTATCATGAGTCTGCAATGTCCAATTTCGTACACAAGAACAGACTCATG | |
| | ATAGTAGATGGTTTTAGAGTGAGACCAGCGTAACTCTGGGTCCGGTTG | |
| S354V | TATCTACACGGGTCTCACCAAAACAAAAAAACACTTCGGGAGGATGAAGAAATGGGCTTGA | 651 |
| | CTACGACGGTTACTATCATGAGTCTGCAATGTCCAATTTCGTACACAAGAACAGACTCATG | |
| | ATAGTAGATGGTTTTAGAGTGAGACCAGCGTAACTCGTCCGGGAGTTG | |
| T355A | TATCTACACGGGTCTCACCAAAACAAAAAAACACTTCGGGAGGATGAAGAAATGGGCTTGA | 652 |
| | CTACGACGTCTGCTATCATGAGTCTGCAATGTCCAATTTCGTACACAAGAATGACAGACTC | |
| | ATGATAGTAGATGGTTTTAGAGTGAGACCAGCGTAACTCTGTCGGATT | |
| T355R | TATCTACACGGGTCTCACCAAAACAAAAAAACACTTCGGGAGGATGAAGAAATGGGCTTGA | 653 |
| | CTACGACGTCTAGAATCATGAGTCTGCAATGTCCAATTTCGTACACAAGAATGACAGACTC | |
| | ATGATAGTAGATGGTTTTAGAGTGAGACCAGCGTAACTCACTGAGCCC | |
| T355N | TATCTACACGGGTCTCACCAAAACAAAAAAACACTTCGGGAGGATGAAGAAATGGGCTTGA | 654 |
| | CTACGACGTCTAATATCATGAGTCTGCAATGTCCAATTTCGTACACAAGAATGACAGACTC | |
| | ATGATAGTAGATGGTTTTAGAGTGAGACCAGCGTAACTCTCGGAGAGC | |
| T355D | TATCTACACGGGTCTCACCAAAACAAAAAAACACTTCGGGAGGATGAAGAAATGGGCTTGA | 655 |
| | CTACGACGTCTGATATCATGAGTCTGCAATGTCCAATTTCGTACACAAGAATGACAGACTC | |
| | ATGATAGTAGATGGTTTTAGAGTGAGACCAGCGTAACTCACAGACACG | |
| T355C | TATCTACACGGGTCTCACCAAAACAAAAAAACACTTCGGGAGGATGAAGAAATGGGCTTGA | 656 |
| | CTACGACGTCTTGTATCATGAGTCTGCAATGTCCAATTTCGTACACAAGAATGACAGACTC | |
| | ATGATAGTAGATGGTTTTAGAGTGAGACCAGCGTAACTCGTGTGATCG | |
| T355Q | TATCTACACGGGTCTCACCAAAACAAAAAAACACTTCGGGAGGATGAAGAAATGGGCTTGA | 657 |
| | CTACGACGTCTCAAATCATGAGTCTGCAATGTCCAATTTCGTACACAAGAATGACAGACTC | |
| | ATGATAGTAGATGGTTTTAGAGTGAGACCAGCGTAACTCAAAAGTCCC | |
| T355E | TATCTACACGGGTCTCACCAAAACAAAAAAACACTTCGGGAGGATGAAGAAATGGGCTTGA | 658 |
| | CTACGACGTCTGAAATCATGAGTCTGCAATGTCCAATTTCGTACACAAGAATGACAGACTC | |
| | ATGATAGTAGATGGTTTTAGAGTGAGACCAGCGTAACTCCCAAAACGC | |
| T355G | TATCTACACGGGTCTCACCAAAACAAAAAAACACTTCGGGAGGATGAAGAAATGGGCTTGA | 659 |
| | CTACGACGTCTGGTATCATGAGTCTGCAATGTCCAATTTCGTACACAAGAATGACAGACTC | |
| | ATGATAGTAGATGGTTTTAGAGTGAGACCAGCGTAACTCAGGCTCATT | |
| T355H | TATCTACACGGGTCTCACCAAAACAAAAAAACACTTCGGGAGGATGAAGAAATGGGCTTGA | 660 |
| | CTACGACGTCTCATATCATGAGTCTGCAATGTCCAATTTCGTACACAAGAATGACAGACTC | |
| | ATGATAGTAGATGGTTTTAGAGTGAGACCAGCGTAACTCTCAACGCTT | |
| T355I | TATCTACACGGGTCTCACCAAAACAAAAAAACACTTCGGGAGGATGAAGAAATGGGCTTGA | 661 |
| | CTACGACGTCTATTATCATGAGTCTGCAATGTCCAATTTCGTACACAAGAATGACAGACTC | |
| | ATGATAGTAGATGGTTTTAGAGTGAGACCAGCGTAACTCTGGTATACT | |
| T355L | TATCTACACGGGTCTCACCAAAACAAAAAAACACTTCGGGAGGATGAAGAAATGGGCTTGA | 662 |
| | CTACGACGTCTTTGATCATGAGTCTGCAATGTCCAATTTCGTACACAAGAATGACAGACTC | |
| | ATGATAGTAGATGGTTTTAGAGTGAGACCAGCGTAACTCGTATAGCGT | |
| T355K | TATCTACACGGGTCTCACCAAAACAAAAAAACACTTCGGGAGGATGAAGAAATGGGCTTGA | 663 |
| | CTACGACGTCTAAAATCATGAGTCTGCAATGTCCAATTTCGTACACAAGAATGACAGACTC | |
| | ATGATAGTAGATGGTTTTAGAGTGAGACCAGCGTAACTCCGGCTAAAG | |
| T355M | TATCTACACGGGTCTCACCAAAACAAAAAAACACTTCGGGAGGATGAAGAAATGGGCTTGA | 664 |
| | CTACGACGTCTATGATCATGAGTCTGCAATGTCCAATTTCGTACACAAGAATGACAGACTC | |
| | ATGATAGTAGATGGTTTTAGAGTGAGACCAGCGTAACTCCGCCGTATG | |
| T355F | TATCTACACGGGTCTCACCAAAACAAAAAAACACTTCGGGAGGATGAAGAAATGGGCTTGA | 665 |
| | CTACGACGTCTTTTATCATGAGTCTGCAATGTCCAATTTCGTACACAAGAATGACAGACTC | |
| | ATGATAGTAGATGGTTTTAGAGTGAGACCAGCGTAACTCGCCTGCGCG | |
| T355P | TATCTACACGGGTCTCACCAAAACAAAAAAACACTTCGGGAGGATGAAGAAATGGGCTTGA | 666 |
| | CTACGACGTCTCCAATCATGAGTCTGCAATGTCCAATTTCGTACACAAGAATGACAGACTC | |
| | ATGATAGTAGATGGTTTTAGAGTGAGACCAGCGTAACTCAGAGCAATT | |
| T355S | TATCTACACGGGTCTCACCAAAACAAAAAAACACTTCGGGAGGATGAAGAAATGGGCTTGA | 667 |
| | CTACGACGTCTTCTATCATGAGTCTGCAATGTCCAATTTCGTACACAAGAATGACAGACTC | |
| | ATGATAGTAGATGGTTTTAGAGTGAGACCAGCGTAACTCCCAATTGAT | |
| T355T | TATCTACACGGGTCTCACCAAAACAAAAAAACACTTCGGGAGGATGAAGAAATGGGCTTGA | 668 |
| | CTACGACGTCTACAATCATGAGTCTGCAATGTCCAATTTCGTACACAAGAATGACAGACTC | |
| | ATGATAGTAGATGGTTTTAGAGTGAGACCAGCGTAACTCTCACAAATG | |
| T355W | TATCTACACGGGTCTCACCAAAACAAAAAAACACTTCGGGAGGATGAAGAAATGGGCTTGA | 669 |
| | CTACGACGTCTTGGATCATGAGTCTGCAATGTCCAATTTCGTACACAAGAATGACAGACTC | |
| | ATGATAGTAGATGGTTTTAGAGTGAGACCAGCGTAACTCCAACCCTTT | |
| T355Y | TATCTACACGGGTCTCACCAAAACAAAAAAACACTTCGGGAGGATGAAGAAATGGGCTTGA | 670 |
| | CTACGACGTCTTATATCATGAGTCTGCAATGTCCAATTTCGTACACAAGAATGACAGACTC | |
| | ATGATAGTAGATGGTTTTAGAGTGAGACCAGCGTAACTCCTCGTAGGA | |
| T355V | TATCTACACGGGTCTCACCAAAACAAAAAAACACTTCGGGAGGATGAAGAAATGGGCTTGA | 671 |
| | CTACGACGTCTGTTATCATGAGTCTGCAATGTCCAATTTCGTACACAAGAATGACAGACTC | |
| | ATGATAGTAGATGGTTTTAGAGTGAGACCAGCGTAACTCGGCTGTCAA | |
| I356A | TATCTACACGGGTCTCACCAAAACAAAAAAACACTTCGGGAGGATGAAGAAATGGGCTTGA | 672 |
| | CTACGACGTCTACTGCTATGAGTCTGCAATGTCCAATTTCGTACACAAGAATGAAATCAGA | |
| | CTCATGATAGTAGATGGTTTTAGAGTGAGACCAGCGTAACTCGGTTGT | |
| I356R | TATCTACACGGGTCTCACCAAAACAAAAAAACACTTCGGGAGGATGAAGAAATGGGCTTGA | 673 |
| | CTACGACGTCTACTAGAATGAGTCTGCAATGTCCAATTTCGTACACAAGAATGAAATCAGA | |
| | CTCATGATAGTAGATGGTTTTAGAGTGAGACCAGCGTAACTCCAGGAA | |
| I356N | TATCTACACGGGTCTCACCAAAACAAAAAAACACTTCGGGAGGATGAAGAAATGGGCTTGA | 674 |
| | CTACGACGTCTACTAATATGAGTCTGCAATGTCCAATTTCGTACACAAGAATGAAATCAGA | |
| | CTCATGATAGTAGATGGTTTTAGAGTGAGACCAGCGTAACTCAGACTA | |
| I356D | TATCTACACGGGTCTCACCAAAACAAAAAAACACTTCGGGAGGATGAAGAAATGGGCTTGA | 675 |
| | CTACGACGTCTACTGATATGAGTCTGCAATGTCCAATTTCGTACACAAGAATGAAATCAGA | |
| | CTCATGATAGTAGATGGTTTTAGAGTGAGACCAGCGTAACTCTCTACC | |
| I356C | TATCTACACGGGTCTCACCAAAACAAAAAAACACTTCGGGAGGATGAAGAAATGGGCTTGA | 676 |
| | CTACGACGTCTACTTGTATGAGTCTGCAATGTCCAATTTCGTACACAAGAATGAAATCAGA | |
| | CTCATGATAGTAGATGGTTTTAGAGTGAGACCAGCGTAACTCCGAGCT | |
| I356Q | TATCTACACGGGTCTCACCAAAACAAAAAAACACTTCGGGAGGATGAAGAAATGGGCTTGA | 677 |
| | CTACGACGTCTACTCAAATGAGTCTGCAATGTCCAATTTCGTACACAAGAATGAAATCAGA | |
| | CTCATGATAGTAGATGGTTTTAGAGTGAGACCAGCGTAACTCCTGTCG | |
| I356E | TATCTACACGGGTCTCACCAAAACAAAAAAACACTTCGGGAGGATGAAGAAATGGGCTTGA | 678 |
| | CTACGACGTCTACTGAAATGAGTCTGCAATGTCCAATTTCGTACACAAGAATGAAATCAGA | |
| | CTCATGATAGTAGATGGTTTTAGAGTGAGACCAGCGTAACTCATAGGC | |
| I356G | TATCTACACGGGTCTCACCAAAACAAAAAAACACTTCGGGAGGATGAAGAAATGGGCTTGA | 679 |
| | CTACGACGTCTACTGGTATGAGTCTGCAATGTCCAATTTCGTACACAAGAATGAAATCAGA | |
| | CTCATGATAGTAGATGGTTTTAGAGTGAGACCAGCGTAACTCAAGTGA | |
| I356H | TATCTACACGGGTCTCACCAAAACAAAAAAACACTTCGGGAGGATGAAGAAATGGGCTTGA | 680 |
| | CTACGACGTCTACTCATATGAGTCTGCAATGTCCAATTTCGTACACAAGAATGAAATCAGA | |
| | CTCATGATAGTAGATGGTTTTAGAGTGAGACCAGCGTAACTCTCGGGC | |
| I356I | TATCTACACGGGTCTCACCAAAACAAAAAAACACTTCGGGAGGATGAAGAAATGGGCTTGA | 681 |
| | CTACGACGTCTACTATTATGAGTCTGCAATGTCCAATTTCGTACACAAGAATGAAATCAGA | |
| | CTCATGATAGTAGATGGTTTTAGAGTGAGACCAGCGTAACTCCCCTCG | |
| I356L | TATCTACACGGGTCTCACCAAAACAAAAAAACACTTCGGGAGGATGAAGAAATGGGCTTGA | 682 |
| | CTACGACGTCTACTTTGATGAGTCTGCAATGTCCAATTTCGTACACAAGAATGAAATCAGA | |
| | CTCATGATAGTAGATGGTTTTAGAGTGAGACCAGCGTAACTCTAGCCT | |
| I356K | TATCTACACGGGTCTCACCAAAACAAAAAAACACTTCGGGAGGATGAAGAAATGGGCTTGA | 683 |
| | CTACGACGTCTACTAAAATGAGTCTGCAATGTCCAATTTCGTACACAAGAATGAAATCAGA | |
| | CTCATGATAGTAGATGGTTTTAGAGTGAGACCAGCGTAACTCATGGAG | |
| I356M | TATCTACACGGGTCTCACCAAAACAAAAAAACACTTCGGGAGGATGAAGAAATGGGCTTGA | 684 |
| | CTACGACGTCTACTATGATGAGTCTGCAATGTCCAATTTCGTACACAAGAATGAAATCAGA | |
| | CTCATGATAGTAGATGGTTTTAGAGTGAGACCAGCGTAACTCCGAGTT | |
| I356F | TATCTACACGGGTCTCACCAAAACAAAAAAACACTTCGGGAGGATGAAGAAATGGGCTTGA | 685 |
| | CTACGACGTCTACTTTTATGAGTCTGCAATGTCCAATTTCGTACACAAGAATGAAATCAGA | |
| | CTCATGATAGTAGATGGTTTTAGAGTGAGACCAGCGTAACTCACTGGA | |
| I356P | TATCTACACGGGTCTCACCAAAACAAAAAAACACTTCGGGAGGATGAAGAAATGGGCTTGA | 686 |
| | CTACGACGTCTACTCCAATGAGTCTGCAATGTCCAATTTCGTACACAAGAATGAAATCAGA | |
| | CTCATGATAGTAGATGGTTTTAGAGTGAGACCAGCGTAACTCTGGTTC | |
| I356S | TATCTACACGGGTCTCACCAAAACAAAAAAACACTTCGGGAGGATGAAGAAATGGGCTTGA | 687 |
| | CTACGACGTCTACTTCTATGAGTCTGCAATGTCCAATTTCGTACACAAGAATGAAATCAGA | |
| | CTCATGATAGTAGATGGTTTTAGAGTGAGACCAGCGTAACTCACCGCT | |
| I356T | TATCTACACGGGTCTCACCAAAACAAAAAAACACTTCGGGAGGATGAAGAAATGGGCTTGA | 688 |
| | CTACGACGTCTACTACTATGAGTCTGCAATGTCCAATTTCGTACACAAGAATGAAATCAGA | |
| | CTCATGATAGTAGATGGTTTTAGAGTGAGACCAGCGTAACTCCTCAAG | |
| I356W | TATCTACACGGGTCTCACCAAAACAAAAAAACACTTCGGGAGGATGAAGAAATGGGCTTGA | 689 |
| | CTACGACGTCTACTTGGATGAGTCTGCAATGTCCAATTTCGTACACAAGAATGAAATCAGA | |
| | CTCATGATAGTAGATGGTTTTAGAGTGAGACCAGCGTAACTCGCTTGA | |
| I356Y | TATCTACACGGGTCTCACCAAAACAAAAAAACACTTCGGGAGGATGAAGAAATGGGCTTGA | 690 |
| | CTACGACGTCTACTTATATGAGTCTGCAATGTCCAATTTCGTACACAAGAATGAAATCAGA | |
| | CTCATGATAGTAGATGGTTTTAGAGTGAGACCAGCGTAACTCGCCATG | |
| I356V | TATCTACACGGGTCTCACCAAAACAAAAAAACACTTCGGGAGGATGAAGAAATGGGCTTGA | 691 |
| | CTACGACGTCTACTGTTATGAGTCTGCAATGTCCAATTTCGTACACAAGAATGAAATCAGA | |
| | CTCATGATAGTAGATGGTTTTAGAGTGAGACCAGCGTAACTCAGCGCC | |
| M357A | TATCTACACGGGTCTCACCAAAACAAAAAAACACTTCGGGAGGATGAAGAAATGGGCTTGA | 692 |
| | CTACGACGTCTACTATCGCTAGTCTGCAATGTCCAATTTCGTACACAAGAATGAAATACCC | |
| | AGACTCATGATAGTAGATGGTTTTAGAGTGAGACCAGCGTAACTCTCG | |
| M357R | TATCTACACGGGTCTCACCAAAACAAAAAAACACTTCGGGAGGATGAAGAAATGGGCTTGA | 693 |
| | CTACGACGTCTACTATCAGAAGTCTGCAATGTCCAATTTCGTACACAAGAATGAAATACCC | |
| | AGACTCATGATAGTAGATGGTTTTAGAGTGAGACCAGCGTAACTCGTA | |
| M357N | TATCTACACGGGTCTCACCAAAACAAAAAAACACTTCGGGAGGATGAAGAAATGGGCTTGA | 694 |
| | CTACGACGTCTACTATCAATAGTCTGCAATGTCCAATTTCGTACACAAGAATGAAATACCC | |
| | AGACTCATGATAGTAGATGGTTTTAGAGTGAGACCAGCGTAACTCCAG | |
| M357D | TATCTACACGGGTCTCACCAAAACAAAAAAACACTTCGGGAGGATGAAGAAATGGGCTTGA | 695 |
| | CTACGACGTCTACTATCGATAGTCTGCAATGTCCAATTTCGTACACAAGAATGAAATACCC | |
| | AGACTCATGATAGTAGATGGTTTTAGAGTGAGACCAGCGTAACTCTCA | |
| M357C | TATCTACACGGGTCTCACCAAAACAAAAAAACACTTCGGGAGGATGAAGAAATGGGCTTGA | 696 |
| | CTACGACGTCTACTATCTGTAGTCTGCAATGTCCAATTTCGTACACAAGAATGAAATACCC | |
| | AGACTCATGATAGTAGATGGTTTTAGAGTGAGACCAGCGTAACTCTAG | |
| M357Q | TATCTACACGGGTCTCACCAAAACAAAAAAACACTTCGGGAGGATGAAGAAATGGGCTTGA | 697 |
| | CTACGACGTCTACTATCCAAAGTCTGCAATGTCCAATTTCGTACACAAGAATGAAATACCC | |
| | AGACTCATGATAGTAGATGGTTTTAGAGTGAGACCAGCGTAACTCACC | |
| M357E | TATCTACACGGGTCTCACCAAAACAAAAAAACACTTCGGGAGGATGAAGAAATGGGCTTGA | 698 |
| | CTACGACGTCTACTATCGAAAGTCTGCAATGTCCAATTTCGTACACAAGAATGAAATACCC | |
| | AGACTCATGATAGTAGATGGTTTTAGAGTGAGACCAGCGTAACTCTTA | |
| M357G | TATCTACACGGGTCTCACCAAAACAAAAAAACACTTCGGGAGGATGAAGAAATGGGCTTGA | 699 |
| | CTACGACGTCTACTATCGGTAGTCTGCAATGTCCAATTTCGTACACAAGAATGAAATACCC | |
| | AGACTCATGATAGTAGATGGTTTTAGAGTGAGACCAGCGTAACTCGTG | |
| M357H | TATCTACACGGGTCTCACCAAAACAAAAAAACACTTCGGGAGGATGAAGAAATGGGCTTGA | 700 |
| | CTACGACGTCTACTATCCATAGTCTGCAATGTCCAATTTCGTACACAAGAATGAAATACCC | |
| | AGACTCATGATAGTAGATGGTTTTAGAGTGAGACCAGCGTAACTCCCA | |
| M357I | TATCTACACGGGTCTCACCAAAACAAAAAAACACTTCGGGAGGATGAAGAAATGGGCTTGA | 701 |
| | CTACGACGTCTACTATCATTAGTCTGCAATGTCCAATTTCGTACACAAGAATGAAATACCC | |
| | AGACTCATGATAGTAGATGGTTTTAGAGTGAGACCAGCGTAACTCAAG | |
| M357L | TATCTACACGGGTCTCACCAAAACAAAAAAACACTTCGGGAGGATGAAGAAATGGGCTTGA | 702 |
| | CTACGACGTCTACTATCTTGAGTCTGCAATGTCCAATTTCGTACACAAGAATGAAATACCC | |
| | AGACTCATGATAGTAGATGGTTTTAGAGTGAGACCAGCGTAACTCGAT | |
| M357K | TATCTACACGGGTCTCACCAAAACAAAAAAACACTTCGGGAGGATGAAGAAATGGGCTTGA | 703 |
| | CTACGACGTCTACTATCAAAAGTCTGCAATGTCCAATTTCGTACACAAGAATGAAATACCC | |
| | AGACTCATGATAGTAGATGGTTTTAGAGTGAGACCAGCGTAACTCTTA | |
| M357M | TATCTACACGGGTCTCACCAAAACAAAAAAACACTTCGGGAGGATGAAGAAATGGGCTTGA | 704 |
| | CTACGACTTCTACTATCATGAGTCTGCAATGTCCAATTTCGTACACAAGAATGAAATACCC | |
| | AGACTCATGATAGTAGATGGTTTTAGAGTGAGACCAGCGTAACTCCTA | |
| M357F | TATCTACACGGGTCTCACCAAAACAAAAAAACACTTCGGGAGGATGAAGAAATGGGCTTGA | 705 |
| | CTACGACGTCTACTATCTTTAGTCTGCAATGTCCAATTTCGTACACAAGAATGAAATACCC | |
| | AGACTCATGATAGTAGATGGTTTTAGAGTGAGACCAGCGTAACTCGAG | |
| M357P | TATCTACACGGGTCTCACCAAAACAAAAAAACACTTCGGGAGGATGAAGAAATGGGCTTGA | 706 |
| | CTACGACGTCTACTATCCCAAGTCTGCAATGTCCAATTTCGTACACAAGAATGAAATACCC | |
| | AGACTCATGATAGTAGATGGTTTTAGAGTGAGACCAGCGTAACTCGTC | |
| M357S | TATCTACACGGGTCTCACCAAAACAAAAAAACACTTCGGGAGGATGAAGAAATGGGCTTGA | 707 |
| | CTACGACGTCTACTATCTCTAGTCTGCAATGTCCAATTTCGTACACAAGAATGAAATACCC | |
| | AGACTCATGATAGTAGATGGTTTTAGAGTGAGACCAGCGTAACTCAGT | |
| M357T | TATCTACACGGGTCTCACCAAAACAAAAAAACACTTCGGGAGGATGAAGAAATGGGCTTGA | 708 |
| | CTACGACGTCTACTATCACTAGTCTGCAATGTCCAATTTCGTACACAAGAATGAAATACCC | |
| | AGACTCATGATAGTAGATGGTTTTAGAGTGAGACCAGCGTAACTCCAC | |
| M357W | TATCTACACGGGTCTCACCAAAACAAAAAAACACTTCGGGAGGATGAAGAAATGGGCTTGA | 709 |
| | CTACGACGTCTACTATCTGGAGTCTGCAATGTCCAATTTCGTACACAAGAATGAAATACCC | |
| | AGACTCATGATAGTAGATGGTTTTAGAGTGAGACCAGCGTAACTCGTA | |
| M357Y | TATCTACACGGGTCTCACCAAAACAAAAAAACACTTCGGGAGGATGAAGAAATGGGCTTGA | 710 |
| | CTACGACGTCTACTATCTATAGTCTGCAATGTCCAATTTCGTACACAAGAATGAAATACCC | |
| | AGACTCATGATAGTAGATGGTTTTAGAGTGAGACCAGCGTAACTCAGA | |
| M357V | TATCTACACGGGTCTCACCAAAACAAAAAAACACTTCGGGAGGATGAAGAAATGGGCTTGA | 711 |
| | CTACGACGTCTACTATCGTTAGTCTGCAATGTCCAATTTCGTACACAAGAATGAAATACCC | |
| | AGACTCATGATAGTAGATGGTTTTAGAGTGAGACCAGCGTAACTCTGG | |
| S358A | TATCTACACGGGTCTCACCAAAACTTATTGATTTTGAAGGGTATTTCATTCTTGTGTACGA | 712 |
| | GATCGGACATTGCAGAGCCATGATAGTAGATGTGGTAGTCAAGCCCATTTCTTCATCCTTC | |
| | ATTCTTGTGTACGAAATGTTTTAGAGTGAGACCAGCGTAACTCTTGCC | |
| S358R | TATCTACACGGGTCTCACCAAAACTTATTGATTTTGAAGGGTATTTCATTCTTGTGTACGA | 713 |
| | GATCGGACATTGCAGTCTCATGATAGTAGATGTGGTAGTCAAGCCCATTTCTTCATCCTTC | |
| | ATTCTTGTGTACGAAATGTTTTAGAGTGAGACCAGCGTAACTCTGCCC | |
| S358N | TATCTACACGGGTCTCACCAAAACTTATTGATTTTGAAGGGTATTTCATTCTTGTGTACGA | 714 |
| | GATCGGACATTGCAGATTCATGATAGTAGATGTGGTAGTCAAGCCCATTTCTTCATCCTTC | |
| | ATTCTTGTGTACGAAATGTTTTAGAGTGAGACCAGCGTAACTCGCGCT | |
| S358D | TATCTACACGGGTCTCACCAAAACTTATTGATTTTGAAGGGTATTTCATTCTTGTGTACGA | 715 |
| | GATCGGACATTGCAGATCCATGATAGTAGATGTGGTAGTCAAGCCCATTTCTTCATCCTTC | |
| | ATTCTTGTGTACGAAATGTTTTAGAGTGAGACCAGCGTAACTCGGGTG | |
| S358C | TATCTACACGGGTCTCACCAAAACTTATTGATTTTGAAGGGTATTTCATTCTTGTGTACGA | 716 |
| | GATCGGACATTGCAGACACATGATAGTAGATGTGGTAGTCAAGCCCATTTCTTCATCCTTC | |
| | ATTCTTGTGTACGAAATGTTTTAGAGTGAGACCAGCGTAACTCCGGCC | |
| S358Q | TATCTACACGGGTCTCACCAAAACTTATTGATTTTGAAGGGTATTTCATTCTTGTGTACGA | 717 |
| | GATCGGACATTGCAGTTGCATGATAGTAGATGTGGTAGTCAAGCCCATTTCTTCATCCTTC | |
| | ATTCTTGTGTACGAAATGTTTTAGAGTGAGACCAGCGTAACTCGTTCC | |
| S358E | TATCTACACGGGTCTCACCAAAACTTATTGATTTTGAAGGGTATTTCATTCTTGTGTACGA | 718 |
| | GATCGGACATTGCAGTTCCATGATAGTAGATGTGGTAGTCAAGCCCATTTCTTCATCCTTC | |
| | ATTCTTGTGTACGAAATGTTTTAGAGTGAGACCAGCGTAACTCTGCGG | |
| S358G | TATCTACACGGGTCTCACCAAAACTTATTGATTTTGAAGGGTATTTCATTCTTGTGTACGA | 719 |
| | GATCGGACATTGCAGACCCATGATAGTAGATGTGGTAGTCAAGCCCATTTCTTCATCCTTC | |
| | ATTCTTGTGTACGAAATGTTTTAGAGTGAGACCAGCGTAACTCATAGA | |
| S358H | TATCTACACGGGTCTCACCAAAACTTATTGATTTTGAAGGGTATTTCATTCTTGTGTACGA | 720 |
| | GATCGGACATTGCAGATGCATGATAGTAGATGTGGTAGTCAAGCCCATTTCTTCATCCTTC | |
| | ATTCTTGTGTACGAAATGTTTTAGAGTGAGACCAGCGTAACTCGAGGA | |
| S358I | TATCTACACGGGTCTCACCAAAACTTATTGATTTTGAAGGGTATTTCATTCTTGTGTACGA | 721 |
| | GATCGGACATTGCAGAATCATGATAGTAGATGTGGTAGTCAAGCCCATTTCTTCATCCTTC | |
| | ATTCTTGTGTACGAAATGTTTTAGAGTGAGACCAGCGTAACTCCGCGG | |
| S358L | TATCTACACGGGTCTCACCAAAACTTATTGATTTTGAAGGGTATTTCATTCTTGTGTACGA | 722 |
| | GATCGGACATTGCAGCAACATGATAGTAGATGTGGTAGTCAAGCCCATTTCTTCATCCTTC | |
| | ATTCTTGTGTACGAAATGTTTTAGAGTGAGACCAGCGTAACTCTCCGC | |
| S358K | TATCTACACGGGTCTCACCAAAACTTATTGATTTTGAAGGGTATTTCATTCTTGTGTACGA | 723 |
| | GATCGGACATTGCAGTTTCATGATAGTAGATGTGGTAGTCAAGCCCATTTCTTCATCCTTC | |
| | ATTCTTGTGTACGAAATGTTTTAGAGTGAGACCAGCGTAACTCGCACG | |
| S358M | TATCTACACGGGTCTCACCAAAACTTATTGATTTTGAAGGGTATTTCATTCTTGTGTACGA | 724 |
| | GATCGGACATTGCAGCATCATGATAGTAGATGTGGTAGTCAAGCCCATTTCTTCATCCTTC | |
| | ATTCTTGTGTACGAAATGTTTTAGAGTGAGACCAGCGTAACTCATATA | |
| S358F | TATCTACACGGGTCTCACCAAAACTTATTGATTTTGAAGGGTATTTCATTCTTGTGTACGA | 725 |
| | GATCGGACATTGCAGAAACATGATAGTAGATGTGGTAGTCAAGCCCATTTCTTCATCCTTC | |
| | ATTCTTGTGTACGAAATGTTTTAGAGTGAGACCAGCGTAACTCGTGAC | |
| S358P | TATCTACACGGGTCTCACCAAAACTTATTGATTTTGAAGGGTATTTCATTCTTGTGTACGA | 726 |
| | GATCGGACATTGCAGTGGCATGATAGTAGATGTGGTAGTCAAGCCCATTTCTTCATCCTTC | |
| | ATTCTTGTGTACGAAATGTTTTAGAGTGAGACCAGCGTAACTCGAACC | |
| S358S | TATCTACACGGGTCTCACCAAAACTTATTGATTTTGAAGGGTATTTCATTCTTGTGTACGA | 727 |
| | GATCGGACATTGCAGAGACATGATAGTAGATGTGGTAGTCAAGCCCATTTCTTCATCCTTC | |
| | ATTCTTGTGTACGAAATGTTTTAGAGTGAGACCAGCGTAACTCCGCAT | |
| S358T | TATCTACACGGGTCTCACCAAAACTTATTGATTTTGAAGGGTATTTCATTCTTGTGTACGA | 728 |
| | GATCGGACATTGCAGAGTCATGATAGTAGATGTGGTAGTCAAGCCCATTTCTTCATCCTTC | |
| | ATTCTTGTGTACGAAATGTTTTAGAGTGAGACCAGCGTAACTCTTACG | |
| S358W | TATCTACACGGGTCTCACCAAAACTTATTGATTTTGAAGGGTATTTCATTCTTGTGTACGA | 729 |
| | GATCGGACATTGCAGCCACATGATAGTAGATGTGGTAGTCAAGCCCATTTCTTCATCCTTC | |
| | ATTCTTGTGTACGAAATGTTTTAGAGTGAGACCAGCGTAACTCAGGAG | |
| S358Y | TATCTACACGGGTCTCACCAAAACTTATTGATTTTGAAGGGTATTTCATTCTTGTGTACGA | 730 |
| | GATCGGACATTGCAGATACATGATAGTAGATGTGGTAGTCAAGCCCATTTCTTCATCCTTC | |
| | ATTCTTGTGTACGAAATGTTTTAGAGTGAGACCAGCGTAACTCGGGTG | |
| S358V | TATCTACACGGGTCTCACCAAAACTTATTGATTTTGAAGGGTATTTCATTCTTGTGTACGA | 731 |
| | GATCGGACATTGCAGAACCATGATAGTAGATGTGGTAGTCAAGCCCATTTCTTCATCCTTC | |
| | ATTCTTGTGTACGAAATGTTTTAGAGTGAGACCAGCGTAACTCCAAGC | |

### Example 6. Materials and Methods

### Plasmid Construction

All plasmids for yeast genome editing were constructed by assembling a CHAnGE cassette with pCRCT using Golden Gate assembly. Bao, Z. et al. ACS Synth. Biol. 4, 585-594 (2015).

For human EMX1 editing, pX330A-1×3-EMX1 was similarly constructed using pX330A-1×3 (Addgene #58767). All CHAnGE cassettes were ordered as gBlock fragments (Integrated DNA Technologies, Coralville, Iowa) and the sequences are listed in Tables 3 and 4.

### CHAnGE Library Design and Synthesis

All ORF sequences from S. cerevisiae strain S288c were downloaded from SGD and passed through CRISPRdirect to generate all possible guide sequences. Naito, Y, Hino, K., Bono, H. & Ui-Tei, K. Bioinformatics 31, 1120-1123 (2015). Only guide sequences with hit_20 mer>0 were retained to exclude those targeting exon-intron junctions. A guide-specific 100 bp HR donor was assembled 5′ of each guide sequence. All assembled sequences were passed through four additional filters: no BsaI restriction site (to facilitate Golden Gate assembly), no homopolymer of more than four T's (to prevent early transcription termination), no homopolymer of more than five A's or more than five G's (to maximize oligonucleotide synthesis efficiency). Each guide sequence was then assigned an arbitrary score for assessing both genome editing efficiency and off-target effect (Table 1). Specifically, artificial weights were assigned to each efficacy criterion so that higher scores will be given to guides with 35% to 75% GC content, with high purine content in the last four nucleotides, and targeting earlier regions of the ORF. To ensure targeting specificity, the score of a guide sequence decreases exponentially as the number of its off-target sites increases. An off-target site is defined as a site containing a matching 12 bp seed sequence followed by a PAM. Cong, L. et al. Science 339, 819-823 (2013).

For each ORF, the top four guide sequences with the highest scores were selected for synthesis. For ORFs with less than four unique guide sequences available, less than four guide sequences were selected. The final library contains 24765 unique guide sequences targeting 6459 ORFs (Table 2). For unknown reasons, there are five guide sequences for ORFs YOR343W-A and YBRO89C-A, and six guide sequences for ORF YMR045C. An additional 100 non-targeting guide sequences with random homology arms were randomly generated and added to the library as non-editing control guide sequences. Adapters containing priming sites and BsaI sites were added to the 5′ and 3′ ends of each oligonucleotide for PCR amplification and Golden Gate assembly. The designed oligonucleotide library was synthesized on two 12472 format chips and eluted into two separate pools (CustomArray, Bothell, Wash.).

### Construction of a CHAnGE Plasmid Library

The two oligonucleotide pools were mixed at equal molar ratio. 10 ng of the mixed oligonucleotide pool was used as a template for PCR amplification with primers BsaI-LIB-for and BsaI-LIB-rev (Table 5). The cycling conditions are 98° C. for 5 min, (98° C. for 45 s, 41° C. for 30 s, 72° C. for 6 s)×24 cycles, 72° C. for 10 min, then held at 4° C. 15 ng of the gel purified PCR products were assembled with 50 ng pCRCT using Golden Gate assembly method followed by plasmid-safe nuclease treatment. Bao, Z. et al. ACS Synth. Biol. 4, 585-594 (2015). 25 parallel Golden Gate assembly reactions were performed and the resultant DNA was purified using a PCR purification kit (Qiagen, Valencia, Calif.). The purified DNA was transformed into NEB5α electrocompetent cells (New England Biolabs, Ipswich, Mass.) using Gene Pulser Xcell™ Electroporation System (Bio-Rad, Hercules, Calif.). 20 parallel transformations were conducted and pooled. The pooled culture was plated onto 4 24.5 cm×24.5 cm LB plates supplemented with 100 μg/mL carbenicillin (Corning, N.Y., N.Y.). The plates were incubated at 37° C. overnight. The total number of colony forming units was estimated to be between 1.2×10⁷ and 4×10⁷, which represents a 480 to 1600-fold coverage of the CHAnGE plasmid library. Plasmids were extracted using a Qiagen Plasmid Maxi Kit.

### Generation of Yeast Mutant Libraries

Yeast strain BY4741 was transformed with 20 μg CHAnGE plasmid library per transformation using LiAc/SS carrier DNA/PEG method. Gietz, R. D. & Schiestl, R. H. Nat. Protoc. 2, 31-34 (2007). After heat shock, cells were washed with 1 mL double distilled water once and resuspended in 2 mL synthetic complete minus uracil (SC-U) liquid media. 12 parallel transformations were conducted. 2 μL culture from each of three randomly selected transformations were mixed with 98 μL sterile water and plated onto SC-U plates for assessing transformation efficiency. The total number of colony forming units was estimated to be 9.8×10⁶, which represents a 395-fold coverage of the CHAnGE plasmid library. Using SIZ1Δ1 and BUL1Δ1 as parental strains, a 499- and 129-fold coverage was achieved, respectively. The rest of the cells were cultured in twelve 15 mL falcon tubes at 30° C., 250 rpm. Two days after transformation, 2 units of optical density at 600 nm (OD) of cells from each tube were transferred to a new tube containing 2 mL fresh SC-U liquid media. Four days after transformation, cultures from 12 tubes were pooled. 2 OD of pooled cells were transferred to each of 12 new tubes containing 2 mL fresh SC-U media. Six days after transformation, cultures from 12 tubes were pooled and stored as glycerol stocks in a −80° C. freezer.

### Screening of Yeast Mutant Libraries

A glycerol stock of pooled yeast mutants was thawed on ice. 3.125 OD of cells were inoculated into 50 mL of SC-U liquid media with or without growth inhibitor in a 250 mL baffled flask. Cells were grown at 30° C., 250 rpm and the optical density was measured periodically. 2 OD of cells from each of the untreated and stressed population were collected when the optical density of the stressed population reached 2.

For canavanine resistance, 60 μg/mL L-(+)-(S)-canavanine (Sigma Aldrich, Saint Louis, Mo.) supplemented SC-UR media were used. For furfural tolerance, 5 mM and 10 mM furfural (Sigma Aldrich, Saint Louis, Mo.) supplemented SC-U media were used. For HAc tolerance, the pH of SC-U liquid media was adjusted to 4.5. Glacial acetyl acid was dissolved in double distilled water, adjusted to pH 4.5, and then filtered to make 10% (v/v) HAc stock solution. Appropriate volumes of HAc stock solution were added to SC-U media (pH 4.5) to make 0.5% and 0.6% HAc supplemented SC-U media. The unstressed cells were grown in SC-U media (pH 5.6).

### Next Generation Sequencing

For each untreated or stressed library, 2 OD of cells were collected and plasmids were extracted using Zymoprep™ Yeast Plasmid Miniprep II kit (Zymo Research, Irvine, Calif.). To attach NGS adaptors, a first step PCR was performed using 2×KAPA HiFi HotStart Ready Mix (Kapa Biosystems, Wilmington, Mass.) with primers HiSeq-CHAnGE-for and HiSeq-CHAnGE-rev (Table 5) and 10 ng extracted plasmid as template. The cycling condition is 95° C. for 3 min, (95° C. for 30 s, 46° C. for 30 s, 72° C. for 30 s)×18 cycles, 72° C. for 5 min, and held at 4° C. The PCR product was gel purified using a Qiagen Gel Purification kit. 10 ng PCR product from the first step was used in a second step PCR to attach Nextera indexes using the Nextera Index kit (Illumina, San Diego, Calif.). The cycling condition is 95° C. for 3 min, (95° C. for 30 s, 55° C. for 30 s, 72° C. for 30 s)×8 cycles, 72° C. for 5 min, and held at 4° C. The second step PCR products were gel purified using a Qiagen Gel Purification kit and quantitated with Qubit (ThermoFisher Scientific, Waltham, Mass.). 40 ng of each library were pooled. The pool was quantitated with Qubit. The average size was determined on a Fragment Analyzer (Advanced Analytical, Ankeny, Iowa) and further quantitated by qPCR on a CFX Connect Real-Time qPCR system (Biorad, Hercules, Calif.). The pool was spiked with 30% of a PhiX library (Illumina, San Diego, Calif.), and sequenced on one lane for 161 cycles from one end of the fragments on a HiSeq 2500 using a HiSeq SBS sequencing kit version 4 (Illumina, San Diego, Calif.).

### NGS Data Processing and Analysis

Fastq files were generated and demultiplexed with the bcl2fastq v2.17.1.14 conversion software (Illumina, San Diego, Calif.). 20 bp guide sequences were extracted from NGS reads using fastx_toolkit/0.0.13 (hannonlab.cshl.edu/fastx_toolkit/). A bowtie index was prepared from the 24865 designed guide sequences (Table 3). Extracted guide sequences were mapped to the bowtie index using Map with Bowtie for Illumina (version 1.1.2) command in Galaxy (usegalaxy.org) with commonly used settings. Unmapped reads were removed and reads mapped to each unique guide sequence were counted. The raw read counts per guide sequence were normalized to the total read counts of a library using the following equation Normalized read counts=(Raw read counts×1000000)/Total read counts+1. We used a threshold of two raw read counts in at least two of the four libraries (two biological replicates of untreated library and two biological replicates of stressed library) to keep a guide sequence. Genes with all observed guide sequences enriched (fold change >1.5) were selected for further validation.

### Construction of Single and Double Yeast Mutants

An aliquot of 5 mM furfural stressed library (OD=2) was plated onto a SC-U plate supplemented with 5 mM furfural. 24 random colonies were picked and genotyped by PCR and Sanger sequencing. One colony was confirmed to have a designed 8 bp deletion at SIZ1 target site 1. This colony was stored as strain SIZ1Δ1. BY4741 strains SAP30Δ3, UBC4Δ3, and LCB3Δ1 were constructed using the HI-CRISPR method. Bao, Z. et al. ACS Synth. Biol. 4, 585-594 (2015). The gBlock sequences can be found in Table 3. For constructing double mutants SIZ1Δ1 SAP30Δ83, SIZ1Δ1 UBC4Δ3, and SIZ1Δ1 LCB3Δ1, SIZ1Δ1 was used as the parental strain.

An aliquot of 0.5% HAc stressed library (OD=2) was plated onto a SC-U plate supplemented with 0.5% HAc. 32 random colonies were picked and genotyped by PCR and Sanger sequencing. Three colonies were confirmed to have a designed 8 bp deletion at BUL1 target site 1. One of these colonies was kept and stored as a strain named BUL1Δ1. A BUL1Δ1 strain without HAc exposure and the SUR1Δ1 strain were constructed using the HI-CRISPR method⁵. For constructing double mutants BUL1Δ1 SUR1Δ1, BUL1Δ1 with HAc exposure was used as the parental strain.

All other yeast mutants with non-disruption mutations were constructed using the HI-CRISPR method. The gBlock sequences can be found in Table 4. For each constructed mutant, pCRCT plasm ids were cured as described elsewhere. Hegemann, J. H. & Heick, S. B. Methods Mol. Biol. 765, 189-206 (2011). Briefly, a yeast colony with the desired gene disrupted was inoculated into 5 mL of YPAD liquid medium and cultured at 30° C., 250 rpm overnight. On the next morning, 200 μL of the culture was inoculated into 5 mL of fresh YPAD medium. In the evening, 50 μL of the culture was inoculated into 5 mL of fresh YPAD medium and cultured overnight. On the next day, 100-200 cells were plated onto an YPAD plate and incubated at 30° C. until colonies appear. For each mutant, 20 colonies were streaked onto both YPAD and SC-U plates. Colonies that failed to grow on SC-U plates were selected.

### Characterization of Mutant Strains for Furfural or HAc Tolerance

BY4741 wild type or mutant strains were inoculated from glycerol stocks into 2 mL YPAD medium and cultured at 30° C., 250 rpm overnight, then streaked onto fresh YPAD plates. Three biological replicates of each strain were inoculated in 3 mL synthetic complete (SC) medium and cultured at 30° C., 250 rpm overnight. On the next morning, 50 μL culture was inoculated into 3 mL fresh SC medium and cultured at 30° C., 250 rpm overnight to synchronize the growth phase. After 24 hours, 0.03 OD of cells were inoculated into 3 mL fresh SC medium (pH 5.6) supplemented with appropriate concentrations of furfural or 3 mL fresh SC medium (pH 4.5) supplemented with appropriate concentrations of HAc. Cell densities were measured at appropriate time points.

For spotting assays, each strain was inoculated in 3 mL SC medium and cultured at 30° C., 250 rpm overnight. On the next morning, 50 μL culture was inoculated into 3 mL fresh SC medium and cultured at 30° C., 250 rpm overnight to synchronize the growth phase. After 24 hours, the OD was measured and the culture was diluted to OD 1 in sterile water. 10-fold serial dilutions were performed for each strain. 7.5 μL of each dilution was spotted on appropriate plates. The spotted plates were incubated at 30° C. for 2 to 6 days.

### Tiling Mutagenesis of SIZ1

For the SIZ1 tiling mutagenesis library, the length of homology arms was reduced to 40 bp to accommodate the sequence between the PAM and the targeted codon. The PAM-codon distance was limited to be no more than 20 bp to not exceed the length limit of high throughput oligonucleotide synthesis. For each codon, 20 CHAnGE cassettes were designed for all possible amino acid residues. The SIZ1 oligonucleotide library was synthesized on one 12472 format chip (CustomArray, Bothell, Wash.). The SIZ1 plasmid library was similarly constructed with downscaled numbers of Golden Gate assembly reactions and transformations. The total number of colony forming unit was estimated to be between 3.8×10⁵ and 8×10⁵, which represents a 655 to 1379-fold coverage of the SIZ1 plasmid library. The SIZ1 yeast mutant library was similarly generated with 4 parallel transformations. The total number of colony forming unit was estimated to be 1.9×10⁶, which represents a 3200-fold coverage. Screening of the library and next generation sequencing were performed using the same procedures as the genome-wide disruption library. For NGS data processing, mutation-containing regions were used in the CHAnGE cassettes as genetic barcodes (Table 6) for mapping the reads. Zero mismatches were allowed for the mapping.

### HEK293T Culture, Transfections, and Genotyping

HEK293T cells were purchased from ATCC (CRL-3216) and maintained in DMEM with L-glutamine and 4.5 g/L glucose and without sodium pyruvate (Mediatech, Manassas, Va.) supplemented with 10% FBS and 1% penicillin/streptomycin at 37° C. in a humidified CO₂ incubator. 2×10⁵ cells were plated per well of a 24-well plate one day before transfection. Cells were transfected with Lipofectamine 2000 (ThermoFisher Scientific, Waltham, Mass.) using 800 ng pX330A-1×3-EMX1 and 2.5 μL of reagent per well. Cells were maintained for an additional three days before harvesting. Genomic DNA was extracted using QuickExtract DNA Extraction Solution (Epicentre, Madison, Wis.). 5 μg of genomic DNA was used as template for selective PCR using primers EMX1-selective-for and EMX1-selective-rev (Table 5). PCR amplicons were gel purified and sequenced by Sanger sequencing.

### Statistics

Data is shown as mean±SEM, with n values indicated in the figure legends. All P values were generated from two-tailed t-tests using the GraphPad Prism software package (version 6.0c, GraphPad Software) or Microsoft Excel for Mac 2011 (version 14.7.3, Microsoft Corporation).

### Code Availability

All computational tools used for analyses of the NGS data are available from provided references in Methods. Custom batch scripts used for execution of these computational tools can be found in Supplementary Code below:

module load fastx_toolkit/0.0.13
fastx_trimmer -I 77 -v -i input_file.fastq -o input_file_trm.fastq
fastx_reverse_complement -v -i input_file_trm.fastq -o
input_file_rc.fastq
fastx_clipper -a GTTTTAGAG -I 20 -c -v -i input_file_rc.fastq -o
input_file_clip.fastq

### Data Availability

The raw reads of the NGS data were deposited into the Sequence Read Archive (SRA) database (accession number: SUB3231451) at the National Center for Biotechnology Information (NCBI).

### CONCLUSION

CHAnGE is a trackable method to produce a genome-wide set of host cell mutants with single nucleotide precision. Design of CHAnGE cassettes can be affected by the presence of BsaI sites and polyT sequences. Therefore, optimization using homologous recombination assembly and type II RNA promoters can expand the design space. Increasing the number of experimental replicates and design redundancy of CHAnGE cassettes can reduce false positive rates. CHAnGE can be adopted for genome-scale engineering of higher eukaryotes, as preliminary experiments reveal precise editing of the human EMX1 locus using a CHAnGE cassette (FIG. 20).

We claim:

1. A vector comprising a first promoter upstream of an insertion site and downstream of the insertion site: a terminator, a second promoter, a nucleic acid molecule encoding an RNA-guided DNA endonuclease protein, a third promoter, and a tracrRNA sequence, and in the insertion site a genetic engineering cassette comprising from a 5′ end to a 3′ end:
(i) a first direct repeat sequence;
(ii) a homologous recombination editing template comprising two homology arms with a deletion portion, a substitution portion, or an insertion portion between the two homology arms;
(iii) a guide sequence; and
(iv) a second direct repeat sequence.

2. The vector of claim 1, wherein the homologous recombination editing template comprises a deletion portion that removes a protospacer adjacent motif (PAM) sequence and causes a gene disruption.

3. The vector of claim 1, wherein the genetic engineering cassette further comprises a first priming site at a 5′ end of the cassette and a second priming site at a 3′ end of the cassette.

4. (canceled)

5. A pool of vectors comprising 20 or more of the vectors of claim 1, wherein the vectors comprise genetic engineering cassettes specific for 20 or more target nucleic acid molecules.

6. A pool of host cells comprising two or more vectors of claim 1.

7. A method of homology directed repair-assisted engineering comprising delivering the pool of vectors of claim 5 to host cells to generate a pool of unique transformed genetic variant host cells.

8. The method of claim 7, wherein the pool of unique transformed variant host cells comprises host cells that have mutations throughout the host cell genome.

9. The method of claim 7, further comprising isolating transformed genetic variant host cells with one or more phenotypes; and determining a genomic locus of a nucleic acid molecule that causes one or more phenotypes.

10. The method of claim 9, wherein determining the genomic locus comprises using a genetic bar code or a sequence of the homologous recombination editing template.

11. The method of claim 7, wherein more than about 1,000 unique transformed genetic variant host cells are generated.

12. (canceled)

13. A method of engineering a desired phenotype of host cells comprising:
(a) constructing a vector library, wherein the vector library comprises two or more vectors each comprising a genetic engineering cassette in an insertion site of the vector that target one or more target sequences of the host cells at one or more positions, wherein the genetic engineering cassettes comprise from a 5′ end to a 3′ end:
(i) a first direct repeat sequence;
(ii) a homologous recombination editing template comprising two homology arms with a deletion portion, a substitution portion, or an insertion portion between the two homology arms;
(iii) a guide sequence; and
(iv) a second direct repeat sequence;
wherein the vectors comprise a first promoter upstream of the insertion site and downstream of the insertion site: a terminator, a second promoter, a nucleic acid molecule encoding an RNA-guided DNA endonuclease protein, a third promoter, and a tracrRNA sequence;
(b) transforming the host cells with the vector library to form a transformed host cell pool; and
(c) selecting host cells with a desired phenotype.

14. (canceled)

15. (canceled)

16. A genetic engineering cassette comprising from a 5′ end to a 3′ end:
(i) a first direct repeat sequence;
(ii) a first homologous recombination editing template comprising two homology arms with a deletion portion, a substitution portion, or an insertion portion between the two homology arms;
(iii) a first guide sequence;
(iv) a second direct repeat sequence;
(v) a second homologous recombination editing template comprising two homology arms with a deletion portion, a substitution portion, or an insertion portion between the two homology arms;
(vi) a second guide sequence; and
(vii) a third direct repeat sequence.

17. The genetic engineering cassette of claim 16, further comprising a first priming site at a 5′ end of the cassette and a second priming site at a 3′ end of the cassette.

18. (canceled)

19. The genetic engineering editing cassette of claim 16, wherein the first homologous recombination editing template and the second homologous recombination editing template each provide for a first substitution, first insertion, or first deletion, and a second substitution, second insertion, or second deletion in different locations of the same target polynucleotide.

20. The genetic engineering editing cassette of claim 16, wherein the first substitution, first insertion, or first deletion and the second substitution, second insertion, or second deletion site, occur in any two loci across the whole genome of the host cell.

21. The genetic engineering cassette of claim 16, wherein the first substitution is a substitution of 1 to 6 nucleic acids, the first insertion is an insertion of 1 to 6 nucleic acids, the first deletion is a deletion of 1 to 6 nucleic acids, the second substitution is a substitution of 1 to 6 nucleic acids, the second insertion is an insertion of 1 to 6 nucleic acids, and the second deletion is a deletion of 1 to 6 nucleic acids.

22. A vector comprising the genetic engineering cassette of claim 16.

23. The vector of claim 22, wherein the vector comprises a first promoter upstream of the genetic engineering cassette and downstream of the genetic engineering cassette: a terminator, a second promoter, a nucleic acid molecule encoding an RNA-guided DNA endonuclease protein, a third promoter, and a tracrRNA sequence.

24. A pool of vectors comprising two or more of the vectors of claim 22, wherein each of the genetic engineering cassettes is unique.

25. A method of homology directed repair-assisted engineering comprising:
(i) delivering the pool of vectors of claim 24 to host cells; and
(ii) isolating transformed host cells.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
